(19) United States
(12) Patent Application Publication
(10) Pub. No.: US 2010/0222345 A1
(43) Pub. Date: Sep. 2, 2010

(54) NOVEL COMPOUNDS AS ANTAGONISTS OR INVERSE AGONISTS FOR OPIOID RECEPTORS

(76) Inventors: Caroline Jean Diaz, Durham, NC (US); Curt Dale Haffner, Durham, NC (US); Jason Daniel Speake, Durham, NC (US); Cunyu Zhang, Durham, NC (US); Wendy Yoon Mills, Durham, NC (US); Paul Kenneth Spearing, Durham, NC (US); David John Cowan, Durham, NC (US); Gary Martin Green, Durham, NC (US)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 12/376,577

(22) PCT Filed: Aug. 8, 2007
(86) PCT No.: PCT/US07/75424

§ 371 Date: Apr. 14, 2010

Related U.S. Application Data
(60) Provisional application No. 60/821,853, filed on Aug. 9, 2006.

(51) Int. Cl. A61K 31/4184; C07D 235/04; C07D 413/12; C07D 405/12; C07D 471/04; C07D 401/10; A61K 31/5377; A61K 31/4439; A61K 31/4725

(52) U.S. Cl. 514/234.5; 548/304.4; 544/139; 546/118; 546/273.4; 546/148; 514/394; 514/338; 514/307

(57) ABSTRACT

This invention relates to novel compounds which are antagonists or inverse agonists at one or more of the opioid receptors, to pharmaceutical compositions containing them, to processes for their preparation, and to their use in therapy.

### FIELD OF THE INVENTION

This invention relates to novel compounds which are antagonists or inverse agonists at one or more of the opioid receptors, to pharmaceutical compositions containing them, to processes for their preparation, and to their use in therapy.

### BACKGROUND OF THE INVENTION

Obesity is a medical condition that is reaching epidemic proportions among humans throughout the world. It is a condition that is associated with other diseases or conditions that disrupt life and lifestyles. Obesity is recognized as a serious risk factor for other diseases and/or conditions such as diabetes, hypertension, and arteriosclerosis. It is also known that increased body weight due to obesity can place a burden on joints, such as knee joints, causing arthritis, pain, and stiffness.

Because overeating and obesity have become such a problem, many individuals are interested in weight reduction and/or maintaining a healthy body weight.

The ability to bind antagonistically to opioid receptors has been suggested to be useful for treatment of many other diseases or conditions not related to obesity including drug and/or substance addiction, depression, opiate overdose, irritable bowel syndrome, schizophrenia, compulsive disorders, septic shock, nausea, vomiting, and stroke. This ability may be useful for the treatment of obesity as well. It has been suggested that the opioid receptors may play a role in control of food intake and food selection. (See, for example, Bodnar, R. J., in Peptides, 25, (2004), p. 697). Antagonists or inverse agonists of the opioid receptors have been shown to reduce body weight in obese rats.

There is, therefore, an ongoing need for new opioid antagonists for the treatment of obesity, diseases and/or conditions associated with obesity, as well as the above-mentioned non-obesity related diseases and/or conditions.

### SUMMARY OF THE INVENTION

The present invention provides a compound of Formula I

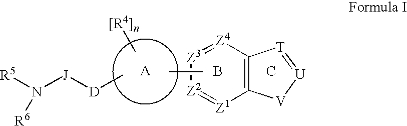

a salt, a solvate, or a physiologically functional derivative thereof wherein:
Ring A is selected from the group consisting of aryl and heteroaryl, and ring A is attached to Z² or Z³;
D is selected from the group consisting of —CH₂— and —O— and is attached to a carbon atom of ring A, with the proviso that D is not attached to the atom adjacent to the bond joining ring A and fused ring BC;
J is a bond or a C₁₋₄alkylene;
each Z¹, Z², Z³, and Z⁴ is the same or different and is selected from the group consisting of CH, N, and CR³ with the proviso that Z² or Z³ is a carbon atom to which Ring A is attached, and that no more than two of Z¹, Z², Z³, and Z⁴ are N;
T and U are each independently selected from the group consisting of N, CH, C(NR¹R²), and C(R²); V is selected from the group consisting of NH, O, S, and NR¹; wherein R¹ and R² are each independently selected from the group consisting of a C₁₋₆ alkyl and a fluoroalkyl;
R³ and R⁴ are each independently selected from the group consisting of —F, —Cl, —Br, —OH, —CN, —OC₁₋₃ alkyl, —C₁₋₃ fluoroalkyl, and —C₁₋₃ alkyl; n is 0, 1, or 2;
R⁵ is selected from the group consisting of hydrogen, C₁₋₁₂ alkyl, C₃₋₁₀ cycloalkyl, arylalkyl, heterocyclyl, heterocycloalkyl, heteroarylalkyl, cycloalkenyl, C₂₋₁₂ fluoroalkyl, and heteroalkyl; and
R⁶ is selected from the group consisting of C₃₋₁₂ alkyl, C₃₋₁₀ cycloalkyl, arylalkyl, heterocyclyl, heterocycloalkyl, heteroarylalkyl, cycloalkenyl, C₃₋₁₂ fluoroalkyl, and heteroalkyl.

The present invention also provides a pharmaceutical composition comprising a compound of Formula I, a salt, solvate, or physiologically functional derivative thereof and one or more excipients.

And the present invention further provides method of treatment comprising the administering to a mammal, particularly a human, a pharmaceutical composition comprising (i) a compound of Formula I, a pharmaceutically acceptable salt, solvate, or physiologically functional derivative thereof and (ii) at least one carrier, wherein said treatment is for a disease or condition selected from the group consisting of obesity, diabetes, hypertension, depression, anxiety, drug addiction, substance addiction, or a combination thereof.

There is further provided processes for making a compound of Formula I, salts, solvates, and physiologically functional derivatives thereof.

### DETAILED DESCRIPTION OF THE INVENTION

In Formula I, Ring A is selected from the group consisting of aryl and heteroaryl, and Ring A is attached to Z² or Z³ atom of fused ring BC. In one embodiment, Ring A is selected from the group consisting of as phenyl, thienyl, furanyl, pyrrolyl, thiazolyl, oxazolyl, oxadiazolyl, thiadiazolyl, imidazolyl, pyridyl, pyrimidinyl, pyrazinyl, triazinyl, naphthalyl, quinolinyl, isoquinolinyl, indolyl, benzthiophenyl, benzimidazolyl, benzoxazolyl, benzthiazolyl, benzisoxazolyl, indazolyl, pyrazolopyridinyl, pyrazolopyrimidinyl, pyrazolopyrazinyl, imidazopyridinyl, purinyl, thiazolopyridinyl, thiazolopyrimidinyl, thiazolopyrazinyl, oxazolopyridinyl, oxazolopyrimidinyl, and oxazolopyrazinyl. In a preferred embodiment, Ring A is phenyl or pyridyl. Preferably, Ring A is a phenyl group.

D of Formula I is selected from the group consisting of —CH₂— and —O— and is attached to a carbon atom of ring A, with the proviso that D is not attached to an atom adjacent to the bond joining Ring A and the fused ring BC. That is, D is attached to a carbon atom positioned para or meta, but not ortho, to the bond connecting Ring A to the fused ring BC.

In Formula I, J is a bond or C₁₋₄alkylene that joins D and N. In one embodiment, when D is —CH₂—, J is a bond or a C₁₋₂alkylene. Alternatively, in another embodiment, when D is —O—, J is a C₂₋₃ alkylene. In a preferred embodiment D is —O— and J is a C₂alkylene.

Each Z¹, Z², Z³, and Z⁴ in Formula I is the same or different and is selected from the group consisting of CH, N, and CR³ with the proviso that Z² or Z³ is a carbon atom to which Ring A is attached, and that no more than two of Z¹, Z², Z³, and Z⁴ are N.

In Formula I, T and U, are each independently selected from the group consisting of N, CH, C(NR¹R²), and C(R²). V is selected from the group consisting of NH, O, S, and NR¹. R¹ and R² are each independently selected from the group consisting of a C₁₋₆ alkyl and a fluoroalkyl. In one preferred embodiment, T is N, V is NH, and U is CH and ring A is attached to Z³. Another preferred embodiment occurs when T is N, V is NH, and U is CH and ring A is attached to Z².

R³ and R⁴ in Formula I can be substituted or unsubstituted. R³ and R⁴ are each independently selected from the group consisting of —H, —F, —Cl, —Br, —OH, —CN, —OC₁₋₃ alkyl, —C₁₋₃ fluoroalkyl, and —C₁₋₃ alkyl. In a preferred embodiment, R³ and R⁴ are independently selected from the group consisting of —H, —F, —Cl, —CH₃, —CF₃, —OCH₃, and —OCF₃. In [R⁴]ₙ, n is 0, 1, or 2.

In Formula I, R⁵ is selected from the group consisting of hydrogen, C₁₋₁₂ alkyl, C₃₋₁₀ cycloalkyl, arylalkyl, heterocyclyl, heterocyclylalkyl, heteroarylalkyl, cycloalkenyl, C₂₋₁₂ fluoroalkyl, and heteroalkyl. Preferably, R⁵ is hydrogen. Note, that when D is —CH₂— and J is a bond, then R⁵ may optionally be a C₁₋₂alkyl which then is joined to Ring A to form a tetrahydroisoquinoline.

In Formula I, R⁶ is selected from the group consisting of C₃₋₁₂ alkyl, C₃₋₁₀ cycloalkyl, arylalkyl, heterocyclyl, heterocyclylalkyl, heteroarylalkyl, cycloalkenyl, C₃₋₁₂ fluoroalkyl, and heteroalkyl. In one preferred embodiment, R⁶ is selected from the group consisting of arylmethyl, arylethyl, C₄₋₁₀ alkyl, cycloalkenyl, C₃₋₁₀cycloalkyl, heterocyclylmethyl, and heterocyclylethyl, such as, but not limited to 3-fluorophenylethyl, 3-fluorobenzyl, 2-trifluoromethylbenzyl, 2-trifluoromethoxybenzyl, 4-trifluoromethylbenzyl, 4-fluorobenzyl, 3-methoxyphenylethyl, 3-thiophenylmethyl, 2-thiophenylethyl, 4,4-dimethylcyclohexyl, 3,3-dimethylcyclohexyl, 2-indanyl, 5-cyano-2-indanyl, 5-methoxy-2-indanyl, 5-fluoro-2-indanyl, 4-fluoro-2-indanyl, 4-methoxy-2-indanyl, 4-methoxy-2-indanyl, 4,8-diflouro-2-indanyl, 5,6-difluoro-2-indanyl, 5,6-dimethoxy-2-indanyl, 2-methyl-2-indanyl, cyclohexylmethyl, cyclohexylethyl, 4,4-difluorocyclohexyl, 1-cyclohexenylmethyl, 1-cyclohexenylethyl, cyclooctyl, cycloheptylmethyl, 3-methylbutyl, adamantyl, morpholinoethyl, piperidinylethyl, 4-tert-butylcyclohexyl, 3,3,5,5-tetramethylcyclohexyl, 3,5-difluorobenzyl, 3,5-difluorophenylethyl, 2-diphenylmethyl, methoxyethyl, dimethylaminoethyl, 3-pyridinylethyl, 3-pyridinylmethyl, and phenyloxyethyl. Of these, preferably R⁶ is selected from among the group consisting of 2-indanyl, 5-fluoro-2-indanyl, 4,4-dimethylcyclohexyl, cyclohexylethyl, cyclohexylmethyl, 2-thiophenylethyl, 3-fluorophenylethyl, 3-methylbutyl, and 4,4-difluorocyclohexyl. In a preferred embodiment of

Formula I, R⁵ is hydrogen and R⁶ is selected from the group consisting of arylmethyl, arylethyl, C₄₋₁₀ alkyl, cycloalkenyl, C₃₋₁₀cycloalkyl, heterocyclylmethyl, and heterocyclylethyl.

In a particularly preferred embodiment of Formula I, Ring A is phenyl or pyridyl; D is —CH₂— attached para to the bond joining Ring A and said fused ring BC; J is a bond or a C₁₋₂alkylene; T is N, V is NH; U is CH; R³ and R⁴ are each independently selected from the group consisting of —H, —F, —Cl, —CH₃, —CF₃, —OCH₃, and —OCF₃; R⁵ is hydrogen; and R⁶ is selected from the group consisting or arylmethyl, arylethyl, C₃₋₁₀alkyl, C₃₋₁₀cycloalkyl, and heteroarylalkyl. Alternatively, in a particularly preferred embodiment of Formula I, Ring A is phenyl or pyridyl; D is —O— attached meta or para to the bond joining Ring A and said fused ring BC; J is a bond or a C₁₋₂alkylene; T is N, V is NH; U is CH; R³ and R⁴ are each independently selected from the group consisting of —H, —F, —Cl, —CH₃, —CF₃, —OCH₃, and —OCF₃; R⁵ is hydrogen; and R⁶ is selected from the group consisting or arylmethyl, arylethyl, C₃₋₁₀alkyl, C₃₋₁₀cycloalkyl, and heteroarylalkyl.

Preferred structures for a fused Ring BC of Formula I include:

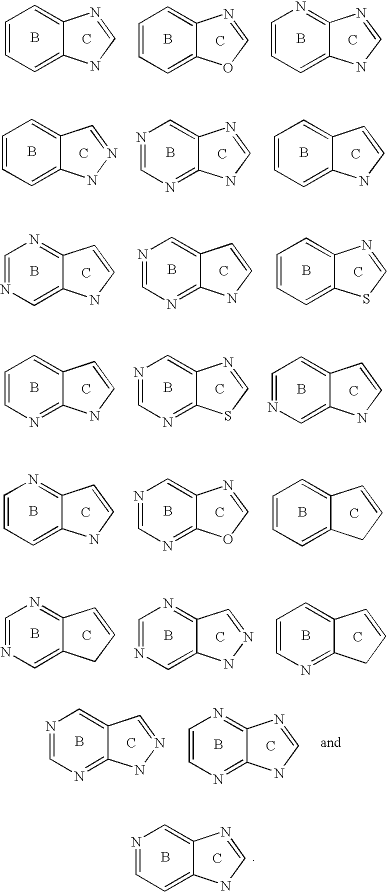

Of these, the first six listed above are especially preferred:

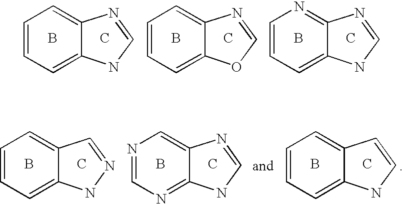

A most preferred fused Ring BC is

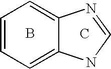

in which Z¹, Z², Z³, Z⁴ and U are C, T is N, and V is NH.

Particularly preferred compounds and their salts include
{[4-(1H-benzimidazol-5-yl)phenyl]methyl}(4,4-dimethylcyclohexyl)amine,
N-{[4-(1H-benzimidazol-5-yl)phenyl]methyl}-2,3-dihydro-1H-inden-2-amine,
N-{[3-(1H-benzimidazol-5-yl)phenyl]methyl}-2,3-dihydro-1H-inden-2-amine,
N-{[4-(1H-benzimidazol-5-yl)-2-fluorophenyl]methyl}-2-cyclohexanamine,
N-{[4-(1H-benzimidazol-5-yl)-2-fluorophenyl]methyl}-2,3-dihydro-1H-inden-2-amine,
N-{[4-(1H-benzimidazol-5-yl)-2-fluorophenyl]methyl}-4,4-dimethylcyclo hexanamine,
N-{[4-(1H-benzimidazol-5-yl)-3-fluorophenyl]methyl}-4,4-dimethylcyclo hexanamine,
(b 2-cyclohexylethyl){[3-fluoro-4-(4-methyl-1H-benzimidazol-5-yl)phenyl]methyl}amine,
N-{[3-fluoro-4-(4-methyl-1H-benzimidazol-5-yl)phenyl]methyl}-4,4-dimethylcyclo hexanamine,
N-{[2,6-difluoro-4-(4-methyl-1H-benzimidazol-5-yl)phenyl]methyl}-2,3-dihydro-1H-inden-2-amine,
(2-cyclohexylethyl){[2,6-difluoro-4-(4-methyl-1H-benzimidazol-5-yl)phenyl]methyl}amine,
N-{[4-(1H-benzimidazol-5-yl)-2,6-difluorophenyl]methyl}-2,3-dihydro-1H-inden-2-amine, and
N-{[4-(1H-benzimidazol-5-yl)-2,6-difluorophenyl]methyl}-4,4-dimethylcyclo hexanamine.
The most preferred salt of these named compounds is the hydrochloride salt.

The present invention provides a pharmaceutical composition comprising a compound of Formula I, a salt, a solvate, or physiologically functional derivative thereof and at least one excipient, diluent, or carrier, preferably at least one pharmaceutically acceptable excipient, diluent, or carrier.

Additionally, there is provided a method for treatment of a disease or condition comprising administering to a patient (e.g., a mammal such as a human) a compound of Formula I, a salt, a solvate, or physiologically functional derivative thereof. There is also provided a method for treatment of a disease or condition comprising administering to a patient (e.g., a mammal such as a human) a pharmaceutical composition comprising a compound of Formula I, a salt, a solvate, or physiologically functional derivative thereof and at least one excipient, diluent, or carrier, preferably a pharmaceutically acceptable excipient, diluent or carrier. The treatment can be for one or more of the following diseases or conditions: obesity, diabetes, hypertension, depression, anxiety, drug addiction, substance addiction. Preferably, the disease/condition is obesity.

A compound, salt, solvate, or physiologically functional derivative of Formula I can also be used as an active therapeutic substance. Further, a compound, salt, solvate, or physiologically functional derivative of Formula I can be used in the treatment of a disease or condition such as obesity, diabetes, hypertension, depression, anxiety, drug addiction, substance addiction. Preferably, the disease/condition is obesity. And, a compound, salt, solvate, or physiologically functional derivative of Formula I can be used in the manufacture of a medicine for use in the treatment of obesity, diabetes, hypertension, depression, anxiety, drug addiction, substance addiction, or a combination thereof. Preferably, the compound, salt, solvate, or physiologically functional derivative of Formula I is use in the manufacture of a medicine for use in treating obesity.

Terms are used within their accepted meanings. The following definitions are meant to clarify, but not limit, the terms defined.

As used herein, the term “alkyl” refers to a straight or branched chain alkyl, preferably having from one to twelve carbon atoms, which may be unsubstituted or substituted, with multiple degrees of substitution included within the present invention. Examples of “alkyl” as used herein include, but are not limited to, methyl, ethyl, propyl, isopropyl, isobutyl, n-butyl, isopentyl, n-pentyl, and the like, as well as substituted versions thereof.

As used herein, the term “alkylene” refers to a straight or branched chain divalent alkyl radical, preferably having from one to ten carbon atoms. Alkylene groups as defined herein may be unsubstituted or substituted, with multiple degrees of substitution included within the present invention. Examples of “alkylene” as used herein include, but are not limited to, methylene, ethylene, n-propylene, n-butylene, and the like, as well as substituted versions thereof.

As used herein, the term “cycloalkyl” refers to an unsubstituted or substituted mono- or polycyclic non-aromatic saturated ring, which optionally includes an alkylene linker through which the cycloalkyl may be attached. Exemplary “cycloalkyl” groups include, but are not limited to, cyclopropyl, cyclobutyl, cyclopentyl, cyclohexyl, cycloheptyl, and the like, as well as unsubstituted and substituted versions thereof. As used herein, the term “cycloalkyl” includes an optionally substituted fused polycyclic hydrocarbon saturated ring and aromatic ring system, namely polycyclic hydrocarbons with less than maximum number of non-cumulative double bonds, for example where a saturated hydrocarbon ring (such as a cyclopentyl ring) is fused with an aromatic ring (herein “aryl,” such as a benzene ring) to form, for example, groups such as indane.

As used herein, the term “cycloalkenyl” refers to an unsubstituted and substituted non-aromatic cyclic hydrocarbon ring containing one or more carbon-to-carbon double bonds which optionally includes an alkylene linker through which the cycloalkenyl may be attached, with multiple degrees of substitution included within the present invention. Exemplary “cycloalkenyl” groups include, but are not limited to, cyclopropenyl, cyclobutenyl, cyclopentenyl, cyclohexenyl, cycloheptenyl, and the like, as well as unsubstituted and substituted versions thereof.

As used herein, the term “heterocycle” or “heterocyclyl” refers to an unsubstituted and substituted mono- or polycyclic non-aromatic ring system containing one or more heteroatoms. Preferred heteroatoms include N, O, and/or S, including N-oxides, sulfur oxides, and dioxides. Preferably the ring is three to twelve-membered and is either fully saturated or has one or more degrees of unsaturation. Multiple degrees of substitution are included within the present definition. Such rings may be optionally fused to one or more of another “heterocyclic” ring(s) or cycloalkyl ring(s). Examples of “heterocyclic” groups include, but are not limited to, tetrahydrofuranyl, pyranyl, 1,4-dioxanyl, 1,3-dioxanyl, piperidinyl, pyrrolidinyl, morpholinyl, tetrahydrothiopyranyl, and tetrahydrothiophenyl.

As used herein, the term “heterocyclylalkyl” refers to a heterocycle, as defined herein, bonded to an alkyl group, as defined herein.

As used herein, the term “arylalkyl” refers to an aryl group, as defined herein, bonded to an alkyl group, as defined herein.

As used herein, the term “heteroalkyl” refers to an alkyl group, as defined herein, wherein one or more of the carbon atoms of the alkyl group is replaced by a heteroatom. Preferred heteroatoms include N, O, and/or S, including N-oxides, sulfur oxides, and dioxides.

As used herein, the term “aryl” refers to unsubstituted and substituted benzene ring or to an optionally substituted fused benzene ring system, for example anthracene, phenanthrene, or naphthalene ring systems. Multiple degrees of substitution are included within the present definition. Examples of “aryl” groups include, but are not limited to, phenyl, 2-naphthyl, 1-naphthyl, and the like, as well as substituted derivatives thereof.

As used herein, the term “heteroaryl” refers to unsubstituted and substituted monocyclic five to seven membered aromatic ring, or to an unsubstituted or substituted fused bicyclic aromatic ring system comprising two of such aromatic rings. These heteroaryl rings contain one or more heteroatoms such as nitrogen, sulfur, and/or oxygen atoms, where N-oxides, sulfur oxides, and dioxides are permissible heteroatom substitutions. Multiple degrees of substitution are included within the present definition. Examples of “heteroaryl” groups used herein include, but should not be limited to, furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, quinolinyl, isoquinolinyl, benzofuranyl, benzothiophenyl, indolyl, indazolyl, benzimidizolyl, imidazopyridinyl, pyrazolopyridinyl, pyrazolopyrimidinyl, and the like, as well as substituted versions thereof.

As used herein, the term “heteroarylalkyl” refers to a heteroaryl as defined herein bonded to an alkyl as defined herein.

As used herein, the term “halogen” or “halo” refers to fluorine (or fluoro), chlorine (or chloro), bromine (or bromo), or iodine (or iodo). Preferably, each halogen when present is individually either fluorine or chlorine.

As used herein, the term “fluoroalkyl” refers to an alkyl group, as defined herein, that is substituted with at least one fluorine atom. Examples of branched or straight chained “fluoroalkyl” groups useful in the present invention include, but are not limited to, methyl, ethyl, propyl, isopropyl, n-butyl, and t-butyl substituted independently with one or more fluorine. The term “fluoroalkyl” should be interpreted to include such substituents as perfluoroalkyl groups and the like.

As used herein, the term “alkoxy” refers to the group —OR^{a}, where R^{a} is alkyl as defined above.

As used herein, the term “nitro” refers to the group —NO₂.

As used herein, the term “cyano” refers to the group —CN.

As used herein, the term “azido” refers to the group —N₃.

As used herein, the term “acyl” refers to the group R^{b}C(O)—, where R^{b} is alkyl, aryl, heteroaryl, or heterocyclyl, as each is defined herein.

As used herein, the term “oxo” refers to the group ═O.

The terms “members” (and variants thereof, e.g., “membered”) in the context of heterocyclic, heteroaryl, heteroaromatic, aryl, and aromatic groups refers to the total atoms, carbons and heteroatoms (e.g., N, O, and S) which form the ring. Thus, an example of a 6-membered heterocyclic ring is piperidine; an example of a 6-membered heteroaryl is pyridine; and an example of a 6-membered aryl ring is benzene.

As used herein, the term “optionally” means that the subsequently described event(s) may or may not occur, and includes both event(s) that occur and event(s) that do not occur.

Also, as used herein throughout the present specification, the phrase “optionally substituted” or variations thereof denote an optional substitution, including multiple degrees of substitution, with one or more substitutent group. The phrase should not be interpreted as duplicative of the substitutions herein described and depicted. Exemplary optional substituent groups include acyl; alkyl; alkoxy; cyano; halogen; haloalkyl; hydroxy; oxo; nitro; aryl, which may be further substituted with acyl, alkoxy, alkyl, cyano, halogen, haloalkyl, hydroxy, or nitro; heteroaryl, which may be further substituted with acyl, alkoxy, alkyl, cyano, halogen, haloalkyl, hydroxy, or nitro; or —N(R*)₂; where for each occurrence R* is independently selected from hydrogen, alkyl, cycloalkyl, heterocyclyl, aryl, aralkyl, heteroaryl, heteroaralkyl, where each occurrence of such aryl or heteroaryl may be substituted with one or more acyl, alkoxy, alkyl, cyano, halogen, haloalkyl, hydroxy, oxo, or nitro, or the two R*s may combine to form a ring, optionally having additional heteroatoms (e.g., N, O, S, etc.), optionally having one or more degrees of unsaturation, and optionally being further substituted with acyl, alkoxy, alkyl, cyano, halogen, haloalkyl, hydroxy, or nitro.

The compounds of Formula I may crystallize in more than one form, a characteristic known as polymorphism, and such polymorphic forms (“polymorphs”) are within the scope of Formula I. Polymorphism generally can occur as a response to changes in temperature, pressure, or both. Polymorphism can also result from variations in the crystallization process. Polymorphs can be distinguished by various physical characteristics known in the art such as x-ray diffraction patterns, solubility, and melting point.

Certain compounds of Formula I may exist in stereoisomeric forms (e.g., they may contain one or more asymmetric carbon atoms or may exhibit cis-trans isomerism). The individual stereoisomers (enantiomers and diastereomers) and mixtures of these are included within the scope of the present invention. The present invention also covers the individual isomers of the compounds represented by Formula I as mixtures with isomers thereof in which one or more chiral centers are inverted. Certain compounds of Formula I may be prepared as regioisomers. The present invention covers both the mixture of regioisomers as well as individual compounds. Likewise, it is understood that compounds of Formula I may exist in tautomeric forms other than that shown in the formula and these are also included within the scope of the present invention. It is to be understood that the present invention includes all combinations and subsets of the particular groups defined hereinabove. The scope of the present invention includes mixtures of stereoisomers as well as purified enantiomers or enantiomerically/diastereomerically enriched mixtures. Also included within the scope of the invention are the individual isomers of the compounds represented by Formula I, as well as any wholly or partially equilibrated mixtures thereof. The present invention also includes the individual isomers of the compounds represented by the formula as well as mixtures with isomers thereof in which one or more chiral centers are inverted.

Typically, but not absolutely, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts. In general, the salts are formed from pharmaceutically acceptable inorganic and organic acids. More specific examples of suitable acid salts include maleic, hydrochloric, hydrobromic, sulphuric, phosphoric, nitric, perchloric, fumic, acetic, propionic, succinic, glycolic, formic, lactic, aleic, tartaric, citric, palmoic, malonic, hydroxymaleic, phenylacetic, glutamic, benzoic, salicylic, fumaric, toluenesulfonic, methansulfonic (mesylate), naphthaliene-2-sulfonic, benzenesulfonic, hydroxynaphthoic, hydroiodic, malic, teroic, tannic, and the like.

Other representative salts include acetate, benzenesulfonate, benzoate, bicarbonate, bisulfate, bitartrate, borate, calcium edetate, camsylate, carbonate, clavulanate, citrate, dihydrochloride, edisylate, estolate, esylate, fumarate, gluceptate, gluconate, glutamate, glycollylarsanilate, hexylresorcinate, hydrobromide, hydrochloride, hydroxynaphthoate, iodide, isethionate, lactate, lactobionate, laurate, malate, maleate, mandelate, mesylate, methylsulfate, monopotassium maleate, mucate, napsylate, nitrate, oxalate, pamoate (embonate), palmitate, pantothenate, phosphate/diphosphate, polygalacturonate, salicylate, stearate, subacetate, succinate, sulfate, tannate, tartrate, teoclate, tosylate, triethiodide, and valerate salts.

Other salts, which are not pharmaceutically acceptable, may be useful in the preparation of compounds of this invention and these should be considered to form a further aspect of the invention. These salts, such as oxalic or trifluoroacetate, while not in themselves pharmaceutically acceptable, may be useful in the preparation of salts useful as intermediates in obtaining the compounds of the invention and their pharmaceutically acceptable salts.

As used herein, the term “solvate” refers to a complex of variable stoichiometry formed by a solute (in this invention, a compound of Formula I, or a salt or physiologically functional derivative thereof) and a solvent. Such solvents, for the purpose of the invention, should not interfere with the biological activity of the solute. Non-limiting examples of suitable solvents include, but are not limited to water, methanol, ethanol, and acetic acid. Preferably the solvent used is a pharmaceutically acceptable solvent. Most preferably the solvent used is water and the solvate is a hydrate.

As used herein, the term “physiologically functional derivative” refers to any pharmaceutically acceptable derivative of a compound of the present invention that, upon administration to a mammal, is capable of providing (directly or indirectly) a compound of the present invention or an active metabolite thereof. Such derivatives, for example, esters and amides, will be clear to those skilled in the art, without undue experimentation. Reference may be made to the teaching of Burger's Medicinal Chemistry And Drug Discovery, 5^{th} Edition, Vol 1: Principles and Practice, which is incorporated herein by reference to the extent that it teaches physiologically functional derivatives.

Processes for preparing pharmaceutically acceptable salts, solvates, and physiologically functional derivatives of the compounds of Formula I are generally known in the art. See, for example, Burger's Medicinal Chemistry and Drug Discovery, 5^{th} Edition, Volume 1: Principles and Practice.

As used herein, the term “effective amount” means that amount of a drug or pharmaceutical agent that will elicit the biological or medical response of a tissue, system, animal, or human that is being sought, for instance, by a researcher or clinician. The term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function. For use in therapy, therapeutically effective amounts of a compound of Formula I, as well as salts, solvates, and physiologically functional derivatives thereof, may be administered as the raw chemical. Additionally, the active ingredient may be presented as a pharmaceutical composition.

As used herein, the term “treatment” includes prophylaxis and refers to alleviating the specified condition, eliminating or reducing one or more symptoms of the condition, slowing or eliminating the progression of the condition, and preventing or delaying the reoccurrence of the condition in a previously afflicted or diagnosed patient or subject. Prophylaxis (or prevention or delay of disease onset) is typically accomplished by administering a drug in the same or similar manner as one would to a patient with the developed disease or condition.

Accordingly, the invention further provides pharmaceutical compositions (also referred to herein as “pharmaceutical formulations”) that include effective amounts of a compound of the Formula I, a salt, a solvate, or a physiologically functional derivative thereof, and one or more pharmaceutically acceptable excipients (including carriers and/or diluents). The compounds of Formula I, salts, solvates, and physiologically functional derivatives thereof, are as herein described. The carrier(s), diluent(s) or excipient(s) must be acceptable, in the sense of being compatible with the other ingredients of the formulation and not deleterious to the recipient of the pharmaceutical composition.

In accordance with another aspect of the invention there is also provided a process for the preparation of a pharmaceutical formulation including admixing a compound of the Formula I or a salt, solvate, or physiologically functional derivative thereof, with one or more pharmaceutically acceptable carriers, diluents or excipients.

A therapeutically effective amount of a compound of the present invention will depend upon a number of factors. For example, the species, age, and weight of the recipient, the precise condition requiring treatment and its severity, the nature of the formulation, and the route of administration are all factors to be considered. The therapeutically effective amount ultimately should be at the discretion of the attendant physician or veterinarian. Regardless, an effective amount of a compound of Formula I for the treatment of humans suffering from frailty, generally, should be in the range of 0.1 to 100 mg/kg body weight of recipient (mammal) per day. More usually the effective amount should be in the range of 1 to 10 mg/kg body weight per day. Thus, for a 70 kg adult mammal the actual amount per day would usually be from 70 to 700 mg. This amount may be given in a single dose per day or in a number (such as two, three, four, five, or more) of sub-doses per day such that the total daily dose is the same. An effective amount of a salt, solvate, or physiologically functional derivative thereof, may be determined as a proportion of the effective amount of the compound of Formula I per se. Similar dosages should be appropriate for treatment (including prophylaxis) of the other conditions referred to herein.

Pharmaceutical formulations may be presented in unit dose forms containing a predetermined amount of active ingredient per unit dose. Such a unit may contain, as a non-limiting example, 0.5 mg to 1 g of a compound of the Formula I, depending on the condition being treated, the route of administration, and the age, weight, and condition of the patient. Preferred unit dosage formulations are those containing a daily dose or sub-dose, as herein above recited, or an appropriate fraction thereof, of an active ingredient. Such pharmaceutical formulations may be prepared by any of the methods well known in the pharmacy art.

Pharmaceutical formulations may be adapted for administration by any appropriate route, for example by an oral (including buccal or sublingual), rectal, nasal, topical (including buccal, sublingual or transdermal), vaginal, or parenteral (including subcutaneous, intramuscular, intravenous or intradermal) route. Such formulations may be prepared by any method known in the art of pharmacy, for example by bringing into association the active ingredient with the carrier(s) or excipient(s). In the present invention oral routes are preferred.

Pharmaceutical formulations adapted for oral administration may be presented as discrete units such as capsules or tablets; powders or granules; solutions or suspensions, each with aqueous or non-aqueous liquids; edible foams or whips; or oil-in-water liquid emulsions or water-in-oil liquid emulsions. For instance, for oral administration in the form of a tablet or capsule, the active drug component can be combined with an oral, non-toxic pharmaceutically acceptable inert carrier such as ethanol, glycerol, water, and the like. Generally, powders are prepared by comminuting the compound to a suitable fine size and mixing with an appropriate pharmaceutical carrier such as an edible carbohydrate, as, for example, starch or mannitol. Flavorings, preservatives, dispersing agents, sweetening agents, and coloring agents can also be present.

Capsules are made by preparing a powder, liquid, or suspension mixture and encapsulating with gelatin or some other appropriate shell material. Glidants and lubricants such as colloidal silica, talc, magnesium stearate, calcium stearate, or solid polyethylene glycol can be added to the mixture before the encapsulation. A disintegrating or solubilizing agent such as agar-agar, calcium carbonate or sodium carbonate can also be added to improve the availability of the medicament when the capsule is ingested. Moreover, when desired or necessary, suitable binders, lubricants, disintegrating agents, and coloring agents can also be incorporated into the mixture. Examples of suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants useful in these dosage forms include, for example, sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

Tablets are formulated, for example, by preparing a powder mixture, granulating or slugging, adding a lubricant and disintegrant, and pressing into tablets. A powder mixture may be prepared by mixing the compound, suitably comminuted, with a diluent or base as described above. Optional ingredients include binders such as carboxymethylcellulose, aliginates, gelatins, or polyvinyl pyrrolidone, solution retardants such as paraffin, resorption accelerators such as a quaternary salt, and/or absorption agents such as bentonite, kaolin, or dicalcium phosphate. The powder mixture can be wet-granulated with a binder such as syrup, starch paste, acadia mucilage or solutions of cellulosic or polymeric materials, and forcing through a screen. As an alternative to granulating, the powder mixture can be run through the tablet machine and the result is imperfectly formed slugs broken into granules. The granules can be lubricated to prevent sticking to the tablet-forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material, and a polish coating of wax can be provided. Dyestuffs can be added to these coatings to distinguish different unit dosages.

Oral fluids such as solutions, syrups, and elixirs can be prepared in dosage unit form so that a given quantity contains a predetermined amount of the compound. Syrups can be prepared, for example, by dissolving the compound in a suitably flavored aqueous solution, while elixirs are prepared through the use of a non-toxic alcoholic vehicle. Suspensions can be formulated generally by dispersing the compound in a non-toxic vehicle. Solubilizers and emulsifiers such as ethoxylated isostearyl alcohols and polyoxy ethylene sorbitol ethers, preservatives; flavor additives such as peppermint oil, or natural sweeteners, saccharin, or other artificial sweeteners; and the like can also be added.

Where appropriate, dosage unit formulations for oral administration can be microencapsulated. The formulation can also be prepared to prolong or sustain the release as for example by coating or embedding particulate material in polymers, wax or the like.

The compounds may also be coupled with soluble polymers as targetable drug carriers. Such polymers can include polyvinylpyrrolidone (PVP), pyran copolymer, polyhydroxypropylmethacrylamide-phenol, polyhydroxyethyl-aspartamidephenol, or polyethyleneoxidepolylysine substituted with palmitoyl residues. Furthermore, the compounds may be coupled to a class of biodegradable polymers useful in achieving controlled release of a drug; for example, polylactic acid, polyepsilon caprolactone, polyhydroxy butyric acid, polyorthoesters, polyacetals, polydihydropyrans, polycyanoacrylates, and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986), incorporated herein by reference as related to such delivery systems.

Pharmaceutical formulations adapted for topical administration may be formulated as ointments, creams, suspensions, lotions, powders, solutions, pastes, gels, sprays, aerosols, or oils.

For treatments of the eye or other external tissues, for example mouth and skin, the formulations may be applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles, and mouthwashes.

Pharmaceutical formulations adapted for nasal administration, where the carrier is a solid, include a powder having a particle size for example in the range 20 to 500 microns. The powder is administered in the manner in which snuff is taken, i.e., by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered dose pressurized aerosols, nebulizers, or insufflators.

Pharmaceutical formulations adapted for rectal administration may be presented as suppositories or as enemas.

Pharmaceutical formulations adapted for vaginal administration may be presented as pessaries, tampons, creams, gels, pastes, foams, or spray formulations.

Pharmaceutical formulations adapted for parenteral administration include aqueous and non-aqueous sterile injection solutions which may contain anti-oxidants, buffers, bacteriostats, and solutes that render the formulation isotonic with the blood of the intended recipient; and aqueous and non-aqueous sterile suspensions which may include suspending agents and thickening agents. The formulations may be presented in unit-dose or multi-dose containers, for example sealed ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets.

In addition to the ingredients particularly mentioned above, the formulations may include other agents conventional in the art having regard to the type of formulation in question. For example, formulations suitable for oral administration may include flavouring, sweetening, or coloring agents.

The compounds of the present invention, their salts, solvates, or physiologically functional derivatives thereof, may be employed alone or in combination with other therapeutic agents. The compound(s) of Formula I and the other pharmaceutically active agent(s) may be administered together or separately and, when administered separately, administration may occur simultaneously or sequentially, in any order. The amounts of the compound(s) of Formula I and the other pharmaceutically active agent(s) and the relative timings of administration will be selected in order to achieve the desired combined therapeutic effect. The administration in combination of a compound of Formula I (salt, solvate, or physiologically functional derivative thereof) with other treatment compounds or agents may be in combination by administration concomitantly in: (1) a unitary pharmaceutical composition including both compounds or (2) separate pharmaceutical compositions each including one of the compounds. Alternatively, the combination may be administered separately in a sequential manner wherein one treatment agent is administered first and the other second or vice versa. Such sequential administration may be close in time or remote in time.

The compounds of the present invention may be used in the treatment of a variety of disorders and conditions and, as such, the compounds of the present invention may be used in combination with a variety of other suitable therapeutic agents useful in the treatment (including prophylaxis) of obesity and/or associated diseases, disorders, or conditions. More specifically, the present invention includes the treatment (including prophylaxis) of obesity. Other disorders, conditions, and/or diseases associated with obesity which may be benefited by the compounds and pharmaceutical compositions of the invention can include diabetes, depression (major and bipolar), anxiety, hypertension, drug and substance addiction, and arteriosclerosis.

One aspect of the present invention comprises a compound of Formula I (a salt, solvate, or physiologically functional derivative thereof) in combination with at least one other species selected from the group consisting of at least one agent or drug for treating obesity, diabetes, hypertension, and arteriosclerosis. In particular, a compound of Formula I (a salt, solvate , or physiologically functional derivative thereof) may be combined with at least one species for the treatment of obesity selected from the group of human ciliary neurotropic factor, a CB-1 antagonist or inverse agonist (such as rimonabant), a neurotransmitter reuptake inhibitor (such as sibutramine, bupropion, or bupropion HCl, radafaxine), a lipase inhibitor (such as orlistat), an MC4R agonist, a 5-HT2c agonist, a ghrelin receptor antagonist, a CCK-A receptor agonist, an NPY Y1 antagonist, PYY₃₋₃₆ and a PPAR activator.

The compounds of this invention may be made by a variety of methods, including well-known standard synthetic methods. Illustrative general synthetic methods are set out below and then specific compounds of the invention are prepared in the working examples.

Those skilled in the art will recognize if a stereocenter exists in compounds of Formula I. Accordingly, the present invention includes all possible stereoisomers and includes not only racemic compounds but the individual enantiomers as well. When a compound is desired as a single enantiomer, such may be obtained by stereospecific synthesis, by resolution of the final product or any convenient intermediate, or by chiral chromatographic methods as are known in the art. Resolution of the final product, an intermediate, or a starting material may be affected by any suitable method known in the art. See, for example, Stereochemistry of Organic Compounds by E. L. Eliel, S. H. Wilen, and L. N. Mander (Wiley-Interscience, 1994), incorporated by reference with regard to stereochemistry.

In all of the following synthetic descriptions protecting groups for sensitive or reactive groups were employed where necessary in accordance with general principles of synthetic chemistry. Protecting groups are manipulated according to standard methods of organic synthesis (T. W. Green and P. G. M. Wuts (1999) Protecting Groups in Organic Synthesis, 3^{rd} edition, John Wiley and Sons, incorporated by reference with regard to protecting groups). These groups are removed at a convenient stage of the compound synthesis using methods that are readily apparent to those skilled in the art. The selection of processes as well as the reaction conditions and order of their execution shall be consistent with the preparation of the compounds of Formula I.

In all of the following synthetic descriptions, the variables T, U, V, Z (e.g., Z¹, Z², Z³, and Z⁴), A, D, J, R¹, R², R³, R⁴, R⁵, R⁶ and n are as described for Formula I unless otherwise noted.

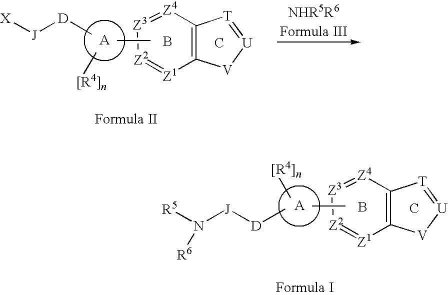

Compounds of Formula I can be prepared from compounds of Formula II (where X is a suitable leaving group such as a halogen (e.g., Cl, Br or I), triflate, or tosylate) by reaction with a compound of Formula III in a suitable organic solvent such as EtOH or MeOH, with or without a promoter such as NaI, at a temperature ranging from room temperature to 160 degrees C. using conventional or microwave heating. When NHR⁵R⁶ is a salt (e.g., HCl or trifluoroacetate), a base (e.g., Et₃N and/or (iPr)₂NEt) is added to the reaction mixture.

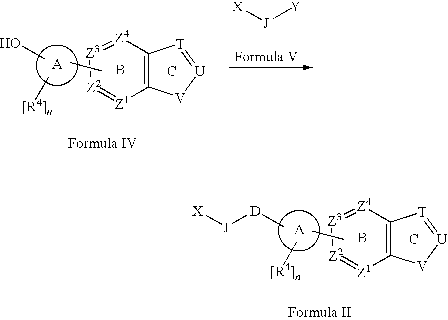

When D is O and J is (CH₂)ₙ where n is 1, 2, 3, or 4, compounds of Formula II can be prepared from compounds of Formula IV by reaction with compounds of Formula V where X is a suitable leaving group as defined above. Y can be the same as X or different and can be chosen from a group consisting of, for example, a halogen atom (e.g., Cl, Br or I), triflate, or tosylate in a suitable solvent (e.g., CH₃CN), in the presence of a base (e.g., K₂CO₃), at a temperature ranging from room temperature to 85 degrees C.

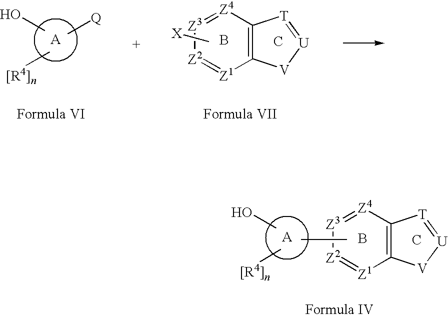

Compounds of Formula IV can be prepared by reaction of compounds of Formula VI (where Q can be chosen from a group consisting of, for example, —B(OH)₂ or —B(OCMe₂CMe₂O)) with compounds of Formula VII (where X is as defined herein) under Suzuki reaction conditions, in a suitable organic solvent such as DME, in the presence of a suitable catalyst such as Pd(PPh₃)₄, at a temperature ranging from room temperature to 85 degrees C.

Compounds of Formula VI are commercially available or can be prepared from commercially available starting materials by one skilled in the art of organic synthesis.

Compounds of Formula VII are commercially available or can be prepared from commercially available starting materials by one skilled in the art of organic synthesis.

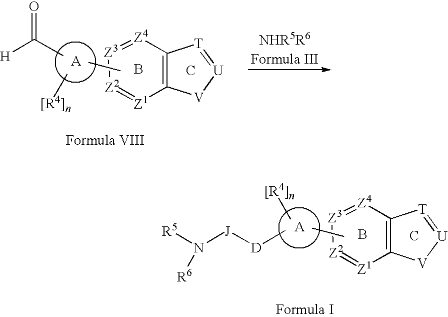

Compounds of Formula I (where D is CH₂ and J is a bond) can be prepared from compounds of VIII by reaction with compounds of Formula III in the presence of a suitable reducing agent such as NaBH(OAc)₃ or NaCNBH₃, in a suitable organic solvent such as MeOH or dichloromethane, with or without acetic acid, at a temperature ranging from room temperature to 50 degrees C.

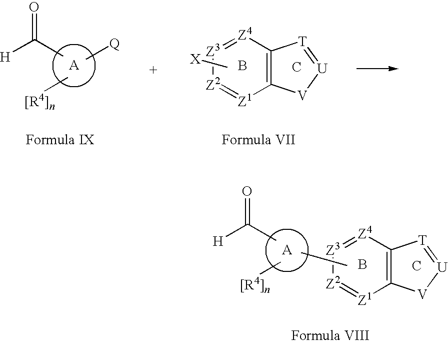

Compounds of Formula VIII can be prepared by reaction of a compound of Formula IX with a compound of Formula VII under Suzuki reaction conditions, in a suitable solvent such as DME, toluene, EtOH, or a combination thereof, in the presence of a suitable catalyst such as Pd(PPh₃)₄, at a temperature ranging from room temperature to 110 degrees C.

Compounds of Formula IX are commercially available or can be prepared from commercially available starting materials by someone skilled in the art of organic synthesis.

### EXPERIMENTAL

Non-commercial amines used for the reductive aminations described in the syntheses herein were prepared as described in the section entitled “Synthesis of Non-commerically Available Amines.”

### Preparation of Biaryl-benzimidazole Compounds

5-Bromo-1-(triphenylmethyl)-1H-benzimidazole or 6-bromo-1-(triphenylmethyl)-1H-benzimidazole was prepared according to the procedure in María L. López-Rodríguez, Bellinda Benhamú, David Ayala, J. Luis Rominguera, Marta Murcia, José A. Ramos and Alma Viso, Tetrahedron, Vol. 56(20), pp. 3245-3253, 2000. Both compounds, either the single isomer or the mixture, can be used in the following preparations.

General Method A:

### Example A-1

### Step 1: 3-[1-(Triphenylmethyl)-1H-benzimidazol-6-yl]phenol

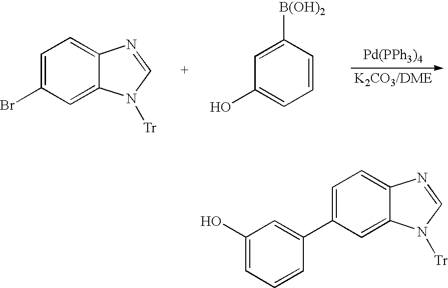

6-Bromo-1-(triphenylmethyl)-1H-benzimidazole (0.75 g, 1.71 mmol), 3-hydroxyphenylboronic acid (0.35 g, 2.56 mmol) and tetrakis(triphenylphosphine)palladium(0) (0.231 g, 0.035 mmol) in 20 ml of dimethoxyethane were degassed by vacuum-nitrogen backfilling cycles. Potassium carbonate (0.592 g, 4.3 mmol) was added. The mixture was heated to reflux for 15 h, cooled and partitioned between ethyl acetate and water. The organic phase was separated, washed with brine, dried over magnesium sulfate and concentrated in vacuo. The residue was purified by silica gel column chromatography (10 to 60% ethyl acetate in hexanes) to afford the title compound as a white solid.

¹H NMR (400 MHz, CDCl₃) delta ppm 5.98 (s, 1H) 6.60-6.78 (m, 4H) 7.10-7.40 (m, 17H) 7.79 (d, 1H) 7.96 (s, 1H)

### Step 2: 6-{3-[(2-Chloroethyl)oxy]phenyl}-1-(triphenylmethyl)-1H-benzimidazole

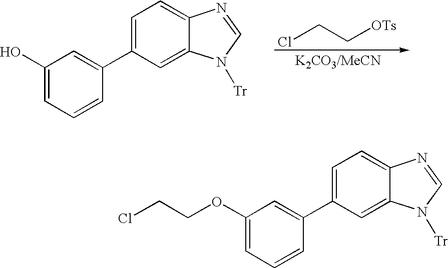

3-[1-(Triphenylmethyl)-1H-benzimidazol-6-yl]phenol (0.68 g, 1.5 mmol), 2-chloroethyl p-toluenesulfonate (1.41 g, 6.0 mmol) and potassium carbonate (0.83 g, 6.0 mmol) in 20 ml of acetonitrile were heated to reflux for 18 h. The reaction mixture was filtered to remove the solids, concentrated to dryness in vacuo and purified by silica gel column chromatography (20 to 60% ethyl acetate in hexanes) to give the title compound as a white solid.

¹H NMR (400 MHz, DMSO-d₆) delta ppm 3.80 (t, 2H) 4.18 (t, 2H) 6.58 (s, 2H) 6.80 (m, 2H) 7.18-7.40 (m, 17H) 7.80 (d, 1H) 7.92 (s, 1H); (M+) 515.20, (Ph₃C⁺) 243.22, 3.13 min (LC/MS method A)

### Step 3: (2-{[3-(1H-Benzimidazol-5-yl)phenyl]oxy}-ethyl)[2-(3-fluorophenyl)ethyl]amine Hydrochloride

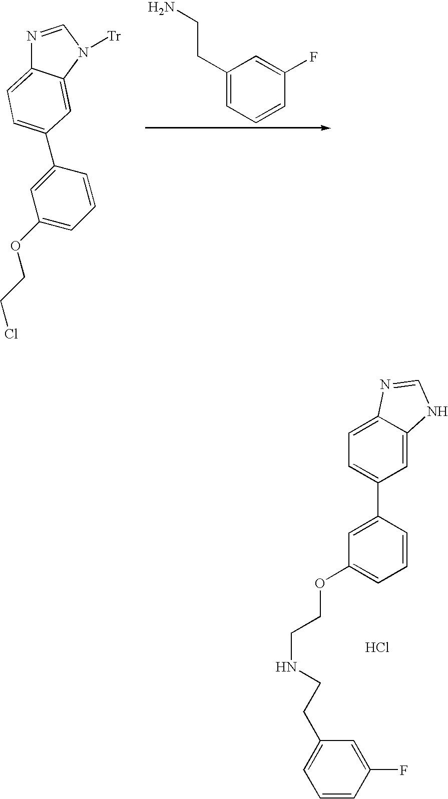

6-{3-[(2-Chloroethyl)oxy]phenyl}-1-(triphenylmethyl)-1H-benzimidazole (0.121 g, 0.234 mmol), and 3-fluorophenethylamine (0.123 ml, 0.938 mmol) in 1.5 ml of ethanol were heated via microwave at 160 degrees C. for 1.5 h. The reaction mixture was concentrated in vacuo, dissolved in dichloromethane, washed with brine, dried over magnesium sulfate and evaporated under reduced pressure. The residue was purified by silica gel column chromatography (0 to 10% methanol in dichloromethane) to afford the free base. The base was dissolved in 10 ml of dichloromethane and 1 ml of 1M hydrogen chloride diethyl ether solution was added. The mixture was stirred and concentrated to dryness to give the title compound as a white solid.

¹H NMR (400 MHz, DMSO-d₆) delta ppm 3.02 (m, 2H) 3.24 (m, 2H) 3.40 (m, 2H) 4.40 (m, 2H) 7.02-7.18 (m, 4H) 7.30-7.47 (m, 4H) 7.81 (d, 1H) 7.90 (d, 1H) 8.01 (s, 1H) 9.32 (br., 2H) 9.50 (s, 1H); (M+1) 376.23, 1.24 min (LC/MS method A)

TABLE 1
Compounds Prepared by General Method A

| | | Name and Characterization | |
|---|---|---|---|
| Ex. | Structure | Data | Method/Comments¹ |
| A-2 | 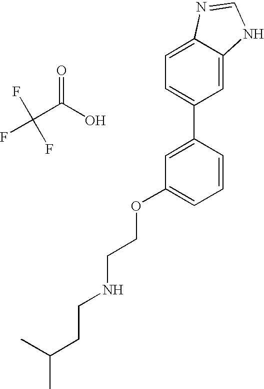 | N-(2-{[3-(1H-benzimidazol-5- yl)phenyl]oxy}ethyl)-3-methyl-1- butanamine trifluoroacetic acid salt (M + 1) 324.33, 1.06 min (LC/MS method A) | Method A reaction time 1 h; trifluoroacetic (TFA) acid was used after amination; purified by preparative HPLC |
| A-3 | 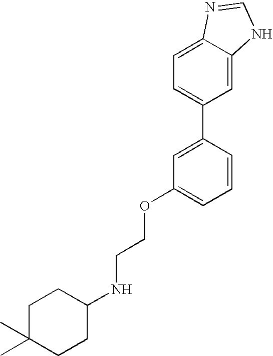 | N-(2-{[3-(1H-benzimidazol-5- yl)phenyl]oxy}ethyl)-4,4- dimethylcyclohexanamine (M + 1) 364.32, 1.38 min (LC/MS method A) | Method A reaction time 1 h; purified by preparative HPLC and flash column chromatography (10% methanol in dichloromethane) |
| A-4 | 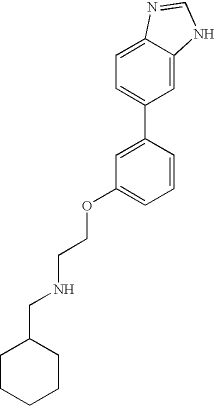 | (2-{[3-(1H-benzimidazol-5- yl)phenyl]oxy}ethyl)(cyclohexylmeth- yl)amine (M + 1) 350.32, 1.26 min (LC/MS method A) | Method A reaction time 1 h; TFA was used after amination; purified by preparative HPLC and flash column chromatography (10% methanol in dichloromethane) |
| A-5 | 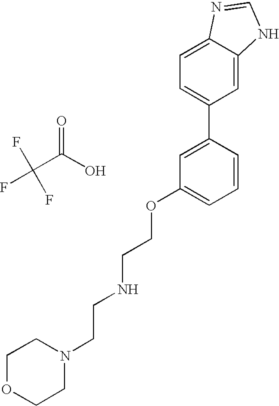 | (2-{[3-(1H-benzimidazol-5- yl)phenyl]oxy}ethyl)[2-(4- morpholinyl)ethyl]amine trifluoroacetic acid salt (M + 1) 367.28, 0.92 min (LC/MS method C) | Method A purified by preparative HPLC |
| A-6 | 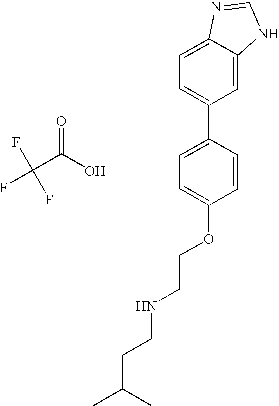 | N-(2-{[4-(1H-benzimidazol-5- yl)phenyl]oxy}ethyl)-3-methyl-1- butanamine trifluoroacetic acid salt (M + 1) 324.24, 1.08 min (LC/MS method A) | Method A 4-hydroxyphenyl- boronic acid was used at Step 1: preparative HPLC was applied at Step 3 with the reaction time 2 h. |
| A-7 | 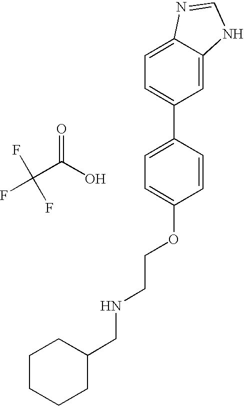 | (2-{[4-(1H-benzimidazol-5- yl)phenyl]oxy}ethyl)(cyclohexylmeth- yl)amine trifluoroacetic acid salt (M + 1) 350.30, 1.24 min (LC/MS method A) | Method A 4-hydroxyphenyl- boronic acid was used at Step 1: preparative HPLC was applied at Step 3 with the reaction time 2 h. |
| A-8 | 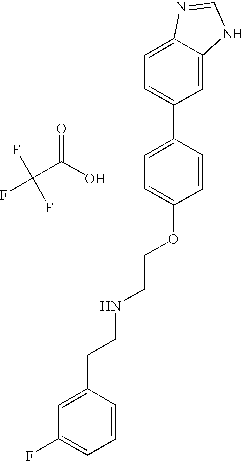 | (2-{[4-(1H-benzimidazol-5- yl)phenyl]oxy}ethyl)[2-(3- fluorophenyl)ethyl]amine trifluoroacetic acid salt (M + 1) 376.24, 1.29 min (LC/MS method A) | Method A 4-hydroxyphenyl- boronic acid was used at Step 1: preparative HPLC was applied at Step 3 with the reaction time 2 h. |
| A-9 | 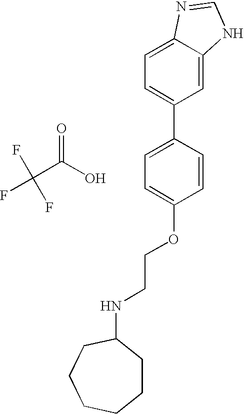 | N-(2-{[4-(1H-benzimidazol-5- yl)phenyl]oxy}ethyl)cycloheptana mine trifluoroacetic acid salt (M + 1) 350.25, 1.22 min (LC/MS method A) | Method A 4-hydroxyphenyl- boronic acid was used at Step 1: preparative HPLC was applied at Step 3 with the reaction time 2 h. |
| A-10 | 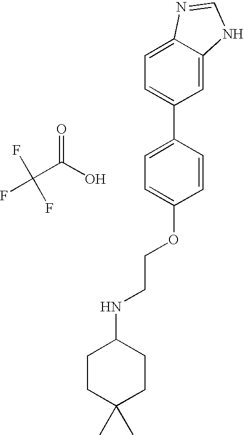 | N-(2-{[4-(1H-benzimidazol-5- yl)phenyl]oxy}ethyl)-4,4- dimethylcyclohexanamine trifluoroacetic acid salt (M + 1) 364.28, 1.37 min (LC/MS method A) | Method A 4-hydroxyphenyl- boronic acid was used at Step 1: preparative HPLC was applied at Step 3 with the reaction time 2 h. |
| A-11 | 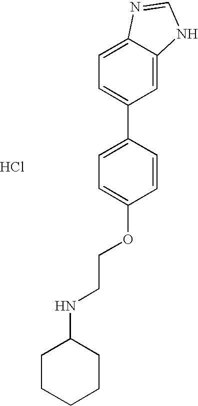 | N-(2-{[4-(1H-benzimidazol-5- yl)phenyl]oxy}ethyl)cyclohexana mine hydrochloride (M + 1) 336.26, 1.03 min (LC/MS method A) | Method A 4-hydroxyphenyl- boronic acid was used at Step 1: preparative HPLC was applied at Step 3 with the reaction time 2 h. |
| A-12 | 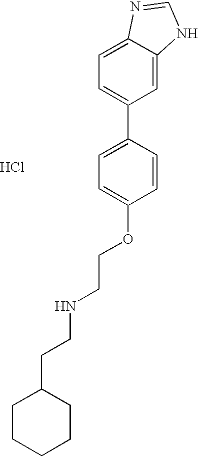 | (2-{[4-(1H-benzimidazol-5- yl)phenyl]oxy}ethyl)(2- cyclohexylethyl)amine hydrochloride (M + 1) 364.34, 1.40 min (LC/MS method A) | Method A 4-hydroxyphenyl- boronic acid was used at Step 1: preparative HPLC was applied at Step 3 with the reaction time 2 h. |
| A-13 | 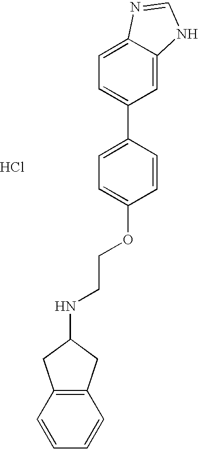 | N-(2-{[4-(1H-benzimidazol-5- yl)phenyl]oxy}ethyl)-2,3-dihydro- 1H-inden-2-amine hydrochloride (M + 1) 370.13, 1.20 min (LC/MS method B) | Method A 4-hydroxyphenyl- boronic acid was used at Step 1: preparative HPLC was applied at Step 3 with the reaction time 2 h. |
| A-14 | 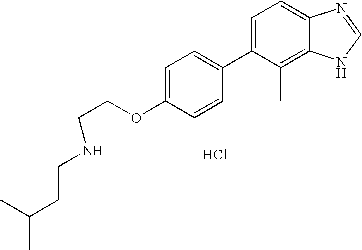 | (2-{[4-(4-Methyl-1H- benzimidazol-5-yl)phenyl] oxy}ethyl)(4-methylpentyl)amine Hydrochloride white solid. ¹H NMR (400 MHz, DMSO-d₆) delta ppm 0.85(d, 6 H) 1.50- 1.68(m, 3 H) 2.50(s, 3 H) 2.98 (m, 2 H) 3.35(m, 2 H) 4.38(m, 2 H) 7.05(dd, 2 H) 7.32-7.42(m, 3 H) 7.66(d, 1H) 7.68(d, 1H) 9.16(br., 2 H) 9.60(s, 1 H); (M + 1) 338.14, 1.19 min (LC/MS method A) | Method A Used Intermediate-1 Reaction time 2 h Reaction mixture was purified without work up. Residue from chromatography was treated with CH₂Cl₂/Et₂O Mixtures. Residue dissolved in CH₂Cl₂ containing 10% MeOH before adding 1 M HCl in Et₂O |
| A-15 | 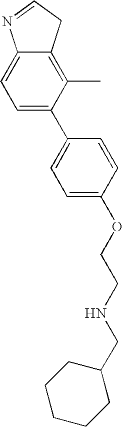 | N-(cyclohexylmethyl)-2-{[4-(4- methyl-1H-benzimidazol-5- yl)phenyl]oxy}ethanamine (M + 1) 364.14, 1.32 min (LC/MS method A) | Method A Used Intermediate-1 Free base Reaction time 2 h Reaction mixture was purified without work up. Residue from chromatography was treated with CH₂Cl₂/Et₂O mixtures. |
| A-16 | 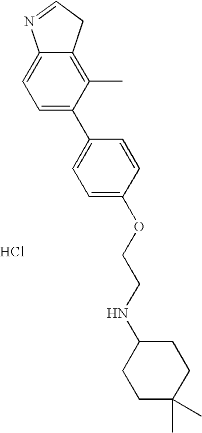 | (4,4-dimethylcyclohexyl)(2-{[4- (4-methyl-1H-benzimidazol-6- yl)phenyl]oxy}ethyl)amine Hydrochloride (M + 1) 378.03, 1.46 min (LC/MS method A) | Method A Used Intermediate-1 Reaction time 2 h Reaction mixture was purified without work up. Residue from chromatography was treated with CH₂Cl₂/Et₂O mixtures. Residue dissolved in CH₂Cl₂ containing 10% MeOH before adding 1 M HCl in Et₂O |
| A-17 | 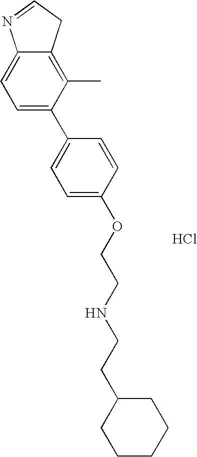 | (2-cyclohexylethyl)(2-{[4-(4- methyl-1H-benzimidazol-5- yl)phenyl]oxy}ethyl)amine Hydrochloride (M + 1) 378.22, 1.60 min (LC/MS method A) | Method A Used Intermediate-1 Reaction time 2 h Reaction mixture was purified without work up. Residue from chromatography was treated with CH₂Cl₂/Et₂O mixtures. Residue dissolved in CH₂Cl₂ containing 10% MeOH before adding 1 M HCl in Et₂O |
| A-18 | 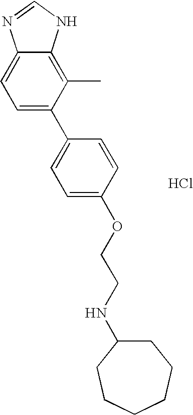 | N-(2-{[4-(4-methyl-1H- benzimidazol-5- yl)phenyl]oxy}ethyl)cycloheptana mine Hydrochloride (M + 1) 364.20, 1.30 min (LC/MS method C) | Method A Used Intermediate-1 Reaction time 2 h Reaction mixture was purified without work up. Residue from chromatography was treated with CH₂Cl₂/Et₂O mixtures. Residue dissolved in CH₂Cl₂ containing 10% MeOH before adding 1 M HCl in Et₂O |
| A-19 | 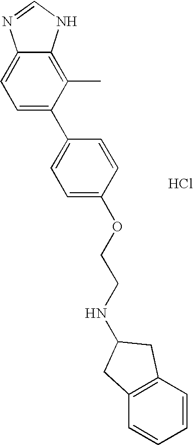 | N-(2-{[4-(4-methyl-1H- benzimidazol-5- yl)phenyl]oxy}ethyl)-2,3-dihydro- 1H-inden-2-amine Hydrochloride (M + 1) 383.99, 1.30 min (LC/MS method A) | Method A Used Intermediate-1 Reaction time 2 h Reaction mixture was purified without work up. Residue from chromatography was treated with CH₂Cl₂/Et₂O mixtures. Residue dissolved in CH₂Cl₂ containing 10% MeOH before adding 1 M HCl in Et₂O |

¹Note: The description for the synthesis of Intermediate 1 referred to in this column can be found in the section entitled, “Synthetic Descriptions of Intermediates Used in General Methods A through K.”

General Method B:

### Example B-1

### Step 1: 4-[1-(Triphenylmethyl)-1H-benzimidazol-6-yl]benzaldehyde and 4-[1-(triphenylmethyl)-1H-benzimidazol-5-yl]benzaldehyde

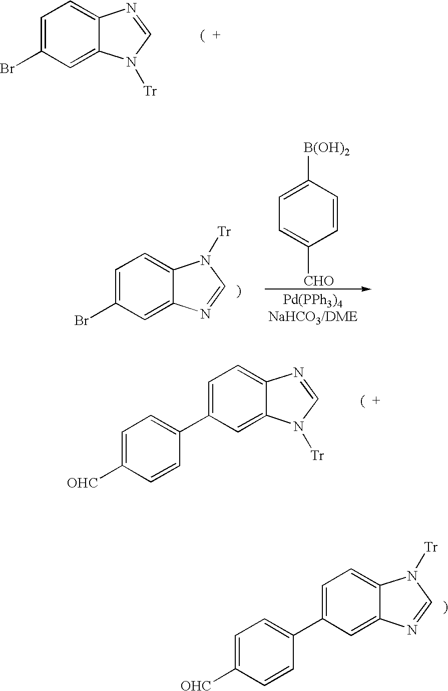

An isomeric mixture of 5-bromo-1-(triphenylmethyl)-1H-benzimidazole and 6-bromo-1-(triphenylmethyl)-1H-benzimidazole (2.17 g, 4.94 mmol, in 1:1 ratio approximately), 4-formylphenylboronic acid (0.9 g, 7.5 mmol) and tetrakis(triphenylphosphine)palladium(0) (0.116 g, 0.1 mmol) in 40 ml of dimethoxyethane and 12.5 ml of saturated aqueous sodium bicarbonate solution were degassed by vacuum-nitrogen backfilling cycles and heated to reflux for 15 h. The mixture was cooled to the room temperature and partitioned between ethyl acetate and water. The organic layer was separated, washed with brine, dried over magnesium sulfate and concentrated in vacuo. The residue was purified by silica gel column chromatography (0 to 50% ethyl acetate in hexanes) to give the title compound as a white solid.

¹H NMR (400 MHz, CDCl₃) delta ppm 6.60 (m, 1H) 7.18-7.45 (m, 19H) 7.72-8.16 (m, 3H) 10.00 (m, 1H); LC-MS: (Ph₃C⁺) 243.17, 2.99 min and (Ph₃C⁺) 243.17, 3.07 min (LC/MS method A)

TABLE 2
Aldehyde Intermediates Prepared by General Method B (Step1)

| | | Name and Characterizing | |
|---|---|---|---|
| Intermediate | Structure | Data | Comments¹ |
| Intermediate-2 | 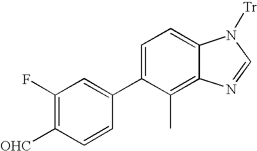 | 2-fluoro-4-[4-methyl-1- (triphenylmethyl)-1H-benz- imidazol-5-yl]benzaldehyde Yellow solid ¹H NMR (400 MHz, CDCl₃) delta ppm 2.63 (s, 3 H) 6.38 (d, 1 H) 6.80 (d, 1 H) 7.12- 7.25 (m, 8 H) 7.25-40 (m, 9 H) 7.86 (t, 1 H) 8.00 (s, 1 H) 10.40 (s, 1 H) | Used commercially available 3- fluoro-4- formylphenyl boronic acid and the product from Intermediate-3, step 4 |
| Intermediate-4 | 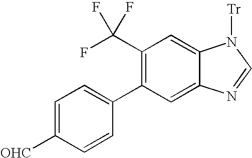 | 4-[6-(Trifluoromethyl)-1- (triphenylmethyl)-1H-benz- imidazol-5-yl]benzaldehyde Pink solid ¹H NMR (400 MHz, CDCl₃) delta ppm 6.30 (s, 1 H) 7.08- 7.40 (m, 17 H) 7.76 (dd, 2 H) 8.10 (s, 1 H) 8.22 (s, 1 H 10.00 (s, 1 H); LC-MS: 243.11 (Ph₃C+), 3.08 min (LC/MS method A) 4-[5-(Trifluoromethyl)-1- (triphenylmethyl)-1H-benz- imidazol-6-yl]benzaldehyde Pale brown solid ¹H NMR (400 MHz, CDCl₃) delta ppm 6.81 (s, 1 H) 7.12- 7.43 (m, 15 H) 7.52 (dd, 2 H) 7.70 (s, 1 H) 7.90 (dd, 2 H) 8.12 (s, 1 H) 10.08 (s, 1 H); LC-MS: 243.11 (Ph₃C+), 3.09 min (LC/MS method A) | Used Intermediate-5 for step 1 The two regioisomers could be separated by silica gel column chromatography (10 to 50% ethylacetate in hexanes). |
| Intermediate-6 | 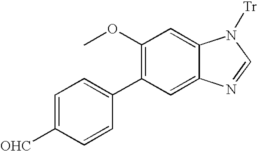 | 4-[6-Methoxy-1-(triphenyl- methyl)-1H-benzimidazol-5- yl]benzaldehyde and 4-[5 methoxy-1-(triphenylmethyl)- 1H-benzimidazol-6-yl] benzaldehyde Pale yellow solid LC-MS: 243.15 (Ph₃C+), 2.99 min (LC/MS method A) | Used Intermediate-7 The two regioisomers were not separated |
| Intermediate-8 | 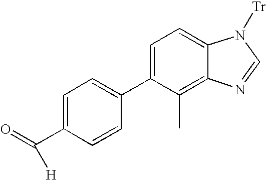 | 4-[4-methyl-1- (triphenylmethyl)-1H- benzimidazol-5- yl]benzaldehyde ¹H NMR (400 MHz, CDCl₃) delta ppm 2.62 (s, 3 H) 6.38 (d, 1 H) 6.82 (d, 1 H) 7.10- 7.40 (m, 15 H) 7.50 (dd, 2 H) 7.89 (dd, 2 H) 7.98 (t, 1 H) 10.05 (s, 1 H) | Prepared from product of Intermediate-3, step 4 and 4- formylphenylbor- onic acid |
| Intermediate-9 | 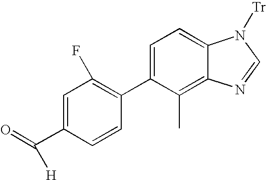 | 3-fluoro-4-[4-methyl-1- (triphenylmethyl)-1H- benzimidazol-5- yl]benzaldehyde ¹H NMR (400 MHz, CDCl₃) delta ppm 2.58 (s, 3 H) 6.38 (d, 1 H) 6.79 (d, 1 H) 7.18- 7.38 (m, 15 H) 7.42 (t, 1 H) 7.62 (d, 1 H) 7.66 (d, 1 H) 8.00 (s, 1 H) 10.00 (s, 1 H) | Prepared from product of Intermediate-3, step 4 (2-fluoro-4- formylphenyl) boronic acid |
| Intermediate-10 | 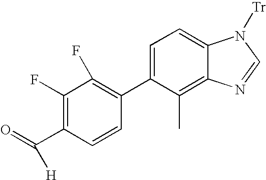 | 2,3-difluoro-4-[4-methyl-1- (triphenylmethyl)-1H- benzimidazol-5- yl]benzaldehyde ¹H NMR (400 MHz, CDCl₃) delta ppm 2.57 (s, 3 H) 6.40 (d, 1 H) 6.75 (d, 1 H) 7.10- 7.38 (m, 16 H) 7.62 (m, 1 H) 7.98 (s, 1 H) 10.38 (s, 1 H) | Prepared from product of Intermediate-3, step 4 and (2,3- difluoro-4- formylphenyl) boronic acid |
| Intermediate-11 | 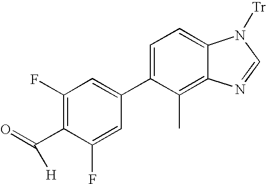 | 2,6-difluoro-4-[4-methyl-1- (triphenylmethyl)-1H- benzimidazol-5- yl]benzaldehyde ¹H NMR (400 MHz, CDCl₃) delta ppm 2.63 (s, 3 H) 6.39 (d, 1 H) 6.80 (d, 1 H) 6.99 (d, 2 H) 7.10-7.38 (m, 15 H) 8.00 (s, 1 H) 10.38 (s, 1 H) | Prepared from product of Intermediate-3, step 4 and (3,5- difluoro-4- formylphenyl) boronic acid |
| Intermediate-12 | 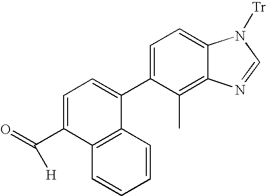 | 4-[4-methyl-1-(triphenyl- methyl)-1H-benzimidazol-5- yl]-1- naphthalenecarbaldehyde ¹H NMR (400 MHz, CDCl₃) delta ppm 2.38 (s, 3 H) 6.41 (d, 1 H) 6.80 (d, 1 H) 7.20- 7.40 (m, 15 H) 7.50 (m, 3 H) 7.65 (m, 1 H) 8.02 (m, 2 H) 9.35 (d, 1 H) 10.42 (s, 1 H) | Prepared from product of Intermediate-3, step 4 and (4- formyl-1- naphthalenyl) boronic acid |
| Intermediate-13 | 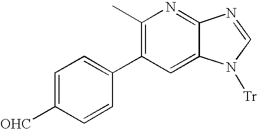 | 4-[5-methyl-1- (triphenylmethyl)-1H- imidazo[4,5-b]pyridin-6- yl]benzaldehyde ¹H NMR (400 MHz, DMSO- d₆) delta ppm 2.43 (s, 3 H) 6.41 (s, 1 H) 7.12-7.42 (m, 17 H) 7.85 (dd, 2 H) 8.20 (s, 1 H) 10.00 (s, 1 H) LC-MS: 243.11 (Ph₃C+), 2.87 min (LC/MS method A) | Prepared from 4- formylphenylbor- onic acid and the product prepared as described in Intermediate-5 starting with 6- bromo-5- methyl-1H- imidazo[4,5- b]pyridine |
| Intermediate-14 | 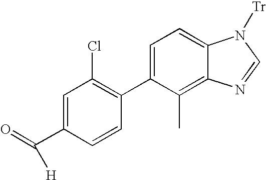 | 3-chloro-4-[4-methyl-1- (triphenylmethyl)-1H- benzimidazol-5- yl]benzaldehyde ¹H NMR (400 MHz, CDCl₃) delta ppm 2.44 (s, 3 H) 6.38 (d, 1 H) 6.70 (d, 1 H) 7.20- 7.38 (m, 15 H) 7.40 (d, 1 H) 7.80 (d, 1 H) 7.98 (s, 2 H) 10.00 (s, 1H) | Prepared using the procedure described for Intermediate-75 using 3-chloro- 4- hydroxybenzalde- hyde |
| Intermediate-15 | 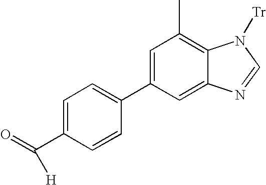 | 4-[7-methyl-1- (triphenylmethyl)-1H- benzimidazol-5- yl]benzaldehyde ¹H NMR (400 MHz, CDCl₃) delta ppm 2.76 (s, 3 H) 6.45 (d, 1 H) 7.18-7.40 (m, 18 H) 7.80 (dd, 2 H) 8.02 (s, 1 H) 9.98 (s, 1 H) LC-MS: 243.15 (Ph₃C+), 3.10 min (LC/MS method A) | Prepared from 4- formylphenylbor- onic acid and the product prepared as described in Intermediate-5 starting with 4- bromo-2- methyl-6- nitroaniline. |
| Intermediate-16 | 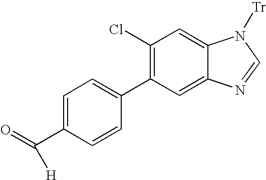 | 4-[6-chloro-1-(triphenyl- methyl)-1H-benzimidazol-5- yl]benzaldehyde Mixture of two regioisomers LC-MS: 243.11 (Ph₃C+), 3.12 min (LC/MS method A) | Prepared from 4- formylphenylbor- onic acid and the product prepared as described in Intermediate-5 starting with 5- chloro-2- nitroaniline |
| Intermediate-17 | 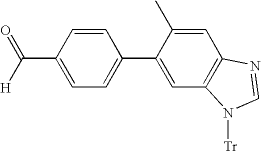 | 4-[5-methyl-1- (triphenylmethyl)-1H- benzimidazol-6- yl]benzaldehyde Mixture of two regioisomers LC-MS: 243.11 (Ph₃C+), 2.99 min and 243.11 (Ph₃C+), 3.04 min (LC/MS method A) | Prepared from 4- formylphenylbor- onic acid and the product prepared as described in Intermediate-5 starting with 5- methyl-2- nitroaniline |
| Intermediate-75 | 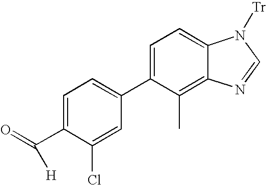 | 2-chloro-4-[4-methyl-1- (triphenylmethyl)-1H- benzimidazol-5- yl]benzaldehyde ¹H NMR (400 MHz, CDCl₃) delta ppm 2.60 (s, 3 H) 6.38 (d, 1 H) 6.76 (d, 1 H) 7.17- 7.40 (m, 17 H) 7.95 (m, 2 H) 10.45 (s, 1 H) | Prepared as described in the section entitled, “Synthetic Descriptions of Intermediates Used in General Methods A through K” |

¹Note:
Descriptions for the synthesis of intermediates referred to in this column and used to synthesize the above aldehydes can be found in the section entitled, “Synthetic Descriptions of Intermediates Used in General Methods A through K.”

### Step 2: {[4-(1H-Benzimidazol-5-yl)phenyl]methyl}(2-cyclohexylethyl)amine Trifluoroacetic Acid Salt

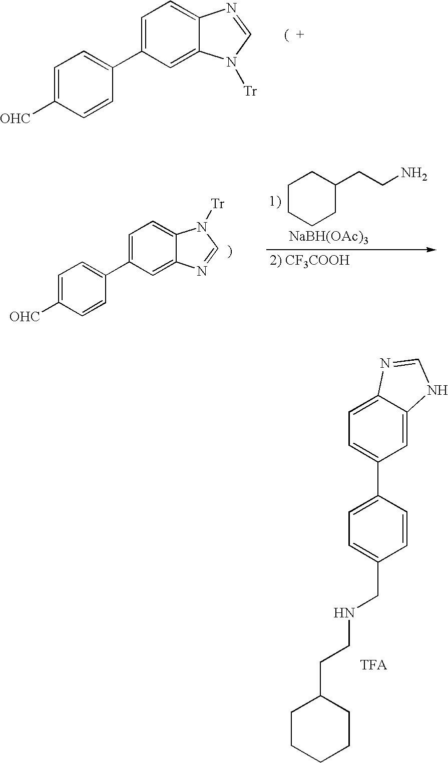

The intermediate from General Method B, Step 1 (0.116 g, 0.25 mmol), and (2-cyclohexylethyl)amine (0.128 g, 1.0 mmol) were stirred in 4 ml of 4% acetic acid in dichloromethane for approximate 1 h before sodium triacetoxyborohydride (0.21 g, 1.0 mmol) was added. The reaction mixture was stirred for 15 h at room temperature, directly loaded on a 12 g ISCO silica gel column and eluted with 0 to 10% methanol in dichloromethane to give a mixture of the two amine isomers. The mixture was dissolved in 2-3 ml of trifluoroacetic acid and stirred for 12 h at the room temperature. The yellow reaction mixture was concentrated in vacuo and the residue was triturated with diethyl ether. The solid was collected by filtration and washed with diethyl ether to afford the title compound as a white solid.

¹H NMR (400 MHz, DMSO-d₆) delta ppm 0.85 (m, 2H) 1.03-1.38 (m, 4H) 1.43-1.68 (m, 7H) 2.98 (m, 2H) 4.20 (m, 2H) 7.58 (m, 2H) 7.70-7.85 (m, 4H) 8.00 (s, 1H) 8.78 (br., 2H) 9.05 (s, 1H); (M+1) 334.28, 1.25 min (LC/MS method A)

TABLE 3
Compounds Prepared by General Method B

| Ex. | Structure | Name and Characterization Data | Method/Comments |
|---|---|---|---|
| B-2 | 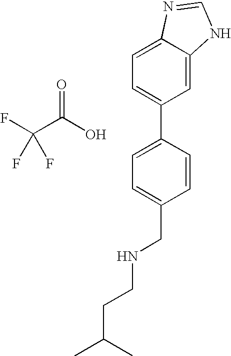 | N-{[4-(1H-benzimidazol-5- yl)phenyl]methyl}-3-methyl-1- butanamine trifluoroacetic acid salt (M + 1) 294.22, 0.90 min (LC/MS method A) | Method B |
| B-3 | 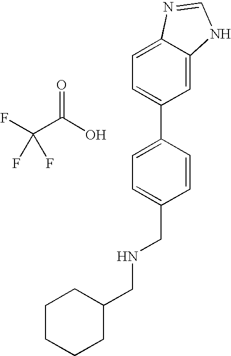 | {[4-(1H-benzimidazol-5- yl)phenyl]methyl}(cyclohexylmethyl) amine trifluoroacetic acid salt (M + 1) 320.12, 1.11 min (LC/MS method B) | Method B |
| B-4 | 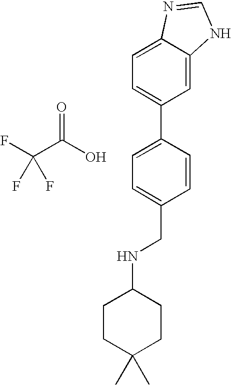 | {[4-(1H-benzimidazol-5- yl)phenyl]methyl}(4,4- dimethylcyclohexyl)amine trifluoroacetic acid salt (M + 1) 334.28, 1.16 min (LC/MS method A) | Method B |
| B-5 | 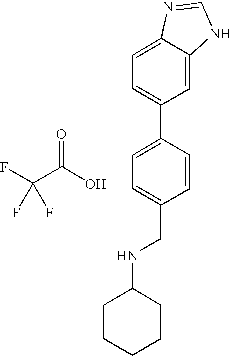 | {[4-(1H-benzimidazol-5- yl)phenyl]methyl}cyclohexylamine trifluoroacetic acid salt (M + 1) 306.19, 0.83 min (LC/MS method A) | Method B |
| B-6 | 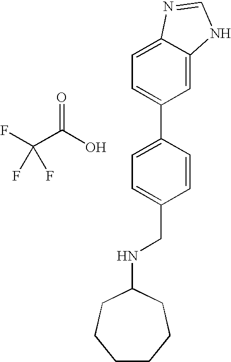 | {[4-(1H-benzimidazol-5- yl)phenyl]methyl}cycloheptylamine trifluoroacetic acid salt (M + 1) 320.23, 1.00 min (LC/MS method A) | Method B |
| B-7 | 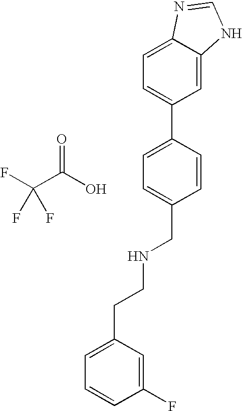 | {[4-(1H-benzimidazol-5- yl)phenyl]methyl}[2-(3- fluorophenyl)ethyl]amine trifluoroacetic acid salt (M + 1) 346.20, 0.96 min (LC/MS method A) | Method B |
| B-8 | 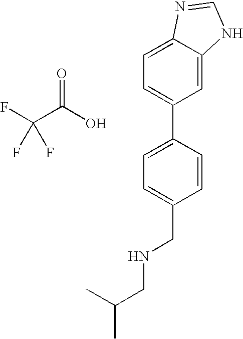 | {[4-(1H-benzimidazol-5- yl)phenyl]methyl}(2- methylpropyl)amine trifluoroacetic acid salt (M + 1) 280.14, 1.15 min (LC/MS method D) | Method B |
| B-9 | 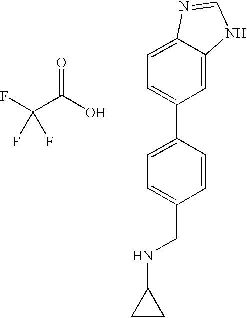 | N-{[4-(1H-benzimidazol-5- yl)phenyl]methyl}cyclopropanamine trifluoroacetic acid salt (M + 1) 264.04, 1.09 min (LC/MS method D) | Method B |
| B-10 | 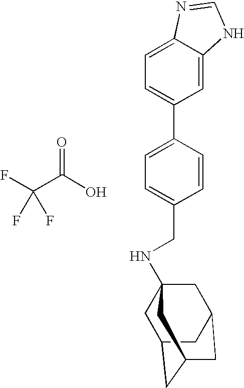 | (1S,3s)-N-{[4-(1H-benzimidazol-5- yl)phenyl]methyl}tricyclo[3.3.1.1^{3,7}] decan-1-amine trifluoroacetic acid salt (M + 1) 358.11, 1.29 min (LC/MS method B) | Method B |
| B-11 | 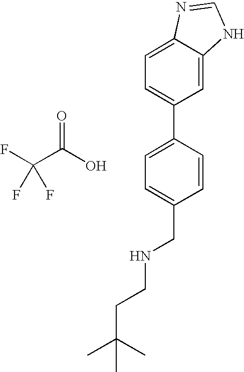 | N-{[4-(1H-benzimidazol-5- yl)phenyl]methyl}-3,3-dimethyl-1- butanamine trifluoroacetic acid salt (M + 1) 308.16, 1.10 min (LC/MS method B) | Method |
| B-12 | 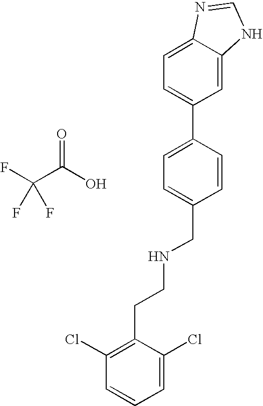 | N-{[4-(1H-benzimidazol-5- yl)phenyl]methyl}-2-(2,6- dichlorophenyl)ethanamine trifluoroacetic acid salt (M +) 396.04, 1.37 min (LC/MS method B) | Method B |
| B-13 | 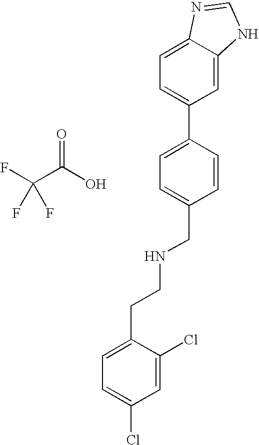 | N-{[4-(1H-benzimidazol-5- yl)phenyl]methyl}-2-(2,4- dichlorophenyl)ethanamine trifluoroacetic acid salt (M +) 396.01, 1.44 min (LC/MS method B) | Method B |
| B-14 | 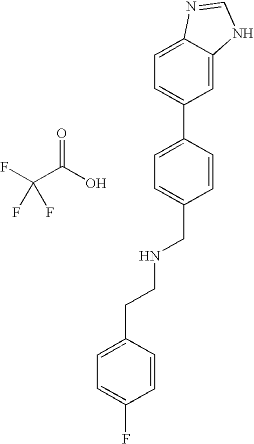 | N-{[4-(1H-benzimidazol-5- yl)phenyl]methyl}-2-(4- fluorophenyl)ethanamine trifluoroacetic acid salt (M + 1) 346.07, 1.08 min (LC/MS method B) | Method B |
| B-15 | 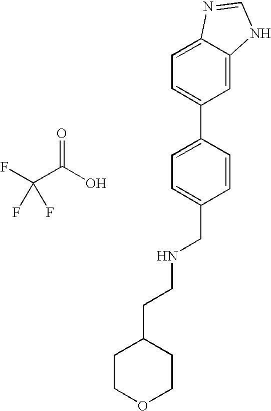 | N-{[4-(1H-benzimidazol-5- yl)phenyl]methyl}-2-(tetrahydro-2H- pyran-4-yl)ethanamine trifluoroacetic acid salt (M + 1) 336.07, 0.78 min (LC/MS method B) | Method B |
| B-16 | 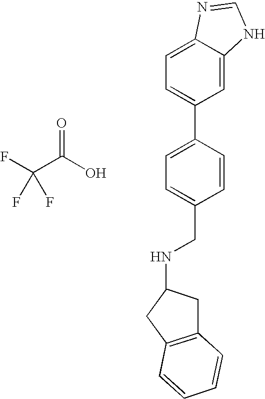 | N-{[4-(1H-benzimidazol-5- yl)phenyl]methyl}-2,3-dihydro-1H- inden-2-amine trifluoroacetic acid salt (M + 1) 340.14, 1.02 min (LC/MS method A) | Method B |
| B-17 | 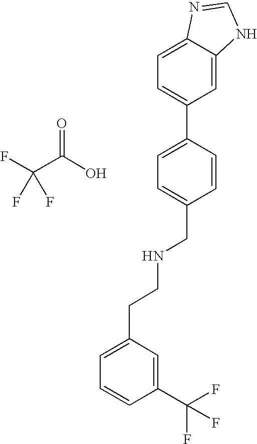 | N-{[4-(1H-benzimidazol-5- yl)phenyl]methyl}-2-[3- (trifluoromethyl)phenyl]ethanamine trifluoroacetic acid salt (M + 1) 396.05 1.32 min (LC/MS method B) | Method B |
| B-18 | 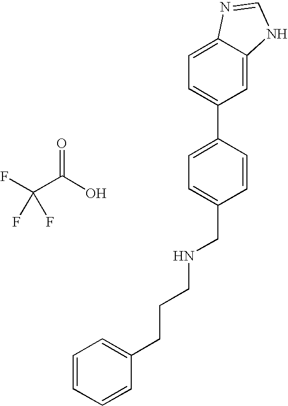 | N-{[4-(1H-benzimidazol-5- yl)phenyl]methyl}-3-phenyl-1- propanamine trifluoroacetic acid salt (M + 1) 342.04 1.15 min (LC/MS method B) | Method B |
| B-19 | 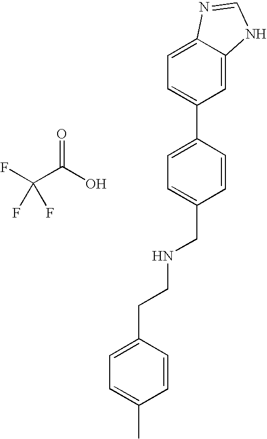 | N-{[4-(1H-benzimidazol-5- yl)phenyl]methyl}-2-(4- methylphenyl)ethanamine trifluoroacetic acid salt (M + 1) 342.15 1.17 min (LC/MS method A) | Method B |
| B-20 | 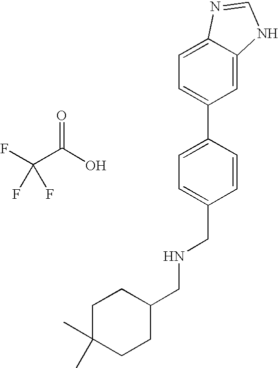 | {[4-(1H-benzimidazol-5- yl)phenyl]methyl}[(4,4- dimethylcyclohexyl)methyl]amine trifluoroacetic acid salt (M + 1) 348.20 1.35 min (LC/MS method A) | Method B |
| B-21 | 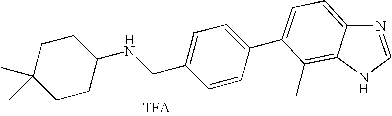 | (4,4-dimethylcyclohexyl){[4-(4- methyl-1H-benzimidazol-5- yl)phenyl]methyl}amine trifluoroacetic acid salt (M + 1) 348.17, 1.27 min (LC/MS method A) | Method B Used Intermediate-8 |
| B-22 | 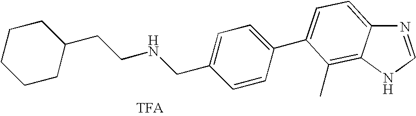 | (2-cyclohexylethyl){[4-(4-methyl-1H- benzimidazol-5- yl)phenyl]methyl}amine trifluoroacetic acid salt (M + 1) 348.20, 1.36 min (LC/MS method A) | Method B Used Intermediate-8 |
| B-23 | 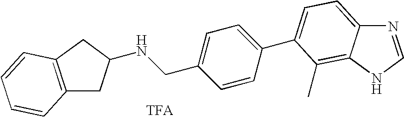 | N-{[4-(4-methyl-1H-benzimidazol-5- yl)phenyl]methyl}-2,3-dihydro-1H- inden-2-amine trifluoroacetic acid salt (M + 1) 354.12, 1.15 min (LC/MS method A) | Method B Used Intermediate-8 |
| B-24 | 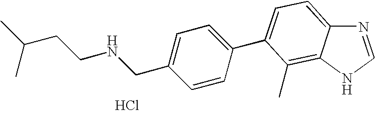 | {[4-(4-methyl-1H-benzimidazol-5- yl)phenyl]methyl}(3- methylbutyl)amine hydrochloride (M + 1) 308.13, 0.94 min (LC/MS method A) | Method B Used Intermediate-8 |
| B-25 | 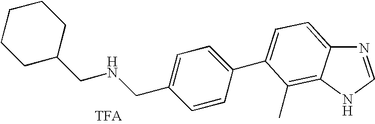 | (cyclohexylmethyl){[4-(4-methyl-1H- benzimidazol-5- yl)phenyl]methyl}amine trifluoroacetic acid salt (M + 1) 334.15, 1.16 min (LC/MS method A) | Method B Used Intermediate-8 |
| B-26 | 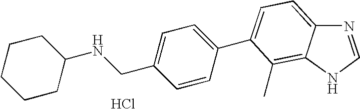 | N-{[4-(4-methyl-1H-benzimidazol-5- yl)phenyl]methyl}cyclohexanamine hydrochloride (M + 1) 320.13, 0.96 min (LC/MS method A) | Method B Used Intermediate-8 |
| B-27 | 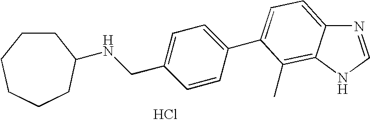 | N-{[4-(4-methyl-1H-benzimidazol-5- yl)phenyl]methyl}cycloheptanamine hydrochloride (M + 1) 334.06, 1.09 min (LC/MS method A) | Method B Used Intermediate-8 |
| B-28 | 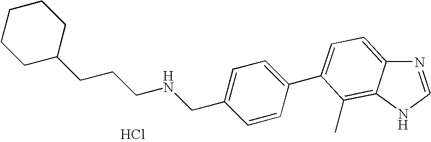 | (3-cyclohexylpropyl){[4-(4-methyl- 1H-benzimidazol-5- yl)phenyl]methyl}amine hydrochloride (M + 1) 362.07, 1.57 min (LC/MS method A) | Method B Used Intermediate-8 |
| B-29 | 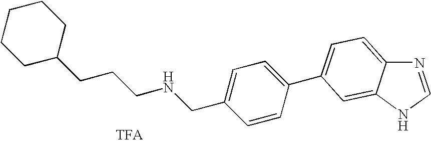 | {[4-(1H-benzimidazol-5- yl)phenyl]methyl}(3-cyclohexyl propyl)amine trifluoroacetic acid salt (M + 1) 348.22 1.49 min (LC/MS method A) | Method B 4 M HCl in dioxane was used for deprotection in step 2 |
| B-30 | 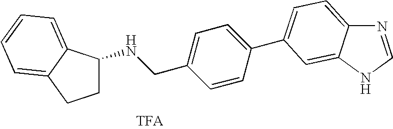 | (1R)-N-{[4-(1H-benzimidazol-5- yl)phenyl]methyl}-2,3-dihydro-1H- inden-1-amine trifluoroacetic acid salt (M + 1) 339.96, 1.15 min (LC/MS method A) | Method B 4 M HCl in dioxane was used for deprotection in step 2 |
| B-31 | 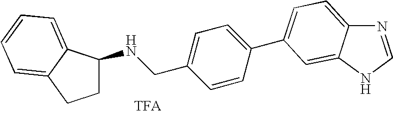 | (1S)-N-{[4-(1H-benzimidazol-5- yl)phenyl]methyl}-2,3-dihydro-1H- inden-1-amine trifluoroacetic acid salt (M + 1) 340.14, 1.07 min (LC/MS method A) | Method B 4 M HCl in dioxane was used for deprotection in step 2 |
| B-32 | 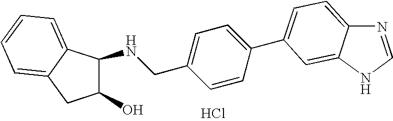 | (1R,2S)-1-({[4-(1H-benzimidazol-5- yl)phenyl]methyl}amino)-2,3- dihydro-1H-inden-2-ol hydrochloride (M + 1) 356.07, 0.96 min (LC/MS method A) | Method B 4 M HCl in dioxane was used for deprotection in step 2 |
| B-33 | 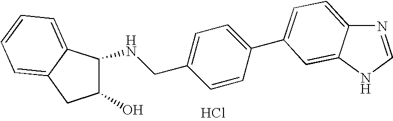 | (1S,2R)-1-({[4-(1H-benzimidazol-5- yl)phenyl]methyl}amino)-2,3- dihydro-1H-inden-2-ol hydrochloride (M + 1) 356.13, 1.03 min (LC/MS method A) | Method B 4 M HCl in dioxane was used for deprotection in step 2 |
| B-34 | 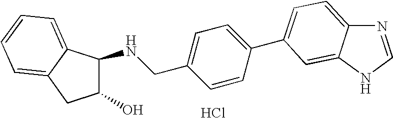 | (1R,2R)-1-({[4-(1H-benzimidazol-5- yl)phenyl]methyl}amino)-2,3- dihydro-1H-inden-2-ol hydrochloride (M + 1) 356.13, 0.88 min (LC/MS method A) | Method B 4 M HCl in dioxane was used for deprotection in step 2 |
| B-35 | 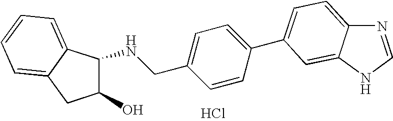 | (1S,2S)-1-({[4-(1H-benzimidazol-5- yl)phenyl]methyl}amino)-2,3- dihydro-1H-inden-2-ol hydrochloride (M + 1) 356.10, 0.91 min (LC/MS method A) | Method B 4 M HCl in dioxane was used for deprotection in step 2 |
| B-36 | 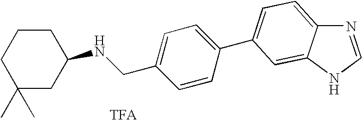 | (1R)-N-{[4-(1H-benzimidazol-5- yl)phenyl]methyl}-3,3- dimethylcyclohexanamine trifluoroacetic acid salt (M + 1) 334.21, 1.15 min (LC/MS method A) | Method B 4 M HCl in dioxane was used for deprotection in step 2 |
| B-37 | 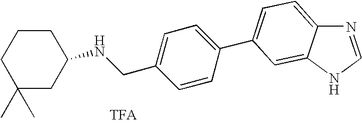 | (1S)-N-{[4-(1H-benzimidazol-5- yl)phenyl]methyl}-3,3- dimethylcyclohexanamine trifluoroacetic acid salt (M + 1) 334.15, 1.19 min (LC/MS method A) | Method B 4 M HCl in dioxane was used for deprotection in step 2 |
| B-38 | 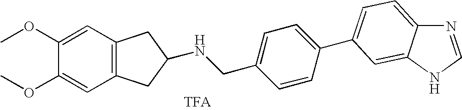 | N-{[4-(1H-benzimidazol-5- yl)phenyl]methyl}-5,6- bis(methyloxy)-2,3-dihydro-1H- inden-2-amine trifluoroacetic acid salt (M +) 400.11, 1.00 min (LC/MS method A) | Method B 4 M HCl in dioxane was used for deprotection in step 2 |
| B-39 | 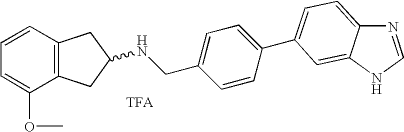 | N-{[4-(1H-benzimidazol-5- yl)phenyl]methyl}-4-(methyloxy)-2,3- dihydro-1H-inden-2-amine trifluoroacetic acid salt (M +) 370.07, 1.21 min (LC/MS method A) | Method B 4 M HCl in dioxane was used for deprotection in step 2 |
| B-40 | 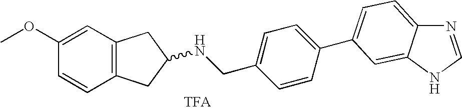 | N-{[4-(1H-benzimidazol-5- yl)phenyl]methyl}-5-(methyloxy)-2,3- dihydro-1H-inden-2-amine trifluoroacetic acid salt (M + 1) 370.11, 1.17 min (LC/MS method A) | Method B 4 M HCl in dioxane was used for deprotection in step 2 |
| B-41 | 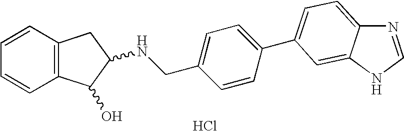 | (cis) 2-({[4-(1H-benzimidazol-5- yl)phenyl]methyl}amino)-2,3- dihydro-1H-inden-1-ol hydrochloride (M + 1) 356.14, 0.93 min (LC/MS method A) | Method B 4 M HCl in dioxane was used for deprotection in step 2 |
| B-42 | 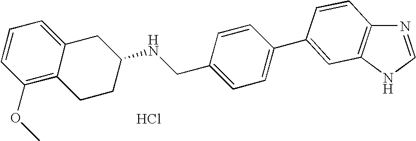 | (2R)-N-{[4-(1H-benzimidazol-5- yl)phenyl]methyl}-5-(methyloxy)- 1,2,3,4-tetrahydro-2- naphthalenamine hydrochloride (M + 1) 384.23, 1.32 min (LC/MS method A) | Method B 4 M HCl in dioxane was used for deprotection in step 2 |
| B-43 | 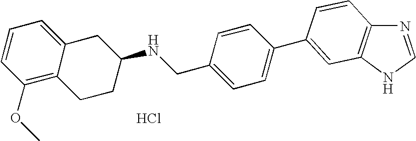 | (2S)-N-{[4-(1H-benzimidazol-5- yl)phenyl]methyl}-5-(methyloxy)- 1,2,3,4-tetrahydro-2- naphthalenamine hydrochloride (M + 1) 384.23, 1.27 min (LC/MS method A) | Method B 4 M HCl in dioxane was used for deprotection in step 2 |
| B-44 | 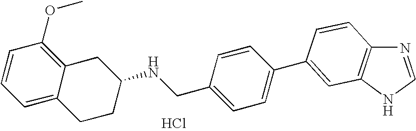 | (2R)-N-{[4-(1H-benzimidazol-5- yl)phenyl]methyl}-8-(methyloxy)- 1,2,3,4-tetrahydro-2- naphthalenamine hydrochloride (M + 1) 384.23, 1.28 min (LC/MS method A) | Method B 4 M HCl in dioxane was used for deprotection in step 2 |
| B-45 | 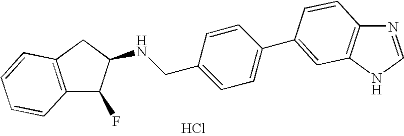 | (cis)-N-{[4-(1H-benzimidazol-5- yl)phenyl]methyl}-1-fluoro-2,3- dihydro-1H-inden-2-amine hydrochloride (M + 1) 358.21, 0.97 min (LC/MS method A) | Method B 4 M HCl in dioxane was used for deprotection in step 2 |
| B-46 | 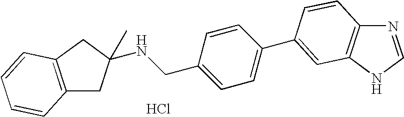 | N-{[4-(1H-benzimidazol-5- yl)phenyl]methyl}-2-methyl-2,3- dihydro-1H-inden-2-amine hydrochloride (M + 1) 354.18 1.18 min (LC/MS method A) | Method B 4 M HCl in dioxane was used for deprotection in step 2 |
| B-47 | 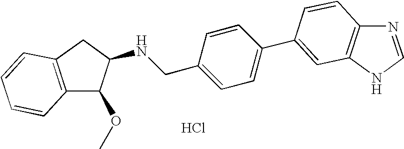 | (cis)-N-{[4-(1H-benzimidazol-5- yl)phenyl]methyl}-1-(methyloxy)-2,3- dihydro-1H-inden-2-amine hydrochloride (M + 1) 370.32 0.46 min (LC/MS method E) | Method B 4 M HCl in dioxane was used for deprotection in step 2 |
| B-48 | 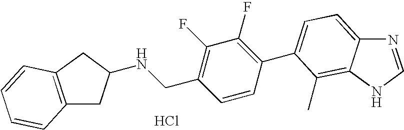 | N-{[2,3-difluoro-4-(4-methyl-1H- benzimidazol-5-yl)phenyl]methyl}- 2,3-dihydro-1H-inden-2-amine hydrochloride (M + 1) 390.22, 1.22 min (LC/MS method A) | Method B Used Intermediate- 10 4 M HCl in dioxane was used for deprotection in step 2 |
| B-49 | 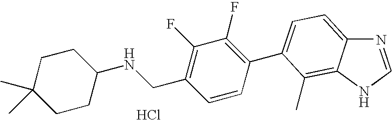 | N-{[2,3-difluoro-4-(4-methyl-1H- benzimidazol-5-yl)phenyl]methyl}- 4,4-dimethylcyclo hexanamine hydrochloride (M + 1) 384.26, 1.38 min (LC/MS method A) | Method B Used Intermediate- 10 4 M HCl in dioxane was used for deprotection in step 2 |
| B-50 | 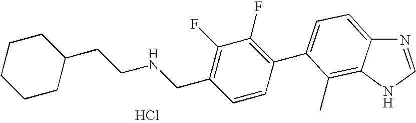 | (2-cyclohexylethyl){[2,3-difluoro-4- (4-methyl-1H-benzimidazol-5- yl)phenyl]methyl}amine hydrochloride (M + 1) 384.23, 1.46 min (LC/MS method A) | Method B Used Intermediate- 10 4 M HCl in dioxane was used for deprotection in step 2 |
| B-51 | 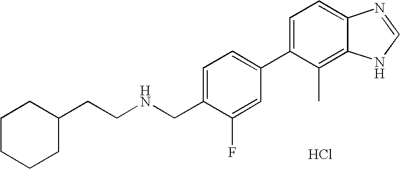 | 2-Cyclohexyl-N-{[2-fluoro-4-(4- methyl-1H-benzimidazol-5- yl)phenyl]methyl}ethanamine Hydrochloride white solid ¹H NMR (400 MHz, DMSO-d₆) delta ppm 0.87 (m, 2 H) 1.03-1.40 (m, 4 H) 1.55-1.70 (m, 7 H) 2.57 (s, 3 H) 3.00 (m, 2 H) 4.21 (m, 2 H) 7.25- 7.42 (m, 3 H) 7.68-7.82 (m, 2 H) 9.21-9.38 (br., 2 H) 9.58 (s, 1 H); (M + 1) 366.28, 1.56 min (LC/MS method A) | Method B Used Intermediate-2 4 M HCl in dioxane was used for deprotection in step 2 |
| B-52 | 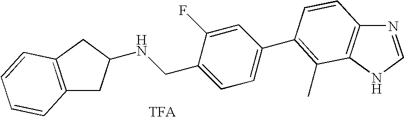 | N-{[2-fluoro-4-(4-methyl-1H- benzimidazol-5-yl)phenyl]methyl}- 2,3-dihydro-1H-inden-2-amine trifluoroacetic acid salt (M + 1) 372.20, 1.28 min (LC/MS method A) | Method B Used Intermediate-2 4 M HCl in dioxane was used for deprotection in step 2 |
| B-53 | 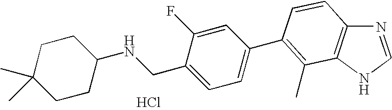 | N-{[2-fluoro-4-(4-methyl-1H- benzimidazol-5-yl)phenyl]methyl}- 4,4-dimethylcyclo hexanamine hydrochloride (M + 1) 366.26, 1.39 min (LC/MS method A) | Method B Used Intermediate-2 4 M HCl in dioxane was used for deprotection in step 2 |
| B-54 | 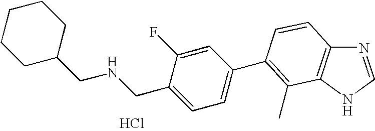 | (cyclohexylmethyl){[2-fluoro-4-(4- methyl-1H-benzimidazol-5- yl)phenyl]methyl}amine hydrochloride (M + 1) 352.23, 1.23 min (LC/MS method A) | Method B Used Intermediate-2 4 M HCl in dioxane was used for deprotection in step 2 |
| B-55 | 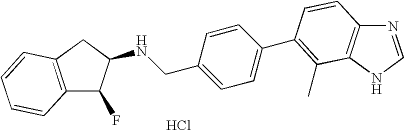 | (cis-1-fluoro-2,3-dihydro-1H-inden- 2-yl){[4-(4-methyl-1H-benzimidazol- 5-yl)phenyl]methyl}amine hydrochloride (M + 1) 372.26, 0.98 min (LC/MS method A) | Method B Used Intermediate-8 4 M HCl in dioxane was used for deprotection in step 2 |
| B-56 | 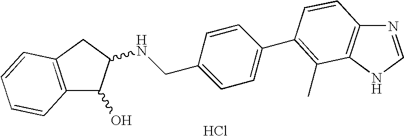 | 2-({[4-(4-methyl-1H-benzimidazol-5- yl)phenyl]methyl}amino)-2,3- dihydro-1H-inden-1-ol hydrochloride (M + 1) 370.23, 0.88 min; 370.23, 0.95 min (LC/MS method A) | Method B Used Intermediate-8 4 M HCl in dioxane was used for deprotection in step 2 The compound was obtained as a side product during the preparation of B-55. |
| B-57 | 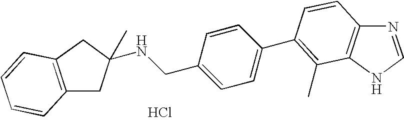 | {[4-(4-methyl-1H-benzimidazol-5- yl)phenyl]methyl}(2-methyl-2,3- dihydro-1H-inden-2-yl)amine hydrochloride (M + 1) 368.26, 1.22 min (LC/MS method A) | Method B Used Intermediate-8 4 M HCl in dioxane was used for deprotection in step 2 |
| B-58 | 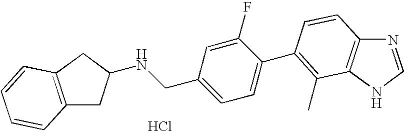 | N-{[3-fluoro-4-(4-methyl-1H- benzimidazol-5-yl)phenyl]methyl}- 2,3-dihydro-1H-inden-2-amine hydrochloride (M + 1) 372.12, 1.23 min (LC/MS method A) | Method B Used Intermediate-9 4 M HCl in dioxane was used for deprotection in step 2 |
| B-59 | 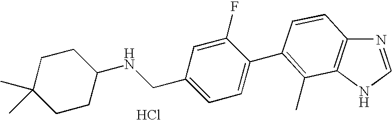 | N-{[3-fluoro-4-(4-methyl-1H- benzimidazol-5-yl)phenyl]methyl}- 4,4-dimethylcyclo hexanamine hydrochloride (M + 1) 366.23, 1.34 min (LC/MS method A) | Method B Used Intermediate-9 4 M HCl in dioxane was used for deprotection in step 2 |
| B-60 | 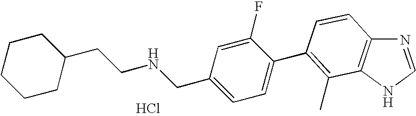 | (2-cyclohexylethyl){[3-fluoro-4-(4- methyl-1H-benzimidazol-5- yl)phenyl]methyl}amine hydrochloride (M + 1) 366.26, 1.40 min (LC/MS method A) | Method B Used Intermediate-9 4 M HCl in dioxane was used for deprotection in step 2 |
| B-61 | 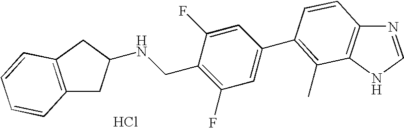 | N-{[2,6-difluoro-4-(4-methyl-1H- benzimidazol-5-yl)phenyl]methyl}- 2,3-dihydro-1H-inden-2-amine hydrochloride (M + 1) 390.22, 1.23 min (LC/MS method A) | Method B Used Intermediate- 11 4 M HCl in dioxane was used for deprotection in step 2 |
| B-62 | 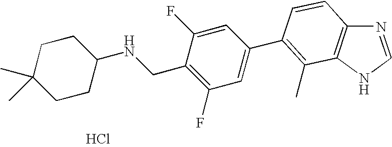 | N-{[2,6-difluoro-4-(4-methyl-1H- benzimidazol-5-yl)phenyl]methyl}- 4,4-dimethylcyclo hexanamine hydrochloride (M + 1) 384.30, 1.36 min (LC/MS method A) | Method B Used Intermediate- 11 4 M HCl in dioxane was used for deprotection in step 2 |
| B-63 | 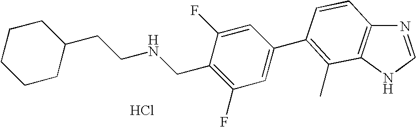 | (2-cyclohexylethyl){[2,6-difluoro-4- (4-methyl-1H-benzimidazol-5- yl)phenyl]methyl}amine hydrochloride (M + 1) 384.30, 1.48 min (LC/MS method A) | Method B Used Intermediate- 11 4 M HCl in dioxane was used for deprotection in step 2 |
| B-64 | 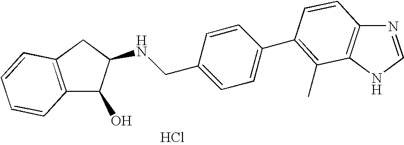 | cis-2-({[4-(4-Methyl-1H- benzimidazol-5-yl)phenyl] methyl}amino)-2,3-dihydro-1H- inden-1-ol hydrochloride ¹H NMR (400 MHz, DMSO-d₆) delta ppm 2.55 (s, 3 H) 3.20 (d, 2 H) 3.82 (m, 1 H) 4.31 (m, 2 H) 5.19 (m, 1 H) 7.25 (m, 3 H) 7.38-7.50 (m, 4 H) 7.73 (m, 3 H) 9.18 (br., 1 H) 9.62 (s, 1 H) 9.71 (br., 1 H) | Method B Used Intermediate-8 4 M HCl in dioxane was used for deprotection in step 2 |
| B-65 | 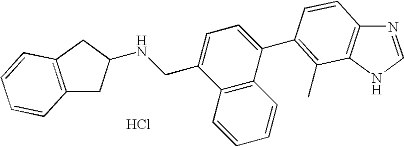 | N-{[4-(4-methyl-1H-benzimidazol-5- yl)-1-naphthalenyl]methyl}-2,3- dihydro-1H-inden-2-amine hydrochloride (M + 1) 404.22, 0.50 min (LC/MS method E) | Method B Used Intermediate- 12 4 M HCl in dioxane was used for deprotection in step 2 |
| B-66 | 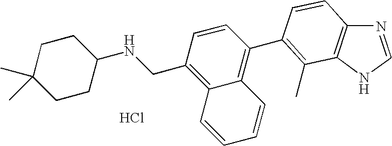 | (4,4-dimethylcyclohexyl){[4-(4- methyl-1H-benzimidazol-5-yl)-1- naphthalenyl]methyl}amine hydrochloride (M + 1) 398.27, 0.54 min (LC/MS method E) | Method B Used Intermediate- 12 4 M HCl in dioxane was used for deprotection in step 2 |
| B-67 | 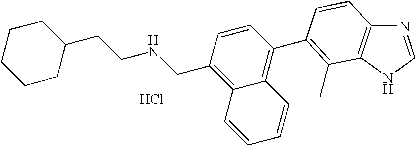 | (2-cyclohexylethyl){[4-(4-methyl-1H- benzimidazol-5-yl)-1- naphthalenyl]methyl}amine hydrochloride (M + 1) 398.27, 0.54 min (LC/MS method E) | Method B Used Intermediate- 12 4 M HCl in dioxane was used for deprotection in step 2 |
| B-68 | 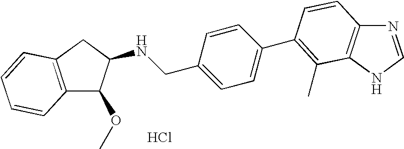 | cis-N-{[4-(4-methyl-1H- benzimidazol-5-yl)phenyl]methyl}-1- (methyloxy)-2,3-dihydro-1H-inden-2- amine hydrochloride (M + 1) 384.27, 0.46 min (LC/MS method E) | Method B Used Intermediate-8 4 M HCl in dioxane was used for deprotection in step 2 |
| B-69 | 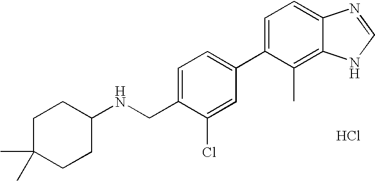 | N-{[2-Chloro-4-(4-methyl-1H- benzimidazol-5-yl)phenyl]methyl}- 4,4-dimethylcyclo hexanamine Hydrochloride white solid. ¹H NMR (400 MHz, DMSO-d₆) delta ppm 0.87 (m, 2 H) 1.03-1.40 (m, 4 H) 1.55-1.70 (m, 7 H) 2.57 (s, 3 H) 3.00 (m, 2 H) 4.21 (m, 2 H) 7.25- 7.42 (m, 3 H) 7.68-7.82 (m, 2 H) 9.21-9.38 (br., 2 H) 9.58 (s, 1 H); (M + 1) 366.28, 1.56 min (LC/MS method A) | Method B Used Intermediate- 75 4 M HCl in dioxane was used for deprotection in step 2 |
| B-70 | 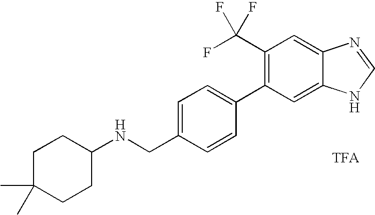 | N-({4-[5-(Trifluoromethyl)-1H- benzimidazol-6-yl]phenyl}methyl)- 2,3-dihydro-1H-inden-2-amine Trifluoroacetic Acid Salt pale yellow solid. At the deprotection stage, trifluoroacetic acid (3 ml) was used, instead of 4 M HCl in dioxane solution. ¹H NMR (400 MHz, DMSO-d₆) delta ppm 3.15 (m, 2 H) 3.38 (m, 2 H) 4.11 (m, 1 H) 4.38 (m, 2 H) 7.20- 7.25 (m, 4 H) 7.40-7.60 (m, 5 H) 8.08 (s, 1 H) 8.59 (s, 1 H) 9.10 (br., 2 H); (M + 1) 408.28, 1.65 min (LC/MS method A) | Method B Used Intermediate-4 in step 2 |
| B-71 | 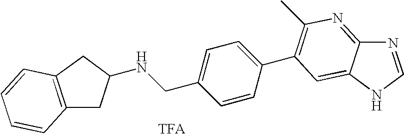 | N-{[4-(5-methyl-1H-imidazo[4,5- b]pyridin-6-yl)phenyl]methyl}-2,3- dihydro-1H-inden-2-amine trifluoroacetic acid salt (M + 1) 355.13, 1.16 min (LC/MS method A) | Method B Used Intermediate- 13 |
| B-72 | 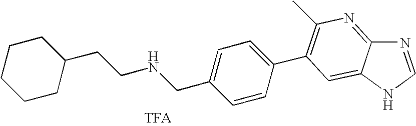 | (2-cyclohexylethyl){[4-(5-methyl-1H- imidazo[4,5-b]pyridin-6-yl)phenyl] methyl}amine trifluoroacetic acid salt (M + 1) 349.23, 1.49 min (LC/MS method A) | Method B Used Intermediate- 13 |
| B-73 | 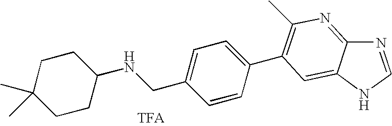 | (4,4-dimethylcyclohexyl){[4-(5- methyl-1H-imidazo[4,5-b]pyridin-6- yl)phenyl]methyl}amine trifluoroacetic acid salt (M + 1) 349.27, 1.35 min (LC/MS method A) | Method B Used Intermediate- 13 |
| B-74 | 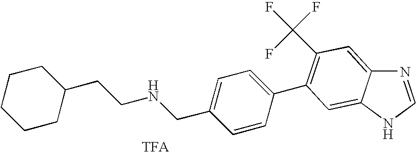 | (2-cyclohexylethyl)({4-[5- (trifluoromethyl)-1H-benzimidazol-6- yl]phenyl}methyl)amine trifluoroacetic acid salt (M − 1) 400.33, 1.88 min (LC/MS method A) | Method B Used Intermediate-4 in step 2 |
| B-75 | 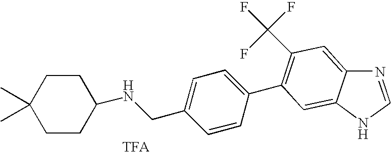 | (4,4-dimethylcyclohexyl)({4-[5- (trifluoromethyl)-1H-benzimidazol-6- yl]phenyl}methyl)amine trifluoroacetic acid salt (M − 1) 400.30, 1.80 min (LC/MS method A) | Method B Used Intermediate-4 in step 2 |
| B-76 | 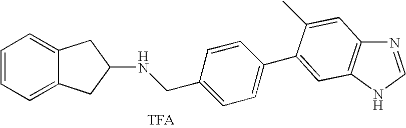 | N-{[4-(5-methyl-1H-benzimidazol-6- yl)phenyl]methyl}-2,3-dihydro-1H- inden-2-amine trifluoroacetic acid salt (M + 1) 354.18, 1.13 min (LC/MS method A) | Method B Used Intermediate- 17 |
| B-77 | 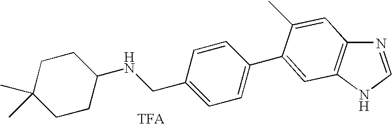 | (4,4-dimethylcyclohexyl){[4-(5- methyl-1H-benzimidazol-6- yl)phenyl]methyl}amine trifluoroacetic acid salt (M + 1) 348.23, 1.26 min (LC/MS method A) | Method B Used Intermediate- 17 |
| B-78 | 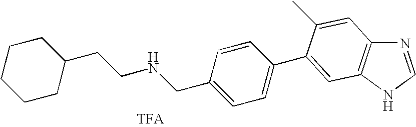 | (2-cyclohexylethyl){[4-(5-methyl-1H- benzimidazol-6- yl)phenyl]methyl}amine trifluoroacetic acid salt (M + 1) 348.24, 1.36 min (LC/MS method A) | Method B Used Intermediate- 17 |
| B-79 | 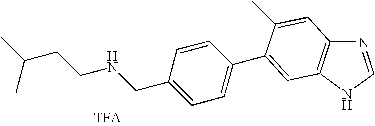 | {[4-(5-methyl-1H-benzimidazol-6- yl)phenyl]methyl}(3- methylbutyl)amine trifluoroacetic acid salt (M + 1) 308.16, 0.96 min (LC/MS method A) | Method B Used Intermediate- 17 |
| B-80 | 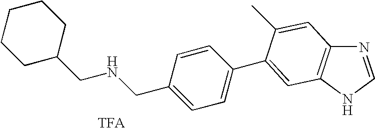 | (cyclohexylmethyl){[4-(5-methyl-1H- benzimidazol-6- yl)phenyl]methyl}amine trifluoroacetic acid salt (M + 1) 334.21, 1.12 min (LC/MS method A) | Method B Used Intermediate- 17 |
| B-81 | 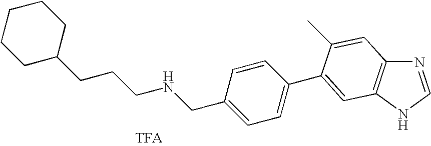 | (3-cyclohexylpropyl){[4-(5-methyl- 1H-benzimidazol-6- yl)phenyl]methyl}amine trifluoroacetic acid salt (M + 1) 362.25, 1.52 min (LC/MS method A) | Method B Used Intermediate- 17 |
| B-82 | 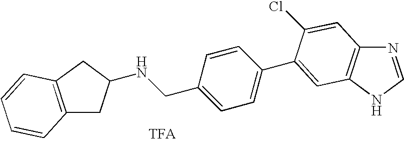 | N-{[4-(5-chloro-1H-benzimidazol-6- yl)phenyl]methyl}-2,3-dihydro-1H- inden-2-amine trifluoroacetic acid salt (M + 1) 374.14, 1.39 min (LC/MS method A) | Method B Used Intermediate- 16 |
| B-83 | 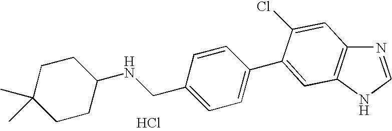 | N-{[4-(5-chloro-1H-benzimidazol-6- yl)phenyl]methyl}-4,4- dimethylcyclohexanamine hydrochloride (M + 1) 368.21, 1.54 min (LC/MS method A) | Method B Used Intermediate- 16 |
| B-84 | 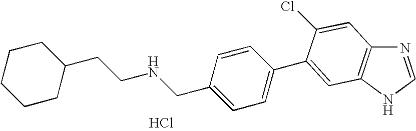 | N-{[4-(5-chloro-1H-benzimidazol-6- yl)phenyl]methyl}-2- cyclohexylethanamine hydrochloride (M + 1) 368.21, 1.64 min (LC/MS method A) | Method B Used Intermediate- 16 |
| B-85 | 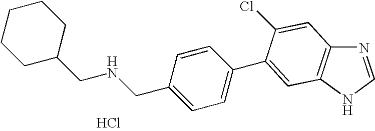 | {[4-(5-chloro-1H-benzimidazol-6- yl)phenyl]methyl}(cyclohexyl methyl)amine hydrochloride (M + 1) 354.21, 1.41 min (LC/MS method A) | Method B Used Intermediate- 16 |
| B-86 | 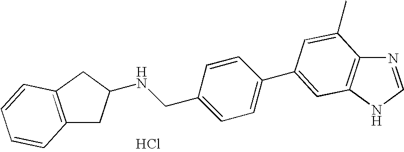 | N-{[4-(4-methyl-1H-benzimidazol-6- yl)phenyl]methyl}-2,3-dihydro-1H- inden-2-amine hydrochloride (M + 1) 354.20, 1.25 min (LC/MS method A) | Method B Used Intermediate- 15 4 M HCl in dixoane was used for deprotection in step 2 |
| B-87 | 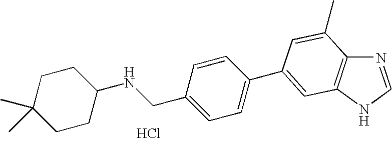 | (4,4-dimethylcyclohexyl){[4-(4- methyl-1H-benzimidazol-6- yl)phenyl]methyl}amine hydrochloride (M +) 348.26, 1.40 min (LC/MS method A) | Method B Used Intermediate- 15 4 M HCl in dixoane was used for deprotection in step 2 |
| B-88 | 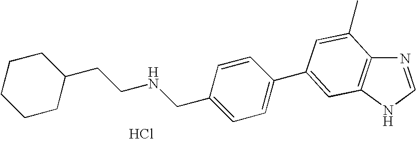 | (2-cyclohexylethyl){[4-(4-methyl-1H- benzimidazol-6- yl)phenyl]methyl}amine hydrochloride M + 1) 348.26, 1.45 min (LC/MS method A) | Method B Used Intermediate- 15 4 M HCl in dixoane was used for deprotection in step 2 |
| B-89 | 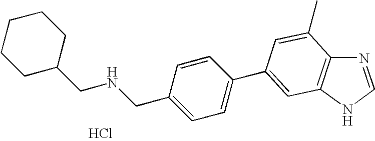 | (cyclohexylmethyl){[4-(4-methyl-1H- benzimidazol-6- yl)phenyl]methyl}amine hydrochloride (M + 1) 334.21, 1.25 min (LC/MS method A) | Method B Used Intermediate- 15 4 M HCl in dixoane was used for deprotection in step 2 |
| B-90 | 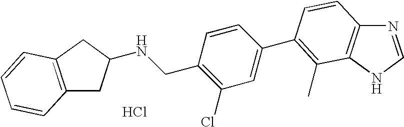 | N-{[2-chloro-4-(4-methyl-1H- benzimidazol-5-yl)phenyl]methyl}- 2,3-dihydro-1H-inden-2-amine hydrochloride (M + 1) 388.18, 0.50 min (LC/MS method E) | Method B Used Intermediate- 75 4 M HCl in dixoane was used for deprotection in step 2 |
| B-91 | 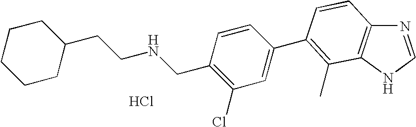 | N-{[2-chloro-4-(4-methyl-1H- benzimidazol-5-yl)phenyl]methyl}-2- cyclohexylethanamine hydrochloride (M + 1) 382.21, 0.54 min (LC/MS method E) | Method B Used Intermediate- 75 4 M HCl in dixoane was used for deprotection in step 2 |
| B-92 | 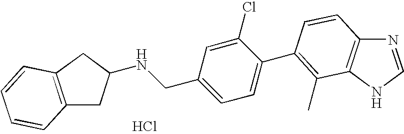 | N-{[3-chloro-4-(4-methyl-1H- benzimidazol-5-yl)phenyl]methyl}- 2,3-dihydro-1H-inden-2-amine hydrochloride (M + 1) 388.21, 0.48 min (LC/MS method E) | Method B Used Intermediate-14 4 M HCl in dixoane was used for deprotection in step 2 |
| B-93 | 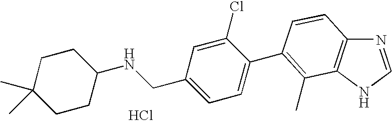 | N-{[3-chloro-4-(4-methyl-1H- benzimidazol-5-yl)phenyl]methyl}- 4,4-dimethylcyclo hexanamine hydrochloride (M + 1) 382.19, 0.53 min (LC/MS method E) | Method B Used Intermediate-14 4 M HCl in dixoane was used for deprotection in step 2 |
| B-94 | 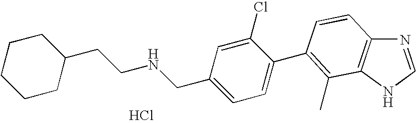 | N-{[3-chloro-4-(4-methyl-1H- benzimidazol-5-yl)phenyl]methyl}-2- cyclohexylethan amine hydrochloride (M + 1) 382.30, 1.48 min (LC/MS method B) | Method B Used Intermediate-14 4 M HCl in dixoane was used for deprotection in step 2 |
| B-95 | 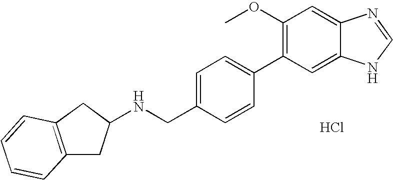 | N-({4-[5-(Methoxy)-1H-benz- imidazol-6-yl]phenyl}methyl)-2,3- dihydro-1H-inden-2-amine hydrochloride off-white solid ¹H NMR (400 MHz, DMSO-d₆) delta ppm 3.20-3.45 (m, 4 H) 3.82 (s, 3 H) 4.03 (m, 1 H) 4.24 (m, 2 H) 7.12- 7.30 (m, 4 H) 7.40 (s, 1 H) 7.52-7.78 (m, 5 H) 8.08 (s, 1 H) 9.40 (s, 1 H) 9.78 (br., 2 H); (M + 1) 370.19, 1.12 min (LC/MS method A) | Method B Used Intermediate-6 4 M HCl in dixoane was used for deprotection in step 2 |
| B-96 | 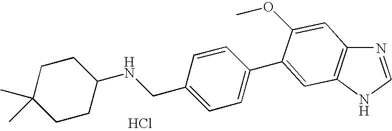 | (4,4-dimethylcyclohexyl)({4-[5- (methyloxy)-1H-benzimidazol-6- yl]phenyl}methyl)amine hydrochloride (M + 1) 364.26, 1.32 min (LC/MS method A) | Method B Used Intermediate-6 4 M HCl in dixoane was used for deprotection in step 2 |
| B-97 | 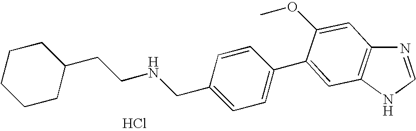 | (2-cyclohexylethyl)({4-[5- (methyloxy)-1H-benzimidazol-6- yl]phenyl}methyl)amine hydrochloride (M + 1) 364.26, 1.34 min (LC/MS method A) | Method B Used Intermediate-6 4 M HCl in dixoane was used for deprotection in step 2 |
| B-98 | 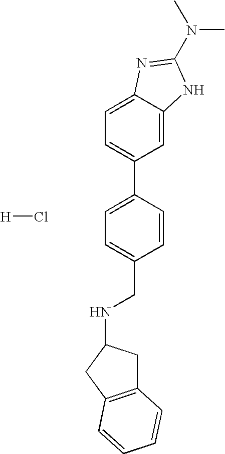 | 5-{4-[(2,3-dihydro-1H-inden-2- ylamino)methyl]phenyl}-N,N- dimethyl-1H-benzimidazol-2-amine hydrochloride (M + 1, free base) 383.33, 0.56 min (LC/MS Method E) | Method B Used Intemediate-18 in step 1 The protection/ deprotection of the benzimidazole NH was not necessary in this case. The final compound was isolated as the free base then treated with 1 equivalent of HCl (1.0 M in Et₂O.) |
| B-99 | 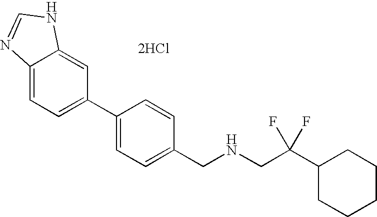 | N-{[4-(1H-benzimidazol-5- yl)phenyl]methyl}-2-cyclo hexyl-2,2- difluoroethan amine dihydrochloride ¹H NMR (400 MHz, methanol-d₄) delta ppm 1.12-1.39 (m, 5H) 1.68- 2.02 (m, 6H) 3.35 (t, J = 16.11 Hz, 2H) 4.38 (s, 2H) 7.67 (d, J = 8.30 Hz, 2H) 7.85 (d, J = 8.30 Hz, 2H) 7.93-7.95 (m, 2H) 8.07-8.08 (m, 1 H) 9.39 (s, 1 H); (M + 1) 370.25 ES, 0.63 minutes (LC/MS method E) | Method B Used Amine-6 Reductive amination reaction time 4 h. Deprotection was accomplished using 6 M HCl (aq) in EtOH at 70 degrees Celsius for 2 hr. Product crystallized out of solution on cooling. |

General Method C:

### Example C-1

### Step 1: Preparation of 5-bromo-1H-benzimidazole

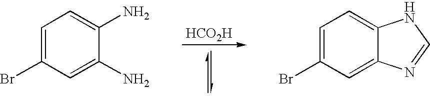

[symbol]

means “at reflux”.

A solution of 4-bromo-o-phenylenediamine (2.0 g, 10.7 mmol) in formic acid (5 mL) was heated at reflux for 2 h. After cooling to room temperature, 10% aqueous NaOH was added slowly until the mixture was basic, then the mixture was extracted with ethyl acetate (3×). The combined organic extracts were washed with brine, dried (Na₂SO₄), and concentrated in vacuo. Chromatography (5% to 10% of 2M NH₃/MeOH in CH₂Cl₂) of the residue provided the product 5-bromo-1H-benzimidazole as a brown foam. ¹H NMR (400 MHz, CDCl₃): δ—8.45 (br s, 1H), 8.14 (s, 1H), 7.81 (d, 1H, J=1.8 Hz), 7.52 (d, 1H, J=8.7 Hz), 7.39 (dd, 1H, J=1.8, 8.7 Hz).

### Step 2: Preparation of 1,1-dimethylethyl 6-bromo-1H-benzimidazole-1-carboxylate and 1,1-dimethylethyl 5-bromo-1H-benzimidazole-1-carboxylate

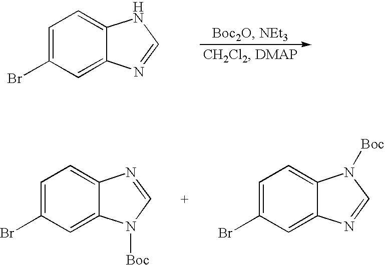

To a solution of 5-bromo-1H-benzimidazole (1.0 g, 5.1 mmol) in dichloromethane (25 mL) was added triethylamine (3.6 mL, 25.5 mmol) followed by di-t-butyldicarbonate (3.3 g, 15.2 mmol) and 4-(dimethylamino)-pyridine (cat). The reaction was stirred at room temperature for 30 min, then was quenched with H₂O. The layers were separated and the aqueous extracted 2× CH₂Cl₂. The combined organics were dried (Na₂SO₄), concentrated in vacuo, and chromatographed (10-20% EtOAc/hexanes). This provided the product as the two Boc regioisomers which were separated and characterized but not assigned as to the specific regioisomer. The more polar isomer was used for the subsequent reactions.

Isomer a (less polar): ¹H NMR (400 MHz, CDCl₃): δ—8.40 (s, 1H), 7.92 (s, 1H), 7.86 (d, 1H, J=8.5 Hz), 7.49 (d, 1H, J=8.5 Hz), 1.69 (s, 9H).

Isomer b (more polar): ¹H NMR (400 MHz, CDCl₃): δ—8.38 (s, 1H), 8.18 (s, 1H), 7.63 (d, 1H, J=8.5 Hz), 7.46 (d, 1H, J=8.5 Hz), 1.69 (9H).

### Step 3: Preparation of 1,1-dimethylethyl 5-(3-formylphenyl)-1H-benzimidazole-1-carboxylate

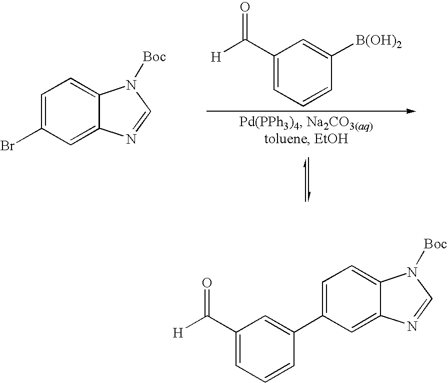

To a solution of 1,1-dimethylethyl 5-bromo-1H-benzimidazole-1-carboxylate (1.4 g, 3.2 mmol) in toluene (10 mL) was added Pd(PPh₃)₄ (0.18 g, 0.16 mmol), followed by a solution of Na₂CO₃ (0.85 g, 8.0 mmol) in water (5 mL) under N₂ atmosphere. To the reaction mixture was then added a solution of 3-formylphenylboronic acid (0.53 g, 3.5 mmol) in ethanol (8 mL). The reaction was heated at reflux overnight, then the solvent was removed in vacuo and the residue was partitioned between ethyl acetate and water. The organics were dried (Na₂SO₄), concentrated in vacuo, and chromatographed (35-50% EtOAc/hexanes) which provided the product 1,1-dimethylethyl 5-(3-formylphenyl)-1H-benzimidazole-1-carboxylate as a white solid.

¹H NMR (400 MHz, CDCl₃): δ—10.10 (s, 1H), 8.46 (s, 1H), 8.31 (s, 1H), 8.16 (s, 1H), 7.94-7.86 (m, 3H), 7.65-7.61 (m, 2H), 1.71 (s, 9H).

### Step 4: Preparation of 1,1-dimethylethyl 5-(3-{[(cyclohexylmethyl)amino]methyl}phenyl)-1H-benzimidazole-1-carboxylate

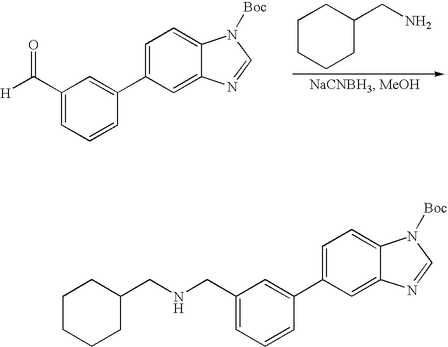

To a solution of 1,1-dimethylethyl 5-(3-formylphenyl)-1H-benzimidazole-1-carboxylate (0.20 g, 0.62 mmol) in methanol (5 mL) was added cyclohexanemethylamine (89 μL, 0.68 mmol). The reaction was stirred 2 h at room temperature, then sodium cyanoborohydride (97 mg, 1.55 mmol) was added and the reaction was stirred at room temperature overnight. The reaction was quenched with H₂O and the mixture was extracted 2× CHCl₃. The combined organics were washed with sat. NaHCO₃, dried over Na₂SO₄ and concentrated in vacuo. Chromatography (5-10% MeOH/CH₂Cl₂) provided the product, 1,1-dimethylethyl 5-(3-{[(cyclohexylmethyl)amino]methyl}phenyl)-1H-benzimidazole-1-carboxylate, as a white foam. ¹H NMR (400 MHz, CDCl₃): δ—8.43 (s, 1H), 8.25 (s, 1H), 7.82 (d, 1H, J=8.3 Hz), 7.63-7.53 (m, 3H), 7.43-7.43 (m, 2H), 3.87 (s, 2H), 2.51 (d, 2H, J=8.0 Hz), 1.80-1.50 (m, 15H), 1.27-1.12 (m, 3H), 0.92 (m, 2H).

### Step 5: Preparation of {[3-(1H-benzimidazol-5-yl)phenyl]methyl}-(cyclohexylmethyl)amine hydrochloride

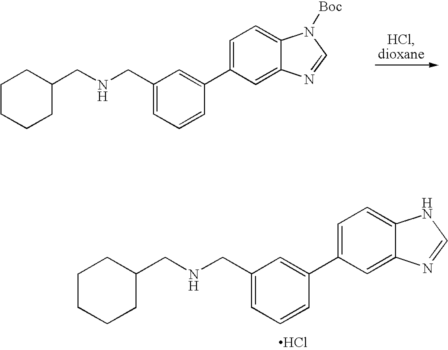

To a solution of 5-(3-{[(cyclohexylmethyl)amino]methyl}phenyl)-1H-benzimidazole-1-carboxylate (0.16 g, 0.38 mmol) in dioxane (2.5 mL) was added 4M HCl in dioxane (2.5 mL) and the reaction was stirred at room temperature overnight. The solvent was removed in vacuo, then the white solid was washed with hot ethyl acetate. The resulting white solid was dried under vacuum to provide the product, {[3-(1H-benzimidazol-5-yl)phenyl]methyl}-(cyclohexylmethyl)amine hydrochloride. (M+1) 320.2 ES, 1.17 min (LC/MS Method A).

TABLE 4
Compounds Prepared by General Method C

| | | Name and Characterization | |
|---|---|---|---|
| Ex. | Structure | Data | Method/Comments |
| C-2 | 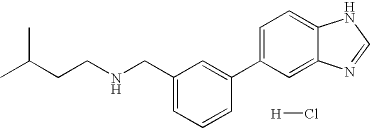 | N-{[3-(1H-benzimidazol-5- yl)phenyl]methyl}-3-methyl-1- butanamine hydrochloride (M + 1) 294.2 ES, 0.89 min. (LC/MS method A) | Method C Boc deprotection occurred under reductive amination conditions |

General Method D:

### Example D-1

### Step 1: Preparation of 5-bromo-1-(triphenylmethyl)-1H-benzimidazole and 6-bromo-1-(triphenylmethyl)-1H-benzimidazole

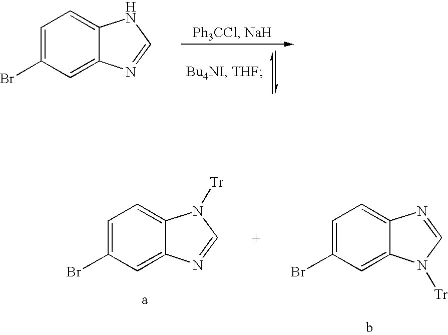

To a solution of 5-bromo-1H-benzimidazole (2.0 g, 10.2 mmol) in tetrahydrofuran (50 mL) at 0 degrees C. was added sodium hydride (0.49 g, 12.2 mmol) as a solid in 3 portions. The reaction mixture was stirred 30 min. at 0 degrees C., then trityl chloride (3.68 g, 13.2 mmol) was added, followed by tetra-n-butylammonium iodide (cat.). The reaction was then heated at reflux for 1.5 h. After cooling to room temperature, the reaction was quenched with H₂O and the mixture was extracted with CHCl₃ (3×). The combined organic extracts were dried (Na₂SO₄), concentrated in vacuo, and chromatographed (20-35% EtOAc/hexanes), to provide 5-bromo-1-(triphenylmethyl)-1H-benzimidazole (isomer a) and 6-bromo-1-(triphenylmethyl)-1H-benzimidazole (isomer b), each as white solids (˜1:1 mixture). Alternatively, a mixture of the two isomers could be obtained by suspension of the crude product in hot ethyl acetate. Isolation of the solid via filtration provided a mixture of a and b as an off-white powder.

a: ¹H NMR (400 MHz, CDCl₃): δ—7.92 (d, 1H, J=1.7 Hz), 7.87 (s, 1H), 7.35-7.29 (m, 10H), 7.16-7.12 (m, 5H), 6.99 (dd, 1H, J=1.7, 8.8 Hz), 6.34 (d, 1H, J=8.8 Hz).

b: ¹H NMR (400 MHz, CDCl₃): δ—7.87 (s, 1H), 7.63 (d, 1H, J=8.5 Hz), 7.36-7.30 (m, 10H), 7.26 (m, 1H), 7.17-7.12 (m, 5H), 6.57 (d, 1H, J=1.8 Hz).

### Step 2: Preparation of 3-[1-(triphenylmethyl)-1H-benzimidazol-5-yl]benzaldehyde and 3-[1-(triphenylmethyl)-1H-benzimidazol-6-yl]benzaldehyde

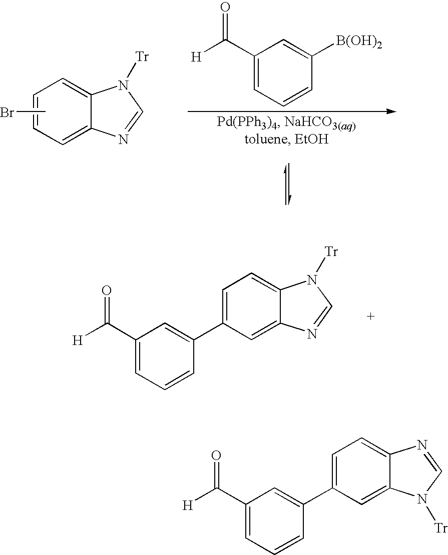

To a solution of 5-bromo-1-(triphenylmethyl)-1H-benzimidazole and 6-bromo-1-(triphenylmethyl)-1H-benzimidazole (˜1:1 mixture) (6.0 g, 13.7 mmol) in toluene (30 mL), was added tetrakis(triphenylphosphine) palladium(0) (0.8 g, 0.7 mmol), followed by a solution of sodium bicarbonate (2.9 g, 34.2 mmol) in water (15 mL). A solution of 3-formylphenylboronic acid (2.3 g, 15.0 mmol) in ethanol (8 mL) was then added and the reaction was heated at reflux overnight. After cooling, the bulk of the solvent was removed in vacuo, and the residue was partitioned between ethyl acetate and water. The organics were washed with brine, dried (Na₂SO₄), and concentrated in vacuo. Chromatography of the residue on silica gel (35-50% EtOAc/hexanes) provided the product as an inseparable mixture of trityl regioisomers, 3-[1-(triphenylmethyl)-1H-benzimidazol-5-yl]benzaldehyde and 3-[1-(triphenylmethyl)-1H-benzimidazol-6-yl]benzaldehyde. (M+1) 465.2 ES, 3.01 min and 3.05 min (LC-MS Method A).

### Step 3: Preparation of 4,4-dimethylcyclohexyl)({3-[1-(triphenylmethyl)-1H-benzimidazol-6-yl]phenyl}methyl)amine and (4,4-dimethylcyclohexyl)({3-[1-(triphenylmethyl)-1H-benzimidazol-5-yl]phenyl}methyl)amine

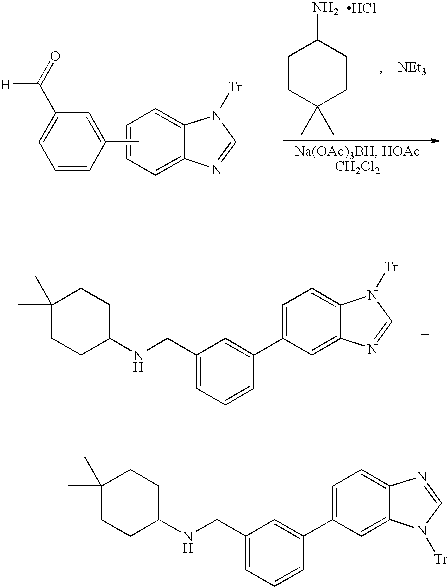

To a solution of 3-[1-(triphenylmethyl)-1H-benzimidazol-5-yl]benzaldehyde and 3-[1-(triphenylmethyl)-1H-benzimidazol-6-yl]benzaldehyde (0.22 g, 0.47 mmol) in dichloromethane (5 mL) was added 1 drop of acetic acid, followed by 4,4-dimethylcyclohexylamine hydrochloride (78 mg, 0.47 mmol) and triethylamine (66 μL, 0.47 mmol). NOTE: If a free amine was used, triethylamine was not added. After stirring 60 min at room temperature, sodium triacetoxyborohydride (0.40 g, 1.9 mmol) was added and the reaction was stirred an additional 3 h. The reaction mixture was diluted with H₂O and CH₂Cl₂. The layers were separated and the aqueous layer was extracted 2× CH₂Cl₂. The combined organics were washed with brine, dried over Na₂SO₄, and concentrated in vacuo. Chromatography provided the reductive amination product as a mixture of trityl regioisomers, (4,4-dimethylcyclohexyl)({3-[1-(triphenylmethyl)-1H-benzimidazol-6-yl]phenyl}methyl)amine and (4,4-dimethylcyclohexyl)({3-[1-(triphenylmethyl)-1H-benzimidazol-5-yl]phenyl}methyl)amine. (M+1) 576.4 ES, 2.40 min and 2.51 min (LC/MS Method A).

### Step 4: Preparation of {[3-(1H-benzimidazol-5-yl)phenyl]methyl}(4,4-dimethylcyclohexyl)amine hydrochloride

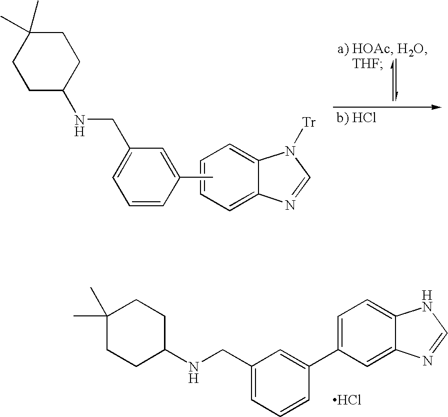

To a solution of (4,4-dimethylcyclohexyl)({3-[1-(triphenylmethyl)-1H-benzimidazol-6-yl]phenyl}methyl)amine and (4,4-dimethylcyclohexyl)({3-[1-(triphenylmethyl)-1H-benzimidazol-5-yl]phenyl}methyl)amine (0.18 g, 0.31 mmol) in tetrahydrofuran (3 mL) were added water (3 mL) and acetic acid (3 mL). The reaction mixture was heated at reflux for 1 h, then was allowed to cool to room temperature and was treated with 10% HCl (aq) until pH 2. The mixture was washed 2× EtOAc, then the aqueous phase was treated with solid K₂CO₃ until it reached pH 10, at which point it was extracted 3× CHCl₃. The combined CHCl₃ extracts were dried (Na₂SO₄) and concentrated in vacuo. The residue was taken up in Et₂O and enough acetone to dissolve completely, then 4M HCl in dioxane was added until no more solid crashed out. The white solid was collected by filtration and dried under vacuum, providing the product {[3-(1H-benzimidazol-5-yl)phenyl]methyl}(4,4-dimethylcyclohexyl)amine hydrochloride (M+1) 334.2 ES, 1.30 min (LC/MS Method A).

TABLE 5
Compounds Prepared by General Method D

| | | | |
|---|---|---|---|
| | | Name and Characterization | Method |
| Ex. | Structure | Data | Comments |

| | | | |
|---|---|---|---|
| D-2 | 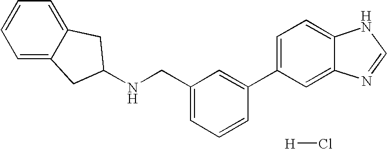 | N-{[3-(1H-benzimidazol-5- yl)phenyl]methyl}-2,3- dihydro-1H-inden-2-amine hydrochloride (M + 1) 340.1 AP, 1.10 min. (LC/MS method B) | Method D Imine preformation for 20 minutes in red. amination |
| D-3 | 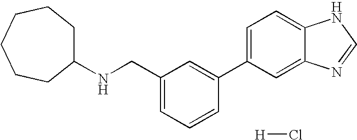 | N-{[3-(1H-benzimidazol-5- yl)phenyl]methyl}cycloheptan amine hydrochloride (M + 1) 320.2 AP, 1.11 min. (LC/MS method B) | Method D Imine preformation for 20 minutes in red. amination |
| D-4 | 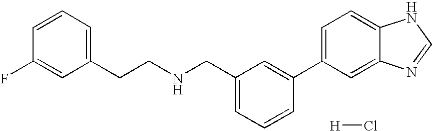 | N-{[3-(1H-benzimidazol-5- yl)phenyl]methyl}-2-(3- fluorophenyl)ethanamine hydrochloride (M + 1) 346.1.2 AP, 1.16 min. (LC/MS method B) | Method D Imine preformation for 20 minutes in red. amination |
| D-5 | 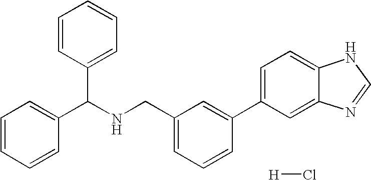 | N-{[3-(1H-benzimidazol-5- yl)phenyl]methyl}-1,1- diphenylmethanamine hydrochloride (M + 1) 390.1 AP, 1.42 min. (LC/MS method B) | Method D |
| D-6 | 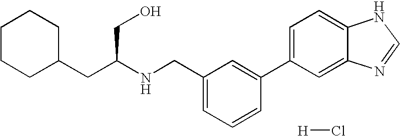 | (2S)-2-({[3-(1H- benzimidazol-5- yl)phenyl]methyl}amino)-3- cyclohexyl-1-propanol hydrochloride (M + 1) 364.2 AP, 1.63 min. (LC/MS method B) | Method D Imine preformation for 20 minutes in red. amination |
| D-7 | 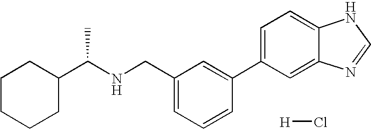 | {[3-(1H-benzimidazol-5- yl)phenyl]methyl}[(1S)-1- cyclohexylethyl]amine hydrochloride (M + 1) 334.2 AP, 1.28 min. (LC/MS method B) | Method D Imine preformation for 20 minutes in red. amination |
| D-8 | 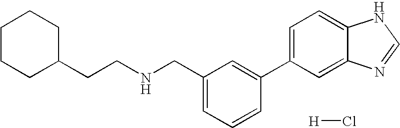 | N-{[3-(1H-benzimidazol-5- yl)phenyl]methyl}-2- cyclohexylethanamine hydrochloride (M + 1) 334.2 ES, 1.37 min. (LC/MS method A) | Method D Imine preformation for 20 minutes in red. amination |

General Method E:

### Example E-1

### Step 1: Preparation of 2-fluoro-4-[1-(triphenylmethyl)-1H-benzimidazol-5-yl]benzaldehyde and 2-fluoro-4-[1-(triphenylmethyl)-1H-benzimidazol-6-yl]benzaldehyde

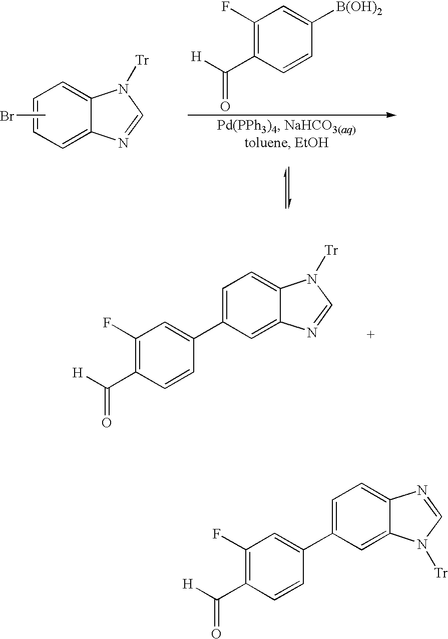

To a solution of 5-bromo-1-(triphenylmethyl)-1H-benzimidazole and 6-bromo-1-(triphenylmethyl)-1H-benzimidazole (˜1:1 mixture) (4.0 g, 9.1 mmol) in toluene (30 mL), was added tetrakis(triphenylphosphine) palladium(0) (0.5 g, 0.5 mmol), followed by a solution of sodium bicarbonate (1.9 g, 22.8 mmol) in water (15 mL). A solution of 3-fluoro-4-formylphenylboronic acid (1.7 g, 10.0 mmol) in ethanol (8 mL) was then added and the reaction was heated at reflux overnight. After cooling, the bulk of the solvent was removed in vacuo, and the residue was partitioned between ethyl acetate and water. The organics were washed with brine, dried (Na₂SO₄), and concentrated in vacuo. Chromatography of the residue on silica gel (35-50% EtOAc/hexanes) provided the product as an inseparable mixture of trityl regioisomers 2-fluoro-4-[1-(triphenylmethyl)-1H-benzimidazol-5-yl]benzaldehyde and 2-fluoro-4-[1-(triphenylmethyl)-1H-benzimidazol-6-yl]benzaldehyde. (M+1) 483.2 ES, 3.12 min and 3.18 min (LC/MS Method A).

The following biaryl aldehydes were synthesized in an analogous manner:

TABLE 6
Aldehyde Intermediates Prepared by General Method E (Step 1)

| Intermediate | Structure | Name | Comments¹ |
|---|---|---|---|
| Intermediate-19 | 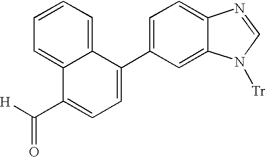 | 4-[1-(triphenylmethyl)-1H- benzimidazol-6-yl]-1- naphthalenecarbaldehyde | Prepared using 4- formyl-1- naphthalene boronic acid |
| Intermediate-21 | 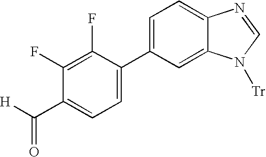 | 2,3-difluoro-4-[1- (triphenylmethyl)-1H- benzimidazol-6-yl] benzaldehyde | Prepared as described above using (2,3- difluoro-4- formylphenyl) boronic acid |
| Intermediate-22 | 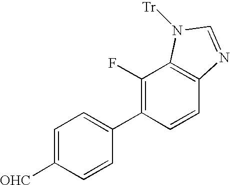 | 4-[7-fluoro-1-(triphenyl- methyl)-1H-benzimidazol- 6-yl]benzaldehyde (M + 1) 483, 2.9 min, (LC/MS Method A) | Used Intermediate-23 Used dppf•PdCl₂• CH₂Cl₂ as catalyst. Used 2.0 M aqueous Na₂CO₃. Reaction was heated to 80° C. for 3.5 hr. Dried over MgSO₄. Chromatography used 10-40% EtOAc/hexanes |
| Intermediate-24 | 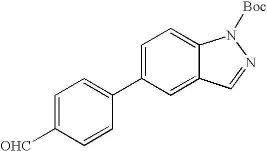 | 1,1-dimethylethyl 5-(4- formylphenyl)-1H-indazole- 1-carboxylate (M + 1 (− BOC)) 223, 0.84 min, (LC/MS Method A) | Used Intermediate- 25 Used dppf•PdCl₂• CH₂Cl₂ as catalyst. Used 2.0 M aqueous Na₂CO₃. Reaction was heated to 80° C. for 3.5 hr. Dried over MgSO₄. Chromatography used 10-40% EtOAc/hexanes |
| Intermediate-26 | 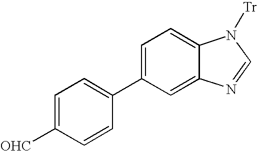 | 4-[1-(triphenylmethyl)-1H- imidazo[4,5-b]pyridin-5-yl] benzaldehyde | Used Intermediate- 27 Used dppf•PdCl₂• CH₂Cl₂ as catalyst. Used 2.0 M aqueous Na₂CO₃. Reaction was heated to 80° C. for 3.5 hr. Dried over MgSO₄. Chromatography used 10-40% EtOAc/hexanes |
| Intermediate-28 | 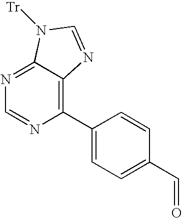 | 4-[9-(triphenylmethyl)-9H- purin-6-yl]benzaldehyde ¹H NMR (400 MHz, CDCl₃) delta ppm 7.18-7.36 (m, 15H) 8.05 (d, J = 8.30 Hz, 2H) 8.16 (s, 1H) 8.77 (s, 1H) 8.98 (d, J = 8.30 Hz, 2H) 10.11 (s, 1H) | Used Intermediate- 29 Used DME as solvent and aqueous Na₂CO₃ Heated at 80 degrees Celsius for 16 hr. |

¹Note:
Descriptions for the synthesis of intermediates referred to in this column and used to synthesize the above aldehydes can be found in the section entitled, “Synthetic Descriptions of Intermediates Used in General Methods A through K.”
* All biaryl aldehydes obtained as mixtures of trityl regioisomers and these mixtures were carried on to the subsequent reductive aminations.

### Step 2: Preparation of (4,4-dimethylcyclohexyl)({2-fluoro-4-[1-(triphenylmethyl)-1H-benzimidazol-6-yl]phenyl}methyl)amine and (4,4-dimethylcyclohexyl)({2-fluoro-4-[1-(triphenylmethyl)-1H-benzimidazol-5-yl]phenyl}methyl)amine

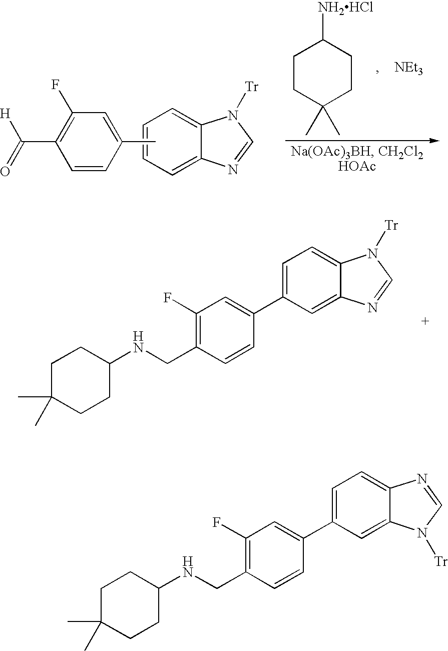

To a suspension of 2-fluoro-4-[1-(triphenylmethyl)-1H-benzimidazol-5-yl]benzaldehyde and 2-fluoro-4-[1-(triphenylmethyl)-1H-benzimidazol-6-yl]benzaldehyde (0.22 g, 0.46 mmol) and 4,4-dimethylcyclohexylamine hydrochloride (75 mg, 0.46 mmol) in dichloromethane (5 mL) was added triethylamine (64 μL, 0.46 mmol) followed by 1 drop of acetic acid. The mixture was stirred 20 min, then sodium triacetoxyborohydride (0.39 g, 1.84 mmol) was added and the reaction was stirred 3 h at room temperature. The reaction mixture was quenched with H₂O and extracted 2× CH₂Cl₂. The combined organic extracts were washed with brine, dried over Na₂SO₄ and concentrated in vacuo. Chromatography (5-10% MeOH/CH₂Cl₂) provided the reductive amination products (a mixture of trityl regioisomers), (4,4-dimethylcyclohexyl)({2-fluoro-4-[1-(triphenylmethyl)-1H-benzimidazol-6-yl]phenyl}methyl)amine and (4,4-dimethylcyclohexyl)({2-fluoro-4-[1-(triphenylmethyl)-1H-benzimidazol-5-yl]phenyl}methyl)amine, as a white solid. (M+1) 594.1 AP, 2.45 min and 2.53 min (LC/MS Method B).

### Step 3: Preparation of {[4-(1H-benzimidazol-5-yl)-2-fluorophenyl]methyl}(4,4-dimethylcyclohexyl)amine

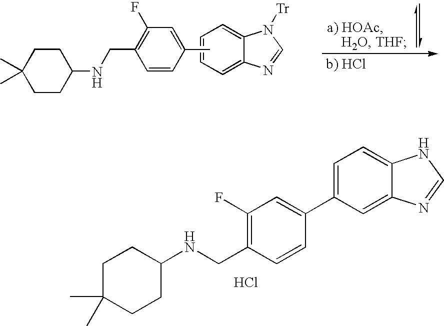

To a solution of (4,4-dimethylcyclohexyl)({2-fluoro-4-[1-(triphenylmethyl)-1H-benzimidazol-6-yl]phenyl}methyl)amine and (4,4-dimethylcyclohexyl)({2-fluoro-4-[1-(triphenylmethyl)-1H-benzimidazol-5-yl]phenyl}methyl)amine (0.15 g, 0.25 mmol) in tetrahydrofuran (3 mL) were added water (3 mL) and acetic acid (3 mL). The reaction mixture was heated at reflux for 1 h, then was allowed to cool to room temperature and treated with 10% HCl (aq) until pH 2. The mixture was washed 2× EtOAc, then the aqueous phase was treated with solid K₂CO₃ until it reached pH 10, at which point it was extracted 3× CHCl₃. The combined CHCl₃ extracts were dried (Na₂SO₄) and concentrated in vacuo. The residue was taken up in Et₂O and enough acetone to dissolve completely, then 4M HCl in dioxane was added until no more solid crashed out. The white solid was collected by filtration and dried under vacuum, providing the product {[4-(1H-benzimidazol-5-yl)-2-fluorophenyl]methyl}(4,4-dimethylcyclohexyl)amine hydrochloride as a white solid. (M+1) 352.2 ES, 1.36 min (LC/MS Method A).

TABLE 7
Compounds Prepared by General Method E

| | | Name and Characterization | Method/ |
|---|---|---|---|
| Ex. | Structure | Data | Comments |
| E-2 | 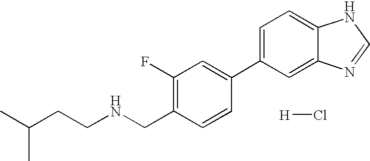 | N-{[4-(1H-benzimidazol-5-yl)- 2-fluorophenyl]methyl}-3- methyl-1-butanamine hydrochloride (M + 1) 312.1 ES, 0.92 min. (LC/MS method A) | Method E |
| E-3 | 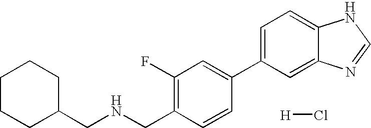 | {[4-(1H-benzimidazol-5-yl)-2- fluorophenyl]methyl}(cyclohexyl- methyl)amine hydrochloride (M + 1) 338.2 ES, 1.14 min. (LC/MS method A) | Method E |
| E-4 | 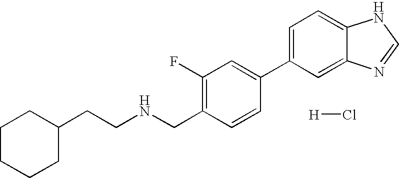 | N-{[4-(1H-benzimidazol-5-yl)- 2-fluorophenyl]methyl}-2- cyclohexylethanamine hydrochloride (M + 1) 352.2 ES, 1.45 min. (LC/MS method A) | Method E |
| E-5 | 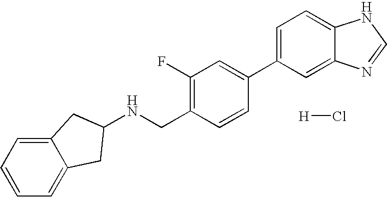 | N-{[4-(1H-benzimidazol-5-yl)- 2-fluorophenyl]methyl}-2,3- dihydro-1H-inden-2-amine hydrochloride (M + 1) 358.2 ES, 1.12 min. (LC/MS method A) | Method E |
| E-6 | 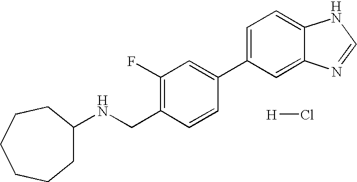 | N-{[4-(1H-benzimidazol-5-yl)- 2- fluorophenyl]methyl}cyclohept- anamine hydrochloride (M + 1) 338.1 AP, 1.17 min. (LC/MS method B) | Method E |
| E-7 | 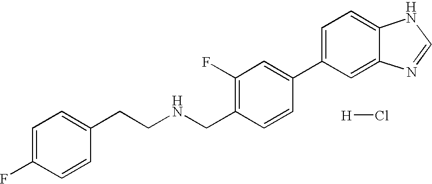 | N-{[4-(1H-benzimidazol-5-yl)- 2-fluorophenyl]methyl}-2-(4- fluorophenyl)ethanamine hydrochloride (M + 1) 364.1 AP, 1.15 min. (LC/MS method B) | Method E |
| E-8 | 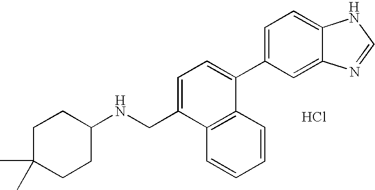 | N-{[4-(1H-benzimidazol-5-yl)- 1-naphthalenyl]methyl}-4,4- dimethylcyclohexanamine hydrochloride (M + 1) 384.2 AP, 1.52 min. (LC/MS method B) | Method E Used intermediate- 19 Only 1 eq. of HCl added to form salt |
| E-9 | 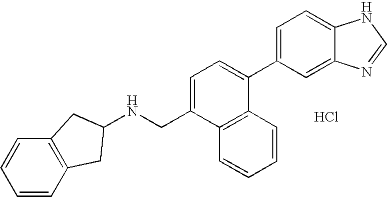 | N-{[4-(1H-benzimidazol-5-yl)- 1-naphthalenyl]methyl}-2,3- dihydro-1H-inden-2-amine hydrochloride (M + 1) 390.2 AP, 1.37 min. (LC/MS method B) | Method E Used intermediate- 19 Only 1 eq. of HCl added to form salt |
| E-10 | 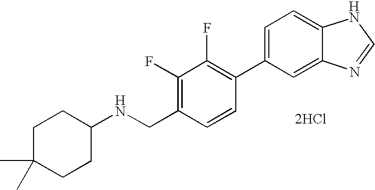 | N-{[4-(1H-benzimidazol-5-yl)- 2,3-difluorophenyl]methyl}-4,4- dimethylcyclohexanamine dihydrochloride (M + 1) 370.3 ES, 1.38 min. (LC/MS method A) | Method E Used intermediate- 21 2 eq. of HCl added to form salt |
| E-11 | 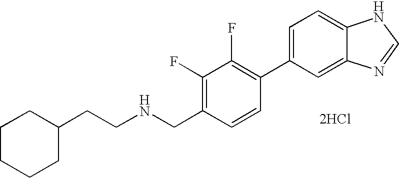 | N-{[4-(1H-benzimidazol-5-yl)- 2,3-difluorophenyl]methyl}-2- cyclohexylethanamine dihydrochloride (M + 1) 370.3 ES, 1.47 min. (LC/MS method A) | Method E Used intermediate- 21 2 eq. of HCl added to form salt |
| E-12 | 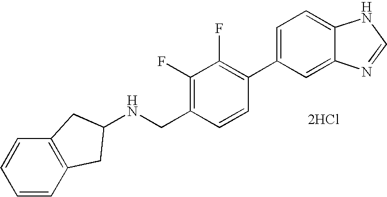 | N-{[4-(1H-benzimidazol-5-yl)- 2,3-difluorophenyl]methyl}-2,3- dihydro-1H-inden-2-amine dihydrochloride (M + 1) 376.2 ES, 1.24 min. (LC/MS method A) | Method E Used intermediate- 21 2 eq. of HCl added to form salt |
| E-13 | 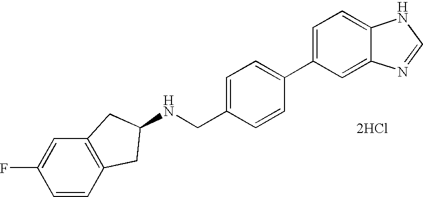 | {[4-(1H-benzimidazol-5- yl)phenyl]methyl}[(2S)-5- fluoro-2,3-dihydro-1H-inden-2- yl]amine dihydrochloride (M + 1) 358.3 ES, 1.10 min. (LC/MS method A) | Method E Used product from Method B, step 1 2 eq. of HCl added to form salt |
| E-14 | 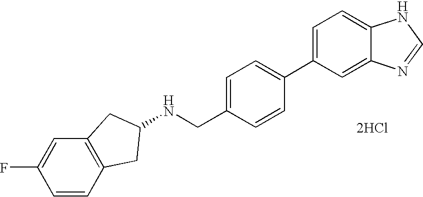 | {[4-(1H-benzimidazol-5- yl)phenyl]methyl}[(2R)-5- fluoro-2,3-dihydro-1H-inden-2- yl]amine dihydrochloride (M + 1) 358.3 ES, 1.07 min. (LC/MS method A) | Method E Used product from Method B, step 1 2 eq. of HCl added to form salt |
| E-15 | 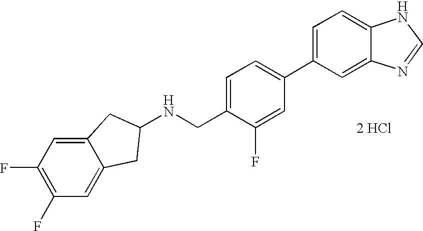 | N-{[4-(1H-benzimidazol-5-yl)- 2-fluorophenyl]methyl}-5,6- difluoro-2,3-dihydro-1H-inden- 2-amine dihydrochloride (M + 1) 394, 1.29 min. (LC/MS Method A) | Method E Used (8% MeOH/NH₃/ CH₂Cl₂) Used Amine-4 |
| E-16 | 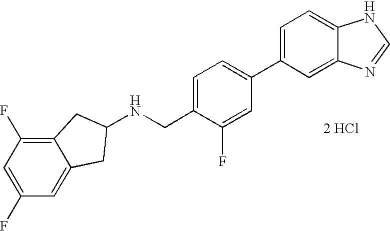 | N-{[4-(1H-benzimidazol-5-yl)- 2-fluorophenyl]methyl}-4,6- difluoro-2,3-dihydro-1H-inden- 2-amine dihydrochloride (M + 1) 394, 1.27. min (LC/MS Method A) | Method E Used (8% MeOH/NH₃/ CH₂Cl₂) Used Amine-5 |
| E-17 | 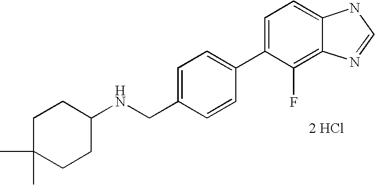 | N-{[4-(4-fluoro-1H-benz- imidazol-5-yl)phenyl]methyl}- 4,4-dimethylcyclohexan amine dihydrochloride (M − 1) 350, 1.3 min. (LC/MS Method A) | Method E Used Intermediate- 22 Aqueous phase was made basic with 1 N NaOH. Organic phase was dried with MgSO₄. CH₂Cl₂ was used as solvent for HCl formation with HCl/ dioxane |
| E-18 | 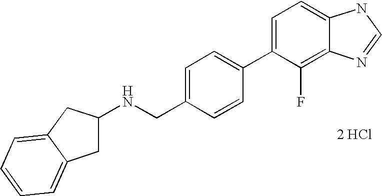 | N-{[4-(4-fluoro-1H-benz- imidazol-5-yl)phenyl]methyl}- 2,3-dihydro-1H-inden-2-amine dihydrochloride (M + 1) 358, 1.2 min. (LC/MS Method A) | Method E Used Intermediate- 22 Chromatography used 7% MeOH/ NH₃/ CH₂Cl₂ CHCl₃ phase was washed with aqueous NaHCO₃ |
| E-19 | 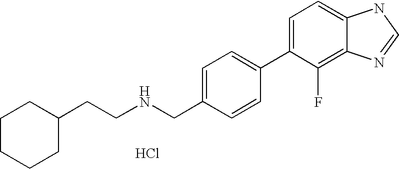 | (2-cyclohexylethyl){[4-(4- fluoro-1H-benzimidazol-5- yl)phenyl]methyl}amine hydrochloride (M − 1) 350, 1.3 min. (LC/MS Method A) | Method E Used Intermediate- 22 Aqueous phase was made basic with 1 N NaOH. Organic phase was dried with MgSO₄. CH₂Cl₂ was used as solvent for HCl formation with HCl/ dioxane |
| E-20 | 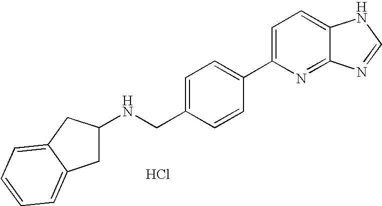 | N-{[4-(1H-indazol-5- yl)phenyl]methyl}-2,3-dihydro- 1H-inden-2-amine (M − 1) 338, 1.4 min. (LC/MS Method A) | Method E Used Intermediate- 24 Deprotection to give the final product was accomplished using CH₂CL₂ containing TFA. |
| E-21 | 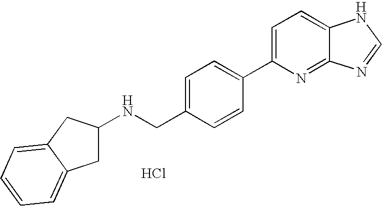 | N-{[4-(1H-imidazo[4,5- b]pyridin-5-yl)phenyl]methyl}- 2,3-dihydro-1H-inden-2-amine hydrochloride (M − 1) 339, 1.2 min. (LC/MS Method A) | Method E Used Intermediate- 26 Used 10% MeOH/NH₃/ CH₂Cl₂ for chromatography |
| E-22 | 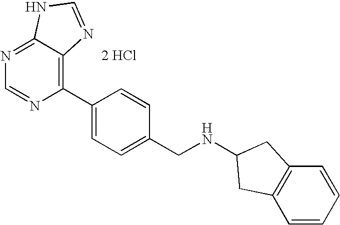 | N-{[4-(1H-purin-6- yl)phenyl]methyl}-2,3-dihydro- 1H-inden-2-amine dihydrochloride ¹H NMR (400 MHz, CDCl₃) delta ppm 2.84 (dd, J = 15.87, 6.59, 2H) 3.19 (dd, J = 15.87, 7.08, 2H) 3.67-3.75 (m, 1H) 3.98 (bs, 2H) 7.12-7.16 (m, 2H) 7.17-7.22 (m, 2H) 7.57 (d, J = 8.06 Hz, 2H) 8.28 (s, 1H) 8.79 (d, J = 7.57 Hz, 2H) 9.05 (s, 1H) (M + 1) 342.30, 0.60 min (LC/MS method E) | Method E Used Intermediate- 28 Reductive amination reaction time 4 h. Deprotection was accomplished using 6 M HCl (aq) in EtOH at 70 degrees Celsius for 2 hr. Product crystallized out of solution on cooling. |

General Method F:

### Example F-1

### Step 1: Preparation of 5-(4,4,5,5-tetramethyl-1,3,2-dioxaborolan-2-yl)-1-(triphenyl methyl)-1H-benzimidazole and 6-(4,4,5,5-tetramethyl-1,3,2-dioxaborolan-2-yl)-1-(triphenylmethyl)-1H-benzimidazole

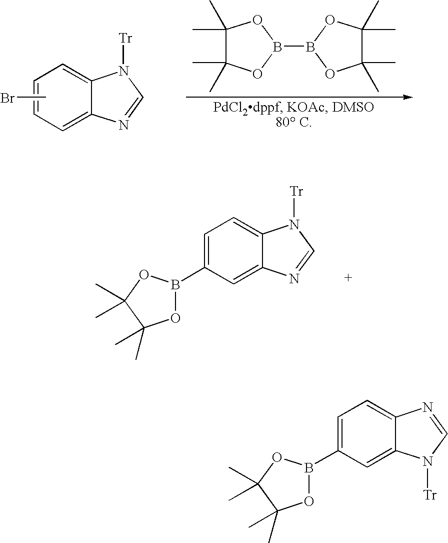

In a 500 mL round bottomed flask, bis(pinacolato)diboron (9.5 g, 37.5 mmol), potassium acetate (10.0 g, 102.4 mmol), and PdCl₂.dppf (1.2 g, 1.7 mmol) were combined. The flask was flushed with nitrogen and a slurry of 5-bromo-1-(triphenylmethyl)-1H-benzimidazole and 6-bromo-1-(triphenylmethyl)-1H-benzimidazole (15.0 g, 34.1 mmol) in dimethylsulfoxide (180 mL) was added and the reaction was heated at 80 degrees C. overnight. After cooling to room temperature, the mixture was diluted with chloroform and washed with water (2×) and brine. The organics were dried over sodium sulfate, concentrated in vacuo, and purified via chromatography (20 to 35% ethyl acetate/hexane), providing the product as a mixture of trityl regioisomers, 5-(4,4,5,5-tetramethyl-1,3,2-dioxaborolan-2-yl)-1-(triphenylmethyl)-1H-benzimidazole and 6-(4,4,5,5-tetramethyl-1,3,2-dioxaborolan-2-yl)-1-(triphenylmethyl)-1H-benzimidazole

Isomer a: ¹H NMR (400 MHz, CDCl₃) δ 1.19 (s, 12H), 6.84 (s, 1H), 7.15-7.18 (m, 6H), 7.29-7.33 (m, 9H), 7.61 (d, J=8.1 Hz, 1H), 7.76 (d, J=8.1 Hz, 1H), 7.97 (s, 1H).

Isomer b: ¹H NMR (400 MHz, CDCl₃) δ 1.25 (s, 12H), 6.45 (d, J=8.3 Hz, 1H), 7.16-7.14 (m, 6H), 7.34-7.27 (m, 10H), 7.90 (s, 1H), 8.28 (s, 1H).

### Step 2: Preparation of 2-methyl-4-[1-(triphenylmethyl)-1H-benzimidazol-6-yl]benzaldehyde and 2-methyl-4-[1-(triphenylmethyl)-1H-benzimidazol-5-yl]benzaldehyde

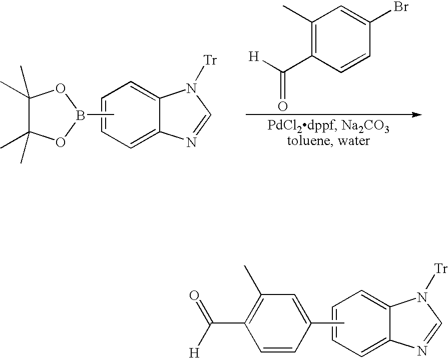

To a solution of 4-bromo-2-methylbenzaldehyde (Intermediate-30) (4.7 g, 23.4 mmol) in toluene (30 mL) under nitrogen atmosphere was added PdCl₂.dppf (0.9 g, 1.2 mmol), followed by a solution of sodium carbonate (6.2 g, 58.8 mmol) in water (40 mL) and a solution of 5-(4,4,5,5-tetramethyl-1,3,2-dioxaborolan-2-yl)-1-(triphenylmethyl)-1H-benzimidazole and 6-(4,4,5,5-tetramethyl-1,3,2-dioxaborolan-2-yl)-1-(triphenylmethyl)-1H-benzimidazole (11.4 g, 23.4 mmol) in toluene (60 mL). The reaction was heated at reflux for 5 h, then the solvent was removed in vacuo and the residue was partitioned between ethyl acetate and water. The organics were dried over sodium sulfate, concentrated in vacuo, and purified via chromatography (20 to 35% ethyl acetate/hexanes) to provide the product as mixture of trityl regioisomers, 2-methyl-4-[1-(triphenylmethyl)-1H-benzimidazol-6-yl]benzaldehyde and 2-methyl-4-[1-(triphenylmethyl)-1H-benzimidazol-5-yl]benzaldehyde, as a yellow solid. The product was carried on to the subsequent reductive amination as a mixture of regioisomers.

Isomer a: ¹H NMR (400 MHz, CDCl₃) δ 2.61 (s, 3H), 6.60 (s, 1H), 6.95 (s, 1H), 7.12 (dd, J=1.2 Hz, 8.1 Hz, 1H), 7.23-7.17 (m, 6H), 7.37-7.30 (m, 9H), 7.43 (dd, J=1.2 Hz, 8.6 Hz, 1H), 7.71 (d, J=8.1 Hz, 1H), 7.84 (d, J=8.6 Hz, 1H), 7.96 (s, 1H), 10.20 (s, 1H).

Isomer b: ¹H NMR (400 MHz, CDCl₃) δ 2.71 (s, 3H), 6.53 (d, J=8.5 Hz, 1H), 7.23-7.17 (m, 7H), 7.37-7.30 (m, 9H), 7.57 (d, J=8.1 Hz, 1H), 7.48 (s, 1H), 7.83 (d, J=8.1 Hz, 1H), 7.96 (s, 1H), 8.05 (s, 1H), 10.27 (s, 1H).

The following biaryl aldehyde intermediates were synthesized in an analogous manner:

TABLE 8
Aldehyde Intermediates Prepared by General Method G (Step 5)

| | | Name and | |
|---|---|---|---|
| Intermediate | Structure | Characterizing Data | Method/Comments¹ |
| Intermediate-66 | 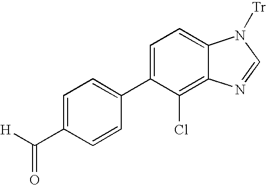 | 4-[4-chloro-1- (triphenylmethyl)-1H- benzimidazol-5-yl] benzaldehyde White solid ¹H NMR (400 MHz, CDCl₃) δ 6.44 (d, J = 8.6 Hz, 1H), 6.89 (d, J = 8.6 Hz, 1H), 7.17- 7.20 (m, 6H), 7.33- 7.35 (m, 9H), 7.64 (d, J = 8.0 Hz, 2H), 7.92 (d, J = 8.0 Hz, 2H), 7.98 (s, 1H), 10.05 (s, 1H). | Prepared as in Step 5 using Intermediate-67 and PdCl2.dppf as catalyst. Chromotography used 20 to 35% EtOAc/hexanes. |
| Intermediate-68* | 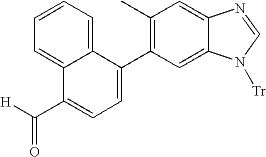 | 4-[5-methyl-1- (triphenyl methyl)-1H- benzimidazol-6-yl]- 1-naphthalene carbaldehyde Yellow solid | Method G Used Intermediates 20 and 69, and used Na₂CO₃ as base. Chromatography used 20-35% EtOAc/hexanes. |
| Intermediate-70 | 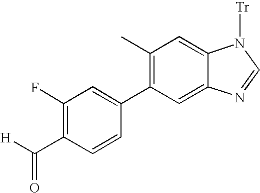 | 2-fluoro-4-[6-methyl-1- (triphenyl methyl)-1H- benz imidazol-5-yl] benzaldehyde (M + 1) 497.3, 3.13 min. (LC/MS Method A). | Method G Used Intermediate-69 and (3-fluoro-4-formylphenyl) boronic acid Chromatography used 20-35% EtOAc/hexanes. |
| Intermediate-71* | 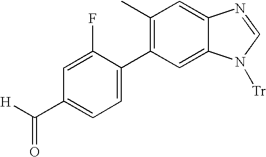 | 3-fluoro-4-[5-methyl-1- (triphenylmethyl)-1H- benzimidazol-6- yl]benzaldehyde | Method G Used Intermediate-69 and (2-fluoro-4-formylphenyl) boronic acid Chromatography used 20-35% EtOAc/hexanes. |
| Intermediate-72 | 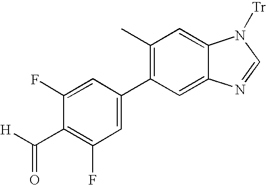 | 2,6-difluoro-4-[5- methyl-1- (triphenylmethyl)-1H- benzimidazol-6- yl]benzaldehyde | Method G Used Intermediate-69 and (3,5-difluoro-4-formyl phenyl)boronic acid Chromatography used 20-35% EtOAc/hexanes. |

¹Note:
Descriptions for the synthesis of intermediates referred to in this column and used to synthesize the above aldehydes can be found in the section entitled, “Synthetic Descriptions of Intermediates Used in General Method A through K.”
*Mixture of trityl regioisomers

### Step 3: Preparation of N-({2-methyl-4-[1-(triphenylmethyl)-1H-benzimidazol-6-yl]phenyl}methyl)-2,3-dihydro-1H-inden-2-amine and N-({2-methyl-4-[1-(triphenylmethyl)-1H-benzimidazol-5-yl]phenyl}methyl)-2,3-dihydro-1H-inden-2-amine

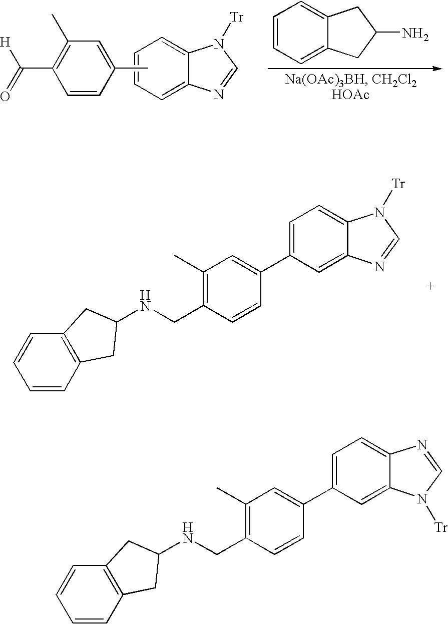

To a solution of 2-methyl-4-[1-(triphenylmethyl)-1H-benzimidazol-5-yl]benzaldehyde and 2-methyl-4-[1-(triphenylmethyl)-1H-benzimidazol-6-yl]benzaldehyde (21.0 g, 43.9 mmol) in dichloromethane (350 mL) was added 2-aminoindan (5.7 mL, 43.9 mmol) followed by acetic acid (10 drops). NOTE: In reactions where the amine hydrochloride salt was used, 1 eq. of NEt₃ was also added. The mixture was stirred 20 min, then sodium triacetoxyborohydride (37.2 g, 176 mmol) was added and the reaction was stirred 3 h at room temperature. The reaction mixture was quenched with H₂O and extracted with dichloromethane (2×). The combined organic extracts were washed with brine, dried over Na₂SO₄ and concentrated in vacuo. Chromatography (0-10% MeOH/CH₂Cl₂) provided the reductive amination products (a mixture of trityl regioisomers), N-({2-methyl-4-[1-(triphenylmethyl)-1H-benzimidazol-5-yl]phenyl}methyl)-2,3-dihydro-1H-inden-2-amine and N-({2-methyl-4-[1-(triphenylmethyl)-1H-benzimidazol-6-yl]phenyl}methyl)-2,3-dihydro-1H-inden-2-amine, as a white solid.

### Step 4: Preparation of N-{[4-(1H-benzimidazol-5-yl)-2-methylphenyl]methyl}-2,3-dihydro-1H-inden-2-amine dihydrochloride

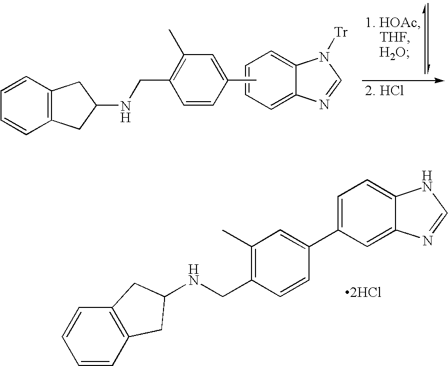

To a solution of N-({2-methyl-4-[1-(triphenylmethyl)-1H-benzimidazol-5-yl]phenyl}methyl)-2,3-dihydro-1H-inden-2-amine and N-({2-methyl-4-[1-(triphenylmethyl)-1H-benzimidazol-6-yl]phenyl}methyl)-2,3-dihydro-1H-inden-2-amine (0.15 g, 0.25 mmol) in tetrahydrofuran (3 mL) were added water (3 mL) and acetic acid (3 mL). The reaction mixture was heated at reflux for 1 h, then was allowed to cool to room temperature and treated with 10% HCl (aq) until pH 2. The mixture was washed with ethyl acetate (2×), then the aqueous phase was treated with solid potassium carbonate until it reached pH 10, at which point it was extracted with chloroform (3×). The combined organic extracts were dried (Na₂SO₄) and concentrated in vacuo. The residue was taken up in diethyl ether and enough acetone to dissolve completely, then 4M HCl in dioxane was added until no more solid crashed out (>2 eq. added). The white solid was collected by filtration and dried under vacuum, providing the product, N-{[4-(1H-benzimidazol-5-yl)-2-methylphenyl]methyl}-2,3-dihydro-1H-inden-2-amine dihydrochloride. (M+1) 352.2 ES, 1.36 min (LC/MS Method A).

TABLE 9
Compounds Prepared by General Method F

| | | | Methods/ |
|---|---|---|---|
| Ex. | Structure | Name | Comments |
| F-2 | 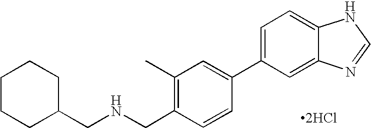 | {[4-(1H-benzimidazol-5-yl)- 2methylphenyl]methyl}- (cyclohexylmethyl)amine dihydrochloride (M + 1) 333.9 AP, 1.26 min. (LC/MS method B) | Method F |
| F-3 | 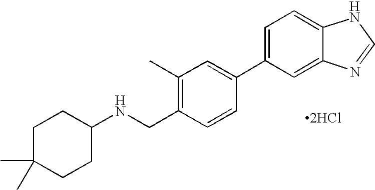 | N-{[4-(1H-benzimidazol-5- yl)-2-methylphenyl]methyl}- 4,4-dimethylcyclohexan- amine dihydrochloride (M + 1) 348.0 AP, 1.39 min. (LC/MS method B) | Method F |
| F-4 | 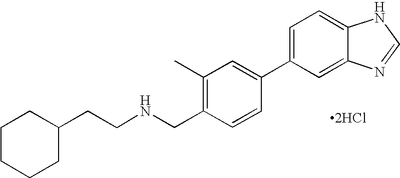 | N-{[4-(1H-benzimidazol-5- yl)-2-methylphenyl]methyl}- 2-cyclohexylethanamine dihydrochloride (M + 1) 348.0 AP, 1.46 min. (LC/MS method B) | Method F |
| F-5 | 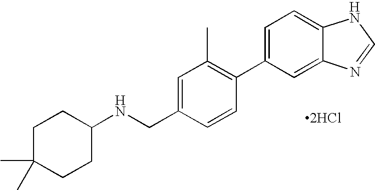 | N-{[4-(1H-benzimidazol-5- yl)-3-methylphenyl]methyl}- 4,4-dimethylcyclohexan- amine dihydrochloride (M + 1) 348.0 AP, 1.38 min. (LC/MS method B) | Method F Used Intermediate-31 |
| F-6 | 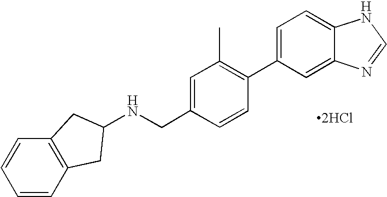 | N-{[4-(1H-benzimidazol-5- yl)-3-methylphenyl]methyl}- 2,3-dihydro-1H-inden-2- amine dihydrochloride (M + 1) 354.2 ES, 1.19 min. (LC/MS method A) | Method F Used Intermediate-31 |
| F-7 | 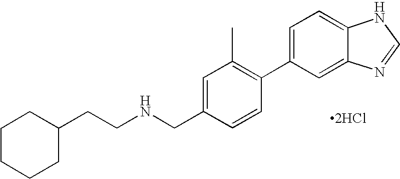 | N-{[4-(1H-benzimidazol-5- yl)-3-methylphenyl]methyl}- 2-cyclohexylethanamine dihydrochloride (M + 1) 348.0 AP, 1.38 min. (LC/MS method B) | Method F Used Intermediate-31 |
| F-8 | 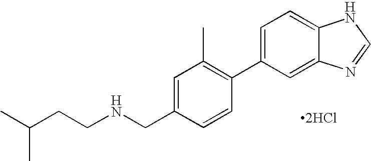 | N-{[4-(1H-benzimidazol-5- yl)-3-methylphenyl]methyl}- 3-methyl-1-butanamine dihydrochloride (M + 1) 308.1 AP, 1.03 min. (LC/MS method B) | Method F Used Intermediate-31 |
| F-9 | 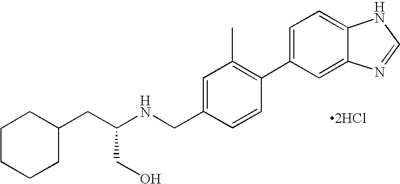 | (2S)-2-({[4-(1H- benzimidazol-5-yl)-3- methylphenyl]methyl}amino)- 3-cyclohexyl-1-propanol dihydrochloride (M + 1) 378.1 AP, 1.39 min. (LC/MS method B) | Method F Used Intermediate-31 |
| F-10 | 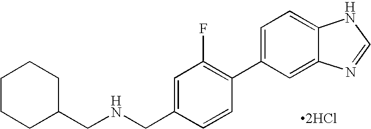 | 1-[4-(1H-benzimidazol-5-yl)- 3-fluorophenyl]-N- (cyclohexylmethyl) methanamine dihydrochloride (M + 1) 338.1 ES, 1.11 min. (LC/MS method A) | Method F Used Intermediate-33 |
| F-11 | 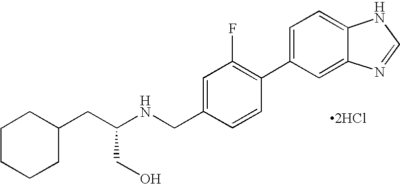 | (2S)-2-({[4-(1H- benzimidazol-5-yl)-3- fluorophenyl]methyl}amino)- 3-cyclohexyl-1-propanol dihydrochloride (M + 1) 382.2 ES, 1.35 min. (LC/MS method A) | Method F Used Intermediate-33 |
| F-12 | 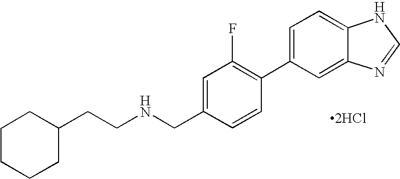 | N-{[4-(1H-benzimidazol-5- yl)-3-fluorophenyl]methyl}-2- cyclohexylethanamine dihydrochloride (M + 1) 352.2 ES, 1.39 min. (LC/MS method A) | Method F Used Intermediate-33 |
| F-13 | 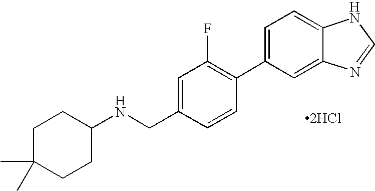 | N-{[4-(1H-benzimidazol-5- yl)-3-fluorophenyl]methyl}- 4,4-dimethylcyclohexan- amine dihydrochloride (M + 1) 352.2 ES, 1.30 min. (LC/MS method A) | Method F Used Intermediate-33 |
| F-14 | 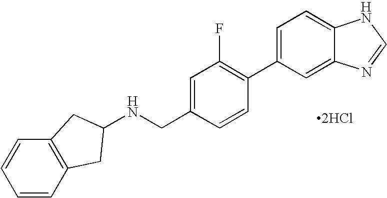 | N-{[4-(1H-benzimidazol-5- yl)-3-fluorophenyl]methyl}- 2,3-dihydro-1H-inden-2- amine dihydrochloride (M + 1) 358.2 ES, 1.10 min. (LC/MS method A) | Method F Used Intermediate-33 |
| F-15 | 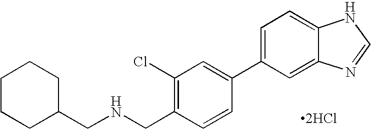 | 1-[4-(1H-benzimidazol-5-yl)- 2-chlorophenyl]-N- (cyclohexylmethyl) methanamine dihydrochloride (M + 1) 354.1 ES, 1.39 min. (LC/MS method A) | Method F Used Intermediate-35 |
| F-16 | 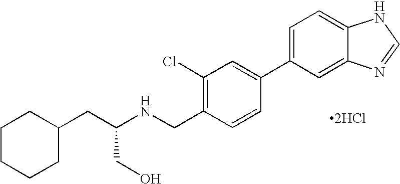 | (2S)-2-({[4-(1H- benzimidazol-5-yl)-2- chlorophenyl]methyl}amino)- 3-cyclohexyl-1-propanol dihydrochloride (M + 1) 398.2 ES, 1.56 min. (LC/MS method A) | Method F Used Intermediate-35 |
| F-17 | 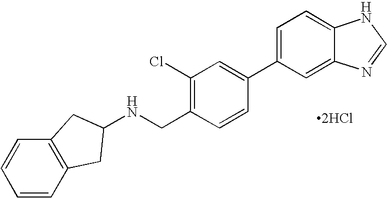 | N-{[4-(1H-benzimidazol-5- yl)-2-chlorophenyl]methyl}- 2,3-dihydro-1H-inden-2- amine dihydrochloride (M + 1) 374.1 ES, 1.37 min. (LC/MS method A) | Method F Used Intermediate-35 |
| F-18 | 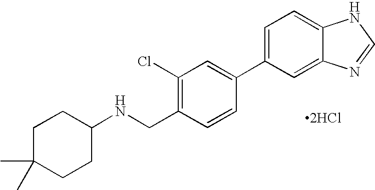 | N-{[4-(1H-benzimidazol-5- yl)-2-chlorophenyl]methyl}- 4,4-dimethylcyclohexanamine dihydrochloride (M + 1) 368.2 ES, 1.54 min. (LC/MS method A) | Method F Used Intermediate-35 |
| F-19 | 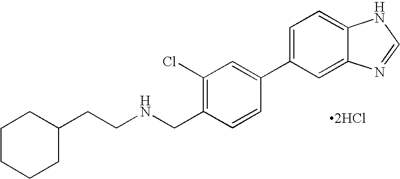 | N-{[4-(1H-benzimidazol-5- yl)-2-chlorophenyl]methyl}-2- cyclohexylethanamine dihydochloride (M + 1) 368.2 ES, 1.58 min. (LC/MS method A) | Method F Used Intermediate-35 |
| F-20 | 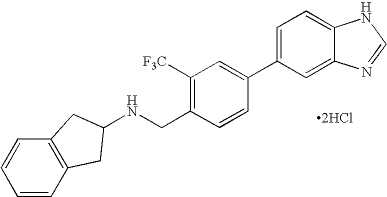 | N-{[4-(1H-benzimidazol-5- yl)-2-(trifluoromethyl)phenyl] methyl}-2,3-dihydro-1H-inden-2- amine dihydrochloride (M + 1) 408.1 ES, 1.45 min. (LC/MS method A) | Method F Used Intermediate-37 |
| F-21 | 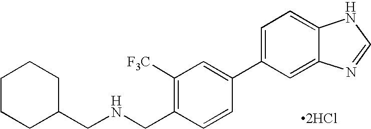 | {[4-(1H-benzimidazol-5-yl)-2- (trifluoromethyl)phenyl]methyl} (cyclohexylmethyl)amine dihydrochloride (M + 1) 388.2 ES, 1.48 min. (LC/MS method A) | Method F Used Intermediate-37 |
| F-22 | 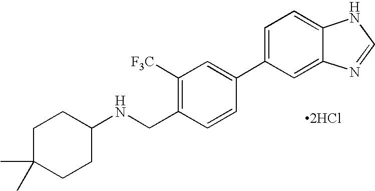 | N-{[4-(1H-benzimidazol-5- yl)-2-(trifluoromethyl)phenyl] methyl}-4,4-dimethylcyclohexan- amine dihydrochloride (M + 1) 402.2 ES, 1.60 min. (LC/MS method A) | Method F Used Intermediate-37 |
| F-23 | 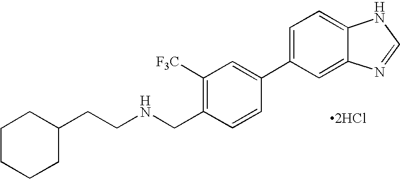 | N-{[4-(1H-benzimidazol-5- yl)-2-(trifluoromethyl) phenyl]methyl}- 2-cyclohexylethanamine dihydrochloride (M + 1) 402.2 ES, 1.70 min. (LC/MS method A) | Method F Used Intermediate-37 |
| F-24 | 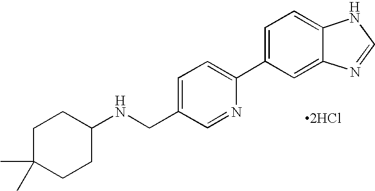 | N-{[6-(1H-benzimidazol-5- yl)-3-pyridinyl]methyl}-4,4- dimethylcyclohexanamine dihydrochloride (M + 1) 335.2 ES, 1.03 min. (LC/MS method A) | Method F Used Intermediate-39 2 eq. of HCl added to form salt |
| F-25 | 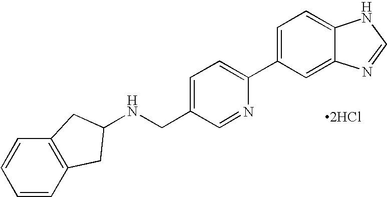 | N-{[6-(1H-benzimidazol-5- yl)-3-pyridinyl]methyl}-2,3- dihydro-1H-inden-2-amine dihydochloride (M + 1) 341.1 ES, 0.87 min. (LC/MS method A) | Method F Used Intermediate-39 2 eq. of HCl added to form salt |
| F-26 | 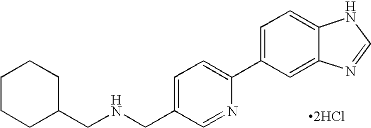 | {[6-(1H-benzimidazol-5-yl)-3- pyridinyl]methyl}(cyclohexyl methyl)amine dihydrochloride (M + 1) 321.2 ES, 0.92 min. (LC/MS method A) | Method F Used Intermediate-39 2 eq. of HCl added to form salt |
| F-27 | 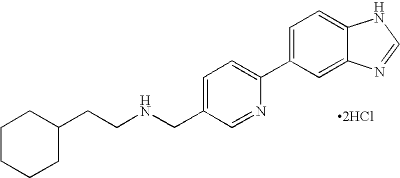 | N-{[6-(1H-benzimidazol-5- yl)-3-pyridinyl]methyl}-2- cyclohexylethanamine dihydrochloride (M + 1) 335.2 ES, 1.13 min. (LC/MS method A) | Method F Used Intermediate-39 2 eq. of HCl added to form salt |
| F-28 | 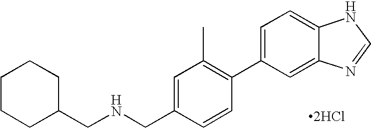 | {[4-(1H-benzimidazol-5-yl)-3- (methylphenyl]methyl}- (cyclohexyl-methyl)amine dihydrochloride (M + 1) 334.2 ES, 1.20 min. (LC/MS method A) | Method F Used Intermediate-31 |
| F-29 | 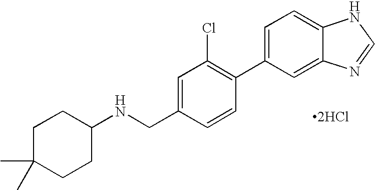 | N-{[4-(1H-benzimidazol-5- yl)-3-chlorophenylymethyl}- 4,4-dimethylcyclohexan- amine dihydrochloride (M + 1) 368.1 AP, 1.37 min. (LC/MS method B) | Method F Used Intermediate-40 |
| F-30 | 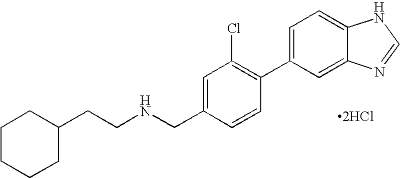 | N-{[4-(1H-benzimidazol-5- yl)-3-chlorophenylymethyl}-2- cyclohexylethanamine dihydrochloride (M + 1) 368.1 AP, 1.46 min. (LC/MS method B) | Method F Used Intermediate-40 |
| F-31 | 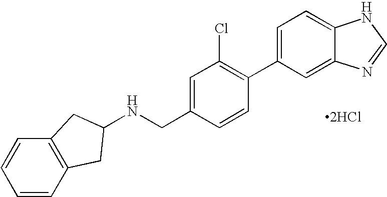 | N-{[4-(1H-benzimidazol-5- yl)-3-chlorophenylymethyl}- 2,3-dihydro-1H-inden-2- amine dihydrochloride (M + 1) 374.1 AP, 1.21 min. (LC/MS method B) | Method F Used Intermediate-40 |
| F-32 | 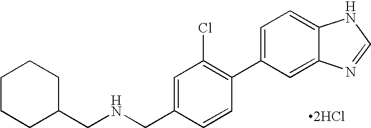 | 1-[4-(1H-benzimidazol-5-yl)- 3-chlorophenyl]-N- (cyclohexylmethyl)methan- amine dihydrochloride (M + 1) 354.0 AP, 1.20 min. (LC/MS method B) | Method F Used Intermediate-40 |
| F-33 | 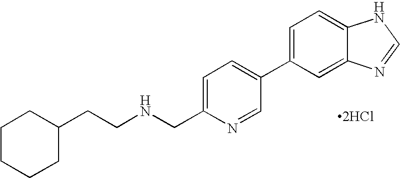 | N-{[5-(1H-benzimidazol-5- yl)-2-pyridinyl]methyl}-2- cyclohexylethanamine dihydrochloride (M + 1) 335.1 AP, 1.29 min. (LC/MS method B) | Method F Used Intermediate-42 2 eq. of HCl added to form salt |
| F-34 | 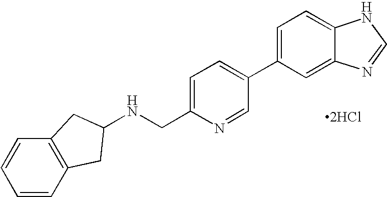 | N-{[5-(1H-benzimidazol-5- yl)-2-pyridinyl]methyl}-2,3- dihydro-1H-inden-2-amine dihydrochloride (M + 1) 341.0 AP, 0.95 min. (LC/MS method B) | Method F Used Intermediate-42 2 eq. of HCl added to form salt |
| F-35 | 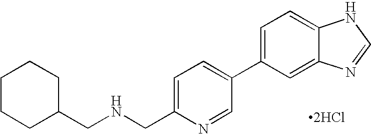 | 1-[5-(1H-benzimidazol-5-yl)- 2-pyridinyl]-N- (cyclohexylmethyl) methanamine dihydrochloride (M + 1) 321.0 AP, 1.01 min. (LC/MS method B) | Method F Used Intermediate-42 2 eq. of HCl added to form salt |
| F-36 | 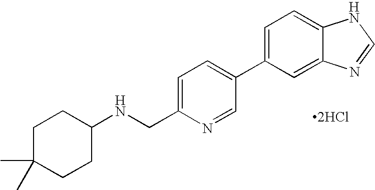 | N-{[5-(1H-benzimidazol-5- yl)-2-pyridinyl]methyl}-4,4- dimethylcyclohexanamine dihydrochloride (M + 1) 335.1 AP, 1.17 min. (LC/MS method B) | Method F Used Intermediate-42 2 eq. of HCl added to form salt |
| F-37 | 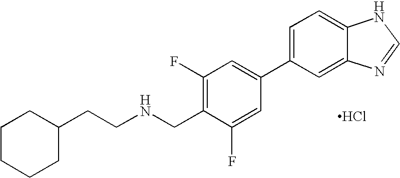 | N-{[4-(1H-benzimidazol-5- yl)-2,6-difluorophenyl] methyl}-2-cyclohexyl ethanamine hydrochloride (M + 1) 370.1 AP, 1.49 min. (LC/MS method B) | Method F Used Intermediate-43 1 eq. of HCl added to form salt |
| F-38 | 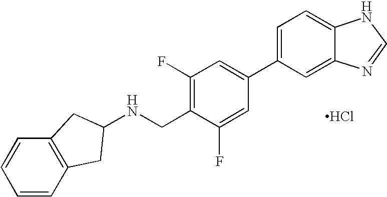 | N-{[4-(1 H-benzimidazol-5- yl)-2,6-difluorophenyl] methyl}-2,3-dihydro-1H- inden-2-amine hydrochloride (M + 1) 376.0 AP, 1.24 min. (LC/MS method B) | Method F Used Intermediate-43 1 eq. of HCl added to form salt |
| F-39 | 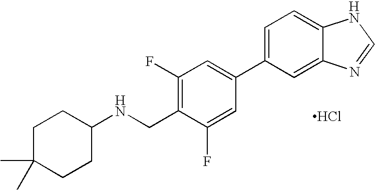 | N-{[4-(1H-benzimidazol-5- yl)-2,6-difluorophenyl] methyl}-4,4-dimethylcyclo- hexanamine hydrochloride (M + 1) 370.0 AP, 1.39 min. (LC/MS method B) | Method F Used Intermediate-43 1 eq. of HCl added to form salt |
| F-40 | 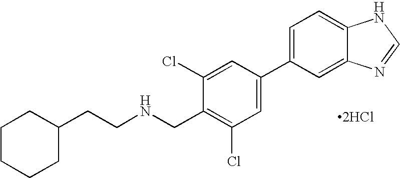 | N-{[4-(1H-benzimidazol-5- yl)-2,6-dichlorophenyl] methyl}-2-cyclohexyl ethanamine dihydrochloride (M + 1) 402.1 AP, 1.71 min. (LC/MS method B) | Method F Used Intermediate-45 2 eq. of HCl added to form salt |
| F-41 | 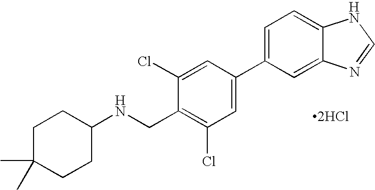 | N-{[4-(1 H-benzimidazol-5- yl)-2,6-dichlorophenyl] methyl}-4,4-dimethylcyclo hexanamine dihydrochloride (M + 1) 402.2 AP, 1.65 min. (LC/MS method B) | Method F Used Intermediate-45 2 eq. of HCl added to form salt |
| F-42 | 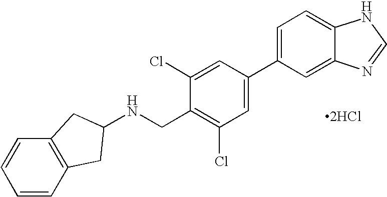 | N-{[4-(1 H-benzimidazol-5- yl)-2,6-dichlorophenyl] methyl}-2,3-dihydro-1H- inden-2-amine dihydrochloride (M + 1) 408.1 AP, 1.48 min. (LC/MS method B) | Method F Used Intermediate-45 2 eq. of HCl added to form salt |
| F-43 | 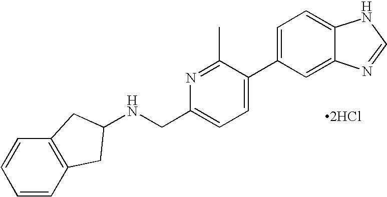 | N-{[5-(1H-benzimidazol-5- yl)-6-methyl-2-pyridinyl] methyl}-2,3-dihydro-1H- inden-2-amine dihydrochloride (M + 1) 355.3 AP, 1.03 min. (LC/MS method B) | Method F Used Intermediate-47 2 eq. of HCl added to form salt |
| F-44 | 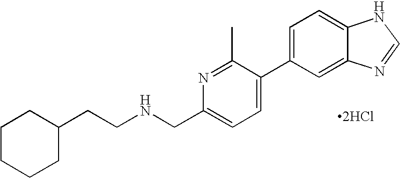 | N-{[5-(1 H-benzimidazol-5- yl)-6-methyl-2-pyridinyl] methyl}-2-cyclohexyl ethanamine dihydrochloride (M + 1) 349.3 AP, 1.35 min. (LC/MS method B) | Method F Used Intermediate-47 2 eq. of HCl added to form salt |
| F-45 | 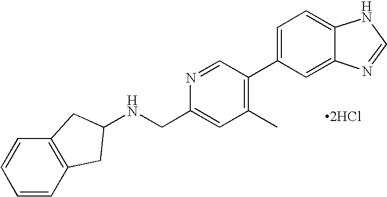 | N-{[5-(1H-benzimidazol-5- yl)-4-methyl-2-pyridinyl] methyl}-2,3-dihydro-1H- inden-2-amine dihydrochloride (M + 1) 355.3 AP, 0.99 min. (LC/MS method B) | Method F Used Intermediate-49 2 eq. of HCl added to form salt |
| F-46 | 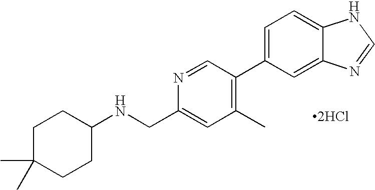 | N-{[5-(1 H-benzimidazol-5- yl)-4-methyl-2-pyridinyl] methyl}-4,4-dimethylcyclo hexanamine dihydrochloride (M + 1) 349.3 ES, 0.48 min. (LC/MS method E) | Method F Used Intermediate-49 2 eq. of HCl added to form salt |
| F-47 | 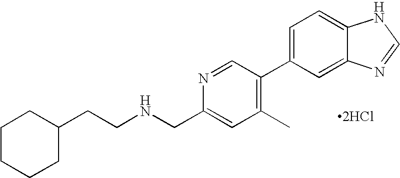 | N-{[5-(1 H-benzimidazol-5- yl)-4-methyl-2-pyridinyl] methyl}-2-cyclohexyl ethanamine dihydrochloride (M + 1) 349.3 ES, 0.50 min. (LC/MS method E) | Method F Used Intermediate-49 2 eq. of HCl added to form salt |
| F-48 | 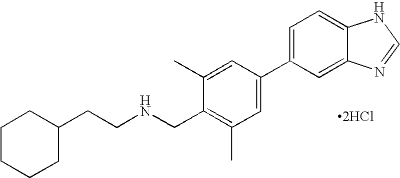 | N-{[4-(1H-benzimidazol-5- yl)-2,6-dimethylphenyl] methyl}-2-cyclohexyl ethanamine (M + 1) 362.3 ES, 0.55 min. (LC/MS method E) | Method F Used Intermediate-51 2 eq. of HCl added to form salt |
| F-49 | 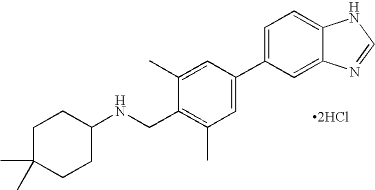 | N-{[4-(1 H-benzimidazol-5- yl)-2,6-dimethylphenyl] methyl}-4,4-dimethylcyclo hexanamine dihydrochloride (M + 1) 362.3 ES, 0.54 min. (LC/MS method E) | Method F Used Intermediate-51 2 eq. of HCl added to form salt |
| F-50 | 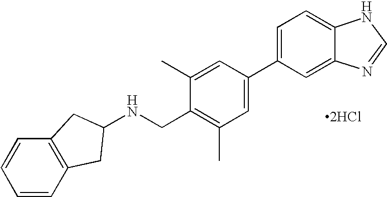 | N-{[4-(1 H-benzimidazol-5- yl)-2,6-dimethylphenyl] methyl}-2,3-dihydro-1H- inden-2-amine dihydrochloride (M + 1) 368.2 ES, 0.50 min. (LC/MS method E) | Method F Used Intermediate-51 2 eq. of HCl added to form salt |
| F-51 | 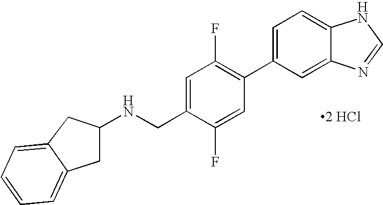 | N-{[4-(1H-benzimidazol-5- yl)-2,5-difluorophenyl] methyl}-2,3-dihydro- 1H-inden-2-amine dihydrochloride (M + 1) 376, 1.05 min. (LC/MS Method A) | Method F Used Intermediate-54 Used 10% MeOH/NH₃/ CH₂Cl₂ for chromatography Used dioxane as solvent for salt formation. |
| F-52 | 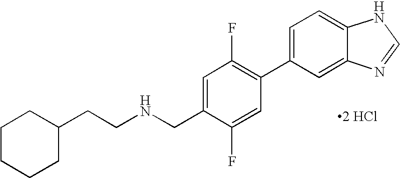 | N-{[4-(1H-benzimidazol-5- yl)-2,5-difluoro phenyl] methyl}-2-cyclohexyl ethanamine dihydrochloride (M + 1) 370, 1.49 min. (LC/MS Method A) | Method F Used Intermediate-54 Used 8% MeOH/NH₃/ CH₂Cl₂ for chromatography Used dioxane as solvent for salt formation. |
| F-53 | 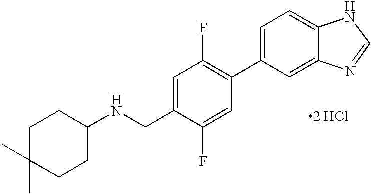 | N-{[4-(1 H-benzimidazol-5- yl)-2,5-difluorophenyl] methyl}-4,4-dimethylcyclo hexanamine dihydrochloride (M + 1) 370, 1.17 min. (LC/MS Method A) | Method F Used Intermediate-54 Used 8% MeOH/NH₃/ CH₂Cl₂ for chromatography Used dioxane as solvent for salt formation |
| F-54 | 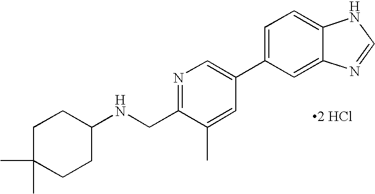 | N-{[5-(1H-benzimidazol-5- yl)-3-methyl-2- pyridinyl]methyl}-4,4- dimethylcyclohexanamine dihydrochloride (M + 1) 349, 1.21 min. (LC/MS Method A) | Method F Used Intermediate-56 Used 10% MeOH/NH₃/ CH₂Cl₂ for chromatography Used dioxane as solvent for salt formation |
| F-55 | 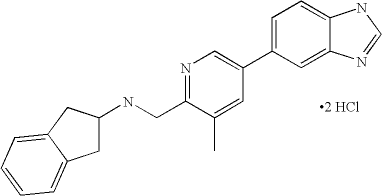 | N-{[5-(1H-benzimidazol-5- yl)-3-methyl-2-pyridinyl] methyl}-2,3-dihydro-1H- inden-2-amine dihydrochloride (M + 1) 355, 0.46 min. (LC/MS Method A) | Method F Used Intermediate-56 Used 10% MeOH/NH₃/ CH₂Cl₂ for chromatography Used dioxane as solvent for salt formation |
| F-56 | 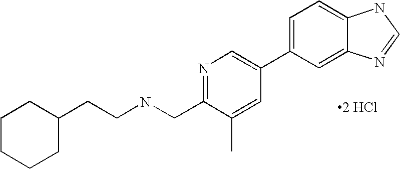 | N-{[5-(1H-benzimidazol-5- yl)-3-methyl-2-pyridinyl] methyl}-2-cyclohexyl ethanamine dihydrochloride (M + 1) 349, 1.2 min. (LC/MS Method A) | Method F Used Intermediate-56 Used 10% MeOH/NH₃/ CH₂Cl₂ for chromatography Used dioxane as solvent for salt formation |
| F-57 | 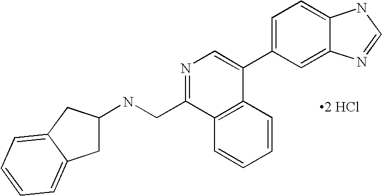 | N-{[4-(1H-benzimidazol-5- yl)-1-isoquinolinyl]methyl}- 2,3-dihydro-1H-inden-2- amine dihydrochloride (M + 1) 391, 1.5 min. (LC/MS Method A) | Method F Used Intermediate-58 Used 7% MeOH/NH₃/ CH₂Cl₂ for chromatography Used dioxane as solvent for salt formation |
| F-58 | 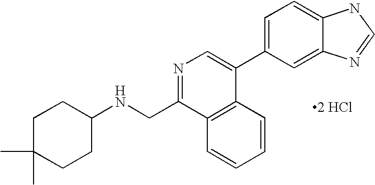 | N-{[4-(1H-benzimidazol-5- yl)-1-isoquinolinyl]methyl}- 4,4-dimethylcyclohexan amine dihydrochloride (M + 1) 385, 1.1 min. (LC/MS Method A) | Method F Used Intermediate-58 Used 8% MeOH/NH₃/ CH₂Cl₂ for chromatography Used dioxane as solvent for salt formation |
| F-59 | 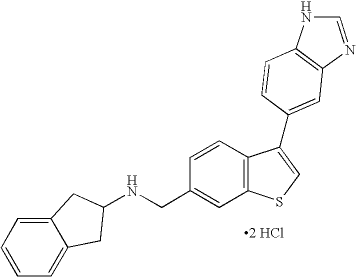 | N-{[3-(1H-benzimidazol-5- yl)-1-benzothien-6-yl] methyl}-2,3-dihydro-1H- inden-2-amine dihydrochloride (M + 1) 396, 1.35 min. (LC/MS Method A) | Method F Used Intermediate-60 |
| F-60 | 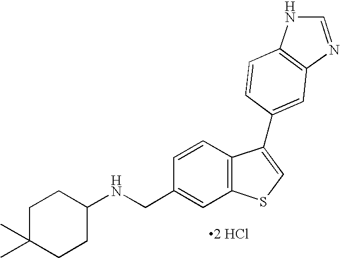 | N-{[3-(1H-benzimidazol-5- yl)-1-benzothien-6-yl] methyl}-4,4-dimethyl cyclohexanamine dihydrochloride (M + 1) 390, 1.48 min. (LC/MS Method A) | Method F Used Intermediate-60 Used 10% MeOH/NH₃/ CH₂Cl₂ for chromatography Used dioxane as solvent for salt formation |
| F-61 | 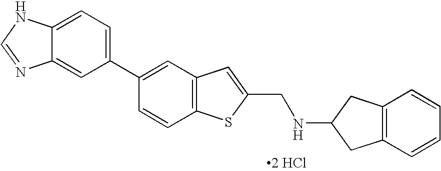 | N-{[5-(1H-benzimidazol-5- yl)-1-benzothien-2-yl] methyl}-2,3-dihydro-1H- inden-2-amine dihydrochloride (M + 1) 396, 1.36 min. (LC/MS Method A) | Method F Used Intermediate-62 Used 10% MeOH/NH₃/ CH₂Cl₂ for chromatography Used dioxane as solvent for salt formation |
| F-62 | 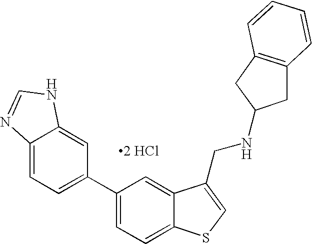 | N-{[5-(1H-benz imidazol-5- yl)-1-benzo thien-3-yl] methyl}-2,3-dihydro-1H- inden-2-amine dihydrochloride (M + 1) 396.04, 1.40 min (LC/MS method A) | Method F Used Intermediate-64 Reductive amination reaction time 4h. Deprotection was accomplished using 6M HCl (aq) in EtOH at 70 degrees Celsius for 2 hr. Product crystallized out of solution on cooling |
| F-63 | 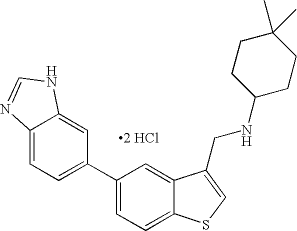 | N-{[5-(1H-benz imidazol-5- yl)-1-benzothien-3-yl] methyl}-4,4-dimethyl cyclohexanamine dihydrochloride (M + 1) 390.20, 0.67 min (LC/MS Method E) | Method F Used Intermediate-64 Reductive amination reaction time 4h. Deprotection was accomplished using 6M HCl (aq) in EtOH at 70 degrees Celsius for 2 hr. Product crystallized out of solution on cooling |

General Method G:

### G-1

### Step 1: Preparation of 4-bromo-5-fluoro-2-nitroaniline

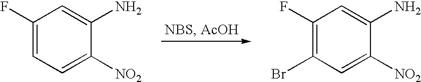

A solution of 5-fluoro-2-nitroaniline (3.0 g, 19.2 mmol) and N-bromosuccinimide (3.4 g, 18.8 mmol) in acetic acid (150 mL) was heated at reflux for 90 min. The reaction mixture was then allowed to cool and was poured into water (1.5 L). After stirring for 10 min., the bright yellow precipitate was isolated via filtration and dried under vacuum overnight. This provided the product, 4-bromo-5-fluoro-2-nitroaniline. ¹H NMR (400 MHz, CDCl₃) δ 6.19 (br s, 2H), 6.58 (d, J=9.8 Hz, 1H), 8.38 (d, J=7.08 Hz, 1H).

### Step 2: Preparation of 4-bromo-5-fluoro-1,2-benzenediamine

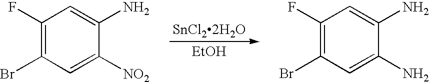

To a solution of 4-bromo-5-fluoro-2-nitroaniline (3.2 g, 13.6 mmol) in ethanol (75 mL) was added tin(II) chloride dihydrate (9.2 g, 40.8 mmol) and the reaction was heated at 70 degrees C. for 3 h. The reaction was allowed to cool, then was diluted with saturated sodium bicarbonate and water, and was extracted with ethyl acetate (3×). The extracts were dried over magnesium sulfate and concentrated in vacuo. The residue was purified via chromatography (50% ethyl acetate/hexanes), providing the product, 4-bromo-5-fluoro-1,2-benzenediamine, as a tan solid.

¹H NMR (400 MHz, CDCl₃) δ 3.5 (br s, 4H), 6.49 (d, J=9.52 Hz, 1H), 6.82 (d, J=6.6 Hz, 1H).

### Step 3: Preparation of 5-bromo-6-fluoro-1H-benzimidazole

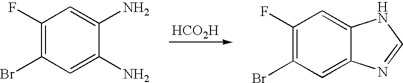

A solution of 4-bromo-5-fluoro-1,2-benzenediamine (2.2 g, 10.7 mmol) in formic acid (5 mL) was heated at reflux for 2 h. The reaction was then cooled to room temperature and treated with 10% aq. NaOH until the mixture was basic. The dark mixture was extracted with ethyl acetate (3×), and the combined organics were washed with brine, dried over sodium sulfate, and concentrated in vacuo. The residue was triturated with dichloromethane, then dried under vacuum, providing the product, 5-bromo-6-fluoro-1H-benzimidazole. The dichloromethane wash was also concentrated in vacuo and purified via chromatography to provide additional product. ¹H NMR (400 MHz, CDCl₃) δ 7.35 (d, J=8.6 Hz, 1H), 7.78 (d, J=6.1 Hz, 1H), 8.02 (s, 1H).

### Step 4: Preparation of 5-bromo-6-fluoro-1-(triphenylmethyl)-1H-benzimidazole and 6-bromo-5-fluoro-1-(triphenylmethyl)-1H-benzimidazole

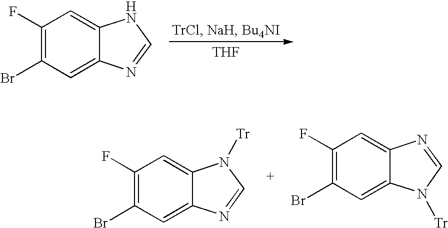

To a solution of 5-bromo-6-fluoro-1H-benzimidazole (2.2 g, 10.2 mmol) in tetrahydrofuran (40 mL) at 0 degrees C. was added sodium hydride (0.5 g, 12.3 mmol) as a solid in 2 portions. After stirring 30 min. at 0° C., trityl chloride (3.7 g, 13.3 mmol) was added, followed by tetrabutylammonium iodide (catalytic). The reaction was heated at reflux for 1.5 h, then was allowed to cool and was quenched with water. The mixture was extracted with chloroform (3×) and the combined organics were dried over sodium sulfate and concentrated in vacuo. The residue was triturated with hot ethyl acetate and the solid was isolated via filtration and dried under vacuum. This provided the product as a mixture of trityl regioisomers, 5-bromo-6-fluoro-1-(triphenylmethyl)-1H-benzimidazole and 6-bromo-5-fluoro-1-(triphenylmethyl)-1H-benzimidazole.

### Step 5: Preparation of 4-[5-fluoro-1-(triphenylmethyl)-1H-benzimidazol-6-yl]benzaldehyde and 4-[6-fluoro-1-(triphenylmethyl)-1H-benzimidazol-5-yl]benzaldehyde

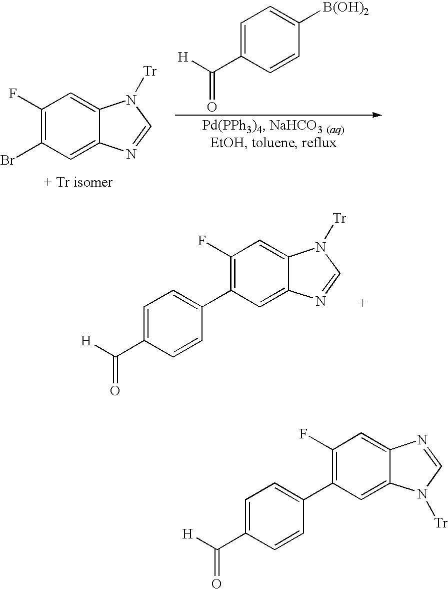

To a solution of 5-bromo-6-fluoro-1-(triphenylmethyl)-1H-benzimidazole and 6-bromo-5-fluoro-1-(triphenylmethyl)-1H-benzimidazole (2.0 g, 4.4 mmol) in toluene (10 mL) under nitrogen atmosphere was added tetrakis(triphenylphosphine) palladium(0) (0.25 g, 0.2 mmol) followed by a solution of sodium bicarbonate (0.92 g, 11.0 mmol) in water (5 mL). A solution of 4-formylphenylboronic acid (0.72 g, 4.8 mmol) in ethanol (3 mL) was then added and the reaction was heated at reflux overnight. After removal of the solvent in vacuo, the residue was partitioned between ethyl acetate and water. The combined organics were dried over sodium sulfate, concentrated in vacuo, and purified via chromatography (20 to 50% ethyl acetate/hexanes) to provide the product as a mixture of trityl regioisomers, 4-[5-fluoro-1-(triphenylmethyl)-1H-benzimidazol-6-yl]benzaldehyde and 4-[6-fluoro-1-(triphenylmethyl)-1H-benzimidazol-5-yl]benzaldehyde.

Isomer a: ¹H NMR (400 MHz, CDCl₃) δ 6.45-6.48 (m, 1H), 7.16-7.26 (m, 8H), 7.35-7.39 (m, 9H), 7.65-7.72 (m, 1H), 7.81 (d, J=7.4 Hz, 1H), 8.12-8.22 (m, 1H), 9.99 (s, 1H).

Isomer b: ¹H NMR (400 MHz, CDCl₃) δ 6.27 (d, J=11.2 Hz, 1H), 7.17-7.21 (m, 6H), 7.34-7.37 (m, 9H), 7.70 (d, J=6.6 Hz, 2H), 7.85 (d, J=7.1 Hz, 1H), 7.92-7.96 (m, 3H), 10.04 (s, 1H).

TABLE 10
Aldehyde Intermediates Prepared by General Method G (Step 5)

| | | Name and | |
|---|---|---|---|
| Intermediate | Structure | Characterizing Data | Method/Comments¹ |
| Intermediate-66 | 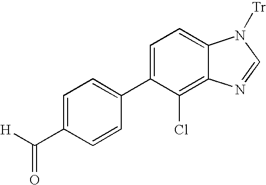 | 4-[4-chloro-1- (triphenylmethyl)-1H- benzimidazol-5-yl] benzaldehyde White solid ¹H NMR (400 MHz, CDCl₃) δ 6.44 (d, J = 8.6 Hz, 1H), 6.89 (d, J = 8.6 Hz, 1H), 7.17- 7.20 (m, 6H), 7.33- 7.35 (m, 9H), 7.64 (d, J = 8.0 Hz, 2H), 7.92 (d, J = 8.0 Hz, 2H), 7.98 (s, 1H), 10.05 (s, 1H). | Prepared as in Step 5 using Intermediate-67 and PdCl2.dppf as catalyst. Chromotography used 20 to 35% EtOAc/hexanes. |
| Intermediate-68* | 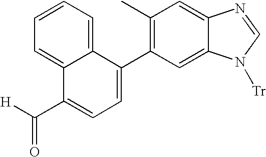 | 4-[5-methyl-1- (triphenyl methyl)-1H- benzimidazol-6-yl]- 1-naphthalene carbaldehyde Yellow solid | Method G Used Intermediates 20 and 69, and used Na₂CO₃ as base. Chromatography used 20-35% EtOAc/hexanes. |
| Intermediate-70 | 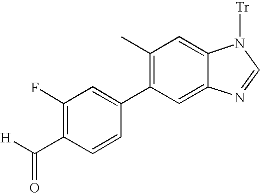 | 2-fluoro-4-[6-methyl-1- (triphenyl methyl)-1H- benz imidazol-5-yl] benzaldehyde (M + 1) 497.3, 3.13 min. (LC/MS Method A). | Method G Used Intermediate-69 and (3-fluoro-4-formylphenyl) boronic acid Chromatography used 20-35% EtOAc/hexanes. |
| Intermediate-71* | 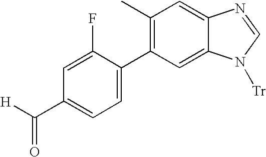 | 3-fluoro-4-[5-methyl-1- (triphenylmethyl)-1H- benzimidazol-6- yl]benzaldehyde | Method G Used Intermediate-69 and (2-fluoro-4-formylphenyl) boronic acid Chromatography used 20-35% EtOAc/hexanes. |
| Intermediate-72 | 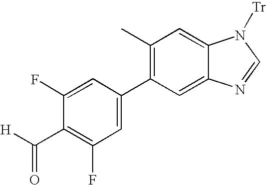 | 2,6-difluoro-4-[5- methyl-1- (triphenylmethyl)-1H- benzimidazol-6- yl]benzaldehyde | Method G Used Intermediate-69 and (3,5-difluoro-4-formyl phenyl)boronic acid Chromatography used 20-35% EtOAc/hexanes. |

¹Note:
Descriptions for the synthesis of intermediates referred to in this column and used to synthesize the above aldehydes can be found in the section entitled, “Synthetic Descriptions of Intermediates Used in General Method A through K.”
*Mixture of trityl regioisomers

### Step 6: Preparation of N-{[4-(5-fluoro-1H-benzimidazol-6-yl)phenyl]methyl}-4,4-dimethylcyclohexanamine dihydrochloride

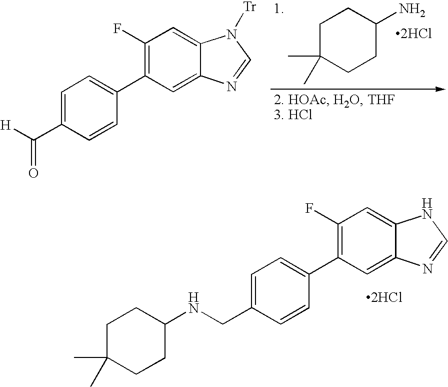

To a suspension of 4-[5-fluoro-1-(triphenylmethyl)-1H-benzimidazol-6-yl]benzaldehyde (0.25 g, 0.52 mmol) and 4,4-dimethylcyclohexylamine hydrochloride (85 mg, 0.52 mmol) in dichloromethane (5 mL) was added triethylamine (72 μL, 0.52 mmol) followed by 2 drops of acetic acid. (NOTE: If the amine free base was used, no triethylamine was added). The mixture was stirred 20 min, then sodium triacetoxyborohydride (0.44 g, 2.08 mmol) was added and the reaction was stirred 3 h at room temperature. The reaction mixture was quenched with H₂O and extracted with dichloromethane (2×). The combined organic extracts were washed with brine, dried over Na₂SO₄ and concentrated in vacuo. Chromatography (5-10% MeOH/CH₂Cl₂) provided the reductive amination product as a white solid. This product was dissolved in a solution of THF:H₂O:acetic acid (3 mL:3 mL:3 mL) and heated at reflux for 1 h. After cooling to room temperature, the mixture was treated with 10% aq. HCl until it reached pH 2. The mixture was washed with ethyl acetate (2×), then the aqueous was treated with solid potassium carbonate to achieve pH 10. This was extracted with chloroform (3×) and the combined organic extracts were dried over sodium sulfate and concentrated in vacuo. The residue was taken up in diethyl ether and a small amount of acetone, and 4.0 M HCl in dioxane was added until no more white solid crashed out (>2 eq. were added). The solid was triturated with hot ethyl acetate, then collected via filtration and dried under vacuum, providing the product, N-{[4-(5-fluoro-1H-benzimidazol-6-yl)phenyl]methyl}-4,4-dimethylcyclohexanamine dihydrochloride, as a white solid. (M+1) 352.3 ES, 1.52 min. (LC/MS method A)

TABLE 11
Compounds Prepared by General Method G

| Ex. | Structure | Name | Method/Comments |
|---|---|---|---|
| G-2 | 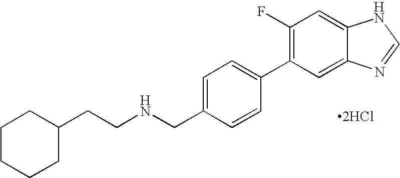 | 2-cyclohexyl-N-{[4-(5-fluoro- 1H-benzimidazol-6- yl)phenyl]methyl} ethanamine dihydrochloride (M + 1) 352.2 ES, 1.54 min. (LC/MS method A) | Method G |
| G-3 | 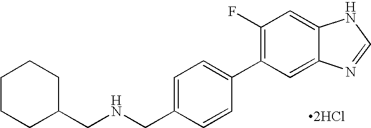 | 1-cyclohexyl-N-{[4-(5-fluoro- 1H-benzimidazol-6- yl)phenyl]methyl}methan- amine dihydrochloride (M + 1) 338.2 ES, 1.23 min. (LC/MS method A) | Method G |
| G-4 | 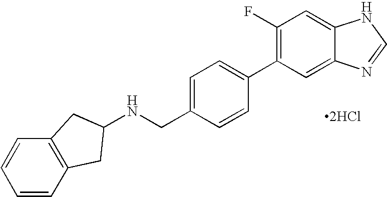 | N-{[4-(5-fluoro-1H- benzimidazol-6- yl)phenyl]methyl}-2,3- dihydro-1H-inden-2-amine dihydrochloride (M + 1) 358.2 ES, 1.22 min. (LC/MS method A) | Method G |
| G-5 | 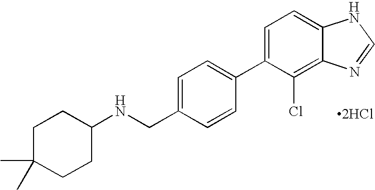 | N-{[4-(4-chloro-1H-benz imidazol-5-yl)phenyl] methyl}-4,4-dimethyl cyclohexanamine dihydrochloride light pink solid. (M + 1) 368.1 ES, 0.56 min. (LC/MS method E) | Method G Used Intermediate-66 Chromatograohy used 0-10% MeOH/CH₂Cl₂ as eluent. No EtOAc treatment of final product was required |
| G-6 | 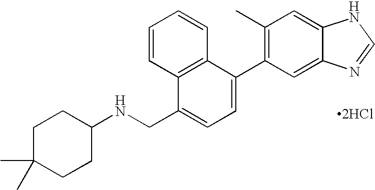 | (4,4-dimethylcyclo hexyl){[4- (5-methyl-1H-benz imidazol- 6-yl)-1-naphthalenyl]methyl} amine dihydrochloride off-white solid. (M + 1) 398.3 ES, 0.53 min. (LC/MS method C) | Method G Used intermediate-68 Chromatography used 0-5% MeOH/CH₂Cl₂ |
| G-7 | 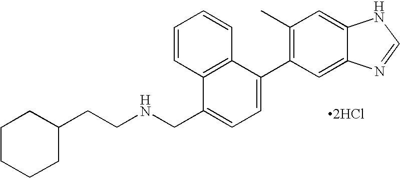 | (2-cyclohexylethyl){[4-(5- methyl-1H-benzimidazol-6- yl)-1-naphthalenyl]methyl}- amine dihydrochloride (M + 1) 398.3 ES, 0.55 min. (LC/MS method E) | Method G Used intermediate-68 Chromatography used 0-5% MeOH/CH₂Cl₂ |
| G-8 | 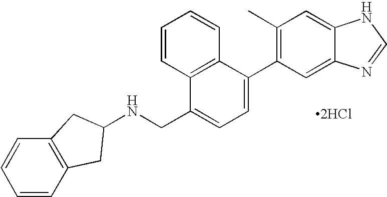 | N-{[4-(5-methyl-1H- benzimidazol-6-yl)-1- naphthalenyl]methyl}-2,3- dihydro-1H-inden-2-amine dihydrochloride (M + 1) 404.2 ES, 0.50 min. (LC/MS method E) | Method G No NEt₃ added to reductive amination Used intermediate-68 Chromatography used 0-5% MeOH/CH₂Cl₂ |
| G-9 | 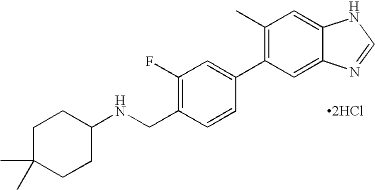 | N-{[2-fluoro-4-(5-methyl-1H- benzimidazol-6- yl)phenyl]methyl}-4,4- dimethylcyclohexanamine dihydrochloride white solid. (M + 1) 366.3 ES, 1.39 min. (LC/MS method A) | Method G Used Intermediate-70 Chromatography used 2-5% MeOH/CH₂Cl₂ at the trityl protected stage |
| G-10 | 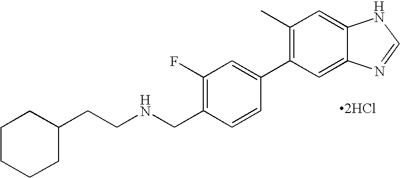 | 2-cyclohexyl-N-{[2-fluoro-4- (5-methyl-1H-benzimidazol- 6-yl)phenyl]methyl}ethan- amine dihydrochloride (M + 1) 366.3 ES, 1.46 min. (LC/MS method A) | Method G Used Intermediate-70 Chromatography used 2-5% MeOH/CH₂Cl₂ at the trityl protected stage |
| G-11 | 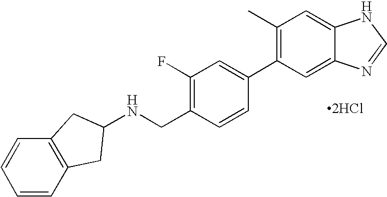 | N-{[2-fluoro-4-(5-methyl-1H- benzimidazol-6- yl)phenyl]methyl}-2,3- dihydro-1H-inden-2-amine dihydrochloride (M + 1) 372.2 ES, 1.19 min. (LC/MS method A) | Method G Used Intermediate-70 NEt₃ not added to reductive amination. Chromatography used 0-5% MeOH/CH₂Cl₂ at the trityl protected stage |
| G-12 | 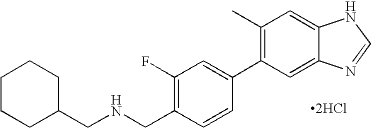 | 1-cyclohexyl-N-{[2-fluoro-4- (5-methyl-1H-benzimidazol- 6-yl)phenyl]methyl}methan- amine dihydrochloride (M + 1) 352.2 ES, 1.22 min. (LC/MS method A) | Method G Used Intermediate-70 NEt₃ not added to reductive amination. Chromatography used 0-5% MeOH/CH₂Cl₂ at the trityl protected stage |
| G-13 | 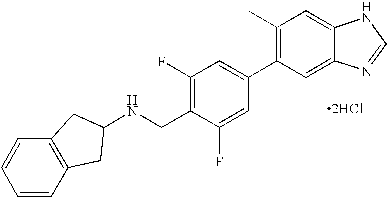 | N-{[2,6-difluoro-4-(5-methyl- 1H-benzimidazol-6- yl)phenyl]methyl}-2,3- dihydro-1H-inden-2-amine dihydrochloride (M + 1) 390.2 ES, 1.26 min. (LC/MS method A) | Method G Used Intermediate-72 NEt₃ not added to reductive amination. Chromatography used 0-5% MeOH/CH₂Cl₂ at the trityl protected stage |
| G-14 | 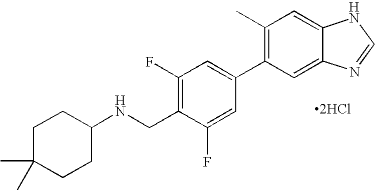 | N-{[2,6-difluoro-4-(5-methyl- 1H-benzimidazol-6- yl)phenyl]methyl}-4,4- dimethylcyclohexanamine dihydrochloride (M + 1) 384.3 ES, 1.41 min. (LC/MS method A) | Method G Used Intermediate-72 Chromatography used 0-5% MeOH/CH₂Cl₂ at the trityl protected stage |
| G-15 | 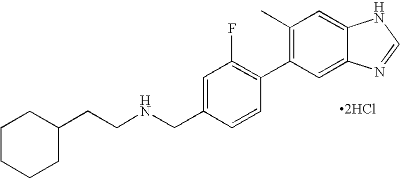 | (2-cyclohexylethyl){[3-fluoro- 4-(5-methyl-1H- benzimidazol-6- yl)phenyl]methyl}amine dihydrochloride (M + 1) 366.5 ES, 0.51 min. (LC/MS method E) | Method G Used Intermediate-71 Chromatography used 0-5% MeOH/CH₂Cl₂ at the trityl protected stage |
| G-16 | 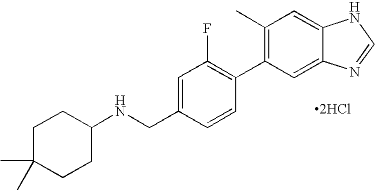 | N-{[3-fluoro-4-(5-methyl-1H- benzimidazol-6- yl)phenyl]methyl}-4,4- dimethylcyclohexanamine dihydrochloride (M + 1) 366.6 ES, 0.50 min. (LC/MS method E) | Method G Used Intermediate-71 Chromatography used 2-5% MeOH/CH₂Cl₂ at the trityl protected stage |
| G-17 | 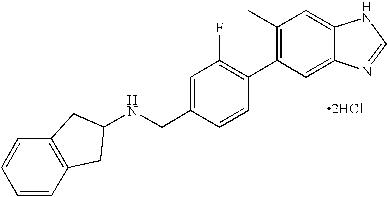 | N-{[3-fluoro-4-(5-methyl-1H- benzimidazol-6- yl)phenyl]methyl}-2,3- dihydro-1H-inden-2-amine dihydrochloride (M + 1) 372.5 ES, 0.46 min. (LC/MS method E) | Method G Used Intermediate-71 NEt₃ not added to reductive amination. Chromatography used 2-5% MeOH/CH₂Cl₂ at the trityl protected stage |
| G-18 | 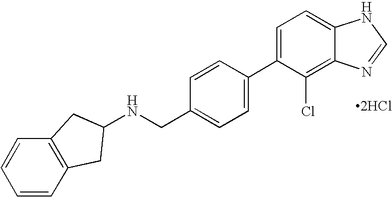 | N-{[4-(4-chloro-1H- benzimidazol-5- yl)phenyl]methyl}-2,3- dihydro-1H-inden-2-amine dihydrochloride (M + 1) 374.1 ES, 0.51 min. (LC/MS method E) | Method G Used Intermediate-66 NEt₃ not added to reductive amination. Chromatography used 0-10% MeOH/CH₂Cl₂ at the trityl protected stage |
| G-19 | 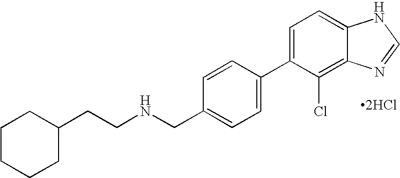 | N-{[4-(4-chloro-1H- benzimidazol-5- yl)phenyl]methyl}-2- cyclohexylethanamine dihydrochloride (M + 1) 368.1 ES, 0.57 min. (LC/MS method E) | Method G Used Intermediate-66 NEt₃ not added to reductive amination. Chromatography used 0-10% MeOH/CH₂Cl₂ at the trityl protected stage |

General Method H:

### Example H-1

### Step 1: Preparation of 6-methyl-5-(4,4,5,5-tetramethyl-1,3,2-dioxaborolan-2-yl)-1-(triphenylmethyl)-1H-benzimidazole and 5-methyl-6-(4,4,5,5-tetramethyl-1,3,2-dioxaborolan-2-yl)-1-(triphenylmethyl)-1H-benzimidazole

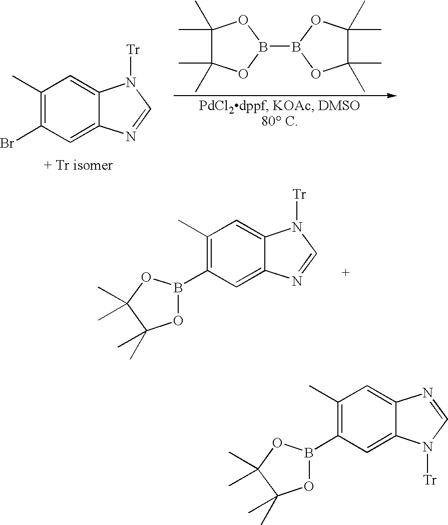

In a 250 mL round bottomed flask, bis(pinacolato)diboron (7.2 g, 28.4 mmol), potassium acetate (7.6 g, 77.4 mmol), and PdCl₂.dppf (0.9 g, 1.3 mmol) were combined. The flask was flushed with nitrogen and a slurry of 5-bromo-6-methyl-1-(triphenylmethyl)-1H-benzimidazole and 6-bromo-5-methyl-1-(triphenylmethyl)-1H-benzimidazole (11.7 g, 25.8 mmol) in dimethylsulfoxide (120 mL) was added and the reaction was heated at 80 degrees C. overnight. After cooling to room temperature, the mixture was diluted with chloroform, then washed with water. The organics were dried over sodium sulfate, concentrated in vacuo, and purified via chromatography (20 to 50% ethyl acetate/hexane), providing the product as a mixture of trityl regioisomers, 6-methyl-5-(4,4,5,5-tetramethyl-1,3,2-dioxaborolan-2-yl)-1-(triphenylmethyl)-1H-benzimidazole and 5-methyl-6-(4,4,5,5-tetramethyl-1,3,2-dioxaborolan-2-yl)-1-(triphenylmethyl)-1H-benzimidazole. (M+1) 501.3 ES, 3.13 and 3.29 min. (LC/MS method A).

### Step 2: Preparation of 2-methyl-4-[5-methyl-1-(triphenylmethyl)-1H-benzimidazol-6-yl]benzaldehyde and 2-methyl-4-[6-methyl-1-(triphenylmethyl)-1H-benzimidazol-5-yl]benzaldehyde

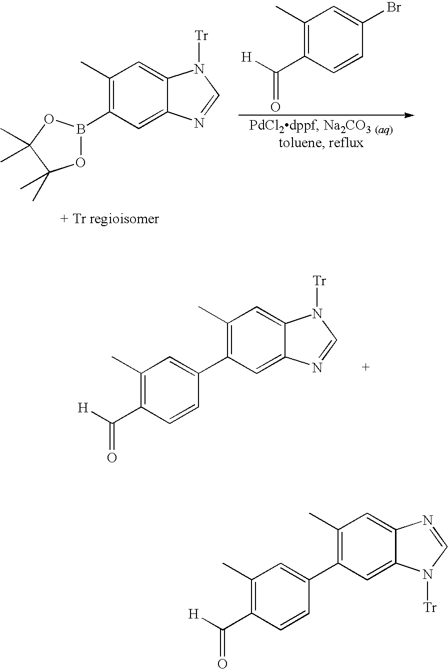

To a solution of 4-bromo-2-methylbenzaldehyde (1.0 g, 5.0 mmol) in toluene (15 mL) was added PdCl₂.dppf (0.18 g, 0.3 mmol), followed by a solution of sodium carbonate (1.3 g, 12.5 mmol) in water (5 mL) and a solution of 6-methyl-5-(4,4,5,5-tetramethyl-1,3,2-dioxaborolan-2-yl)-1-(triphenylmethyl)-1H-benzimidazole and 5-methyl-6-(4,4,5,5-tetramethyl-1,3,2-dioxaborolan-2-yl)-1-(triphenylmethyl)-1H-benzimidazole [Intermediate 69] (2.5 g, 5.0 mmol) in toluene (5 mL). The reaction was heated at reflux overnight, then the solvent was removed in vacuo and the residue was partitioned between ethyl acetate and water. The organics were dried over sodium sulfate and concentrated in vacuo. Chromatography (20 to 35% ethyl acetate/hexanes) provided the product as a mixture of trityl regioisomers, 2-methyl-4-[5-methyl-1-(triphenylmethyl)-1H-benzimidazol-6-yl]benzaldehyde and 2-methyl-4-[6-methyl-1-(triphenylmethyl)-1H-benzimidazol-5-yl]benzaldehyde, as a yellow solid.

The following biaryl aldehydes were synthesized in an analogous manner:

TABLE 12
Aldehyde Intermediates Prepared by General Method H (Step 2)

| Ex. | Structure | Name | Comments¹ |
|---|---|---|---|
| Intermediate- 73 | 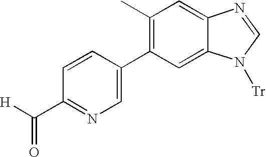 | 5-[5-methyl-1-(triphenylmethyl)- 1H-benzimidazol-6-yl]-2- pyridinecarbaldehyde and 5-[6- methyl-1-(triphenylmethyl)-1H- benzimidazol-6-yl]-2- pyridinecarbaldehyde | Prepared from Intermediate- 53 |
| Intermediate- 74 | 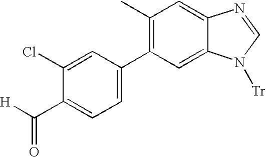 | 2-chloro-4-[5-methyl-1- (triphenylmethyl)-1H- benzimidazol-6-yl]benzaldehyde and 2-chloro-4-[6-methyl-1- (triphenylmethyl)-1H- benzimidazol-6-yl]benzaldehyde | Prepared from Intermediate- 36 |

¹Note: Descriptions for the synthesis of intermediates referred to in this column and used to synthesize the above aldehydes can be found in the section entitled, “Synthetic Descriptions of Intermediates Used in General Methods A through K.
All biaryl aldehydes were obtained as a mixture of trityl regioisomers.

### Step 3: Preparation of (4,4-dimethylcyclohexyl){[2-methyl-4-(5-methyl-1H-benzimidazol-6-yl)phenyl]methyl}amine dihydrochloride

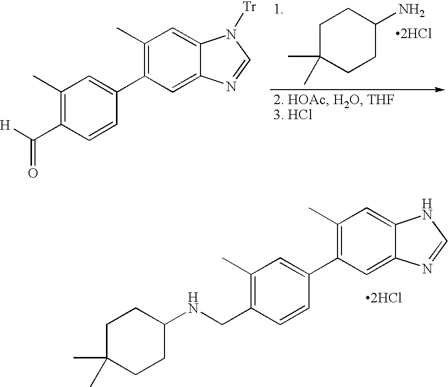

To a solution of 2-methyl-4-[5-methyl-1-(triphenylmethyl)-1H-benzimidazol-6-yl]benzaldehyde and 2-methyl-4-[6-methyl-1-(triphenylmethyl)-1H-benzimidazol-5-yl]benzaldehyde (0.22 g, 0.45 mmol) and 4,4-dimethylcyclohexylamine hydrochloride (73 mg, 0.45 mmol) in dichloromethane (5 mL) was added triethylamine (63 μL, 0.45 mmol) followed by 2 drops of acetic acid. The mixture was stirred 20 min, then sodium triacetoxyborohydride (0.38 g, 1.8 mmol) was added and the reaction was stirred 3 h at room temperature. The reaction mixture was quenched with H₂O and extracted 2× CH₂Cl₂. The combined organic extracts were washed with brine, dried over Na₂SO₄, and concentrated in vacuo. Chromatography (0-5% MeOH/CH₂Cl₂) provided the desired reductive amination products (mixture of trityl regioisomers) as a white foam. This product was dissolved in a solution of THF:H₂O:acetic acid (3 mL:3 mL:3 mL) and heated at reflux for 1 h. After cooling to room temperature, the mixture was treated with 10% aq. HCl until it reached pH 2. After diluting slightly with water, the mixture was washed with ethyl acetate (2×), then the aqueous layer was treated with solid potassium carbonate until basic. This was extracted with chloroform (3×) and the combined organic extracts were dried over sodium sulfate and concentrated in vacuo. The residue was taken up in diethyl ether and a small amount of acetone, and 4.0 M HCl in dioxane (2 eq.) was added. The solid was collected via filtration, then triturated with hot ethyl acetate, and dried under vacuum, providing the product, (4,4-dimethylcyclohexyl){[2-methyl-4-(5-methyl-1H-benzimidazol-6-yl)phenyl]methyl}amine dihydrochloride, as a white solid. (M+1) 362.3 ES, 1.43 min. (LC/MS method A)

TABLE 13
Compounds Prepared by General Method H

| Ex. | Structure | Name | Method/Comments |
|---|---|---|---|
| H-2 | 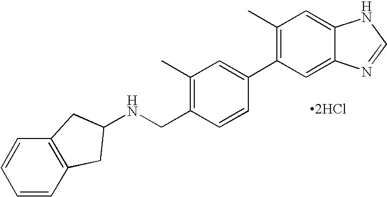 | N-{[2-methyl-4-(5-methyl-1H- benzimidazol-6- yl)phenyl]methyl}-2,3- dihydro-1H-inden-2-amine dihydrochloride (M + 1) 368.2 ES, 1.29 min. (LC/MS method A) | Method H No tritruation no final product |
| H-3 | 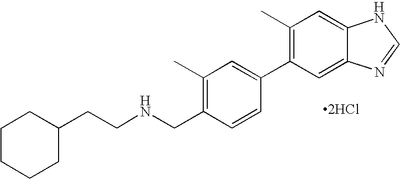 | 2-cyclohexyl-N-{[2-methyl-4- (5-methyl-1H-benzimidazol-6- yl)phenyl]methyl}ethan-amine dihydrochloride (M + 1) 362.2 ES, 1.47 min. (LC/MS method A) | Method H Triturated with mixture of ethyl acetate and methanol |
| H-4 | 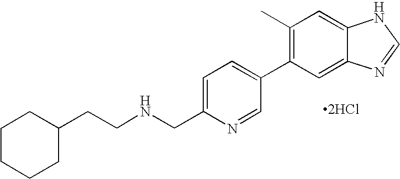 | 2-cyclohexyl-N-{[5-(5-methyl- 1H-benzimidazol-6-yl)-2- pyridinyl]methyl}ethanamine dihydrochloride (M + 1) 349.3 ES, 1.25 min. (LC/MS method A) | Method H Used Intermediate-73 No trituration of final product |
| H-5 | 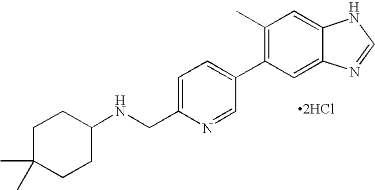 | 4,4-dimethyl-N-{[5-(5-methyl- 1H-benzimidazol-6-yl)-2- pyridinyl]methyl}cyclohexan- amine dihydrochloride (M + 1) 349.3 ES, 1.16 min. (LC/MS method A) | Method H Used Intermediate-73 No trituration of final product |
| H-6 | 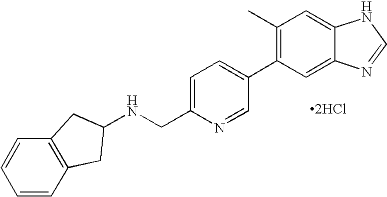 | 2,3-dihydro-1H-inden-2-yl{[5- (5-methyl-1H-benzimidazol-6- yl)-2-pyridinyl]methyl}amine dihydrochloride (M + 1) 355.2 ES, 0.98 min. (LC/MS method A) | Method H Used Intermediate-73 No trituration of final product |
| H-7 | 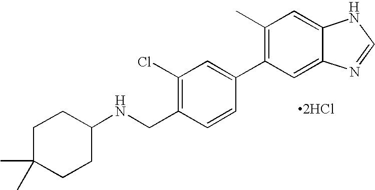 | N-{[2-chloro-4-(5-methyl-1H- benzimidazol-6- yl)phenyl]methyl}-4,4- dimethylcyclohexanamine dihydrochloride (M + 1) 382.2 ES, 1.58 min. (LC/MS method A) | Method H Used Intermediate-74 No trituration of final product |
| H-8 | 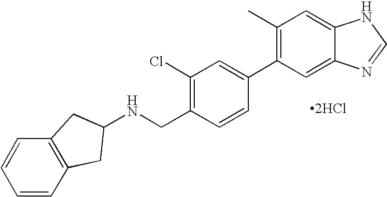 | N-{[2-chloro-4-(5-methyl-1H- benzimidazol-6- yl)phenyl]methyl}-2,3- dihydro-1H-inden-2-amine dihydrochloride (M + 1) 388.2 ES, 1.37 min. (LC/MS method A) | Method H Used Intermediate-74 No trituration of final product |
| H-9 | 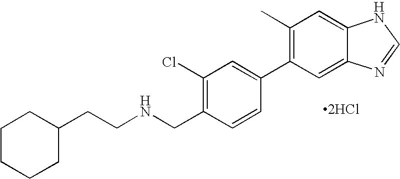 | N-{[2-chloro-4-(5-methyl-1H- benzimidazol-6- yl)phenyl]methyl}-2- cyclohexylethanamine dihydrochloride (M + 1) 382.2 ES, 1.64 min. (LC/MS method A) | Method H Used Intermediate-74 No trituration of final product |

General Method I:

### Example 1-1

### Step 1: 4-(1H-Indol-5-yl)benzaldehyde

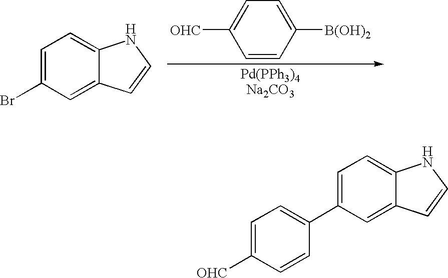

5-Bromo-indole (1.6 g, 8.16 mmol), 4-formylphenylboronic acid (1.47 g, 9.8 mmol) and tetrakis(triphenylphosphine)palladium(0) (0.2 g, 0.172 mmol) in 20 ml of toluene, 20 ml of ethanol and 20 ml of 1M aqueous sodium carbonate solution were degassed by vacuum-nitrogen backfilling cycles and heated to reflux for 1 h. Another portion of tetrakis(triphenylphosphine)palladium(0) (0.2 g) was added. The reaction was continued under reflux for 8 h. The mixture was cooled to the room temperature and partitioned between ethyl acetate and water. The organic layer was separated, and the aqueous layer was extracted with ethyl acetate twice. The combined organics were washed with brine, dried over magnesium sulfate and concentrated in vacuo. The residue was purified by silica gel column chromatography (20 to 50% ethyl acetate in hexanes) to give the title compound as a pinkish solid.

¹H NMR (400 MHz, CDCl₃) delta ppm 6.50 (s, 1H) 7.38 (s, 1H) 7.49 (s, 2H) 7.83-7.98 (m, 5H) 10.00 (s, 1H) 11.22 (s, 1H)

### Step 2: N-{[4-(1H-Indol-5-yl)phenyl]methyl}-2,3-dihydro-1H-inden-2-amine 2,3-dihydro-1H-inden-2-yl{[4-(1H-indol-5-yl)phenyl]methyl}amine Acetic Acid Salt

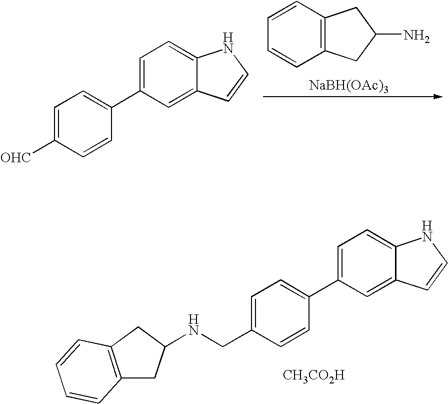

4-(1H-Indol)-5-yl)benzaldehyde (0.113 g, 0.51 mmol), and 2-aminoindane (0.13 g, 1.0 mmol) were stirred in 3 ml of 4% acetic acid in methanol plus a few drops of DMF for approximate 1 h before sodium triacetoxyborohydride (0.19 g, 0.9 mmol) was added. The reaction mixture was stirred for 15 h at room temperature, directly loaded on a 12 g ISCO silica gel column and eluted with 0 to 10% methanol in dichloromethane to give the title product as a light orange solid.

¹H NMR (400 MHz, DMSO-d₆) delta ppm 1.88 (s, 3H) 2.64-2.75 (m, 2H), 3.00-3.10 (m, 2H) 3.50 (m, 1H) 3.78 (s, 2H) 6.42 (s, 1H) 7.05-7.20 (m, 4H) 7.32-7.43 (m, 5H) 7.59 (m, 2H) 7.79 (s, 1H) 11.10 (s, 1H); (M+1) 339.60, 0.62 min (LC/MS method E).

TABLE 14
Compounds Prepared by General Method I

| Ex. | Structure | Name | Method/Comments |
|---|---|---|---|
| I-2 | 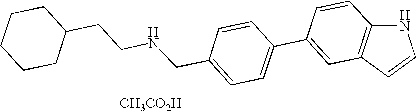 | (2-cyclohexylethyl){[4-(1H- indol-5-yl)phenyl]methyl} amine acetic acid salt (M + 1) 333.59, 0.68 min (LC/MS method E) | Method I |
| I-3 | 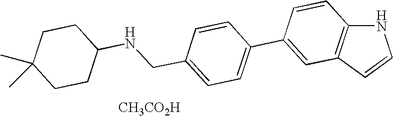 | N-{[4-(1H-indol-5- yl)phenyl]nethyl}-4,4- dimethylcyclohexanamine acetic acid salt (M − 1) 331.56, 0.67 min (LC/MS method E) | Method I |
| I-4 | 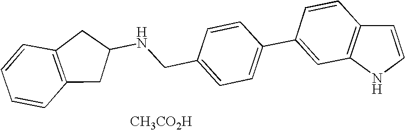 | N-{[4-(1H-indol-6- yl)phenyl]nethyl}-2,3-dihydro- 1H-inden-2-amine acetic acid salt (M + 1) 339.15, 1.91 min (LC/MS method B) | Method I Used 6- bromoindole as starting material |
| I-5 | 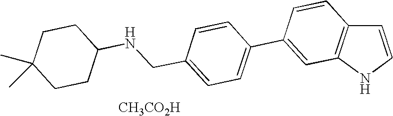 | N-{[4-(1H-indol-6- yl)phenyl]methyl}-4,4- dimethylcyclohexanamine acetic acid salt (M + 1) 333.18, 2.03 min (LC/MS method B) | Method I Used 6- bromoindole as starting material |
| I-6 | 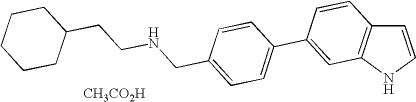 | (2-cyclohexylethyl){[4-(1H- indo1-6-yl)phenyl]methyl}amine acetic acid salt (M + 1) 333.18, 2.10 min (LC/MS method B) | Method I Used 6- bromoindole as starting material |

General Method J:

### Example J-1

### Step 1: 2-(3,5-difluorophenyl)-1,3-dioxolane

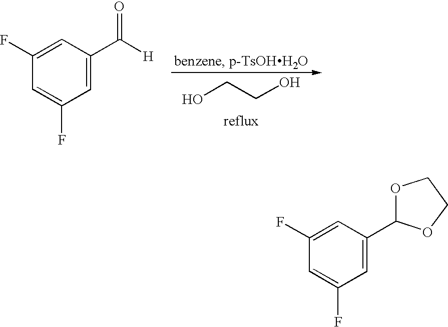

A benzene solution (400 mL) containing 3,5-difluorobenzaldehyde (7.77 g, 54.7 mmol), ethylene glycol (10.2 g, 164.3 mmol), and p-toluenesulfonic acid monohydrate (0.104 g, 0.55 mmol) was heated under reflux while removing water via a Dean-Stark trap for 15 h. The solution was cooled to RT. The organics were washed with water and saturated NaHCO₃ (aq), and dried with anhydrous MgSO₄. The solvent was removed in vacuo, and the residual oil was purified by silica gel column chromatography (EtOAc/hexanes), affording the title compound as a colorless oil (6.25 g, 61%). 1H NMR (400 MHz, CHLOROFORM-d) δ ppm 3.97-4.11 (m, 4H), 5.77 (s, 1H), 6.70-6.81 (m, 1H), 6.91-7.04 (m, 2H).

### Step 2: 2-(4-bromo-3,5-difluorophenyl)-1,3-dioxolane

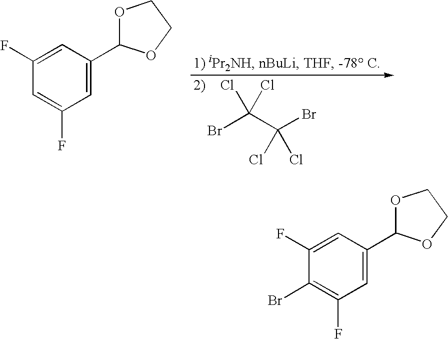

To a −78° C. solution of diisopropylamine (0.56 mL, 4.00 mmol) in THF (5 mL) was added a 2.5 M solution of nBuLi in hexanes (1.6 mL, 4.00 mmol). The mixture was stirred for 15 min followed by addition of 2-(3,5-difluorophenyl)-1,3-dioxolane (0.50 g, 2.69 mmol) in THF (2.5 mL). The reaction mixture was stirred for 1 h, and a solution of 1,2-dibromotetrachloroethane (1.75 g,. 5.37 mmol) in THF (5 mL) was added dropwise. After stirring for 2 h and allowing the reaction mixture to warm to RT, the mixture was quenched with water, and extracted with EtOAc. The combined organic phases were washed with water, dried with anhydrous MgSO₄, and concentrated in vacuo. The crude material was purified by silica gel column chromatography (EtOAc/hexanes), affording the title compound as a colorless liquid (0.34 g, 48%). 1H NMR (400 MHz, CHLOROFORM-d) δ ppm 3.98-4.13 (m, 4H), 5.77 (s, 1H), 7.08-7.10 (m, 2H) (impurities present in NMR).

### Step 3: 5-[4-(1,3-dioxolan-2-yl)-2,6-difluorophenyl-1-(triphenylmethyl)-1H-benzimidazole and 6-[4-(1,3-dioxolan-2-yl)-2,6-difluorophenyl-1-(triphenylmethyl)-1H-benzimidazole

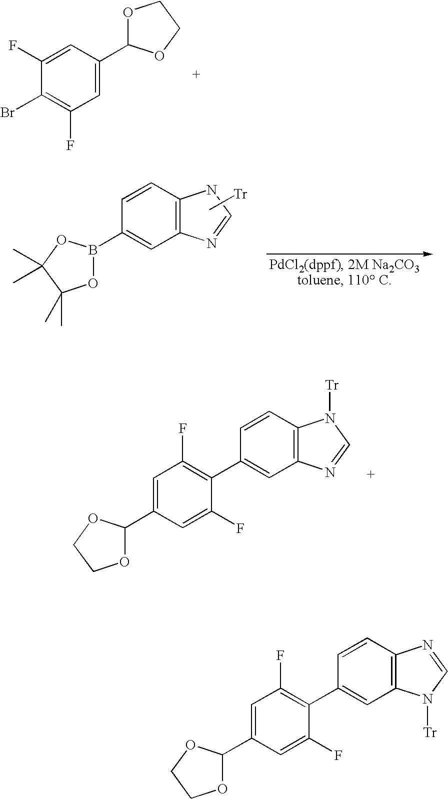

To a mixture of 2-(4-bromo-3,5-difluorophenyl)-1,3-dioxolane (0.583 g, 2.20 mmol), 5-(4,4,5,5-tetramethyl-1,3,2-dioxaborolan-2-yl)-1-(triphenylmethyl)-1H-benzimidazole and 6-(4,4,5,5-tetramethyl-1,3,2-dioxaborolan-2-yl)-1-(triphenylmethyl)-1H-benzimidazole (1.14 g, 2.34 mmol), and dichloro[1,1′-bis(diphenylphosphino)ferrocene]palladium (II) dichloromethane adduct (0.182 g, 0.22 mmol) in toluene was added 2M Na₂CO₃ (aq) (2.8 mL, 5.60 mmol). The mixture was heated at 100 degrees C. for 5 h. Additional PdCl₂(dppf) (95 mg) was added, and the mixture was continued to stir at 95 degrees C. overnight. Upon cooling, water was added, and the mixture was extracted with CH₂Cl₂. The combined organic extracts were dried with anhydrous Na₂SO₄ and concentrated in vacuo. The crude material was purified by silica gel column chromatography, affording the title compounds as a mixture of regioisomers (0.58 g, 48%). (M+1) 545.3 AP, 0.98 min, 545.4 AP, 1.01 min (LC/MS Method F)

### Step 4: 4-(1H-benzimidazol-5-yl)-3,5-difluorobenzaldehyde

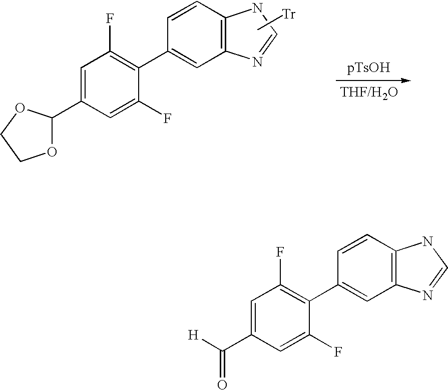

A solution of regioisomers, 5-[4-(1,3-dioxolan-2-yl)-2,6-difluorophenyl-1-(triphenylmethyl)-1H-benzimidazole and 6-[4-(1,3-dioxolan-2-yl)-2,6-difluorophenyl-1-(triphenylmethyl)-1H-benzimidazole (0.579 g, 1.06 mmol) and p-toluenesulfonic acid monohydrate (0.812 g, 4.27 mmol) in a mixture of THF and water (10:1 mL) was stirred at RT for 5 days. Saturated NaHCO₃ (aq) was added, and the mixture was extracted with EtOAc. The combined organic phases were dried with Na₂SO₄ and concentrated in vacuo, affording the title compound as a solid (0.57 g, >100%, crude). The crude material was used without purification. (M+1) 258.9 AP, 0.62 min (LC/MS Method F)

### Step 5: N-{[4-(1H-benzimidazol-5-yl)-3,5-difluorophenyl]methyl}-2,3-dihydro-1H-inden-2-amine dihydrochloride

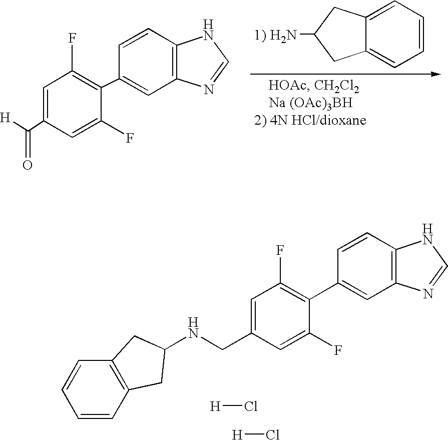

To a mixture of 4-(1H-benzimidazol-5-yl)-3,5-difluorobenzaldehyde (0.117 g, 0.45 mmol) and 2-aminoindan (0.090 mL, 0.69 mmol) was added acetic acid (0.25 mL). After stirring for 1 h at RT, sodium triacetoxyborohydride (0.197 g, 0.93 mmol) was added. The mixture was stirred at RT overnight. A solution of 5% Na₂CO₃ (aq) was added, and the mixture was extracted with EtOAc. The combined organic phases were washed with brine, dried with anhydrous Na₂SO₄, and concentrated in vacuo. The crude material was purified by silica gel column chromatography (MeOH/CH₂Cl₂). The resulting solid was dissolved in CH₂Cl₂ and MeOH, and a solution of 4N HCl in dioxane (0.13 mL) was added. After stirring for 15 min, the solid was filtered, washed with CH₂Cl₂, and dried, affording the title compound as a colorless solid (0.038 g, 20%). (M+1) 376.1 AP, 0.56 min (LC/MS Method F)

TABLE 15
Compounds Prepared by General Method J

| | | Name and Characterization | |
|---|---|---|---|
| Ex. | Structue | Data | Method/Comments |
| J-2 | 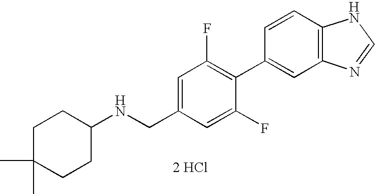 | N-{[4-(1H-benzimidazol-5- yl)-3,5- difluorophenyl]methyl}-4,4- dimethylcyclohexanamine dihydrochloride (M + 1) 370.2 AP, 0.62 min (LC/MS Method F) | Method J Synthesized from 4,4- dimethylcyclohexylamine hydrochloride. Added 95 uL of Et₃N and stirred 15 min before addition of HOAc. |
| J-3 | 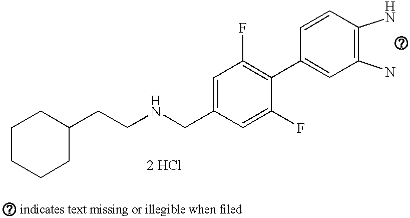 | N-{[4-(1H-benzimidazol-5- yl)-3,5- difluorophenyl]methyl}-2- cyclohexylethanamine dihydrochloride (M + 1) 370.1 AP, 0.64 min (LC/MS Method F) | Method J Synthesized from (2- cyclohexylethyl)amine hydrochloride. Added 95 uL of Et₃N and stirred 15 min before addition of HOAc. |

General Method K:

### Example K-1

### Step 1: 4-(1,3-benzoxazol-5-yl)benzaldehyde

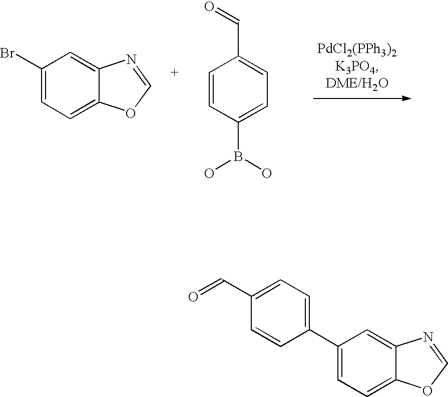

To a 100 mL round bottom was added 5-bromobenzoxazole (1.06 g, 5.35 mmol), 4-formylphenyl boronic acid (883 mg, 5.89 mmol), bistriphenylphosphine palladium chloride (350 mg, 0.5 mmol), potassium phosphate (3.4 g, 6.05 mmol) and DME/H₂O (3/1, 20 mL). The reaction mixture was stirred at 80 degrees C. overnight, filtered through celite and diluted with ethyl acetate. The organic layer was washed with saturated bicarbonate solution and then brine and was dried with MgSO₄. The mixture was concentrated and purified on silica gel column (40 g) using a gradient of hexanes and 40% ethyl acetate/hexanes to afford 4-(1,3-benzoxazol-5-yl)benzaldehyde.

LC/MS: (M+1) 224.03, 2.20 min (LC/MS Method A).

### Step 2: N-{[4-(1,3-benzoxazol-5-Aphenyl]methyl}-2,3-dihydro-1H-inden-2-amine

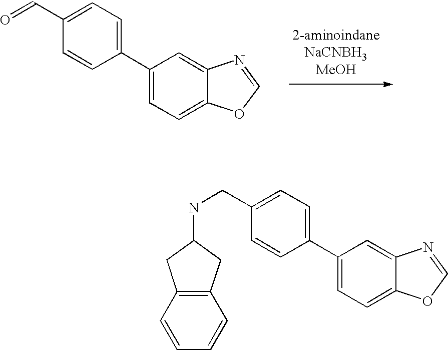

To a 20 mL scintillation vial was added 4-(1,3-benzoxazol-5-yl)benzaldehyde (60 mg, 0.27 mmol), 2-aminoindane (72 mg, 0.54 mmol), acetic acid (0.2 mL) and methanol (5 mL). The reaction mixture was stirred at 50 degrees C. for 30 min and cooled to room temperature. Sodium cyanoborohydride (28 mg, 0.45 mmol) was added, and the mixture was stirred at room temperature overnight. The reaction was quenched with 1 N NaOH, extracted with ethyl acetate and dried with MgSO₄. The mixture was purified on silica gel column (40 g) using a gradient of CH₂Cl₂ and 10% MeOH/CH₂Cl₂ to afford N-{[4-(1,3-benzoxazol-5-yl)phenyl]methyl}-2,3-dihydro-1H-inden-2-amine. LC/MS: (M+1) 341.08, 1.71 min. (LC/MS Method A).

TABLE 16
Compounds Prepared by General Method K

| | | Name and | |
|---|---|---|---|
| Ex. | Structure | Characterization Data | Method |
| K-2 | 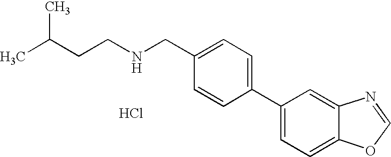 | N-{[4-(1,3-benz oxazol- 5-yl)phenyl]methyl}-3- methyl-1-butanamine hydrochloride (M + 1) 295.10, 1.65 min (LC/MS Method A) | Method K |
| K-3 | 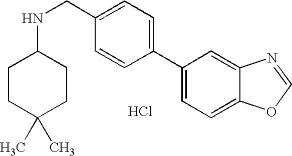 | N-{[4-(1,3-benz oxazol- 5-yl)phenyl]methyl}- 4,4-dimethyl cyclohexanamine hydrochloride (M + 1) 335.16, 1.83 min (LC/MS Method A) | Method K |
| K-4 | 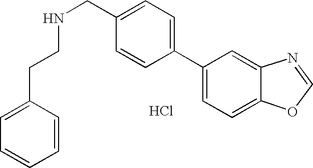 | N-{[4-(1,3-benz oxazol- 5-yl)phenyl]methyl}-2- phenyl ethanamine hydrochloride (M + 1) 329.07, 1.71 min (LC/MS Method A) | Method K |

General Method L:

It should be understood by one skilled in the art that the following procedure can be used to prepare other examples by using substituted benzimidazoles in Step 4 and by varying the aldehyde or ketone used in the reductive amination in Step 5.

### Example L-1

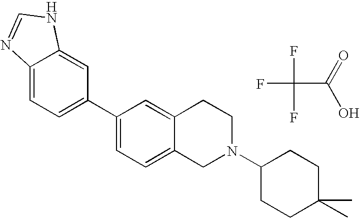

### 6-(1H-benzimidazol-5-yl)-2-(4,4-dimethylcyclohexyl)-1,2,3,4-tetrahydroisoquinoline trifluoroacetate

### Step 1: 1,2,3,4-Tetrahydro-6-isoquinolinol hydrobromide

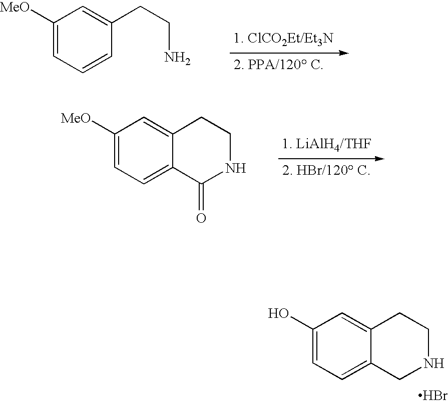

The title compound was synthesized in two steps from m-methoxyphenethylamine, according to the procedure of Sall and Grunewald (J. Med. Chem. 1987, 30, 2208) with the exception that ethyl chloroformate was substituted for methyl chloroformate in the Bischler-Naperalski synthesis of the intermediate 6-(methyloxy)-3,4-dihydro-1(2H)-isoquinolinone. ¹H NMR (400 MHz, DMSO-d₆) δ 2.90 (t, J=6.2 Hz, 2H), 3.32 (m, 2H), 4.13 (t, J=4.6 Hz, 2H), 6.59 (d, J=2.2 Hz, 1H), 6.65 (dd, J=8.4, 2.3 Hz, 1H), 7.00 (d, J=8.3 Hz, 1H), 9.00 (br. s, 2H).

### Step 2: 1,1-Dimethylethyl 6-hydroxy-3,4-dihydro-2(1H)-isoquinolinecarboxylate

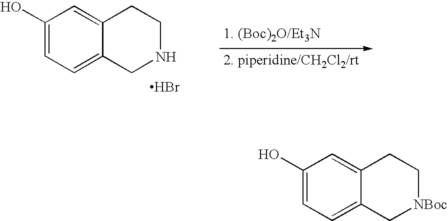

To a slurry of 1,2,3,4-tetrahydro-6-isoquinolinol hydrobromide (1.29 g; 5.63 mmol; step 1 above) and Et₃N (3.13 mL; 22.5 mmol) in CH₂Cl₂ (30 mL) and THF (5 mL) at rt was added a solution of (Boc)₂O (2.46 g; 11.3 mmol) in THF (20 mL). The mixture was stirred 72 h at rt and concentrated in vacuo. The residue was dissolved in CH₂Cl₂ and washed with H₂O. The aqueous wash was back-extracted with CH₂Cl₂ (×2), Combined organics were washed (H₂O, brine), dried over Na₂SO₄, filtered and concentrated in vacuo. The residue was dissolved in CH₂Cl₂ (30 mL), piperidine (30 mL) was added, the mixture was stirred overnight at rt and concentrated in vacuo. The residue was dissolved in EtOAc, washed (3× H₂O, brine), dried over Na₂SO₄, filtered and concentrated in vacuo. The residue was re-dissolved in EtOAc, washed (2×1M KHSO₄, H₂O, brine), dried over Na₂SO₄, filtered and concentrated in vacuo. The residue was purified by flash chromatography (EtOAc/hexanes), affording the title compound as a colorless gum. ¹H NMR (400 MHz, CDCl₃) δ 1.49 (s, 9H), 2.76 (t, J=5.9 Hz, 2H), 3.61 (br. t, J=5.8 Hz, 2H), 4.49 (s, 2H), 5.58 (br. s, 1H), 6.63 (unresolved d, 1H), 6.68 (br. d, J=8.4 Hz, 1H), 6.95 (d, J=8.2 Hz, 1H).

### Step 3: 1,1-Dimethylethyl 6-{[(trifluoromethyl)sulfonyl]oxy}-3,4-dihydro-2(1H)-isoquinolinecarboxylate

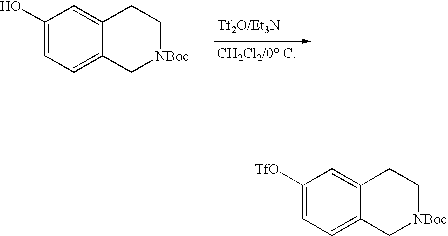

To a solution of 1,1-dimethylethyl 6-hydroxy-3,4-dihydro-2(1H)-isoquinolinecarboxylate (4.92 g; 19.8 mmol, as per step 2 above) and Et₃N (3.31 mL; 23.8 mmol) in CH₂Cl₂ at 0° C. was added Tf₂O (3.65 mL; 21.7 mmol), dropwise over 3 minutes. The mixture was slowly warmed to room temperature (overnight), poured into water and the layers were separated. The aqueous layer was extracted with CH₂Cl₂ (×2), combined organics were washed (water, brine), dried over Na₂SO₄ and concentrated in vacuo. The residue was purified by flash chromatography (EtOAc/hexanes), affording the title compound as a colorless gum which gradually solidified. ¹H NMR (400 MHz, CDCl₃) δ 1.49 (s, 9H), 2.87 (t, J=5.7 Hz, 2H), 3.66 (br. t, J=5.5 Hz, 2H), 4.59 (s, 2H), 7.04-7.12 (m, 2H), 7.18 (d, J=8.4 Hz, 1H).

### Step 4: 1,1-dimethylethyl 6-[1-(triphenylmethyl)-1H-benzimidazol-5-yl-3,4-dihydro-2(1H)-isoquinolinecarboxylate and 1,1-dimethylethyl 6-[1-(triphenylmethyl)-1H-benzimidazol-6-yl]-3,4-dihydro-2(1H)-isoquinolinecarboxylate

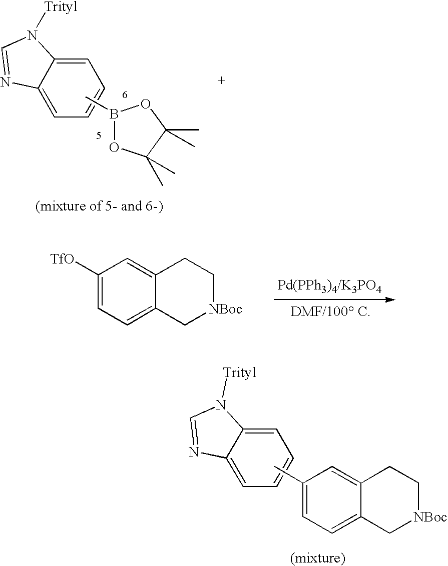

A mixture of 1,1-dimethylethyl 6-{[(trifluoromethyl)sulfonyl]oxy}-3,4-dihydro-2(1H)-isoquinolinecarboxylate (0.180 g; 0.47 mmol, per step 3 above), 5-(4,4,5,5-tetramethyl-1,3,2-dioxaborolan-2-yl)-1-(triphenylmethyl)-1H-benzimidazole and 6-(4,4,5,5-tetramethyl-1,3,2-dioxaborolan-2-yl)-1-(triphenylmethyl)-1H-benzimidazole (0.252 g; 0.519 mmol, ˜1:1 mixture of 5- and 6-regioisomers from General Method F, Example F-1, step 1), Pd(PPh₃)₄ (0.027 g; 0.024 mmol), K₃PO₄ (0.240 g; 1.13 mmol) and DMF (5 mL) was sparged with nitrogen for 10 minutes at room temperature, then heated at 100° C. for 18 h. The mixture was cooled, poured into water and extracted with EtOAc (×3). Combined organics were washed (water, brine), dried over Na₂SO₄ and concentrated in vacuo. The residue was purified by flash chromatography (EtOAc/hexanes), affording a mixture (˜1:1 by LC/MS) of the title compounds as a colorless foam. LC/MS method A; regioisomer 1: 592 ([M+H]⁺, weak), 243 (bp, trityl cation) ES, 3.28 min, regioisomer 2: 592 ([M−H]⁺, weak), 243 (bp, trityl cation) ES, 3.33 min.

### Step 5: 6-(1H-benzimidazol-5-yl)-2-(4,4-dimethylcyclohexyl)-1,2,3,4-tetrahydroisoquinoline trifluoroacetate

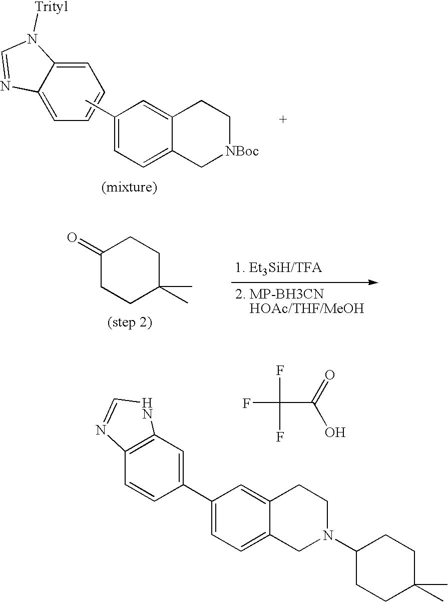

To a solution of 1,1-dimethylethyl 6-[1-(triphenylmethyl)-1H-benzimidazol-5-yl-3,4-dihydro-2(1H)-isoquinolinecarboxylate and 1,1-dimethylethyl 6-[1-(triphenylmethyl)-1H-benzimidazol-6-yl]-3,4-dihydro-2(1H)-isoquinolinecarboxylate (0.152 g; 0.26 mmol, ˜1:1 mixture) and Et₃SiH (0.20 mL; 1.26 mmol) in CH₂Cl₂ (5 mL) was added TFA (1 mL) in one portion. The mixture was stirred 2.5 h at room temperature and concentrated in vacuo. The residue was suspended between CHCl₃/satd Na₂CO₃, the whole was filtered, and the layers were separated. The aqueous layer was extracted with CHCl₃ (×2), combined organics were washed (water, brine), dried over Na₂SO₄ and concentrated in vacuo. The residue was dissolved in THF/MeOH/HOAc (3:2:0.2 mL), 4,4-Dimethylcyclohexanone (0.049 g; 0.39 mmol) was added, followed by MP-BH3CN (0.35 g @ ˜2.3 mmol/g; ˜0.8 mmol). The mixture was agitated overnight, resin was removed by filtration and the filtrate was concentrated in vacuo. The residue was purified by RP-HPLC (MeCN/water; 0.1% ea TFA additive) affording the title compound as a colorless film.

LC/MS method A: 360 ([M+H]⁺) ES 1.73 min.

Synthetic Descriptions of Intermediates Used in General Methods A through K

Intermediate-1

### Step 1: 4-[4-Methyl-1-(triphenylmethyl)-1H-benzimidazol-5-yl]phenol

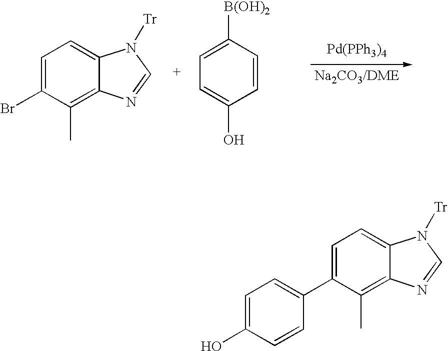

5-Bromo-4-methyl-1-(triphenylmethyl)-1H-benzimidazole [from Intermediate 3, Step 4] (3.115 g, 6.87 mmol), 4-hydroxyphenylboronic acid (1.42 g, 10.3 mmol) and tetrakis(triphenylphosphine)palladium(0) (0.15 g, 0.023 mmol) in 80 ml of dimethoxyethane and 17 ml of 2M sodium carbonate aqueous solution were degassed by vacuum-nitrogen backfilling cycles, heated to reflux for 15 h, cooled and partitioned between ethyl acetate and water. The organic phase was separated, washed with brine, dried over magnesium sulfate and concentrated in vacuo. The residue was purified by silica gel column chromatography (20 to 60% ethyl acetate in hexanes) to afford the title compound as a brownish yellow solid.

¹H NMR (400 MHz, CDCl₃) delta ppm 2.60 (s, 3H) 6.36 (d, 1H) 6.82 (m, 3H) 7.19 (m, 8H) 7.35 (m, 9H) 8.02 (br., 1H); LC-MS: (Ph₃C⁺) 243.02, 2.94 min (LC/MS method A)

### Step 2: 5-{4-[(2-Chloroethyl)oxy]phenyl}-4-methyl-1-(triphenylmethyl)-1H-benzimidazole

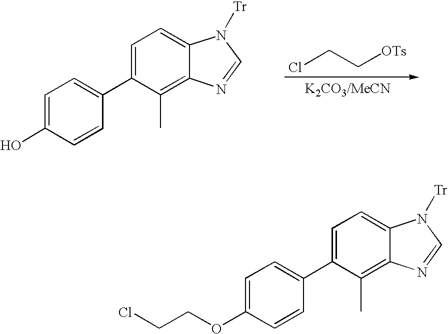

4-[4-Methyl-1-(triphenylmethyl)-1H-benzimidazol-5-yl]phenol (3.046 g, 6.54 mmol), 2-chloroethyl p-toluenesulfonate (4.75 ml, 26.16 mmol) and potassium carbonate (3.615 g, 26.16 mmol) in 90 ml of acetonitrile were heated to reflux for 24 h, concentrated in vacuo and partitioned between ethyl acetate and water. The off-white solid was isolated by filtration and washed with water and ethyl acetate to afford the title product. The organic was separated from the filtrate, washed with brine, dried over magnesium sulfate and concentrated in vacuo. The residue was washed with ethyl acetate and hexanes to give more title compound.

¹H NMR (400 MHz, DMSO-d₆) delta ppm 2.45 (s, 3H) 3.95 (t, 2H) 4.24 (t, 2H) 6.28 (d, 1H) 6.78 (d, 1H) 6.96 (d, 2H) 7.17-7.24 (m, 8H) 7.30-7.42 (m, 9H) 7.86 (s, 1H); (M+) 529.14, (Ph₃C⁺) 243.09, 3.23 min (LC/MS method A)

Intermediate-3

### Step 1: 1-Bromo-2-methyl-3,4-dinitrobenzene

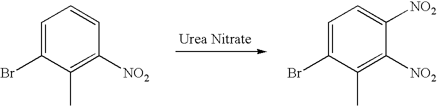

To a pre-cooled (0 degrees C.) solution of 2-bromo-6-nitrotoluene (21.6 g, 0.1 mol) in 216 ml of concentrated sulfuric acid was added urea nitrate (18.46 g, 0.12 mol) in small portions under efficient agitation to maintain the temperature at 0-10 degrees C. The reaction mixture was allowed to slowly warm up to room temperature and stirred overnight, and then was poured onto crushed ice (800 g). The solid was collected by filtration, thoroughly washed with water and dried in air. The crude material was recrystallized in ethyl acetate and hexanes to afford the pure title compound as a pale yellow solid.

¹H NMR (400 MHz, CDCl₃) delta ppm 2.42 (s, 3H) 7.85 (d, 1H) 7.95 (d, 1H)

### Step 2: 4-Bromo-3-methyl-1,2-benzenediamine

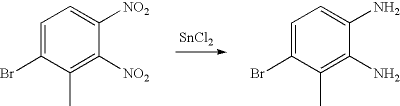

1-Bromo-2-methyl-3,4-dinitrobenzene (10.7 g, 0.041 mol), tin(II) chloride hydrate (64.79 g, 0.287 mol), ethyl acetate (128 ml) and ethanol (64 ml) were heated at 80° C. for 12 h, cooled to room temperature, poured onto crushed ice (1000 g) and adjusted with solid sodium bicarbonate to pH 7-8. The solid was removed by filtration and washed with ethyl acetate. The filtrate was extracted with ethyl acetate three times. The combined extracts were washed with brine, dried over magnesium sulfate and concentrated in vacuo. The residue was purified by silica gel column chromatography (10 to 50% ethyl acetate in hexanes) to give the title compound as a brown oil.

¹H NMR (400 MHz, CDCl₃) delta ppm 2.30 (s, 3H) 3.20-3.40 (br., 4H) 6.48 (d, 1H) 6.90 (d, 1H)

### Step 3: 5-Bromo-4-methyl-1H-benzimidazole

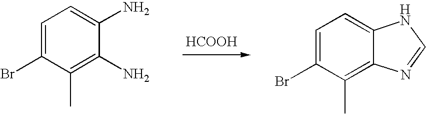

4-Bromo-3-methyl-1,2-benzenediamine (6.89 g, 34.3 mmol), formic acid (60 ml) and 37% concentrated HCl (150 ml) were heated at 60° C. for 12 h, cooled in an ice-water bath, and slowly adjusted with 28% concentrated ammonium solution (280 ml) to pH8-9. The solid was collected by filtration, washed with water and dried in air to afford the title compound as a pinkish white solid.

¹H NMR (400 MHz, DMSO-d₆) delta ppm 2.58 (s, 3H) 7.32 (s, 2H) 8.20 (s, 1H) 12.62 (br., 1H); (M+1) 212.94, 1.11 min (LC/MS method A)

### Step 4: 5-Bromo-4-methyl-1-(triphenylmethyl)-1H-benzimidazole

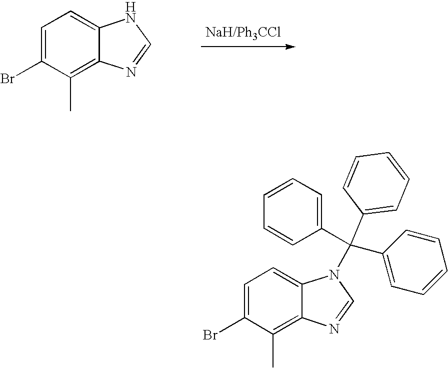

5-Bromo-4-methyl-1H-benzimidazole (6.62 g, 31.36 mmol) was dissolved in anhydrous tetrahydrofuran (150 ml) and cooled to 0° C. Sodium hydride oil dispersion (60%, 1.55 g, 38.75 mmol) was slowly added. The mixture was stirred for 1 h at 0° C. Triphenylmethyl chloride and a catalytic amount of tetrabutylammonium iodide were added. The resultant mixture was heated at reflux for 12 h, cooled to room temperature, quenched with saturated ammonium chloride solution (50 ml) and partitioned between ethyl acetate and water. The organic phase was separated, washed with brine, dried over magnesium sulfate and concentrated in vacuo. The residue was suspended in 50 ml of 50% ethyl acetate in hexanes, heated to reflux, cooled to room temperature and filtered to afford the title compound as a pale brown solid. The filtrate was concentrated and purified by silica gel column chromatography (5 to 20% ethyl acetate in hexanes) to give more title compound.

¹H NMR (400 MHz, CDCl₃) delta ppm 2.70 (s, 3H) 6.19 (d, 1H) 7.00-7.20 (m, 7H) 7.20-7.45 (m, 9H) 7.92 (s, 1H); LC-MS: (Ph₃C⁺) 243.11, 3.27 min (LC/MS method A)

Intermediate-5

### Stept 4-Bromo-2-nitro-5-(trifluoromethyl)aniline

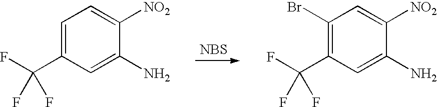

A mixture of 3-amino-4-nitrobenzotrifluoride (5.4 g, 26.2 mmol) and N-bromosuccinimide (4.59 g, 25.8 mmol) in acetic acid was heated under reflux, with stirring, for 4 h. The reaction mixture was cooled and poured into 1.5 L of water with agitation. The precipitate was suction-filtered to give a yellow solid that was further purified by silica gel column chromatography (0 to 40% ethyl acetate in hexanes) to afford the title compound as a yellow solid. LC-MS: 285.03(M−), 2.75 min (LC/MS method A)

### Step 2: 4-Bromo-5-(trifluoromethyl)-1,2-benzenediamine

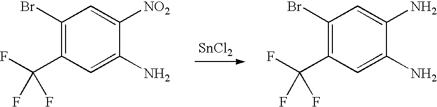

To the solution of 4-bromo-2-nitro-5-(trifluoromethyl)aniline (3.19 g, 11.2 mmol) in 35 ml of ethyl acetate and 17.5 ml of ethanol was added tin(II) chloride hydrate. The mixture was heated at 75-80° C. for 4 h, cooled, poured onto crushed ice (200 g) and neutralized with saturated sodium bicarbonate aqueous solution to pH8-9. The solid was removed by filtration thorough a Celite pad and washed with ethyl acetate (150 ml). The organic was separated from the filtrate and the aqueous phase was extracted with ethyl acetate twice. The organic was combined, washed with brine, dried over magnesium sulfate and concentrated in vacuo to afford the title product as a brown crystalline solid. LC-MS: 280.87(M+), 2.02 min (LC/MS method A)

### Step 3: 5-Bromo-6-trifluoromethyl-1H-benzimidazole

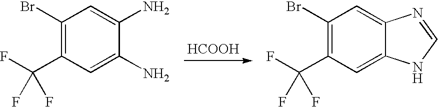

Proceeding in a similar manner to Intermediate 3, Step 3, but using 4-bromo-5-(trifluoromethyl)-1,2-benzenediamine, gave the title compound as a beige solid.

¹H NMR (400 MHz, CDCl₃) delta ppm 8.00 (s, 1H) 8.10 (s, 1H) 8.20 (s, 1H); (M+1) 266.85, 2.29 min (LC/MS method A)

### Step 4: 5-Bromo-6-trifluoromethyl-1-(triphenylmethyl)-1H-benzimidazole & 6-Bromo-5-trifluoromethyl-1-(triphenylmethyl)-1H-benzimidazole

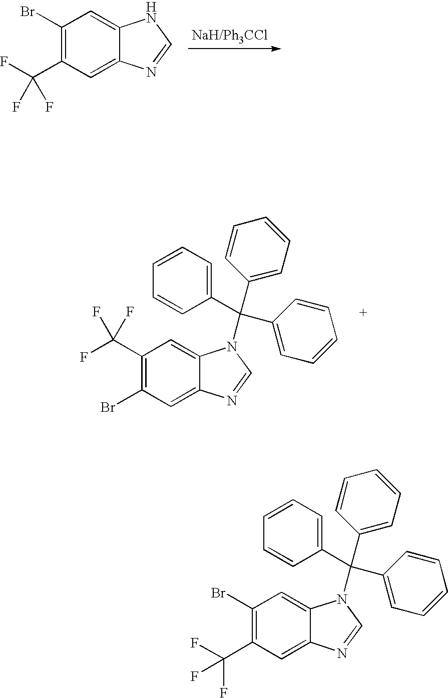

Proceeding in a similar manner to Intermediate 3, Step 4, but using 5-bromo-6-trifluoromethyl-1H-benzimidazole, gave the title product (a mixture of the two regioisomers) as a white to pink solid.

¹H NMR (400 MHz, CDCl₃) delta ppm 6.72 (s, 1H) 7.10-7.42 (m, 15H) 7.98-8.18 (m, 2H); LC-MS: 243.11(Ph₃C+), 3.19 min (LC/MS method A)

Intermediate-7

### Step 1: 5-Bromo-6-(methyloxy)-1H-benzimidazole

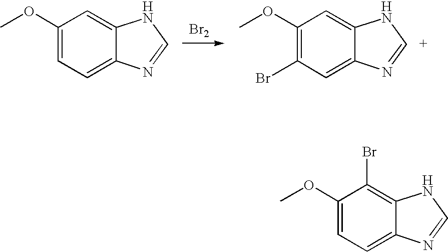

Into a suspension of 5-methoxy-1H-benzimidazole (3.76 g, 25.4 mmol) and potassium acetate (10 g, 102 mmol) in 250 ml of water was added dropwise a bromine solution (1.3 ml, 25.4 mmol in 300 ml of water) at room temperature in the period of 1 h. The reaction mixture was stirred for 3 h and extracted with ethyl acetate 3 times. The organics were combined, washed with brine, dried over magnesium sulfate and concentrated in vacuo. The brown solid residue was purified by silica gel column chromatography (ethyl acetate) to afford the title compound as a brown solid and the 4-bromo isomer as a beige solid.

5-Bromo-6-(methyloxy)-1H-benzimidazole: ¹H NMR (400 MHz, CDCl₃) delta ppm 3.80 (s, 3H) 7.06 (s, 1H) 7.80 (s, 1H) 8.20 (s, 1H) 12.40 (s, 1H); LC-MS: 228.95 (M+1), 0.83 min (LC/MS method A)

### 4-Bromo-5-(methyloxy)-1H-benzimidazole

¹H NMR (400 MHz, CDCl₃) delta ppm 3.80 (s, 3H) 7.00 (m, 1H) 7.40-7.62 (m, 1H) 8.20 (br., 1H) 12.60 (s, 1H); LC-MS: 228.95(M+1), 0.76 min (LC/MS method A)

### Step 2: 5-Bromo-6-methoxy-1-(triphenylmethyl)-1H-benzimidazole & 6-Bromo-5-methoxy-1-(triphenylmethyl)-1H-benzimidazole

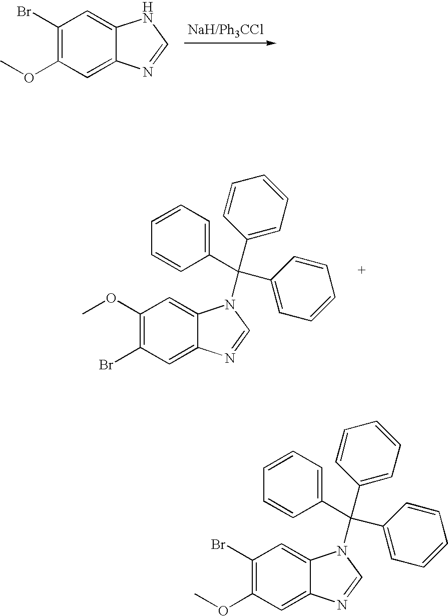

Proceeding in a similar manner to Intermediate 3, Step 4, but using 5-bromo-6-(methyloxy)-1H-benzimidazole, gave the title product (a mixture of the two regioisomers) as a white to pink solid.

LC-MS: 243.11(Ph₃C+), 3.04 min (LC/MS method A)

Intermediate-18

### 5-bromo-N,N-dimethyl-1H-benzimidazol-2-amine

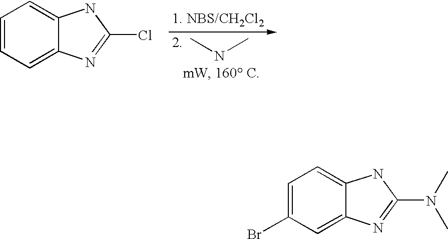

A mixture of 2-chloro -1H-benzimidazole (2.0 g; 13.1 mmol) and NBS (3.50 g; 19.9 mmol) in CH₂Cl₂ (60 mL) was stirred at room temperature for 16 hr. The solution was evaporated and the residue was partitioned between EtOAc/H₂O, the layers were separated, the organic layer was washed (H₂O, brine), dried over Na₂SO₄, filtered and concentrated in vacuo to give a white solid. To 300 mg of this solid was added N,N-dimethylamine (2.5 mL, 2.0 M in THF) and the mixture irradiated in a microwave at 160 degrees C. for 45 min. The solution was then concentrated and the residue partitioned between EtOAc/H₂O, the layers were separated, the organic layer was washed with H₂O then brine, dried over Na₂SO₄, filtered and concentrated in vacuo. The residue was purified by flash chromatography (acetone/CH₂Cl₂), affording the title compound as a white solid. ¹H NMR (400 MHz, DMSO-d₆) δ 3.01 (s, 6H), 6.94 (d, J=8.7 Hz, 1H), 7.04 (d, J=8.7 Hz, 1H), 7.27 (s, 1H).

Intermediate-20

### Step 1: Preparation of 1-bromo-4-(dibromomethyl)naphthalene

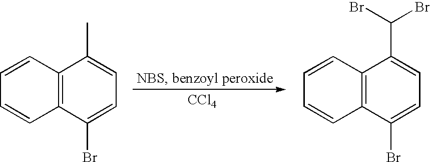

To a solution of 1-bromo-4-methylnaphthalene (5.0 g, 22.6 mmol) in carbon tetrachloride (75 mL) was added N-bromosuccinimide (10.0 g, 56.5 mmol) followed by benzoyl peroxide (0.3 g, 1.1 mmol). The reaction was heated at reflux overnight, then cooled to room temperature and filtered through Celite. The Celite was rinsed with chloroform, then the organics were concentrated in vacuo and purified via chromatography (100% hexanes), providing the product 1-bromo-4-(dibromomethyl)naphthalene.

### Step 2: Preparation of 4-bromo-1-naphthalenecarbaldehyde

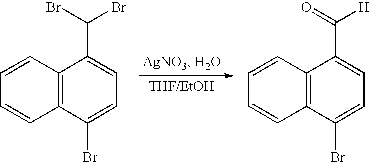

A solution 1-bromo-4-(dibromomethyl)naphthalene (7.45 g, 19.7 mmol) in tetrahydrofuran (60 mL) and ethanol (90 mL) was heated at reflux, and a solution of silver nitrate (10.0 g, 59.0 mmol) in water (30 mL) was added dropwise. The reaction was stirred at reflux for 2 h, then the mixture was filtered through Celite and the filter cake was washed with hot THF. The solvent was removed in vacuo and the residue was purified via chromatography (0 to 5% ethyl acetate/hexanes). This provided the product, 4-bromo-1-naphthalenecarbaldehyde, as a pale yellow oil. ¹H NMR (400 MHz, CDCl₃) δ 7.75-7.70 (m, 2H), 7.82 (d, J=7.6 Hz, 1H), 7.98 (d, J=7.6 Hz, 1H), 8.37 (d, J=8.1 Hz, 1H), 9.28 (d, J=8.1 Hz, 1H), 10.37 (s, 1H).

### Step 3: Preparation of 4-(4,4,5,5-tetramethyl-1,3,2-dioxaborolan-2-yl)-1-naphthalenecarbaldehyde

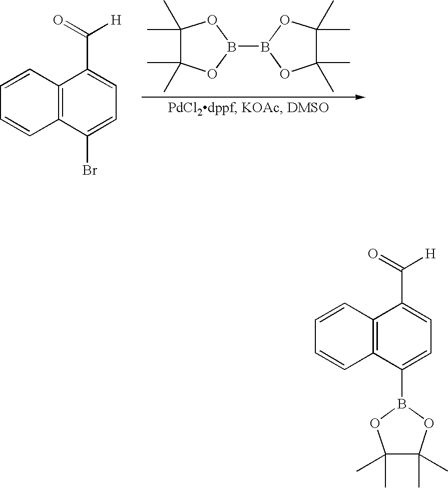

A mixture of 4-bromo-1-naphthalenecarbaldehyde (1.36 g, 5.8 mmol), bis(pinacolato)diboron (1.5 g, 6.4 mmol), potassium acetate (1.7 g, 17.4 mmol), and PdCl₂.dppf (0.2 g, 0.3 mmol) in DMSO (30 mL) was heated at 80° C. overnight. The reaction mixture was poured into water and chloroform and the layers were separated. The organic layer was washed with water and brine, then dried over magnesium sulfate. The solvent was removed in vacuo and the residue was purified by chromatography (5 to 20% ethyl acetate/hexane) to provide the product, 4-(4,4,5,5-tetramethyl-1,3,2-dioxaborolan-2-yl)-1-naphthalenecarbaldehyde. ¹H NMR (400 MHz, CDCl₃) δ 1.44 (s, 12H), 7.68-7.62 (m, 2H), 7.96 (d, J=6.8 Hz, 1H), 8.19 (d, J=6.8 Hz, 1H), 8.22 (d, J=7.8 Hz, 1H), 9.22 (d, J=8.1 Hz, 1H), 10.44 (s, 1H).

Intermediate-23

### Step 1: 3-fluoro-2-nitroaniline

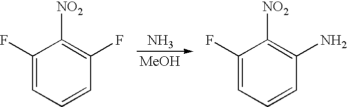

A solution of 7.0 M NH₃ in MeOH (200 mL) containing 2,6-difluoronitrobenzene (8.01 g, 50.3 mmol) was stirred at room temperature for 2 days in a sealed pressure vessel. The solution was then poured into H₂O and the organics were extracted with CH₂Cl₂ (2×). The combined organic layers were dried over MgSO₄ and the solvent removed in vacuo. The residual solid was triterated with hexanes and collected via vacuum filtration yielding 6.20 g (39.7 mmol) of 3-fluoro-2-nitroaniline. ¹H NMR (400 MHz, CDCl₃) δ 7.21 (m, 1H), 6.54 (d, 1H, J=8.6 Hz), 6.45 (m, 1H), 5.61 (s(br), 2H) ppm.

### Step 2: 4-bromo-3-fluoro-2-nitroaniline

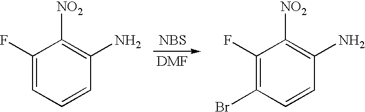

To a DMF solution (15 mL) containing 3-fluoro-2-nitroaniline (1.01 g (6.47 mmol) cooled to 0° C. was added a DMF solution (10 mL) containing NBS (1.15 g, 6.47 mmol). The reaction was stirred at 0 degrees C. for 30 min and then the cold bath removed. After stirring overnight the organics were taken up in EtOAc. The organic layer was washed with H₂O (2×), dried over MgSO₄ and the solvent removed in vacuo yielding 1.51 g (6.41 mmol) of 4-bromo-3-fluoro-2-nitroaniline which was taken on crude. ¹H NMR (400 MHz, CDCl₃)7.38 (dd, 2H, J=91. & 6.8 Hz), 6.50 (dd, 1H, J=9.0 & 1.9 Hz), 5.59 (s(br), 2H) ppm.

### Step 3: 4-bromo-3-fluoro-1,2-benzenediamine

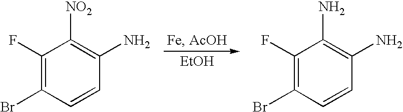

A flask containing 4-bromo-3-fluoro-2-nitroaniline (570 mg, 2.42 mmol) in 12 mL of EtOH had iron powder (339 mg, 6.06 mmol) and acetic acid (2.04 g, 33.9 mmol) added to it. The solution was heated to reflux for 4 hr. Upon cooling the black solution was carefully poured into a sat. Na₂CO₃ solution (˜50 mL). The heterogeneous solution was then filtered through a bed of celite with the celite being rinsed thoroughly with CHCl₃. The filtrate was made basic with 1 N NaOH and the organics were extracted with CHCl₃ (2×). The combined organic layers were dried over MgSO₄ and the solvent removed in vacuo. The residue was purified on the Biotage (30-50% EtOAc/hexanes) yielding 312 mg (1.52 mmol) of 4-bromo-3-fluoro-1,2-benzenediamine. ¹H NMR (400 MHz, CDCl₃) δ 8.6.79 (m, 1H), 6.36 (m, 1H), 3.47 (s(br), 2H), 3.41 (s(br), 2H) ppm.

### Step 4: 5-bromo-4-fluoro-1H-benzimidazole

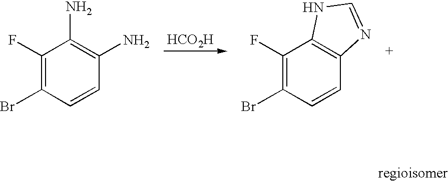

A flask containing 4-bromo-3-fluoro-1,2-benzenediamine (1.12 g, 5.47 mmol) in 10 mL of formic acid was heated to reflux for 7 hr. Upon cooling the solution was made basic with 1 N NaOH and the organics were extracted with EtOAc (2×) followed by drying over MgSO₄. The solvent was removed in vacuo and the residual solid triterated hexanes and collected via vacuum filtration yielding 1.21 g of 5-bromo-4-fluoro-1H-benzimidazole which was taken on crude. (M+1) 215 & 217, 1.8 min. (LC/MS Method A).

### Step 5: 6-bromo-7-fluoro-1-(triphenylmethyl)-1H-benzimidazole

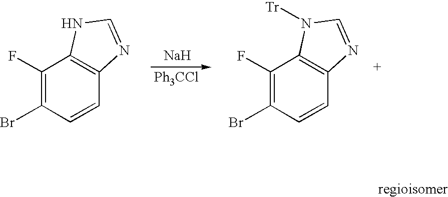

To a THF solution (30 mL) containing 5-bromo-4-fluoro-1H-benzimidazole (1.19 g, 5.53 mmol) cooled to 0 degrees C. was added NaH (266 mg, 6.64 mmol, 60% dispersion in mineral oil). The solution was stirred for 30 min when trityl chloride (2.0 g, 7.19 mmol) and nBu₄NI (20 mg, 0.055 mmol, 1 mole %) were added. The solution was then heated to reflux for 6 hr. Upon cooling the reaction was quenched with H₂O and the organics taken up in EtOAc. The organic layer was washed with H₂O, dried over MgSO₄ and the solvent removed in vacuo. The residual oil was purified on the Biotage (20-40% EtOAc/hexanes) yielding 1.23 g (2.69 mmol) of 6-bromo-7-fluoro-1-(triphenylmethyl)-1H-benzimidazole as a mixture of regioisomers. (M+1) 458, 3.3 min. (LC/MS Method A).

Intermediate-25

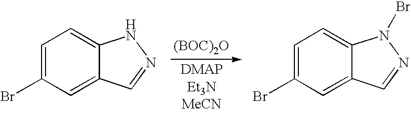

To an CH₃CN solution (100 mL) containing 5-bromoindazole (4.80 g, 24.4 mmol) was added DMAP (595 mg, 4.87 mmol), Et₃N (2.47 g, 24.4 mmol) and di-t-butyl dicarbonate (5.85 g, 26.8 mmol) at room temperature. The resulting solution was stirred overnight at which time the bulk of the CH₃CN was removed in vacuo. The residue was taken up in EtOAc and washed with H₂O and sat. NaHSO₄ followed by drying over MgSO₄. The solvent was removed in vacuo and the residue purified on the Biotage (20-30% EtOAc/hexanes) yielding 7.01 g (23.6 mmol) of 1,1-dimethylethyl 5-bromo-1H-indazole-1-carboxylate. ¹H NMR (400 MHz, CDCl₃) δ 8.08 (s, 1H), 8.05 (d, 1H, J=9.0 Hz), 7.85 (d, 1H, J=1.7 Hz), 7.58 (dd, 1H, J=9.0 & 1.8 Hz), 1.69 (s, 9H) ppm.

Intermediate-27

### Step 1: Preparation of 6-bromo-2,3-pyridinediamine

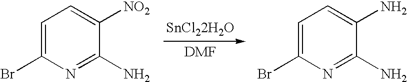

To a DMF solution (40 mL) containing 2-amino-6-bromo-3-nitropyridine (2.43 g, 11.1 mmol, as described in WO2004002986) was added SnCl₂.2H₂O (10.0 g, 44.4 mmol). The solution was then heated to 80 degrees C. for 2 hr. Upon cooling the solution was made basic with 1 N NaOH. The organics were extracted with EtOAc (4×) followed by drying over MgSO₄. The solvent was removed in vacuo and the residue purified on the Biotage (70-90% EtOAc/hexanes) yielding 1.26 g (6.69 mmol) of 6-bromo-2,3-pyridinediamine. ¹H NMR (400 MHz, d₆-DMSO) δ 8.6.56 (d, 1H, J=7.7 Hz), 6.42 (d, 1H, J=7.7 Hz), 5.77 (s(br), 2H), 4.74 (s(br), 2H) ppm

### Step 2: Preparation of 5-bromo-1H-imidazo[4,5-b]pyridine

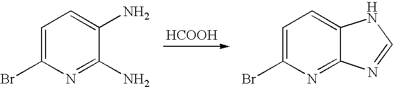

A flask containing 6-bromo-2,3-pyridinediamine (2.14 g, 11.4 mmol) in 30 mL of formic acid was heated to reflux for 4.5 hr. Upon cooling the solution was made basic with 5.0 M NaOH. The organics were extracted with EtOAc (4×) and dried over MgSO₄. The solvent was removed in vacuo yielding 1.84 g (9.29 mmol) of 5-bromo-1H-imidazo[4,5-b]pyridine which was taken on crude. ¹H NMR (400 MHz, d₆-DMSO) δ 8.44 (s, 1H), 7.94 (d, 1H, J=8.2 Hz), 7.37 (d, 1H, J=8.5 Hz) ppm.

### Step 3: Preparation of 5-bromo-1-(triphenylmethyl)-1H-imidazo[4,5-b]pyridine

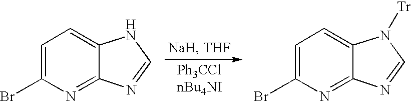

To a THF solution (80 mL) containing 5-bromo-1H-imidazo[4,5-b]pyridine (1.81 g, 9.14 mmol) cooled to 0 degrees C. was added NaH (439 mg, 10.9 mmol, 60% dispersion in mineral oil). The solution was stirred for 30 min and then trityl chloride (3.31 g, 11.9 mmol) and nBu₄NI (34 mg, 0.09 mmol, 1 mole %) were added. The solution was heated to reflux for 4 hr and upon cooling was diluted with H₂O. The organics were extracted with EtOAc (3×), dried over MgSO₄ and the solvent removed in vacuo. The residual oil was purified on the Biotage (30-70%

EtOAc/hexanes) yielding 3.57 g (8.10 mmol) of 5-bromo-1-(triphenylmethyl)-1H-imidazo[4,5-b]pyridine as a mixture of regioisomers.

Intermediate-29

### Step 1: 6-chloro-7-(triphenylmethyl)-7H-purine and 6-chloro-9-(triphenylmethyl)-7H-purine preparation

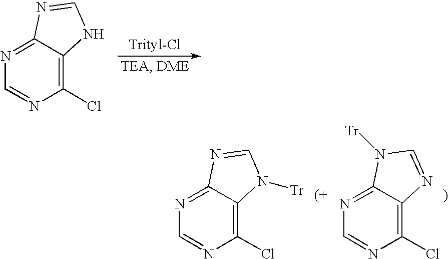

To a flask containing 6-chloropurine (1.55 g, 10.03 mmol) and trityl chloride (3.35 g, 12.03 mmol) in DME (10 mL) at 60 degrees Celsius was added triethylamine (1.82 mL, 13.04 mmol) over 5 min. The reaction was brought to a gentle reflux for 1.5 hours and then cooled. The reaction contents were partitioned between EtOAc and water. The aqueous was extracted an additional time and the combined organics washed with brine, dried (Na₂SO₄), filtered and concentrated. The residue was chromatographed on silica gel using Hexanes-EtOAc to afford the product as a tan solid (3.15 g, 79% yield).

¹H NMR (400 MHz, CDCl₃) delta ppm 7.13-7.34 (m, 15H) 8.11 (s, 1H) 8.44 (s, 1H)

Intermediate-30, 32, 34, 36 and 44.

### Step 1: Preparation of (4-bromo-2-methylphenyl)methanol

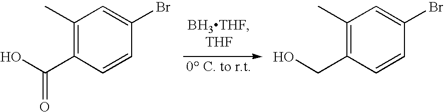

To a solution of 4-bromo-2-methylbenzoic acid (50.0 g, 233 mmol) in tetrahydrofuran (300 mL) at 0° C. was added BH₃THF (1.0 M solution in THF, 465 mL, 465 mmol) dropwise. The reaction was then allowed to warm to room temperature and stir overnight. The reaction mixture was cooled to 0 degrees C. and quenched by dropwise addition of water. The bulk of the solvent was removed in vacuo and the residue was partitioned between ethyl acetate and water. The organics were washed with brine, dried over sodium sulfate, and concentrated in vacuo. Chromatography (20 to 35% ethyl acetate/hexane) provided the product, (4-bromo-2-methylphenyl)methanol, as a white solid. ¹H NMR (400 MHz, CDCl₃) δ 2.31 (s, 3H), 4.64 (s, 2H), 7.18-7.27 (m, 1H), 7.28-7.37 (m, 2H)

### Step 2: Preparation of 4-bromo-2-methylbenzaldehyde (Intermediate 30)

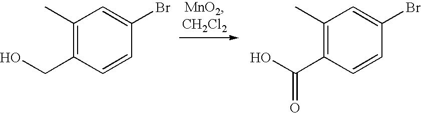

Manganese dioxide (360 g, 4.1 mol) was added to a solution of (4-bromo-2-methylphenyl)methanol (91.5 g, 455 mmol) in dichloromethane (500 mL) and the reaction was stirred at room temperature for 72 h. The reaction mixture was filtered through Celite and the filtrate was concentrated in vacuo. The residue was purified via chromatography (5% ethyl acetate/hex), providing the product, 4-bromo-2-methylbenzaldehyde, as a yellow solid. ¹H NMR (400 MHz, CDCl₃) δ 2.64 (s, 3H), 7.43 (s, 1H), 7.49 (d, J=8.3 Hz, 1H), 7.65 (d, J=8.3 Hz, 1H), 10.20 (s, 1H).

The following substituted 4-bromobenzaldehydes were synthesized in an analogous manner:

| | | Name and Characterization | |
|---|---|---|---|
| Ex. | Structure | Data | Comments |
| Intermediate-32 | 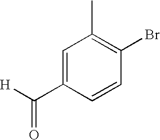 | 4-bromo-3- methylbenzaldehyde ¹H NMR (400 MHz, CDCl₃) δ - 2.48(s, 3H), 7.54(dd, J = 8.12, 1.65 Hz, 1H), 7.62- 7.83(m, 2H), 9.95(s, 1H) | |
| Intermediate-34 | 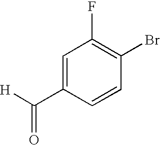 | 4-bromo-3- fluorobenzaldehyde ¹H NMR (400 MHz, CDCl₃) δ - 7.44-7.70(m, 2H), 7.76 (dd, J = 8.06, 6.47 Hz, 1H), 9.95(d, J = 1.71 Hz, 1H) | |
| Intermediate-36 | 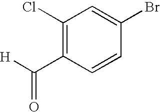 | 4-bromo-2- chlorobenzaldehyde ¹H NMR (400 MHz, CDCl₃) δ - 7.54(d, J = 8.42, 1H), 7.65(d, J = 1.46 Hz, 1H), 7.78(d, J = 8.42 Hz, 1H), 10.41(s, 1H) | |
| Intermediate-44 | 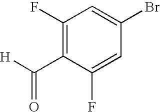 | 4-bromo-2,6- difluorobenzaldehyde ¹H NMR (400 MHz, CDCl₃) δ - 7.21(d, J = 7.81 Hz, 2H), 10.28(s, 1H) | The benzylic alcohol was commercial. |

Intermediates 38 and 41

### Step 1: Preparation of 4-bromo-1-(dibromomethyl)-2-(trifluoromethyl)benzene

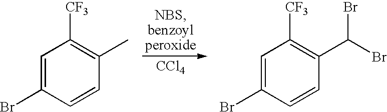

To a solution of 4-methyl-3-trifluoromethyl bromobenzene (1.0 g, 4.2 mmol) in carbon tetrachloride (15 mL) was added N-bromosuccinimide (1.9 g, 10.5 mmol) followed by benzoyl peroxide (50 mg, 0.2 mmol). The reaction was heated at reflux overnight, then was cooled to room temperature and filtered through Celite. The Celite was rinsed with chloroform, then the organics were concentrated in vacuo and the residue was purified via chromatography (0 to 5% ethyl acetate/hexane), providing the product, 4-bromo-1-(dibromomethyl)-2-(trifluoromethyl)benzene, as a colorless oil. ¹H NMR (400 MHz, CDCl₃) δ 6.94 (s, 1H), 7.69 (d, J=1.71 Hz, 1H), 7.79 (dd, J=8.54, 1.83 Hz, 1H), 8.06 (d, J=8.67 Hz, 1H)

### Step 2: Preparation of 4-bromo-2-(trifluoromethyl)benzaldehyde (Intermediate 38)

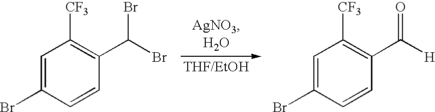

A solution of 4-bromo-1-(dibromomethyl)-2-(trifluoromethyl)benzene (0.50 g, 1.3 mmol) in ethanol (8 mL) and tetrahydrofuran (5 mL) was heated at reflux and a solution of silver nitrate (0.64 g, 3.8 mmol) in water (2 mL) was added dropwise. After stirring 2 h at reflux, the mixture was filtered through Celite and the filter cake was rinsed with hot tetrahydrofuran. The filtrate was concentrated in vacuo and purified via chromatography (5% ethyl acetate/hexanes) to provide the product, 4-bromo-2-(trifluoromethyl)benzaldehyde, as a yellow solid. ¹H NMR (400 MHz, CDCl₃) δ 7.85 (d, J=8.3 Hz, 1H), 7.93 (s, 1H), 8.00 (d, J=8.30 Hz, 1H), 10.33 (d, J=1.59 Hz, 1H).

The following aldehyde was synthesized in an analogous manner:

| Ex. | Structure | Name and Characterization Data |
|---|---|---|
| Intermediate-41 | 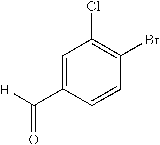 | 4-bromo-3-chlorobenzaldehyde ¹H NMR (400 MHz, CDCl₃) δ - 7.62 (dd, J = 8.18, 1.83 Hz, 1H), 7.81 (d, J = 8.18 Hz, 1H), 7.94 (d, J = 1.83 Hz, 1H), 9.94 (s, 1H) |

Intermediate-46

### Step 1: Preparation of 3,5-dichloro-4-methylaniline

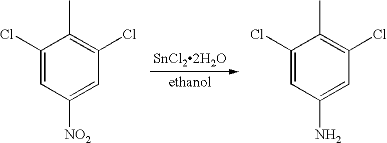

A mixture of 1,3-dichloro-2-methyl-5-nitrobenzene (5.0 g, 24.3 mmol) and tin(II) chloride dihydrate (21.9 g, 97.1 mmol) in ethanol (80 mL) was heated at reflux for 2 h. The reaction was allowed to cool, then was quenched with water and saturated aq. sodium bicarbonate until slightly basic. (Addition of sodium bicarbonate caused vigorous gas emission) The thick mixture was extracted with ethyl acetate (3×) and the combined extracts were dried over sodium sulfate and concentrated in vacuo. Chromatography (20% ethyl acetate/hexanes) provided the product, 3,5-dichloro-4-methylaniline. ¹H NMR (400 MHz, CDCl₃) δ 2.29 (s, 3H), 2.35 (br s, 2H), 6.66 (s, 2H).

### Step 2: Preparation of 5-bromo-1,3-dichloro-2-methylbenzene

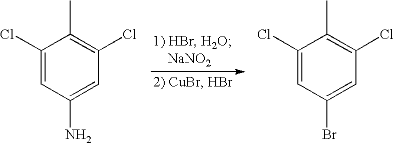

To a solution of HBr (40% in water, 3.4 mL) in water (3 mL) was added 3,5-dichloro-4-methylaniline (2.0 g, 11.4 mmol). The suspension was heated to dissolve as much of the solid as possible, then the mixture was cooled to 0 degrees C. and a solution of sodium nitrite (0.83 g, 12.0 mmol) in water (2 mL) was added dropwise (temperature was maintained <5 degrees C.). After 10 min., the diazonium salt mixture was poured into a mixture of CuBr (8.2 g, 57.0 mmol) in 40% aq. HBr (13 mL) at room temperature. The resulting mixture was heated at 50 degrees C. for 45 min., then cooled to room temperature. The mixture was diluted with water and extracted with dichloromethane (3×). The combined organics were filtered through Celite and dried over sodium sulfate, then concentrated in vacuo to provide the product, 5-bromo-1,3-dichloro-2-methylbenzene, as an orange solid. ¹H NMR (400 MHz, CDCl₃) δ 2.40 (s, 3H), 7.43 (s, 2H).

### Step 3: Preparation of 5-bromo-2-(bromomethyl)-1,3-dichlorobenzene

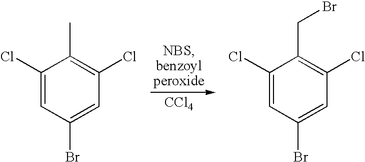

To a solution of 5-bromo-1,3-dichloro-2-methylbenzene (1.4 g, 5.8 mmol) in carbon tetrachloride (20 mL) was added N-bromosuccinimide (2.6 g, 14.6 mmol) followed by benzoyl peroxide (71 mg, 0.3 mmol). The reaction was heated at reflux overnight, then cooled to room temperature and filtered through Celite. The solvent was removed in vacuo and the residue was purified via chromatography (100% hexanes). This provided the product, 5-bromo-2-(bromomethyl)-1,3-dichlorobenzene. ¹H NMR (400 MHz, CDCl₃) δ 7.50 (s, 2H), 4.68 (s, 3H).

### Step 4: Preparation of (4-bromo-2,6-dichlorophenyl)methanol

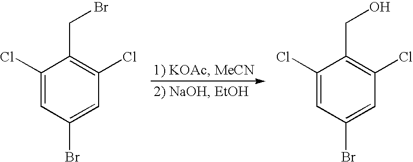

To a solution of 5-bromo-2-(bromomethyl)-1,3-dichlorobenzene (1.6 g, 5.0 mmol) in acetonitrile (25 mL) was added potassium acetate (2.0 g, 20.1 mmol) and the reaction was stirred at room temperature overnight. The solid was then filtered off and the solvent was removed in vacuo.

The resulting residue was dissolved in ethanol (25 mL) and treated with a solution of sodium hydroxide (0.5 g, 12.5 mmol) in water (4 mL). The reaction mixture was heated at reflux for 1 h, then cooled to room temperature and extracted with diethyl ether (2×). The organic extracts were washed with brine, dried over sodium sulfate and concentrated in vacuo. This provided the product, (4-bromo-2,6-dichlorophenyl)methanol, as a yellow solid. ¹H NMR (400 MHz, CDCl₃) δ 2.03 (t, J=6.4 Hz, 1H), 4.90 (d, J=6.4 Hz, 2H), 7.50 (s, 2H).

### Step 5: Preparation of 4-bromo-2,6-dichlorobenzaldehyde

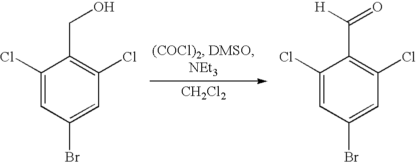

In a dry 50 mL r.b. flask under nitrogen atmosphere, oxalyl chloride (0.36 mL, 4.1 mL) was dissolved in dichloromethane (20 mL) and cooled to −78 degrees C. Dimethylsulfoxide (0.66 mL, 9.3 mmol) in dichloromethane (1 mL) was then added dropwise and the reaction was stirred 10 min. at −78 degrees C. A solution of (4-bromo-2,6-dichlorophenyl)methanol (0.95 g, 3.7 mmol) in dichloromethane (10 mL) was then added dropwise, then the reaction was stirred 30 min. at −78 degrees C. Triethylamine (2.6 mL, 18.5 mmol) was added dropwise and the reaction was stirred 10 min. more at −78 degrees C., then allowed to warm to room temperature and stir 30 min. The reaction mixture was poured into saturated sodium bicarbonate and extracted with dichloromethane (2×). The combined organics were dried over sodium sulfate, concentrated in vacuo, and purified via chromatography (5% ethyl acetate/hexanes) to provide the product, 4-bromo-2,6-dichlorobenzaldehyde. ¹H NMR (400 MHz, CDCl₃) δ 7.58 (s, 2H), 10.42 (s, 1H).

Intermediates 48 and 50

### Step 1: Preparation of 3,6-dibromo-2-methylpyridine

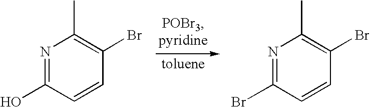

Phosphorus oxybromide (2.3 g, 8.0 mmol) was dissolved in toluene (4 mL) and 3-bromo-6-hydroxy-2-methylpyridine (1.0 g, 5.3 mmol) was added, followed by pyridine (0.43 mL, 5.3 mmol). The reaction was heated at reflux for 4 h, then cooled to room temperature and taken up in ethyl acetate and dilute aqueous sodium bicarbonate. After separating the layers, the aqueous was extracted with ethyl acetate (2×). The organics were dried over sodium sulfate, concentrated in vacuo, and purified via chromatography (20 to 50% ethyl acetate/hexanes). This provided the product, 3,6-dibromo-2-methylpyridine, as a white solid. ¹H NMR (400 MHz, CDCl₃) δ 2.63 (s, 3H), 7.18 (d, J=8.3 Hz, 1H), 7.62 (d, J=8.3 Hz, 1H).

### Step 2: Preparation of 3-bromo-6-iodo-2-methylpyridine

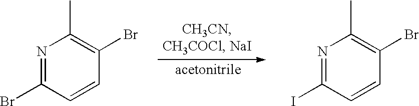

To a solution of 3,6-dibromo-2-methylpyridine (2.0 g, 8.0 mmol) in acetonitrile (40 mL) was added sodium iodide (4.8 g, 31.9 mmol), followed by acetyl chloride (0.85 mL, 12.0 mmol). The reaction was heated at reflux overnight, then was cooled to room temperature and quenched with water and basified with saturated aqueous sodium bicarbonate. The mixture was extracted with ethyl acetate (2×) and the combined extracts were washed with brine and dried over sodium sulfate. The solvent was removed in vacuo and the residue was purified via chromatography (5% ethyl acetate/hexane), providing the product, 3-bromo-6-iodo-2-methylpyridine.

### Step 3: Preparation of 5-bromo-6-methyl-2-pyridinecarbaldehyde (Intermediate 48)

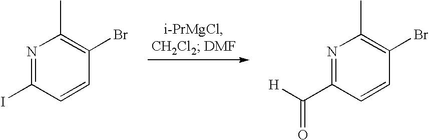

To a solution of 3-bromo-6-iodo-2-methylpyridine (2.2 g, 7.4 mmol) in dichloromethane (7.4 mmol) at −20 degrees C. was added isopropylmagnesium chloride (2M solution in THF, 4.1 mL, 8.1 mmol) and the reaction was stirred 1 h. N,N-dimethylformamide (0.86 mL, 11.1 mmol) was then added and the reaction was allowed to warm gradually to room temperature and stir overnight. The reaction was quenched with water and extracted with dichloromethane (2×). The combined organics were dried over sodium sulfate, concentrated in vacuo, and purified via chromatography (0 to 5% ethyl acetate/hexanes), providing the product, 5-bromo-6-methyl-2-pyridinecarbaldehyde, as a white solid. ¹H NMR (400 MHz, CDCl₃) δ—2.71 (s, 3H), 7.65 (d, J=8.0 Hz, 1H), 7.99 (d, J=8.0 Hz, 1H), 10.00 (s, 1H).

The following aldehyde was synthesized in an analogous manner:

| Ex. | Structure | Name and Characterization Data |
|---|---|---|
| Intermediate-50 | 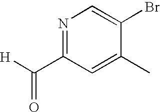 | 5-bromo-4-methyl-2-pyridinecarbaldehyde ¹H NMR (400 MHz, CDCl₃) δ - 2.49 (s, 3H), 7.82 (s, 1H), 8.80 (s, 1H), 10.02 (s, 1H). |

Intermediate 52

### Step 1: Preparation of 4-bromo-2,6-dimethylbenzonitrile

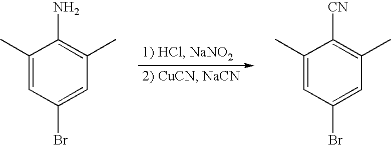

A suspension of 4-bromo-2,6-dimethylaniline (10.0 g, 50.0 mmol) in concentrated hydrochloric acid (13 mL) and water (5 mL) was heated at 90° C. for 1 h, then was cooled to 0 degrees C. A solution of sodium nitrite (3.8 g, 55 mmol) in water (15 mL) was then added dropwise to the reaction which was stirred 30 min. more at 0 degrees C. The mixture was then adjusted to pH 7 with a saturated aqueous sodium carbonate solution. Meanwhile, in a separate flask, copper(I) cyanide (17.9 g, 200 mmol) and sodium cyanide (9.1 g, 185 mmol) were suspended in water (75 mL) and toluene (35 mL) was added to the suspension. This mixture was cooled to 0 degrees C., then the diazonium mixture was poured into it. The reaction was stirred 5 min. at 0 degrees C., then was allowed to warm to room temperature. The thick red mixture was then heated at 50 degrees C. for 2 h. After cooling to room temperature the reaction mixture was filtered through Celite and the solid was rinsed with dichloromethane. The organics were washed with water and dried over sodium sulfate, then concentrated in vacuo to provide the product, 4-bromo-2,6-dimethylbenzonitrile. ¹H NMR (400 MHz, CDCl₃) δ 2.50 (s, 6H), 7.30 (s, 2H).

### Step 2: Preparation of 4-bromo-2,6-dimethylbenzaldehyde

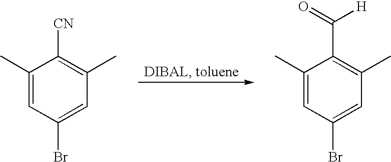

Diisobutylaluminum hydride (1.5 M solution in toluene, 2.1 mL, 3.2 mmol) was added slowly to a solution of 4-bromo-2,6-dimethylbenzonitrile (0.45 g, 2.1 mmol) in toluene (15 mL) at −78 degrees C. The reaction was allowed to stir at −78 degrees C. for 2 h, then was quenched with a mixture of water/tetrahydrofuran (6 mL/3 mL) containing sodium acetate (750 mg) and acetic acid (0.75 mL). The reaction was allowed to warm to room temperature and stir 1 h, then was poured into ethyl acetate and washed with water and saturated sodium bicarbonate. The organics were dried over sodium sulfate and concentrated in vacuo, then purified via chromatography (5% ethyl acetate/hexanes). This provided the product, 4-bromo-2,6-dimethylbenzaldehyde, as a brown solid. ¹H NMR (400 MHz, CDCl₃) δ 2.58 (s, 6H), 7.26 (s, 2H), 10.54 (s, 1H).

Intermediate-53

### Step 1: Preparation of 5-bromo-2-pyridinecarbaldehyde

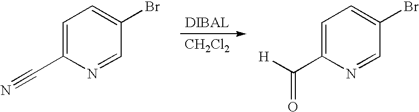

A solution of 5-bromo-2-pyridinecarbonitrile (2.0 g, 10.9 mmol) in dichloromethane (75 mL) was cooled to −78 degrees C., then a solution of DIBAL in toluene (1.5 M, 7.3 mL, 10.9 mmol) was added dropwise, taking care to keep the temperature below −65 degrees C. The resulting orange solution was stirred at −78 degrees C. for 5 h, then was allowed to gradually warm to 0 degrees C. The reaction was cooled back to −78 degrees C., then was quenched by dropwise addition of 20% aq. HCl. After warming to room temperature, the organic layer was separated, concentrated in vacuo and purified by chromatography to provide the product. The aqueous layer was treated with solid sodium bicarbonate until neutral, then this emulsion was extracted with ethyl acetate (3×). These extracts were dried over sodium sulfate and concentrated in vacuo to provide additional product, 5-bromo-2-pyridinecarbaldehyde. ¹H NMR (400 MHz, CDCl₃) δ 7.85 (d, J=8.18 Hz, 1H), 8.02 (d, J=8.18 Hz, 1H), 8.85 (s, 1H), 10.03 (s, 1H).

Intermediate-55

### 4-bromo-2,5-difluorobenzaldehyde

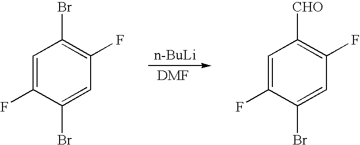

To a Et₂O solution (200 mL) containing 1,4-dibromo-2,5-difluorobenzene (19.72 g, 72.5 mmol) cooled to −78 degrees C. was added 30.5 mL of a 2.5 M hexanes solution of n-BuLi (76.2 mmol). The resulting green solution was stirred for 20 min at −78 degrees C. when DMF (7.95 g, 10.8 mmol) was added. The reaction was stirred for 30 min and then quenched with H₂O. The organics were taken up in EtOAc and washed with sat. NaHSO₄ followed by drying over MgSO₄. The solvent was removed in vacuo and the residual oil purified on the Biotage (2-5% EtOAc/hexanes) yielding 11.4 g (51.7 mmol) of 4-bromo-2,5-difluorobenzaldehyde. ¹H NMR (400 MHz, CDCl₃) δ 10.2 (s, 1H), 7.57 (m, 1H), 7.45 (m, 1H) ppm.

Intermediate-57

### Step 1: 5-bromo-2-iodo-3-methylpyridine

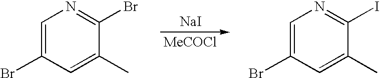

An CH₃CN solution (25 mL) containing 2,5-dibromo-3-methylpyridine (3.93 g, 15.6 mmol), NaI (9.39 g, 62.6 mmol) and acetyl chloride (1.84 g, 23.5 mmol) was refluxed for 23 hr. Upon cooling the reaction was carefully quenched with H₂O and then basified with sat. NaHCO₃. The organics were extracted with EtOAc (2×), dried over MgSO₄ and the solvent removed in vacuo. The residual dark oil was then resubjected to the same reaction conditions and worked up as before. The organics were additionally washed with sat. NaHSO₃, dried over MgSO₄, and the solvent removed in vacuo. The residual oil was purified via column chromatography (20% EtOAc/hexanes) yielding 3.33 g (11.7 mmol) of 5-bromo-2-iodo-3-methylpyridine. ¹H NMR (400 MHz, CDCl₃) δ 8.24 (d, 1H, J=2.4 Hz), 7.56 (d, 1H, J=2.5 Hz), 2.37 (s, 3H) ppm.

### Step 2: 5-bromo-3-methyl-2-pyridinecarbaldehyde

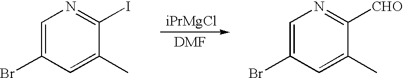

To a CH₂Cl₂ solution (20 mL) containing 5-bromo-2-iodo-3-methylpyridine (1.53 g, 5.39 mmol) cooled to 0 degrees C. was added 2.8 mL of a 2.0M THF solution of iPrMgCl (5.66 mmol). The resulting solution was stirred at 0° C. for 1.5 hr and then cooled to −20 degrees C. To this solution was added DMF (552 mg, 7.54 mmol). The reaction slowly warmed to ambient temperature overnight at which time the reaction was quenched with H₂O. The organic layer was separated and the aqueous layer extracted with CH₂Cl₂. The combined organic layers were dried over MgSO₄ and the solvent removed in vacuo. The residual oil was purified via the Biotage (10-20% EtOAc/hexanes) yielding 819 mg (4.10 mmol) of 5-bromo-3-methyl-2-pyridinecarbaldehyde as a white solid. ¹H NMR (400 MHz, CDCl₃) δ 10.1 (s, 1H), 8.69 (d, 1H, J=1.5 Hz), 7.79 (s, 1H), 2.64 (s, 3H) ppm.

Intermediate-59

### Step 1: 1,4-dibromoisoquinoline

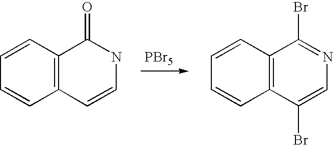

A flask containing isocarbostyril (5.0 g, 34.4 mmol) and PBr₅ (26.7 g, 62.0 mmol) was heated to 140-145 degrees C. The solids melted and after 10-15 min the liquid solidified and lightened in color. The solid was allowed to cool and then broken up and poured onto crushed ice. The resulting solid was collected via vacuum filtration and washed several times with H₂O. It was then pumped on under high vacuum yielding 9.56 g (33.3 mmol) of 1,4-dibromoisoquinoline. ¹H NMR (400 MHz, CDCl₃) δ 8.45 (s, 1H), 8.29 (d, 1H, J=8.4 Hz), 8.16 (d, 1H, J=8.4 Hz), 7.85 (t, 1H, J=7.4 Hz), 7.75 (t, 1H, J=7.5 Hz) ppm.

### Step 2: 4-bromo-1-iodoisoquinoline

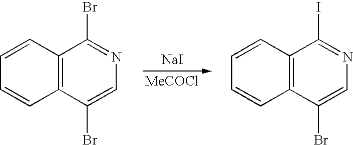

A flask containing 1,4-dibromoisoquinoline (5.0 g, 17.4 mmol), Nal (10.4 g, 69.7 mmol) and acetyl chloride (2.05 g, 26.1 mmol) was heated to reflux overnight. Upon cooling the reaction was quenched with H₂O and then carefully basified with sat. NaHCO₃. The organics were extracted with EtOAc (2×), washed with sat. NaCI and dried over MgSO₄. The solvent was removed in vacuo and the residual oil purified on the Biotage (5-10% EtOAc/hexanes) yielding 3.04 g (9.10 mmol) of 4-bromo-1-iodoisoquinoline. ¹H NMR (400 MHz, CDCl₃) δ 8.45 (s, 1H), 8.12-8.08 (m, 2H), 7.83 (m, 1H), 7.73 (m, 1H) ppm.

### Step 3: 4-bromo-1-isoquinolinecarbaldehyde

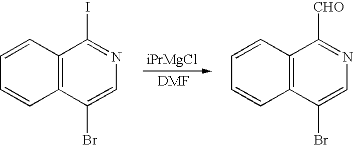

To a CH₂Cl₂ solution (30 mL) containing 4-bromo-1-iodoisoquinoline (2.03 g, 6.08 mmol) cooled to −20 degrees C. was added 3.3 mL of a 2.0 M THF solution of iPrMgCl (6.69 mmol). The resulting solution was stirred for 1 hr when DMF (667 mg, 9.12 mmol) was added. The reaction was stirred overnight at which time the heterogeneous solution was quenched with H₂O. The organics were taken up in CH₂Cl₂ and washed with H₂O. The aqueous layer was extracted with CH₂Cl₂ and then the combined organic layers were dried over MgSO₄. The solvent was removed in vacuo and the residual solid was purified on the Biotage (5-10% EtOAc/hexanes) yielding 1.12 g (4.76 mmol) of 4-bromo-1-isoquinolinecarbaldehyde. (M+1) 237, 2.28 min.

(LC/MS Method A).

Intermediate-61

### Step 1: Preparation of methyl 6-bromo-1-benzothiophene-2-carboxylate

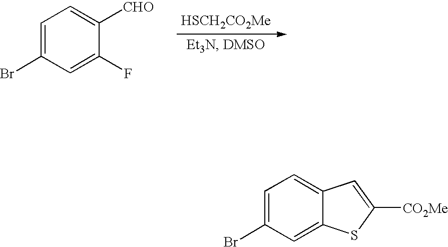

This reaction was carried out analogous to what was reported in Tet. Lett. 1992, 33, 7499. To a DMSO solution (280 mL) containing 4-bromo-2-fluorobenzaldehyde (11.06 g, 54.5 mmol) was added Et₃N (13.8 g, 136.2 mmol) followed by methyl thioglycolate (6.36 g, 60.0 mmol). The solution was then heated to 90° C. for 4 hr and upon cooling H₂O was added. The organics were extracted with EtOAc, washed with H₂O followed by drying over MgSO₄. The solvent was removed in vacuo and the resulting solid was triterated with hexanes and collected via vacuum filtration yielding 13.8 g (51.0 mmol) of methyl 6-bromo-1-benzothiophene-2-carboxylate. ¹H NMR (400 MHz, CDCl₃) δ 8.01-8.00 (m, 2H), 7.72 (d, 1H, J=8.6 Hz), 7.50 (dd, 1H, J=8.6 & 1.5 Hz), 3.94 (s, 3H) ppm

### Step 2: Preparation of 6-bromo-1-benzothiophene-2-carboxylic acid

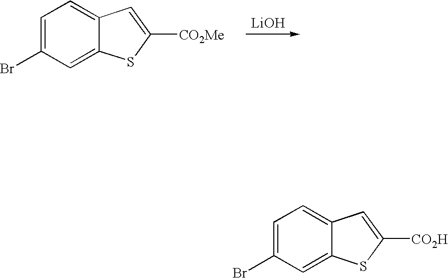

To a dioxane solution (180 mL) containing methyl 6-bromo-1-benzothiophene-2-carboxylate (13.82 g, 51.0 mmol) was added LiOH.H₂O (21.4 g, 510 mmol) followed by 60 mL of H₂O. The solution was stirred overnight at which time it was made acidic with sat. NaHSO₄. The organics were extracted with EtOAc (2×), dried over MgSO₄ and the solvent removed in vacuo yielding 12.94 g (50.4 mmol) of 6-bromo-1-benzothiophene-2-carboxylic acid. ¹H NMR (400 MHz, d₆-DMSO) δ 8.35 (s, 1H), 8.08 (s, 1H), 7.92 (d, 1H, J=8.6 Hz), 7.59 (dd, 1H, J=8.6 & 1.9 Hz) ppm.

### Step 3: Preparation of 6-bromo-1-benzothiophene

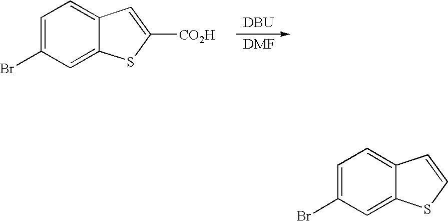

This reaction was carried out analogous to that previously reported in Tet. Lett. 2004, 45, 9645. A solution of DMF (80 mL) containing 6-bromo-1-benzothiophene-2-carboxylic acid (13.4 g, 52.1 mmol) and DBU (31.7 g, 208.4 mmol) was heated to 200 degrees C. at 800 W for 1 hr in the microwave oven. Upon cooling the dark solution was taken up in EtOAc. The organics were washed with sat. NaHSO₄ (2×), H₂O and dried over MgSO₄. The solvent was removed in vacuo and the residual solid was purified on the Biotage (10-20% EtOAc/hexanes) yielding 9.02 g (42.4 mmol) of 6-bromo-1-benzothiophene. ¹H NMR (400 MHz, CDCl₃) δ 8.01 (d, 1H, J=1.0 Hz), 7.66 (d, 1H, J=8.4 Hz), 7.46 (dd, 1H, J=8.4 & 1.7 Hz), 7.41 (d, 1H, J=5.5 Hz), 7.29 (d, 1H, J=5.5 Hz) ppm.

### Step 4: Preparation of 1-benzothiophene-6-carbaldehyde

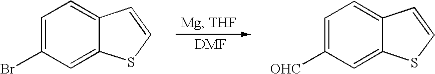

A THF solution (10 mL) containing Mg powder (1.54 g, 63.4 mmol) and a crystal of iodine was heated to reflux when a THF solution (50 mL) containing 6-bromo-1-benzothiophene (9.01 g, 42.3 mmol) was added dropwise. Upon complete addition the reaction was allowed to reflux for 1.5 hr and then cooled to 0 degrees C. To this solution was added DMF (6.18 g, 84.6 mmol) and the reaction slowly warmed to ambient temperature overnight. The reaction was quenched with sat. NaHSO₄, the organics taken up in EtOAc and then washed with sat. NaCl followed by drying over MgSO₄. The solvent was removed in vacuo and the residual oil purified on the Biotage (5-10% EtOAc/hexanes) yielding 5.46 g (33.7 mmol) of 1-benzothiophene-6-carbaldehyde. ¹H NMR (400 MHz, CDCl₃) δ 10.1 (s, 1H), 8.40 (s, 1H), 7.93 (d, 1H, J=8.2 Hz), 7.88 (dd, 1H, J=8.2 & 1.2 Hz), 7.73 (d, 1H, J=5.3 Hz), 7.43 (d, 1H, J=5.3 Hz) ppm.

### Step 5: Preparation of 3-bromo-1-benzothiophene-6-carbaldehyde

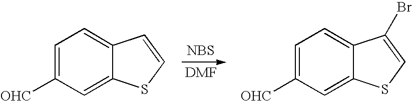

To a DMF solution (50 mL) containing 1-benzothiophene-6-carbaldehyde (5.46 g, 33.7 mmol) cooled to 0 degrees C. was added NBS (6.0 g, 33.7 mmol). The reaction slowly warmed to room temperature overnight and was then taken up in EtOAc. The organics were washed with H₂O (2×) and dried over MgSO₄. The solvent was removed in vacuo and the residue purified on the Biotage (5-10% EtOAc/hexanes) yielding 5.83 g (24.2 mmol) of 3-bromo-1-benzothiophene-6-carbaldehyde. ¹H NMR (400 MHz, CDCl₃) δ 10.1 (s, 1H), 8.38 (s, 1H), 7.99-7.96 (m, 2H), 7.71 (s, 1H) ppm.

Intermediate-63

### Step 1: Preparation of (5-bromo-1-benzothien-2-yl)methanol

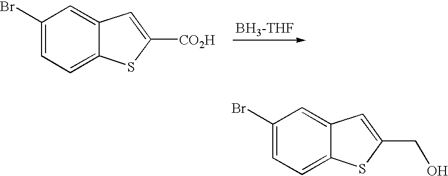

To a THF solution (20 mL) containing 5-bromo-2-benzothiophene carboxylate (2.0 g, 7.78 mmol) cooled to 0 degrees C. was added 16.4 mL of a 1.0 M THF solution of diborane (16.4 mmol). The resulting solution slowly warmed to room temperature overnight. The reaction was quenched with H₂O and then taken up in EtOAc. The organics were washed with sat. NaHSO₄, dried over MgSO₄ and the solvent removed in vacuo yielding 1.81 g (7.45 mmol) of (5-bromo-1-benzothien-2-yl)methanol which was taken on crude. ¹H NMR (400 MHz, CDCl₃) δ 7.85 (d, 1H, J=1.7 Hz), 7.66 (d, 1H, J=8.6 Hz), 7.40 (dd, 1H, J=8.5 & 1.7 Hz), 7.14 (s, 1H), 4.93 (s, 2H), 1.95 (s(br), 1H) ppm.

### Step 2: Preparation of 5-bromo-1-benzothiophene-2-carbaldehyde

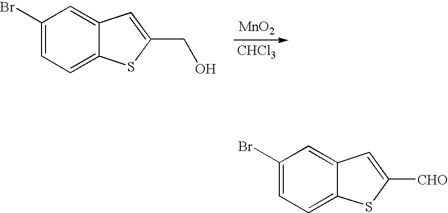

To a CHCl₃ solution (30 mL) containing (5-bromo-1-benzothien-2-yl)methanol (1.81 g, 7.45 mmol) was added MnO₂ (6.48 g, 74.5 mmol). The resulting solution was then heated to reflux for 1.5 hr. Upon cooling the heterogeneous black solution was filtered through a bed of celite. The celite was rinsed thoroughly with CH₂Cl₂ and then the solvent removed in vacuo. The residual solid was purified on the Biotage (5-10% EtOAc/hexanes) yielding 1.0 g (4.18 mmol) of 5-bromo-1-benzothiophene-2-carbaldehyde. ¹H NMR (400 MHz, CDCl₃) δ 10.1 (s, 1H), 8.09 (d, 1H, J=1.5 Hz), 7.95 (s, 1H), 7.77 (d, 1H, J=8.5 Hz), 7.59 (dd, 1H, J=8.8 & 1.7 Hz) ppm.

Intermediate-65

### 5-(4,4,5,5-tetramethyl-1,3,2-dioxaborolan-2-yl)-1-benzothiophene-3-carbaldehyde (pg 140-1)

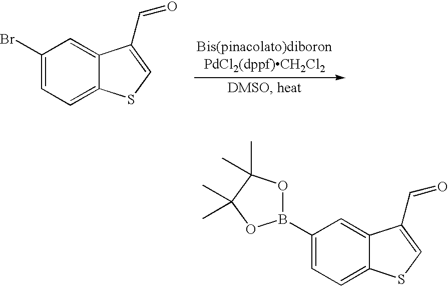

A flask charged with 5-bromo-benzothiophene-3-carboxaldehyde (1.0 g, 4.15 mmol), Bis(pinacolato)diboron (1.16 g, 4.56 mmol), PdCl₂(dppf).CH₂Cl₂ (0.101 g, 0.124 mmol), potassium acetate (1.22 g, 12.44 mmol) and DMSO (25 mL) was flushed briefly with N₂ and then heated to 80 degrees Celsius under N₂ for 2 hours. The reaction was cooled and poured onto water and extracted with EtOAc (×2). The combined organics were washed with brine and then dried (Na₂SO₄), filtered and concentrated. Chromatography on silica gel using Hexanes-EtOAc afforded the title compound as an off-white solid (0.963 g, 81% yield).

¹H NMR (400 MHz, CDCl₃) delta ppm 1.37 (s, 12H) 7.84-7.89 (m, 2H) 8.30 (s, 1H) 9.08 (s, 1H) 10.18 (s, 1H)

Intermediate-67

### Step 1: Preparation of 4-bromo-3-chloro-2-nitroaniline

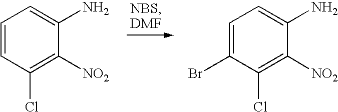

A solution of N-bromosuccinimide (3.76 g, 21.1 mmol) in DMF (20 mL) was added to a solution of 3-chloro-2-nitroaniline (3.65 g, 21.1 mmol) in DMF (40 mL) at 0 degrees C. The resulting solution was stirred at 0 degrees C. for 1 h, then allowed to warm to room temperature overnight. The orange solution was taken up in ethyl acetate and washed with water (3×), then dried over magnesium sulfate. The solvent was removed in vacuo and the solid was rinsed with hexanes and collected via vacuum filtration to provide the product, 4-bromo-3-chloro-2-nitroaniline, as an orange solid. ¹H NMR (400 MHz, CDCl₃) δ 4.73 (br s, 2H), 6.62 (d, J=9.0 Hz, 1H), 7.44 (d, J=9.0 Hz, 1H).

### Step 2: Preparation of 4-bromo-3-chloro-1,2-benzenediamine

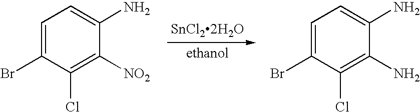

A mixture of 4-bromo-3-chloro-2-nitroaniline (1.0 g, 4.0 mmol) and tin(II) chloride dihydrate (3.6 g, 15.9 mmol) in ethanol (15 mL) was heated at reflux for 4 h. The reaction was allowed to cool, then was quenched with water and saturated aq. sodium bicarbonate until slightly basic. (Addition of sodium bicarbonate caused vigorous gas emission) The thick mixture was extracted with ethyl acetate (3×) and the combined extracts were dried over sodium sulfate and concentrated in vacuo. Chromatography (25 to 35% ethyl acetate/hexanes) provided the product, 4-bromo-3-chloro-1,2-benzenediamine, as a tan solid. ¹H NMR (400 MHz, CDCl₃) δ 3.48 (br s, 2H), 3.86 (br s, 2H), 6.51 (d, J=8.6 Hz, 1H), 6.93 (d, J=8.6 Hz, 1H).

### Step 3: Preparation of 5-bromo-4-chloro-1H-benzimidazole

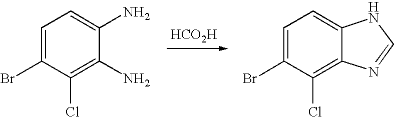

In a 50 mL flask, 4-bromo-3-chloro-1,2-benzenediamine (3.5 g, 15.8 mmol) was dissolved in formic acid (8 mL) and the reaction was heated at reflux for 2 h. The reaction was cooled to room temperature and treated with 10% aq. sodium hydroxide until the mixture was basic. The dark mixture was extracted with ethyl acetate and the organics were washed with brine, dried over sodium sulfate, and concentrated in vacuo. The solid was washed with dichloromethane (3×), then dried under vacuum to provide the product, 5-bromo-4-chloro-1H-benzimidazole, as a yellow solid.

### Step 4: Preparation of 5-bromo-4-chloro-1-(triphenylmethyl)-1H-benzimidazole

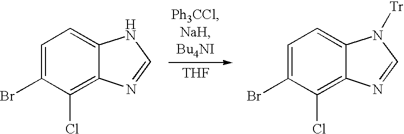

To a solution of 5-bromo-4-chloro-1H-benzimidazole (3.7 g, 16.0 mmol) in tetrahydrofuran (75 mL) at 0° C. was added sodium hydride (0.77 g, 19.2 mmol) as a solid in three portions. The reaction mixture was stirred 30 min. at 0 degrees C., then trityl chloride (5.8 g, 20.8 mmol) was added, followed by tetrabutylammonium iodide (catalytic). The reaction was heated at reflux for 1.5 h, then was allowed to cool to room temperature and was quenched with water. The mixture was extracted with chloroform (3×) and the organics were dried over sodium sulfate and concentrated in vacuo. Chromatography (10 to 20% ethyl acetate/hexanes) provided the product, 5-bromo-4-chloro-1-(triphenylmethyl)-1H-benzimidazole, as a yellow solid. ¹H NMR (400 MHz, CDCl₃) δ 6.24 (d, J=8.8 Hz, 1H), 7.15-7.09 (m, 7H), 7.33-7.29 (m, 9H), 7.89 (s, 1H).

Intermediate-69

### Step 1: Preparation of 4-bromo-5-methyl-2-nitroaniline

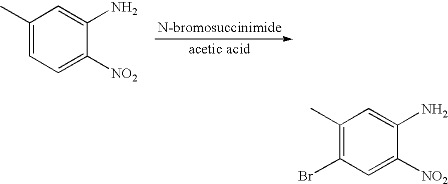

A solution of 5-methyl-2-nitroaniline (10.0 g, 65.7 mmol) and N-bromosuccinimide (11.5 g, 64.4 mmol) in acetic acid (450 mL) was heated at reflux for 90 min., then was cooled and poured into water (˜3 L). The resulting thick yellow mixture was stirred for 10 min., then the precipitate was collected via filtration. The bright yellow solid was dried under vacuum overnight, providing 4-bromo-5-methyl-2-nitroaniline. ¹H NMR (400 MHz, CDCl₃) δ 2.38 (s, 3H), 5.98 (br s, 2H), 6.66 (s, 1H), 8.28 (s, 1H).

### Step 2: Preparation of 4-bromo-5-methyl-1,2-benzenediamine

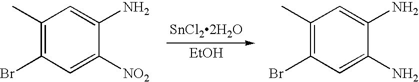

A mixture of 4-bromo-5-methyl-2-nitroaniline (8.0 g, 34.6 mmol) and tin(II) chloride dihydrate (31.2 g, 138.5 mmol) in ethanol (200 mL) was heated at 70 degrees C. overnight. The reaction was allowed to cool and was quenched with water and and saturated aqueous sodium bicarbonate until slightly basic. The thick mixture was extracted with ethyl acetate (3×) and then the extracts were dried over sodium sulfate and concentrated in vacuo. This provided 4-bromo-5-methyl-1,2-benzenediamine as a light yellow solid. ¹H NMR (400 MHz, CDCl₃) δ—2.20 (s, 3H), 2.15-2.25 (br s, 4H), 6.55 (s, 1H), 6.85 (s, 1H).

### Step 3: Preparation of 5-bromo-6-methyl-1H-benzimidazole

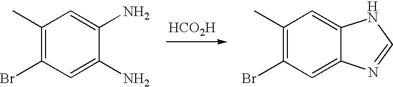

A solution of 4-bromo-5-methyl-1,2-benzenediamine (7.0 g, 34.6 mmol) in formic acid (20 mL) was heated at reflux for 2 h. The reaction was then cooled to room temperature and treated with 10% aq. sodium hydroxide until the mixture was basic. The dark mixture was extracted with ethyl acetate and the combined organics were washed with brine, dried over sodium sulfate and concentrated in vacuo. The resulting powder was washed with dichloromethane and dried under vacuum, providing 5-bromo-6-methyl-1H-benzimidazole as a tan solid. ¹H NMR (400 MHz, CDCl₃) δ 2.47 (s, 3H), 7.45 (s, 1H), 7.80 (s, 1H), 7.94 (s, 1H).

### Step 4: Preparation of 5-bromo-6-methyl-1-(triphenylmethyl)-1H-benzimidazole and 6-bromo-5-methyl-1-(triphenylmethyl)-1H-benzimidazole

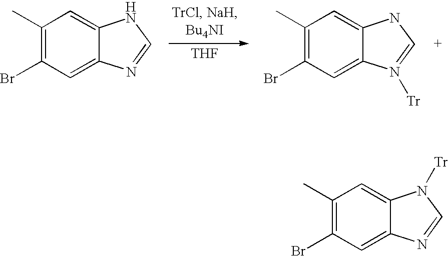

To a solution of 5-bromo-6-methyl-1H-benzimidazole (5.9 g, 27.7 mmol) in tetrahydrofuran (120 mL) at 0 degrees C. was added sodium hydride (1.3 g, 33.3 mmol) as a solid in two separation portions. The cloudy reaction mixture was stirred 30 min. at 0 degrees C., then trityl chloride (10.0 g, 36.0 mmol) was added, followed by a catalytic amount of tetra-n-butylammonium iodide. The reaction was heated at reflux for 1.5 h, then was allowed to cool to room temperature and was quenched with water. The layers were separated and the aqueous was extracted with chloroform (2×). The combined organics were dried over sodium sulfate and concentrated in vacuo. This provided a solid which was suspended in hot ethyl acetate and filtered, providing the product as a mixture of trityl regioisomers, 5-bromo-6-methyl-1-(triphenylmethyl)-1H-benzimidazole and 6-bromo-5-methyl-1-(triphenylmethyl)-1H-benzimidazole, as an off-white solid.

Isomer a: ¹H NMR (400 MHz, CDCl₃) δ 2.15 (s, 3H), 6.28 (s, 1H), 7.16-7.13 (m, 6H), 7.34-7.31 (m, 9H), 7.82 (s, 1H), 7.96 (s, 1H)

Isomer b: ¹H NMR (400 MHz, CDCl₃) δ 2.42 (s, 3H), 6.61 (s, 1H), 7.16-7.13 (m, 6H), 7.34-7.31 (m, 9H), 7.64 (s, 1H), 7.86 (s, 1H)

Intermediate-75

### Step 1: 4-Methyl-5-(4,4,5,5-tetramethyl-1,3,2-dioxaborolan-2-yl)-1-(triphenylmethyl)-1H-benzimidazole

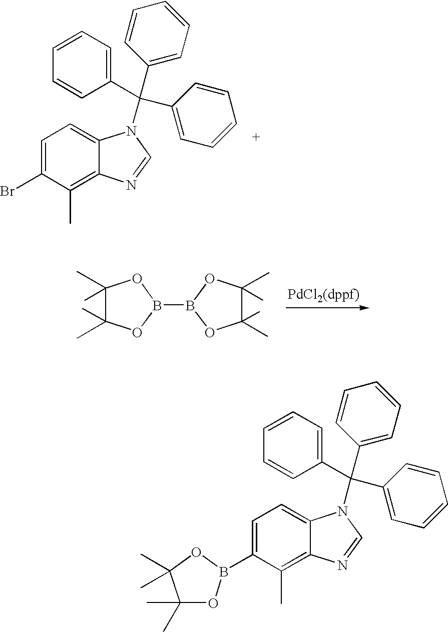

5-Bromo-4-methyl-1-(triphenylmethyl)-1H-benzimidazole [from Intermediate 3, Step 4] (0.453 g, 1 mmol), bis(pinacolato)diboron (0.38 g, 1.5 mmol), 1,1′-bis(diphenylphosphino) ferrocene-palladium dichloride (53 mg, 0.065 mmol) and potassium acetate (0.294 g, 3 mmol) in 6 ml of DMSO were degassed by vacuum-nitrogen backfilling cycles, heated at 80 degrees C. for 15 h, cooled, poured into water and extracted with ethyl acetate three times. The combined organic was washed with brine, dried over magnesium sulfate and concentrated in vacuo. The residue was purified by silica gel column chromatography (20 to 50% ethyl acetate in hexanes) to afford the title compound as a white solid.

¹H NMR (400 MHz, CDCl₃) delta ppm 1.35 (s, 12H) 2.92 (s, 3H) 6.30 (d, 1H) 7.17-7.20 (m, 6H) 7.23-7.38 (m, 10H) 7.83 (s, 1H)

### Step 2: 2-Chloro-4-[4-methyl-1-(triphenylmethyl)-1H-benzimidazol-5-yl]benzaldehyde

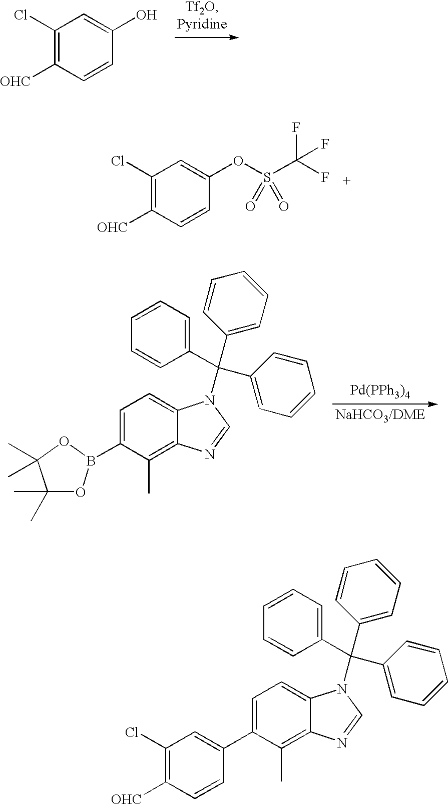

To the solution of 2-chloro-4-hydroxybenzaldehyde (4.15 g, 26.5 mmol) and pyridine (3.48 ml, 43 mmol) in 250 ml of dichloromethane at 0 degrees C. was slowly added trifluoromethanesulfonic anhydride (5.83 ml, 34.7 mmol). The reaction mixture was allowed to warm up to room temperature, stirred for 15 h, cooled back to 0 degrees C. and quenched with 20 ml of water. The mixture was adjusted with saturated sodium bicarbonate solution to pH 7-8. The organic was separated and the aqueous layer was extracted with dichloromethane. The combined extracts were washed with brine, dried over magnesium sulfate and concentrated in vacuo. The residue was purified by silica gel column chromatography (10% ethyl acetate in hexanes) to afford 3-chloro-4-formylphenyl trifluoromethanesulfonate as a colorless oil. 3-Chloro-4-formylphenyl trifluoromethanesulfonate (0.278 g, 0.96 mmol), 4-Methyl-5-(4,4,5,5-tetramethyl-1,3,2-dioxaborolan-2-yl)-1-(triphenylmethyl)-1H-benzimidazole (0.4382 g, 0.88 mmol) from Step 1, and tetrakis(triphenylphosphine)palladium(0) (30 mg, 0.046 mmol) in 10 ml of dimethoxyethane and 3 ml of saturated sodium bicarbonate aqueous solution were degassed by vacuum-nitrogen backfilling cycles, heated to reflux for 15 h, cooled and partitioned between ethyl acetate and water. The organic phase was separated, washed with brine, dried over magnesium sulfate and concentrated in vacuo. The residue was purified by silica gel column chromatography (20 to 50% ethyl acetate in hexanes) to afford the title compound as a colorless oil.

¹H NMR (400 MHz, CDCl₃) delta ppm 2.60 (s, 3H) 6.38 (d, 1H) 6.76 (d, 1H) 7.17-7.40 (m, 17H) 7.95 (m, 2H) 10.45 (s, 1H)

Synthesis of Non-Commercial Amines

Amine-1

### Preparation 1: (±)cis-1-Fluoro-2,3-dihydro-1H-inden-2-amine

### Step 1: (±)cis -2-Azido-2,3-dihydro-1H-inden-1-ol

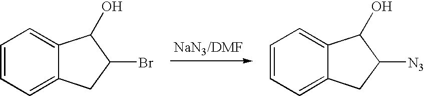

A mixture of trans-2-bromo-1-indanol (10 g, 43.28 mmol, Aldrich) and sodium azide (3.25 g, 50 mmol) in 50 ml of dry DMF was heated at 80 degrees C. for 6 h, cooled to room temperature, poured into 300 ml of water. The suspension was stirred for 1 h, and the solid was isolated by filtration and washed with water to afford the title compound as a fluffy tan solid.

¹H NMR (400 MHz, CDCl₃) delta ppm 2.33 (br., 1H) 3.08 (m, 2H) 4.38 (m, 1H) 5.15 (m, 1H) 7.29 (m, 3H) 7.42 (m, 1H)

### Step 2: (±)trans-2-Azido-1-fluoro-2,3-dihydro-1H-indene & (±)cis-2-Azido-1-fluoro-2,3-dihydro-1H-indene

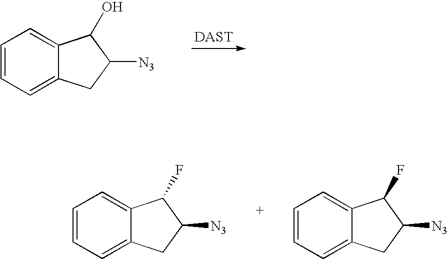

Into a pre-cooled (0 degrees C.) solution of 2-azido-2,3-dihydro-1H-inden-1-ol (1.05 g, 6 mmol) in 30 ml of dichloromethane was added DAST (0.825 ml, 6.24 mmol). The reaction mixture was stirred for 2 h at 0 degrees C., quenched by adding methanol and concentrated in vacuo. The residue was directly purified by silica gel column chromatography (0 to 5% diethyl ether in hexanes) to afford the title compounds:

(±)trans-2-Azido-1-fluoro-2,3-dihydro-1H-indene: light brown oil, ¹H NMR (400 MHz, CDCl₃) delta ppm 2.90 (m, 1H) 3.41 (m, 1H) 4.38 (m, 1H) 5.78 and 5.97 (m, 1H) 7.22-7.60 (m, 4H); ¹⁹F NMR (400 MHz, CDCl₃) delta ppm −172.5

(±)cis-2-Azido-1-fluoro-2,3-dihydro-1H-indene: light brown oil, ¹H NMR (400 MHz, CDCl₃) delta ppm 3.20 (m, 2H) 3.97 (m, 1H) 5.68 and 5.82 (m, 1H) 7.20-7.50 (m, 4H); ¹⁹F NMR (400 MHz, CDCl₃) delta ppm −180.4

### Step 3: (±)cis-1-Fluoro-2,3-dihydro-1H-inden-2-amine

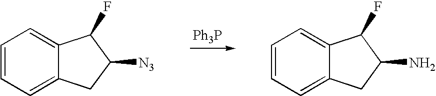

The mixture of (±)cis-2-Azido-1-fluoro-2,3-dihydro-1H-indene (0.296 g, 1.67 mmol) and triphenylphosphine (0.479 g, 1.83 mmol) in methanol (33 ml) was heated at 40 degrees C. for 2 h, cooled to room temperature, concentrated in vacuo and directly purified by silica gel column chromatography (5 to 10% methanol in dichloromethane) to afford the title compound as a light brown oil.

¹H NMR (400 MHz, CDCl₃) delta ppm 2.90 (m, 1H) 3.20 (m, 1H) 3.75 (m, 1H) 5.50 and 5.62 (m, 1H) 7.22-7.50 (m, 4H); ¹⁹F NMR (400 MHz, CDCl₃) delta ppm −183.7

Amine-2

### Preparation 2: 2-Amino-2,3-dihydro-1H-inden-1-ol Hydrochloride

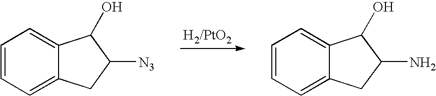

The mixture of 2-azido-2,3-dihydro-1H-inden-1-ol (2.0 g) and platinum oxide (0.10 g) in absolute ethanol was hydrogenated with hydrogen in a balloon at room temperature for 12 h. The reaction mixture was filtered and concentrated in vacuo. The residue was dissolved in dichloromethane and 1M HCl in diethyl ether was added until it became acidic. The solid was isolated by filtration and washed with ether to afford the title compound as a pale yellow solid.

¹H NMR (400 MHz, DMSO-d₆) delta ppm 2.99 (m, 1H) 3.15 (m, 1H), 3.81 (m, 1H) 5.06 (m, 1H) 6.12 (br., 1H) 7.20-7.41 (m, 4H) 8.01 (br., 3H)

Amine-3

### Preparation 3: (±)cis-1-(Methyloxy)-2,3-dihydro-1H-inden-2-amine

### Step 1: (±)cis-2-Azido-1-(methyloxy)-2,3-dihydro-1H-indene

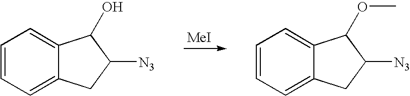

The mixture of (±)cis -2-azido-2,3-dihydro-1H-inden-1-ol (1.02 g, 5.82 mmol), methyl iodide (1.0 ml, 16 mmol) and silver oxide (1.5 g, 12.1 mmol) in 10 ml of acetonitrile was heated at reflux for 3 h, cooled to room temperature, filtered thorough a Celite pad and concentrated in vacuo.

The brown residue was purified by silica gel column chromatography (5% ethyl acetate in hexanes) to afford the title compound as a white wax-like solid.

¹H NMR (400 MHz, CDCl₃) delta ppm 3.15 (m, 2H) 3.56 (s, 3H) 4.13 (m, 1H) 4.67 (m, 1H) 7.22-7.37 (m, 3H) 7.40 (m, 1H)

### Step 2: (±)cis-1-(Methyloxy)-2,3-dihydro-1H-inden-2-amine

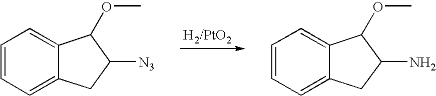

The mixture of (±)cis-2-Azido-1-(methyloxy)-2,3-dihydro-1H-indene (0.76 g) and platinum oxide (0.06 g) in absolute ethanol was hydrogenated with hydrogen in a balloon at room temperature for 12 h. The reaction mixture was filtered and concentrated in vacuo to dryness to afford the title compound as a brown solid.

¹H NMR (400 MHz, CDCl₃) delta ppm 2.05 (br., 2H) 2.80 (m, 1H) 3.02 (m, 1H) 3.45 (s, 3H) 3.75 (m, 1H) 4.39 (m, 1H) 7.18-7.30 (m, 3H) 7.38

Amine-4

### Step 1: 3-(3,4-difluorophenyl)propanoyl chloride

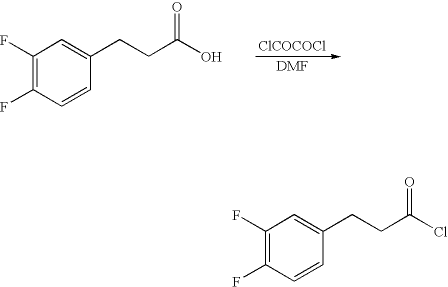

To a CH₂Cl₂ solution (200 mL) containing 3,4-difluorophenyl propionic acid (30.45 g, 163.6 mmol) and 2 drops of DMF was added oxalyl chloride (41.4 g, 327.1 mmol) over 20 min. The resulting solution was stirred for 24 hr at which time the solvent was removed in vacuo. The residual oil was then evaporated with toluene (˜100 mL) yielding 33.4 g of 3-(3,4-difluorophenyl)propanoyl chloride as a yellow liquid. This was taken directly into the next step.

### Step 2: 5,6-difluoro-2,3-dihydro-1H-inden-1-one

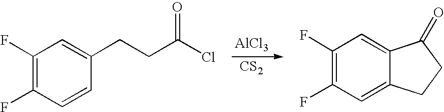

To a carbon disulfide solution (300 mL) containing AlCl₃ (76.4 g, 572.6 mmol) cooled to 0 degrees C. was added a carbon disulfide solution (120 mL) of 3-(3,4-difluorophenyl)propanoyl chloride (33.4 g, 163.6 mmol) over 10 min. The solution was stirred for 30 min at 0 degrees C. and then heated to reflux for 4 hr. Upon cooling to room temperature the solution was carefully poured onto crushed ice. The carbon disulfide layer was separated and the aqueous layer extracted with EtOAc. The combined organic layers were dried over MgSO₄ and the solvent removed in vacuo. The residual solid was purified on the Biotage (5-10% EtOAc/hexanes) yielding 19.3 g (114.9 mmol) of 5,6-difluoro-2,3-dihydro-1H-inden-1-one as a white solid. ¹H NMR (400 MHz,CDCl₃) δ 7.50 (t, 1H, J=8.0 Hz), 7.24 (t, 1H, J=6.6 Hz), 3.09 (t, 2H, J=5.5 Hz), 2.72-2.69 (m, 2H).

### Step 3: (2E)-5,6-difluoro-1H-indene-1,2(3H)-dione 2-oxime

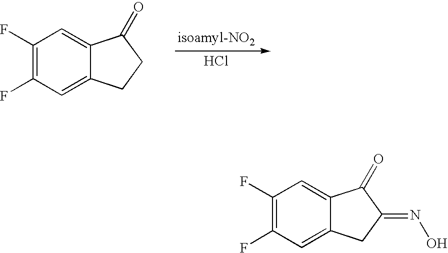

To a MeOH solution (90 mL) containing 5,6-difluoro-2,3-dihydro-1H-inden-1-one (4.60 g, 27.4 mmol), which had been heated to 40 degrees C., was added isoamyl nitrite (4.17 g, 35.6 mmol) followed by concentrated HCl (2.7 mL). Upon heating for 45 min the solution was allowed to cool to room temperature and H₂O added. The resulting precipitate was collected via vacuum filtration. The solid was rinsed thoroughly with H₂O yielding 3.97 g (20.2 mmol) of (2E)-5,6-difluoro-1H-indene-1,2(3H)-dione 2-oxime as a light orange solid. The crude solid was taken directly into the next step.

### Step 4: (5,6-difluoro-2,3-dihydro-1H-inden-2-yl)amine hydrochloride

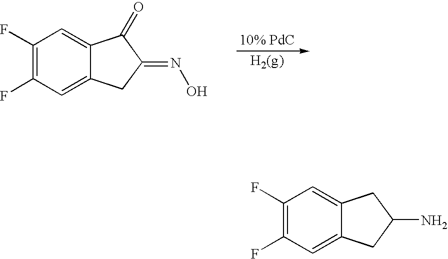

To an HOAc solution (100 mL) containing (2E)-5,6-difluoro-1H-indene-1,2(3H)-dione 2-oxime (3.97 g, 20.2 mmol) in a Parr bottle was added 8 mL of concentrated HCl followed by 10% Pd/C (1.07 g). The solution was hydrogenated at 50 psi for 24 hr. The heterogeneous solution was filtered through a bed of celite with the celite being rinsed thoroughly with CHCl₃. The solvent was then removed in vacuo and the residual dark oil dissolved in H₂O. The aqueous solution was then basified with solid K₂CO₃ and the organics extracted with CHCl₃ (3×) followed by drying over MgSO4. The solvent was removed in vacuo and the residual amber oil purified via column chromatography (10% MeOH/CH₂Cl₂) yielding 1.06 g (6.26 mmol, 30% yield) of the desired amine as a brown oil. ¹H NMR (400 MHz, CDCl₃) δ 6.95 (t, 2H, J=8.9 Hz), 3.83 (m, 1H), 3.10 (dd, 2H, J=15.8 & 6.8 Hz), 2.60 (dd, 2H, J=15.8 & 5.0 Hz); (M+1) 170.1, 0.68 min (LC/MS method A). The oil was dissolved in Et₂O (˜5 mL) and 4.0 mL of a 4.0 M dioxane solution of HCl (16.0 mmol) was added. The resulting precipitate was triterated with Et₂O and collected via vacuum filtration yielding 795 mg (3.87 mmol) of (5,6-difluoro-2,3-dihydro-1H-inden-2-yl)amine hydrochloride.

Amine-5

### Step 1: (2E)-5,7-difluoro-1H-indene-1,2(3H)-dione 2-oxime

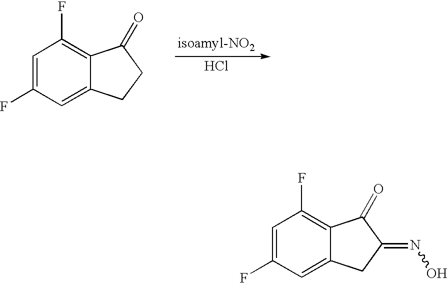

To a MeOH solution (70 mL) containing 4,6-difluoro-1-indanone (4.70 g, 28.0 mmol) was added isoamyl nitrite (4.26 g, 36.4 mmol) at 40 degrees C. followed by 2.3 mL of concentrated HCl. The resulting solution was stirred at 40 degrees C. for 1.5 hr and upon cooling H₂O was added. The precipitated solid was collected via vacuum filtration. The solid was rinsed thoroughly with H₂O followed by drying under high vacuum yielding 4.26 g (21.6 mmol) of (2E)-5,7-difluoro-1H-indene-1,2(3H)-dione 2-oxime which was taken on crude.

### Step 2: 4,6-difluoro-2,3-dihydro-1H-inden-2-amine hydrochloride

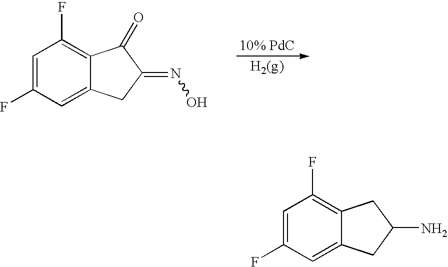

An HOAc solution (100 mL) containing (2E)-5,7-difluoro-1H-indene-1,2(3H)-dione 2-oxime (4.26 g, 21.6 mmol), 7 mL of concentrated H₂SO₄ and 1.1 g of 10% Pd/C was placed under 50 psi of H₂ in a Parr aparartus for 20 hr. The heterogeneous solution was filtered through a bed of celite and the celite was rinsed thoroughly with CHCl₃. The solvent was removed in vacuo and the residual dark oil diluted with H₂O. The solution was made basic by cautious addition of solid K₂CO₃. The organics were extracted with CHCl₃ (3×), dried over MgSO₄ and the solvent removed in vacuo. The residual oil was purified via column chromatography (10% MeOH/CH₂Cl₂) yielding 368 mg of the free base amine. The amine was dissolved in 5 mL of Et₂O and 3 mL of a 4.0 M dioxane solution of HCl was added. The precipitated solid was triterated with Et₂O and collected via vacuum filtration. The collected solid was rinsed thoroughly with Et₂O yielding 374 mg (1.82 mmol) of 4,6-difluoro-2,3-dihydro-1H-inden-2-amine as a HCl salt. ¹H NMR (400 MHz, d₆-DMSO) 8.40 (s(br), 2H), 7.05-7.00 (m, 2H), 4.02 (s(br), 1H), 3.31-3.19 (m, 2H), 3.04-2.91 (m, 2H) ppm.

Amine-6

### Preparation of (2-cyclohexyl-2,2-difluoroethyl)amine hydrochloride

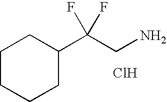

### Step 1: Ethyl Cyclohexyl(oxo)acetate Preparation

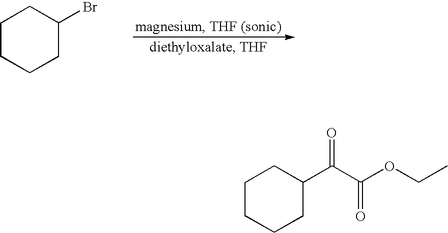

To a THF (100 mL) suspension of magnesium turnings (2.20 g, 90.32 mmol) was added cyclohexyl bromide (9.27 mL, 75.27 mmol). After 30 min of sonication the pale yellow turbid solution was decanted to an awaiting addition funnel and added over one hour to a 240 mL THF solution of diethyloxalate (22.0 g, 146.14 mmol) cooled to −10 degrees Celsius. After an additional 30 minutes 75 mL of a 10% HCl v/v solution was added and stirred for 15 minutes. The layers were separated and the aqueous extracted with 100 mL diethyl ether and the combined organics washed 100 mL brine, then dried (Na₂SO₄), filtered and concentrated. Chromatography on silica using Hexanes-Ethyl acetate afforded the product as a clear oil (8.3 g, 60% yield).

¹H NMR (400 MHz, CDCl₃) delta ppm 1.15-1.40 (m, 5H) 1.56-1.94 (m, 8H) 2.97-3.05 (m, 1H) 4.30 (q, J=7.32Hz, 2H)

### Step 2: Ethyl Cyclohexyl(difluoro)acetate Preparation

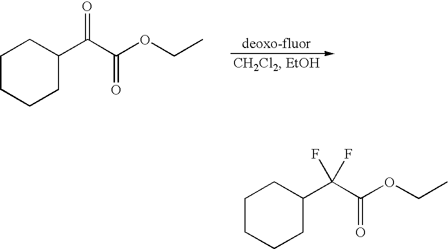

To a solution of ethyl cyclohexyl(oxo)acetate (2.94 g, 15.95 mmol) from Step 1 in 5 mL dichloromethane cooled to −5 degrees Celsius was added Bis(2-methoxyethyl)aminosulfur trifluoride [deoxo-fluor] (5.0 mL, 27.12 mmol) in 5 mL of dichloromethane while stirring. EtOH (0.185 mL, 0.78 mmol) was added and the reation contents were stirred for 16 hours at ambient temperature and then poured onto ice. The layers were separated and the aqueous extracted using 10 mL dichloromethane. The combined organics were washed once with aqueous saturated NaHCO₃, once with brine and then dried (Na₂SO₄), filtered and concentrated. Chromatography on silica gel using Hexanes-Ethyl acetate afforded the product as a clear oil (2.63 g, 79% yield).

¹H NMR (400 MHz, CDCl₃) delta ppm 1.13-1.37 (m, 5H) 1.48-1.85 (m, 8H) 1.98-2.08 (m, 1H) 4.30 (q, J=7.08 Hz, 2H)

### Step 3: 2-cyclohexyl-2,2-difluoroacetamide Preparation

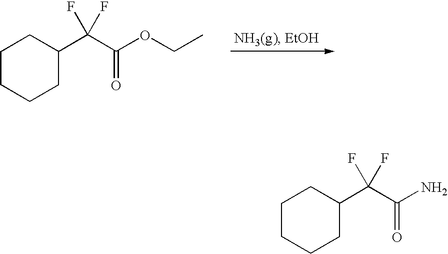

The ester (2.63 g, 12.75 mmol) was dissolved in 6 mL of ethanol and saturated with gaseous ammonia for 15 minutes at ambient temperature. The pressure tube was sealed and allowed to stand overnight. The solvents were removed and the solid recrystallized from dichloromethane-Hexanes to afford the product as a waxy solid (2.06 g, 91% yield).

¹H NMR (400 MHz, CDCl₃) delta ppm 1.13-1.37 (m, 5H) 1.48-1.85 (m, 8H) 1.98-2.08 (m, 1H) 4.30 (q, J=7.08 Hz, 2H)

### Step 4: (2-cyclohexyl-2,2-difluoroethyl)amine hydrochloride Preparation

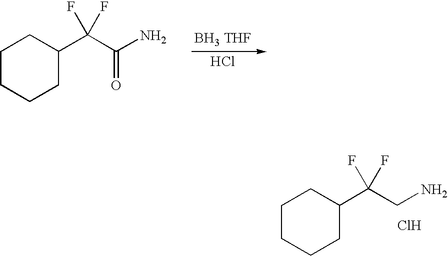

To a solution of the amide in 20 mL of THF at ambient temperature under nitrogen was added borane-tetrahydrofuran complex (56.44 mL, 56.44 mmol). The reaction mixture was heated to reflux for 18 hours and then cooled to ambient temperature. Methanol was added slowly with stirring and the mixture heated to reflux for 30 minutes, then cooled and concentrated. 6M HCl (5 mL) was added and the mixture heated to reflux for a brief minute then cooled. The mixture was brought to pH 10 using aqueous saturated NaHCO₃ and the organics extracted with 50 mL dichloromethane twice. The organics were dried (Na₂SO₄), filtered and concentrated. The residue was taken up in EtOH and gaseous HCl bubbled through for a scant minute. The solid was filtered and dried to give the title product (2.0 g, 89% yield) as a white solid.

¹H NMR (400 MHz, DMSO-d₆) delta ppm 1.04-1.25 (m, 5H) 1.56-1.65 (m, 1H) 1.67-1.81 (m, 4H) 1.87-1.98 (m, 1H) 3.35 (t, J=16.4 Hz, 2H) 8.37 (bs, 2H)

Amine-7:

### 4,4-dimethylcyclohexylmethylamine hydrochloride

### Step 1: (4,4-dimethylcyclohexylidene)methyl methyl ether

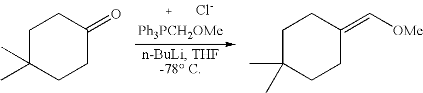

To a mixture of methoxymethyl triphenylphosphonium chloride (35.5 g, 0.104 mol) in THF (400 mL) at 0 degrees C. was added n-BuLi (33.1 mL, 0.095 mol) as a 2.87M solution in hexanes. The mixture was stirred at 0° C. for 30 min., cooled to −78° C. and 4,4-dimethyl cyclohexanone (10.0 g, 0.079 mol) in THF (100 mL) was added dropwise. After 1 hr at −78 degrees C., the mixture was allowed to slowly warm to 0° C. and saturated ammonium chloride (aq) (400 mL) and ethyl acetate (100 mL) was added. After stirring at room temperature for 48 hr, the mixture was separated and the aqueous phase was extracted with ethyl acetate. The combined organic phase was washed with brine, dried (Na₂SO₄), filtered and concentrated in vacuo. The residue was triturated with hexanes, filtered and concentrated in vacuo. NMR showed the residue to be a mixture of the desired product and starting ketone in a ratio of 4:1. An attempt to purify by passing through a silica gel column using hexanes/ethyl acetate (3:1) as eluent failed. The residue was dissolved in dichloromethane (40 mL) and PS-TsNHNH₂ (3.7 mmol/g) (8 g) and acetic acid (2 drops) were added and the mixture was stirred at room temperature for 24 hr. The resin was filtered off and washed with dichloromethane, methanol then dichoromethane. The combined organic phase was concentrated in vacuo to give (4,4-dimethyl cyclohexylidene)methyl methyl ether as an oil. 1H NMR (400 MHz, CDCl₃) delta ppm 0.91 (s, 6H); 1.27 (m, 5H); 1.95 (m, 2H); 2.18 (m, 2H); 3.52 (s, 3H).

### Step 2: 4,4-dimethylcyclohexanecarbaldehyde

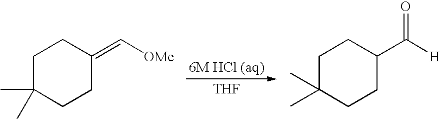

A solution of (4,4-dimethyl cyclohexylidene)methyl methyl ether (6.7 g, 0.043 mol) in THF (200 mL) containing 6M HCl (aq) (60 mL) was stirred at room temperature for 24 hr. The reaction mixture was diluted with a mixture of ethyl ether, hexanes, brine and water. The mixture was separated and the aqueous phase was extracted with ethyl ether. The combined organic phase was washed with brine, dried (Na₂SO₄), and concentrated in vacuo to give 4,4-dimethylcyclohexanecarbaldehyde as a yellow oil. This material was used without further purification.

### Step 3: [(4,4-dimethylcyclohexyl)methyl](phenylmethyl)amine

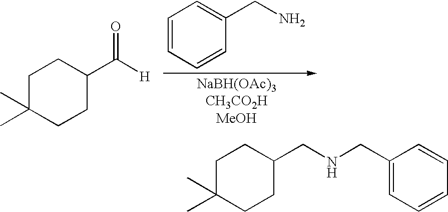

A solution of 4,4-dimethylcyclohexanecarbaldehyde (6.6 g, 0.047 mol), benzylamine (5.0 g, 0.047 mol) and acetic acid (1 mL) in methanol (60 mL) was stirred for 30 min at room temperature. Sodium triacetoxyborohydride (10.0 g, 0.047 mol) was added in one portion and the mixture was stirred at room temperature for 16 hr. The reaction mixture was concentrated in vacuo and partitioned between dichloromethane and water. The organic phase was washed with brine, silica gel was added and the mixture was concentrated in vacuo. The residue was purified by silica gel chromatography using dichloromethane (100%), then 7% MeOH/dichloromethane as eluent. The fractions containing the major component were concentrated in vacuo to give [(4,4-dimethyl cyclohexyl)methyl](phenylmethyl)amine as a white solid. (M+1) 232.2 AP, 1.76 min. (LC/MS method B).

### Step 4: [(4,4-dimethylcyclohexyl)methyl]amine hydrochloride

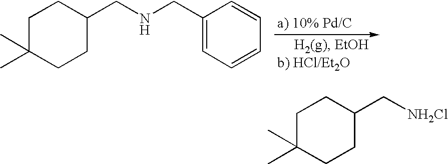

A mixture of [(4,4-dimethylcyclohexyl)methyl](phenylmethyl)amine (4.37 g, 0.019 mol) and 10% Pd/C (50% w/w with water) (0.75 g) in ethanol (100 mL) was placed on a Parr hydrogenation apparatus. After 24 hr, the reaction mixture was filtered through celite. To the filtrate was added 1.0N HCl in ethyl ether (30 mL) and the mixture was concentrated in vacuo. The residue was triturated with ethyl ether, filtered, washed with ethyl ether and dried to give [(4,4-dimethylcyclohexyl)methyl]amine hydrochloride as a white solid. 1H NMR (400 MHz, DMSO-d₆) delta ppm 0.83 (s, 3H); 0.86 (s, 3H); 1.11 (m, 4H); 1.33 (m, 2H); 1.46 (m, 1H); 1.53 (m, 2H); 2.64 (br s, 2H); 7.91 (br s, 3H).

Amine-8

### Preparation of 2-cyclohexylethylamine hydrochloride:

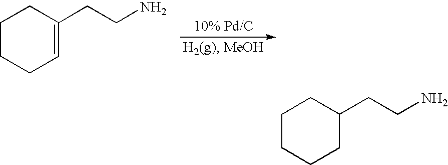

2-(1-Cyclohexenyl)ethylamine (5.60 g) was dissolved in 60 ml of methanol. The solution, along with 0.6 g of 10% Palladium on Carbon (Degussa type E101), was hydrogenated under 55 psi for 5 h at room temperature. The mixture was passed through a Celite® pad to remove the catalyst and then was concentrated in vacuum to give the free base product as tan oil. The free base (0.51 g) was dissolved in dichloromethane (5 ml) and 3 ml of 1M HCl in diethyl ether was added. The title compound was obtained by filtration as a white solid.

¹H NMR (400 MHz, DMSO-d₆) delta ppm 0.85 (m, 2H), 1.02-1.36 (m, 5H), 1.41 (m, 2H), 1.55-1.76 (m, 4H), 2.75 (m, 2H), 7.90 (br, 3H)

Amines-9, 10 and 11

### 3,3-dimethylcyclohexylamine hydrochloride

### Step 1: 3,3-dimethylcyclohexanone oxime

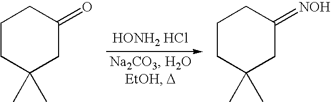

To a mixture of 3,3-dimethylcyclohexanone (4.0 g, 0.032 mol) and hydroxylamine hydrochloride (2.9 g, 0.041 mol) in ethanol (20 mL) was added dropwise a solution of sodium carbonate (4.3 g, 0.041 mol) in water (25 mL). The mixture was heated at reflux for 3 hr. The mixture was concentrated in vacuo to remove the ethanol and the aqueous residue was extracted with ethyl acetate. The organic phase was dried (MgSO₄), filtered and concentrated in vacuo to give 3,3-dimethylcyclohexanone oxime as a yellow oil. This material was used without further purification.

### Step 2: (3,3-dimethylcyclohexyl)amine hydrochloride

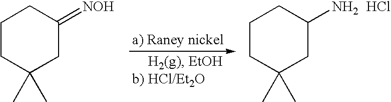

A mixture of 3,3-dimethylcyclohexanone oxime (4.41 g, 0.031 mol) and Raney nickel in water (1.0 g) in ethanol (100 mL) was placed on a Parr hydrogenation apparatus. After 11 days, the reaction mixture was filtered through celite. To the filtrate was added 1.0N HCl in ethyl ether (50 mL) and the mixture was concentrated in vacuo. The residue was triturated with ethyl ether, filtered, washed with ethyl ether and dried to give (3,3-dimethylcyclohexyl)amine hydrochloride as a white solid. ¹H NMR (400 MHz, DMSO-d₆) delta ppm: 0.85 (s, 3H); 0.90 (s, 3H); 0.97-1.16 (m, 3H); 1.29 (br d, 1H); 1.34-1.46 (m, 1H); 1.53-1.63 (m, 2H); 1.90 (br d, 1H); 3.05 (m, 1H); 7.99 (br s, 3H).

### [(1S)-3,3-dimethylcyclohexyl]amine Hydrochloride and [(1R)-3,3-dimethylcyclohexyl]amine Hydrochloride

### Step 1: phenylmethyl (3,3-dimethylcyclohexyl)carbamate

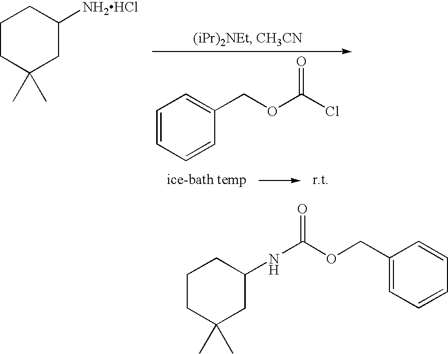

To a solution of (3,3-dimethylcyclohexyl)amine hydrochloride (10.0 g, 0.06 mol) and N,N-diisopropylethylamine (15.8 g, 0.12 mol) in acetonitrile (125 mL) chilled in an ice bath was added dropwise a solution of benzylchloroformate (11.4 g, 0.067 mol) in acetonitrile (25 mL). The mixture was stirred and allowed to come to ambient temperature overnight. The reaction mixture was concentrated in vacuo and the residue was partitioned between ethyl acetate and 5% aqueous citiric acid solution. The organic phase was washed with saturated aqueous sodium chloride solution and dried over sodium sulfate. The mixture was filtered, silica gel was added to the residue and the mixture was concentrated in vacuo. The residue was purified by column chromatography using dichloromethane/hexanes (4:1) as eluent to give 11.2 g (70%) of phenylmethyl (3,3-dimethylcyclohexyl) carbamate as a colorless oil. ¹H NMR (400 MHz, DMSO-d₆) delta ppm: 0.85 (s, 6H); 0.92-1.02 (m, 3H); 1.25 (d, 1H); 1.36-1.52 (m, 3H); 1.76 (br d, 1H); 3.36-3.44 (m, 1H); 4.96 (s, 2H); 7.10 (d, 1H); 7.27-7.36 (m, 5H).

### Step 2: Phenylmethyl [(1S)-3,3-dimethylcyclohexyl]carbamate and Phenylmethyl [(1R)-3,3-dimethylcyclohexyl]carbamate

phenylmethyl (3,3-dimethylcyclohexyl)carbamate (11.2 g) was separated by supercritical fluid chromatography (SFC) on a Chiralpak AS column (30 mm). The flow rate was 75 gr/min. CO₂ and 4 mL/min. ethanol. The pressure was 140 bar and the temperature was 40 degrees C. The material eluting at 4.41 min. was combined and concentrated in vacuo to give 3.98 g of a colorless oil. Analytical SFC showed this enantiomer to be >99% pure. Ab Initio Vibrational Circular Dichroism determined this to be the S-enantiomer. [α]ₚ=−17.2° (c=0.01, MeOH), optical rotation was obtained at 25° C. ¹H NMR (400 MHz, DMSO-d₆) delta ppm: 0.85 (s, 6H); 0.93-1.01 (m, 3H); 1.25 (d, 1H); 1.33-1.52 (m, 3H); 1.75 (br d, 1H); 3.35-3.44 (m, 1H); 4.96 (s, 2H); 7.08 (d, 1H); 7.25-7.35 (m, 5H). (M+1) 262 ES, 2.8 min. (LC/MS Method A).

The material eluting at 5.51 min. was combined and concentrated in vacuo to give 3.57 g of a colorless oil. Analytical SFC showed this enantiomer to be >96% pure. Ab Initio Vibrational Circular Dichroism determined this to be the R-enantiomer. [α]_{D}=+16.3° (c=0.011, MeOH), optical rotation was obtained at 25° C. ¹H NMR (400 MHz, DMSO-d₆) delta ppm: 0.85 (s, 6H); 0.92-1.01 (m, 3H); 1.25 (d, 1H); 1.33-1.52 (m, 3H); 1.75 (br d, 1H); 3.36-3.44 (m, 1H); 4.96 (s, 2H); 7.08 (d, 1H); 7.26-7.35 (m, 5H). (M+1) 262 APCI, 2.86 min. (LC/MS Method B).

Amine-10

### Step 3: [(1S)-3,3-dimethylcyclohexyl]amine Hydrochloride

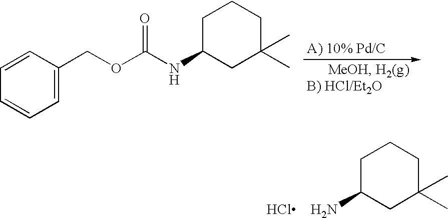

phenylmethyl [(1S)-3,3-dimethylcyclohexyl]carbamate (1.0 g, 0.004 mol) and 10% Pd/C (0.15 g) were placed in 10 mL of MeOH and the mixture was placed under a balloon of H₂ (g) for 24 hrs. The catalyst was removed by filtration through celite. To the filtrate was added 1N HCl in ethyl ether (2.5 mL) and the mixture was left at room temperature overnight. The mixture was concentrated in vacuo. The residue was triturated with ethyl ether. The resulting solid was filtered, washed with ethyl ether and dried in vacuo to give 0.41 g (66%) of [(1S)-3,3-dimethylcyclohexyl]amine Hydrochloride as a white solid. ¹H NMR (400 MHz, DMSO-d₆) delta ppm: 0.89 (s, 3H); 0.93 (s, 3H); 1.00-1.17 (m, 3H); 1.33 (br d, 1H); 1.38-1.49 (br q, 1H); 1.58-1.63 (m, 2H); 1.92 (br d, 1H); 3.06-3.14 (m, 1H); 7.86 (s, 3H). This compound was used without further purification.

Amine-11

### Step 4: [(1R)-3,3-dimethylcyclohexyl]amine Hydrochloride

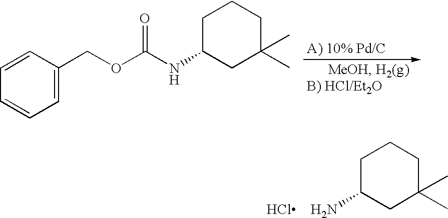

Phenylmethyl [(1R)-3,3-dimethylcyclohexyl]carbamate (1.0 g, 0.004 mol) and 10% Pd/C (0.15 g) were placed in 10 mL of MeOH and the mixture was placed under a balloon of H₂ (g) for 20 hrs. The catalyst was removed by filtration through celite. To the filtrate was added 1N HCl in ethyl ether (3.0 mL) and the mixture was concentrated in vacuo. The residue was triturated with ethyl ether containing 1N ethereal hydrogen chloride (0.5 mL). The resulting solid was filtered, washed with ethyl ether and dried in vacuo to give 0.56 g (90%) of [(1R)-3,3-dimethylcyclohexyl]amine Hydrochloride as a white solid. ¹H NMR (400 MHz, DMSO-d₆) delta ppm: 0.85 (s, 3H); 0.90 (s, 3H); 0.96-1.14 (m, 3H); 1.29 (br d, 1H); 1.35-1.45 (br q, 1H); 1.53-1.61 (m, 2H); 1.89 (br d, 1H); 3.02-3.09 (m, 1H); 7.88 (s, 3H). This compound was used without further purification.

Amine-12

### Preparation of 4,4-dimethylcyclohexylamine hydrochloride

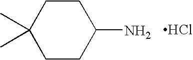

This amine was prepared according to the procedure described in J. Med. Chem. 1971, 14 (7), 600-614.

Amine-13

### Preparation of [5-(methyloxy)-2,3-dihydro-1H-inden-2-yl]amine hydrochloride

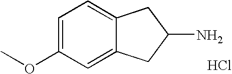

Prepared similar to the procedure of Susanne R. Haadsma-Svensson, Kerry A. Cleek, Dac M. Dinh, J. Neil Duncan, Christopher L. Haber, Rita M. Huff, Mary E. Lajiness, Nanette F. Nichols, Martin W. Smith, Kjell A. Svensson, Matt J. Zaya, Arvid Carlsson, and Chiu-Hong Lin J Med Chem 44, (26) 4716-4732.

### Step 1: (2E)-6-(methyloxy)-1H-indene-1,2(3H)-dione 2-oxime Preparation

To a solution of the indanone (1.0 g, 6.2 mmol) in Methanol (15 mL) at 40 degrees C. was added n-butyl nitrite (0.8 mL, 6.25 mmol) followed by concentrated HCl (0.6 mL). The reaction was stirred for 30 min during which time a precipitate formed, was collected and dried. This was used without further purification.

¹H NMR (400 MHz, DMSO-d₆) delta ppm 3.60 (bs, 2H) 3.86 (s, 3H) 6.99 (dd, J=8.54, 2.2 Hz, 1H) 7.12 (d, J=1.71 Hz, 1H) 7.66 (d, J=8.55 Hz , 1H) 12.45 (s, 1H)

### Step 2: [5-(methyloxy)-2,3-dihydro-1H-inden-2-yl]amine hydrochloride Preparation

To a solution of the (2E)-6-(methyloxy)-1H-indene-1,2(3H)-dione 2-oxime (0.96 g, 5.02 mmol) in glacial AcOH (25 mL) and concentrated H₂SO₄ (2 mL) was added 10% Palladium on carbon (50% H₂O, 0.200 g). This mixture was hydrogenated on a Parr apparatus at 50 psi for 7 h, then filtered over Celite to remove the catalyst and washed 2×10 mL methanol and concentrated to remove the AcOH, basified to pH 12 at which point a solid formed and extracted with CH₂Cl₂ (2×100 mL). The combined organics were dried (Na₂SO₄) and concentrated to ½ volume. Gaseous HCL was bubbled through the remaining solution for a scant minute and the resulting mixture concentrated after 15 min standing at ambient temperature to give 0.906 g of desired product as the HCl salt.

¹H NMR (400 MHz, DMSO-d₆) delta ppm 2.84-2.98 (m, 2H) 3.09-3.24 (m, 2H) 3.75 (s, 3H) 3.92 (bs, 1H) 6.73 (dd, J=8.3, 2.44 Hz, 1H) 6.83 (d, J=2.2 Hz, 1H) 7.13 (d, J=8.06Hz , 1H) 8.40 (bs, 2H)

Amine-14 and Amine-15

The [4-(methyloxy)-2,3-dihydro-1H-inden-2-yl]amine and [5,6-bis(methyloxy)-2,3-dihydro-1H-inden-2-yl]amine were synthesized in the same manner as amine-13 from the corresponding indanone.

Amine-14

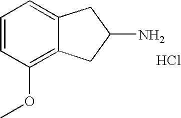

¹H NMR (400 MHz, DMSO-d₆) delta ppm 2.84-2.98 (m, 2H) 3.10-3.27 (m, 2H) 3.75 (s, 3H) 3.95 (bs, 1H) 6.79 (d, J=8.06 Hz, 1H) 6.84 (d, J=7.32 Hz, 1H) 7.16 (t, J=7.81 Hz, 1H) 8.31 (bs, 2H)

Amine-15

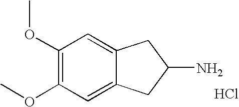

¹H NMR (400 MHz, DMSO-d₆) delta ppm 2.84-2.98 (m, 2H) 3.10-3.27 (m, 2H) 3.75 (s, 3H) 3.95 (bs, 1H) 6.79 (d, J=8.06 Hz, 1H) 6.84 (d, J=7.32 Hz, 1H) 7.16 (t, J=7.81 Hz, 1H) 8.31 (bs, 2H)

Amine-16

### Preparation of (2-methyl-2,3-dihydro-1H-inden-2-yl)amine hydrochloride

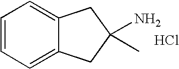

### Step 1: methyl 2-methyl-1-oxo-2,3-dihydro-1H-indene-2-carboxylate Preparation

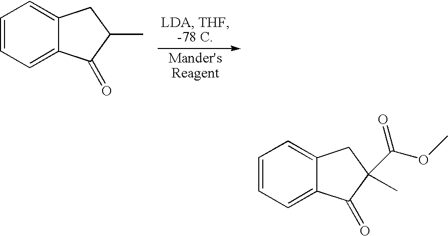

To a flask cooled to 0 degrees C. containing diisopropylamine (2.06 mL, 14.58 mmol) in Tetrahydrofuran (14 mL) was added dropwise over 15 min a solution of n-butyl lithium (5.55 mL of 2.5 M in Hexanes, 14.58 mmol). This mixture was stirred for 30 min and then a second flask containing the 2-methyl-1-indanone (2.03 g, 13.89 mmol) in Tetrahydrofuran (10 mL) was cooled to −78 degrees C. under N₂. The freshly prepared LDA was cooled to −78 degrees C. and added dropwise via cannula. The orangish mixture became somewhat heterogeneous over 30 min and then neat methyl cyanoformate (1.32 mL, 16.66 mmol) was added and the reaction mixture stirred an additional 40 min while allowing the reaction to warm to −20 degrees C. The reaction was quenched with saturated aqueous NH₄Cl solution and the organics extracted 2×25 mL diethyl ether, then washed 1× brine, then dried (Na₂SO₄), filtered and concentrated to an orange oil. No further purification necessary.

¹H NMR (400 MHz, CDCl₃) delta ppm 1.52 (s, 3H) 3.00 (d, J=17.32 Hz, 1H) 3.67-3.73 (m, 4H) 7.41 (t, J=7.57 Hz, 1H) 7.47 (m, 1H) 7.63 (m, 1H) 7.79 (d, J=7.57 Hz, 1H)

### Step 2: methyl 2-methyl-2,3-dihydro-1H-indene-2-carboxylate

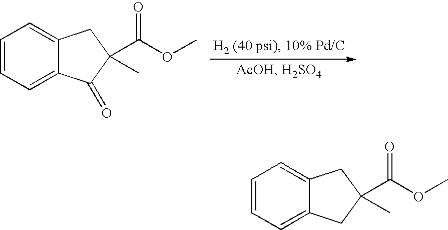

A solution of the methyl 2-methyl-1-oxo-2,3-dihydro-1H-indene-2-carboxylate (2.04 g, 9.99 mmol) in glacial AcOH (22 mL) and concentrated H₂SO₄ (2 mL) was hydrogenated on a Parr apparatus under 50 psi of hydrogen using 10% palladium on carbon (50% H₂O, 0.200 g) as the catalyst. After 4 h the reaction was filtered to remove the catalyst and washed 2× methanol, then concentrated to remove most of the AcOH. The residue was neutralized using saturated Na₂CO₃ and the organics extracted 2×25 mL ethyl acetate, then the combined organics washed 1× brine, dried (Na₂SO₄), filtered and concentrated to 2.0 g of a clear oil.

¹H NMR (400 MHz, CDCl₃) delta ppm 1.35 (s, 3H) 2.81 (d, J=15.63 Hz, 2H) 3.47 (d, J=15.63 Hz, 2H) 3.71 (s, 3H) 7.12-7.23 (m, 4H)

### Step 3: 2-methyl-2,3-dihydro-1H-indene-2-carboxylic acid

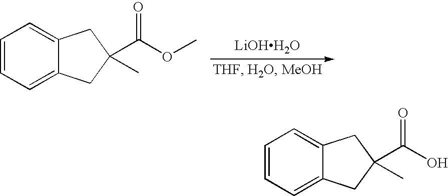

To a tetrahydrofuran/H₂O/methanol (4 mL/1 mL/1 mL) of the methyl 2-methyl-2,3-dihydro-1H-indene-2-carboxylate (1.80 g, 9.46 mmol) was added lithium hydroxide monohydrate (1.19 g, 28.39 mmol). The reation mixture stirred at ambient temperature for 4 h and then the mixture was acidified to pH 3 using 1N HCl and the organics extracted 2×25 mL diethyl ether, then the combined organics were washed 1× brine and then dried (Na₂SO₄), filtered and concentrated to a white solid.

¹H NMR (400 MHz, CDCl₃) delta ppm 1.39 (s, 3H) 2.83 (d, J=15.87 Hz, 2H) 3.50 (d, J=15.87 Hz, 2H) 7.12-7.23 (m, 4H)

### Step 4: phenylmethyl (2-methyl-2,3-dihydro-1H-inden-2-yl)carbamate

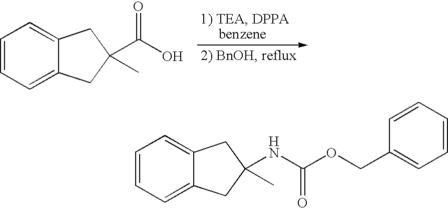

To a solution of 2-methyl-2,3-dihydro-1H-indene-2-carboxylic acid (0.200 g, 1.14 mmol) and triethylamine (0.166 mL, 1.19 mmol) in benzene (2 mL) cooled to 0 degrees C. was added diphenyl phosphorylazide (0.257 g, 1.19 mmol). The reaction mixture was stirred for 15 min and benzyl alcohol (0.123 mL, 1.19 mmol) was added and the reaction was heated to reflux for 16 h, then cooled and 10% HCl added. The organics were extracted 2×25 mL Ethyl Acetate, then washed 1× brine, dried (Na₂SO₄), filtered and concentrated. The residue was chromatographed on silica using 5:1 Hexanes/Ethyl Acetate to give 0.271 g of the phenylmethyl (2-methyl-2,3-dihydro-1H-inden-2-yl)carbamate.

¹H NMR (400 MHz, CDCl₃) delta ppm 1.55 (s, 3H) 2.98 (d, J=15.87 Hz, 2H) 3.28 (d, J=15.87 Hz, 2H) 7.12-7.18 (m, 4H) 7.29-7.37 (m, 5H)

### Step 5: (2-methyl-2,3-dihydro-1H-inden-2-yl)amine hydrochloride

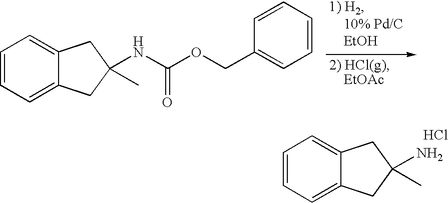

A Parr vessel containing the phenylmethyl (2-methyl-2,3-dihydro-1H-inden-2-yl)carbamate (0.271 g, 0.963 mmol) and 10% Palladium on carbon (50% H₂O, 0.050 g) in Ethanol (2 mL) was charged and evacuated several times with hydrogen before maintaining a final pressure of 40 psi while shaking for 4 h. The catalyst was removed by filtration over Celite and the filtrate concentrated to an oil. The residue was redissolved in Ethyl Acetate and cooled to −70 degrees C. and then gaseous HCl was bubbled through the solution until saturated. The reaction mixture stirred for 1 h and then concentrated to give 0.175 g of (2-methyl-2,3-dihydro-1H-inden-2-yl)amine hydrochloride as a white solid.

¹H NMR (400 MHz, methanol-d₄) delta ppm 1.56 (s, 3H) 3.17 (bs, 4H) 7.19-7.29 (m, 4H)

Amines-17 and 18

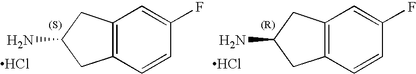

### (2S)-5-fluoro-2,3-dihydro-1H-inden-2-amine hydrochloride (Amine 17) and (2R)-5-fluoro-2,3-dihydro-1H-inden-2-amine hydrochloride (Amine 18)

### Step 1: rac-5-Fluoro-2,3-dihydro-1H-inden-2-amine

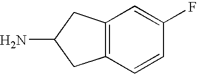

To a solution of 5-fluoro-1-indanone (10.0 g; 66.7 mmol) in MeOH at 40° C. was added n-BuONO (13.2 mL; 113 mmol) dropwise over 3 minutes, followed by conc HCl (10 mL), dropwise at such a rate that the internal temp was maintained below 55 degrees C. The mixture was stirred 30 min and concentrated in vacuo. The residue was diluted with EtOAc and sat'd NaHCO₃, filtered, and the layers were separated. The aqueous layer was extracted with EtOAc (×1), combined organics were washed (H₂O, brine), dried over Na₂SO₄, filtered and concentrated in vacuo. The residue was purified by flash chromatography (EtOAc/hexanes), affording 7.59 g of the keto-oxime intermediate as an orange solid. The solid was dissolved in HOAc/H₂SO₄ (250 mL/12.5 mL), Pd-C was added (4.5 g; 10 wt % dry basis, wet, DeGussa type E101) and the mixture was hydrogenated using a Parr apparatus (50 psi H₂) for 18 h. The mixture was filtered through Celite (H₂O wash) and partially concentrated to an aqueous mixture. The mixture was adjusted to pH 11 (1 N NaOH) and extracted with CHCl₃ (×5). Combined organics were washed (brine), dried over Na₂SO₄, filtered and concentrated in vacuo, affording 5.79 g of the title compound as an amber oil which was used without further purification. ¹H NMR (400 MHz, DMSO-d₆) δ 1.69 (br. s, 2H), 2.53 (m, 2H, overlapping solvent), 2.99 (m, 2H), 3.69 (quint, J=6.2 Hz, 1H), 6.89 (partially resolved ddd, J=9.8, ˜7.7, 2.5 Hz, 1H), 6.99 (partially resolved dd, J=9.3, ˜2.3 Hz, 1H), 7.16 (partially resolved dd, J=8.3, 5.6 Hz, 1H).

### Step 2: rac-(5-fluoro-2,3-dihydro-1H-inden-2-yl)benzyl carbamate

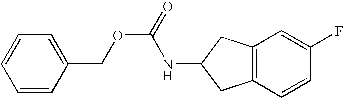

To a mixture of 5-fluoro-2,3-dihydro-1H-inden-2-amine (5.79 g; 38.3 mmol; step 1 above) and sat'd Na₂CO₃ (200 mL) at room temp was added benzyl chloroformate (6.9 mL; 46 mmol). The mixture was stirred 1 h at room temp and extracted with EtOAc (×3). Combined organics were washed (H₂O, brine), dried over Na₂SO₄, filtered and concentrated in vacuo. The residue was purified by flash chromatography (EtOAc/hexanes), affording 8.33 g of the title compound as an off-white solid. ¹H NMR (400 MHz, DMSO-d₆) δ 2.77 (m, 2H), 3.12 (m, 2H), 4.29 (app. sext, J=7.1 Hz, 1H), 5.02 (s, 2H), 6.94 (m, 1H), 7.03 (partially resolved dd, J=9.2, ˜2.4 Hz, 1H), 7.19 (partially resolved dd, J=8.2, 5.5 Hz, 1H), 7.28-7.40 (m, 5H), 7.64 (d, J=6.8 Hz, 1H).

### Step 3: Resolution of rac-(5-Fluoro-2,3-dihydro-1H-inden-2-yl)benzyl carbamate into [(2S)-5-fluoro-2,3-dihydro-1H-inden-2-yl]benzyl carbamate and [(2R)-5-fluoro-2,3-dihydro-1H-inden-2-yl]benzyl carbamate

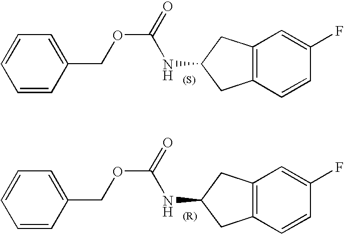

rac-(5-Fluoro-2,3-dihydro-1H-inden-2-yl)benzyl carbamate was separated into individual enantiomers by supercritical fluid chromatography using an AD—H prep column (30 mm ID×25 mm, 5 μm particle size), MeOH/CO₂ (17/83) at 140 bar, 90 g/min total flow, 33° C. Chromatographic peaks were detected at 215 nm.

Absolute configuration assignments for the enantiomers obtained above were made by comparison of experimentally measured vibrational circular dichroism (VCD) spectra with the calculated (ab initio) VCD spectrum for [(2S)-5-fluoro-2,3-dihydro-1H-inden-2-yl]benzyl carbamate. The earlier-eluting enantiomer from the chiral separation described above was found to have VCD bands of the same relative sign as the (S)-configuration model used for ab initio calculations, and thus assigned the (S)-configuration. In contrast, the latter-eluting enantiomer was found to have VCD bands of the opposite relative sign as the (S)-configuration model used for ab initio calculations, and thus assigned the (R)-configuration.

### Step 4: (S)- and (R)-5-fluoro-2,3-dihydro-1H-inden-2-amine hydrochloride

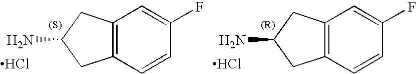

The preparation of (S)-5-fluoro-2,3-dihydro-1H-inden-2-amine hydrochloride is given as representative. To a solution of [(2S)-5-fluoro-2,3-dihydro-1H-inden-2-yl]benzyl carbamate (2.26 g; 7.93 mmol) in EtOAc/EtOH (40 mL ea) was added Pd—C (0.85 g; 10 wt % (dry basis), wet, DeGussa type E101). The mixture was stirred under an atmosphere of H₂ for 5 h and catalyst was removed by filtration through a 0.45 μm PTFE membrane filter. HCl (5 mL of a 4N solution in dioxane) was added to the filtrate and the whole was concentrated to dryness, affording 1.41 g of the title compound as a tan solid. ¹H NMR (400 MHz, DMSO-d₆) δ 2.97 (m, 2H), 3.24 (m, 2H), 4.00 (m, 1), 7.01 (m, 1H), 7.13 (partially resolved dd, J=9.2, ˜2.4 Hz, 1H), 7.28 (dd, J=8.4, 5.4 Hz, 1H), 8.40 (br. s, 2H). (M+H) 152, 0.73 min (LC/MS method C). (R)-5-Fluoro-2,3-dihydro-1H-inden-2-amine hydrochloride was prepared in an identical fashion, and exhibited an identical ¹H NMR spectrum and LC/MS retention time.

### ABBREVIATIONS

Boc₂O or (BOC)₂O di-t-butyldicarbonate

Boc or BOC 1,1-dimethylethyloxycarbonyl

DAST (diethylamino)sulfur trifluoride

DBU 1,8-diazabicyclo[5.4.0]undec-7-ene

Deoxo-fluor bis(2-methoxyethyl)aminosulfur trifluoride

DIBAL diisobutylaluminum hydride

DMAP 4-dimethylaminopyridine

DME 1,2-dimethoxyethane

DMF dimethylformamide

DMSO dimethylsulfoxide

DPPA diphenyl phosphorylazide

HOAc acetic acid

LDA lithium diisopropylamine

Mander's Reagent methyl cyanoformate

MP—BH₃CN macroporous resin-cyanoborohydride

NBS N-bromosuccinimide

PdCl₂.dppf.CH₂Cl₂ dichloro[1,1′-bis(diphenylphosphino)ferrocene]palladium (II) dichloromethane

PPA polyphosphoric acid

PS-TsNHNH₂ polystyrene resin-p-toluenesulfonyl hydrazide

PTFE (poly)tetrafluoroethylene polymer

pTsOH p-toluenesulfonic acid

TEA or Et₃N triethylamine

TFA trifluoroacetic acid

Tf₂O trifluoromethanesulfonic anhydride

THF tetrahydrofuran

TrCl trityl chloride

Tr trityl

LC/MS Methods

LC/MS Method A (Standard Electrospry Method): Mass Spectrometry is used to confirm peak identity with electrospray+/−ionization scanning from 100-1000 m/z and DAD from 220-400 nm. Phenomenex Luna column 4.6 mm by 2 cm, particle size 3 um, ambient temperature. Solvent flow at 2 ml/min. Gradient begins at 10% MeOH and goes linearly to 100% MeOH in 3 minutes, holds 100% MeOH for 1 minute, making total chromatogram time 4 minutes. 2 ul sample injection. Aqueous mobile phase contains 0.1% v/v Formic Acid and MeOH contains 0.075% v/v Formic Acid.

LC/MS Method B (Standard APCI Method): Mass Spectrometry is used to confirm peak identity with APCI+/−ionization scanning from 100-1000 m/z and DAD from 220-400 nm. Phenomenex Luna column 4.6 mm by 2 cm, particle size 3 um, ambient temperature. Solvent flow at 2 ml/min. Gradient begins at 10% MeOH and goes linearly to 100% MeOH in 3 minutes, holds 100% MeOH for 1 minute, making total chromatogram time 4 minutes. 2 ul sample injection. Aqueous mobile phase contains 0.1% v/v Formic Acid and MeOH contains 0.075% v/v Formic Acid.

LC/MS Method C (Polar APCI Method): Mass Spectrometry is used to confirm peak identity with APCI+/−ionization scanning from 100-1000 m/z and DAD from 220-400 nm. Phenomenex Luna column 4.6 mm by 2 cm, particle size 3 um, ambient temperature. Solvent flow at 2 ml/min. Gradient begins at 2% MeOH and goes linearly to 26% MeOH in 1 minute, then goes linearly from 26% MeOH to 100% MeOH in 2 min., then holds 100% MeOH for 1 minute, making total chromatogram time 4 minutes. 2 ul sample injection. Aqueous mobile phase contains 0.1% v/v Formic Acid and MeOH contains 0.075% v/v Formic Acid.

LC/MS Method D (Polar Electrospray Method): Mass Spectrometry is used to confirm peak identity with electrospray+/−ionization scanning from 100-1000 m/z and DAD from 220-400 nm. Phenomenex Luna column 4.6 mm by 2 cm, particle size 3 um, ambient temperature. Solvent flow at 2 ml/min. Gradient begins at 2% MeOH and goes linearly to 26% MeOH in 1 minute, then goes linearly from 26% MeOH to 100% MeOH in 2 min., then holds 100% MeOH for 1 minute, making total chromatogram time 4 minutes. 2 ul sample injection. Aqueous mobile phase contains 0.1% v/v Formic Acid and MeOH contains 0.075% v/v Formic Acid.

LC-MS Method E (Standard Electrospray Fast Mass Spec Method): Electrospray+ionization scanning from 100-800 m/z with DAD sum from 220-400 nm. Waters Acquity UPLC column 2.1 mm by 5 cm, particle size 1.7 um, temperature at 40 degrees C. Solvent flow at 1 ml/min. Gradient begins at 6% ACN and goes linearly to 70% ACN in 0.57 minute; gradient then goes linearly to 99% ACN from 0.57 minute to 1.06 minute, holds 99% ACN until 1.5 minute, making total chromatogram time 1.5 minutes. 1.5 ul sample injection. Aqueous mobile phase contains 0.1% v/v Formic Acid and ACN contains trace v/v Formic Acid.

LC-MS Method F (Standard APCI Fast Mass Spec Method): Mass Spectrometry is used to confirm peak identity with APCI+/−ionization scanning from 100-1000 m/z and DAD from 220-400nm. Column is Waters Acquity BEH UPLC column 2.1 mm by 5 cm, particle size 1.7 um, temperature at 25 degrees C. Solvent flow at 1 ml/min. Gradient begins at 6% ACN and goes linearly to 70% ACN in 0.57 minute; gradient then goes linearly to 99% ACN from 0.57 minute to 1.06 minute, holds 99% ACN until 1.5 minute, making total chromatogram time 1.5 minutes. 1.5 ul sample injection. Aqueous mobile phase contains 0.1% v/v Formic Acid and ACN contains trace v/v Formic Acid.

Method of Testing Compounds of the Invention

Materials

LEADSeeker WGA™ beads and GTPgS35 were purchased from Amersham Bioscience (Piscataway, N.J.). GDP, Saponin™, DAMGO™, Met-Enkephalin, Dynorphin A, NaCl and HEPES™ were purchased from SIGMA (St Louis, Mo.). MgCl2 was purchased from J. T. Baker (Pillipsburg, N.J.). Opioid membranes, hOPRD, hOPRK and hOPRM were prepared at GlaxoSmithkline (Harlow, UK). Assay buffer; 20 mM HEPES, 10 mM MgCL2, and 100 mM NaCl dissolved in labgrade water, pH 7.4 with KOH

[35S]GTPgammaS Binding Assay Measured by LEADseeker SPA (384 Well)

Dilute GTPgS35 1:900 in assay buffer in half of required final assay volume (volume A). Add the corresponding standard agonist, Met-Enkephalin (hOPRD), Dynorphin A (hOPRK) or DAMGO (hOPRM) to give a solution concentration of 8×[EC50], for a final assay concentration of 4×[EC50] to volume A. Resuspend LEADSeeker beads in assay buffer in order to generate a 40 mg/mL stock solution. GDP is dissolved in assay buffer at 1 mM. Add beads (100 microgram/well final) to assay buffer containing saponin (60 microgram/mL) in half of final assay volume (volume B). Mix well by vortexing. Add opioid membranes to each respective volume B, for a final assay concentration of 1.5 microgram/well (hOPRD), 1.0 microgram/well (hOPRK), and 1.5 microgram/well (hOPRM). Continuously mix the bead/membrane solution (volume B) for 30 min prior to adding to the GTPgS35 solution (volume A) in a 1:1 ratio using a stir plate. Just prior to adding bead/membrane solution to the GTPgS35 solution, add GDP to volume B at 20 microMolar (10 microMolar final assay concentration). Add the bead/membrane solution to the GTPgS35 solution in a 1:1 ratio. Add 10 microLiters of the bead/membrane/GTPgS35 mix to the assay plate using a Multidrop (Titertek™). Agitation of the solution is needed to prevent the beads/membrane from settling at the bottom. Plates are sealed, spun at 1000 rpm for 2 mins, tapped on side to agitate and incubated at room temperature for 5 hours. Plates are then imaged using a Viewlux Plus™ Imager (Perkin Elmer).

Acceptable compounds of the invention have an activity of 30 micromolar or higher using this test method.

What is claimed is:

1. A compound of Formula I 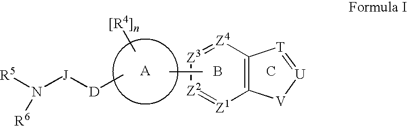
a salt, a solvate, or a physiologically functional derivative thereof wherein:
Ring A is selected from the group consisting of aryl or heteroaryl, and ring A is attached to Z² or Z³;
D is selected from the group consisting of —CH₂—, and —O— and is attached to a carbon atom of ring A, with the proviso that D is not attached to the atom adjacent to the bond joining ring A and fused ring BC;
J is a bond or a C₁₋₄alkylene;
each Z¹, Z², Z³, and Z⁴ is the same or different and is selected from the group consisting of CH, N, and CR³ with the proviso that Z² or Z³ is a carbon atom to which Ring A is attached, and that no more than two of Z¹, Z², Z³, and Z⁴ are N;
T and U are each independently selected from the group consisting of N, CH, C(NR¹R²), and C(R²); V is selected from the group consisting of NH, O, S, and
NR¹; wherein R¹ and R² are each independently selected from the group consisting of a C₁₋₆ alkyl and a fluoroalkyl;
R³ and R⁴ are each independently selected from the group consisting of —F, —Cl, —Br, —OH, —CN, —OC₁₋₃ alkyl, —C₁₋₃ fluoroalkyl, and —C₁₋₃ alkyl; n is 0, 1, or 2;
R⁵ is selected from the group consisting of hydrogen, C₁₋₁₂ alkyl, C₃₋₁₀ cycloalkyl, arylalkyl, heterocyclyl, heterocycloalkyl, heteroarylalkyl, cycloalkenyl, C₂₋₁₂ fluoroalkyl, and heteroalkyl; and
R⁶ is selected from the group consisting of C₃₋₁₂ alkyl, C₃₋₁₀ cycloalkyl, arylalkyl, heterocyclyl, heterocycloalkyl, heteroarylalkyl, cycloalkenyl, C₃₋₁₂ fluoroalkyl, and heteroalkyl.

2. The compound of claim 1 wherein Ring A is selected from the group consisting of phenyl, thienyl, furanyl, pyrrolyl, thiazolyl, oxazolyl, oxadiazolyl, thiadiazolyl, imidazolyl, pyridyl, pyrimidinyl, pyrazinyl, triazinyl; naphthalyl, quinolinyl, isoquinolinyl, indolyl, benzthiophenyl, benzimidazolyl, benzoxazolyl, benzthiazolyl, benzisoxazolyl, indazolyl, pyrazolopyridinyl, pyrazolopyrimidinyl, pyrazolopyrazinyl, imidazopyridinyl, purinyl, thiazolopyridinyl, thiazolopyrimidinyl, thiazolopyrazinyl, oxazolopyridinyl, oxazolopyrimidinyl, and oxazolopyrazinyl.

3. The compound of claim 2 wherein Ring A is phenyl or pyridyl.

4. The compound of claim 1 wherein D is —CH₂— and J is a bond.

5. The compound of claim 1 wherein when D is —O—, J is a C₂₋₃alkylene.

6. The compound of claim 1 wherein when D is —O—, and J is a C₂alkylene.

7. The compound of claim 1 wherein T is N, V is NH, and U is CH and ring A is attached to Z³.

8. The compound of claim 1 wherein T is N, V is NH, and U is CH and ring A is attached to Z².

9. The compound of claim 1 wherein R⁵ is hydrogen.

10. The compound of claim 1 wherein R⁶ is selected from the group consisting of arylmethyl, arylethyl, C₄₋₁₀ alkyl, cycloalkenyl, C₃₋₁₀cycloalkyl, heterocyclylmethyl, and heterocyclylethyl.

11. The compound of claim 10 wherein R⁶ is selected from the group consisting of 3-fluorophenylethyl, 3-fluorobenzyl, 2-trifluoromethylbenzyl, 2-trifluoromethoxybenzyl, 4-trifluoromethylbenzyl, 4-fluorobenzyl, 3-methoxyphenylethyl, 3-thiophenylmethyl, 2-thiophenylethyl, 4,4-dimethylcyclohexyl, 3,3-dimethylcyclohexyl, 2-indanyl, 5-cyano-2-indanyl, 5-methoxy-2-indanyl, 5-fluoro-2-indanyl, 4-fluoro-2-indanyl, 4-methoxy-2-indanyl, 4-methoxy-2-indanyl, 4,8-diflouro-2-indanyl, 5,6-difluoro-2-indanyl, 5,6-dimethoxy-2-indanyl, 2-methyl-2-indanyl, cyclohexylmethyl, cyclohexylethyl, 4,4-difluorocyclohexyl, 1-cyclohexenylmethyl, 1-cyclohexenylethyl, cyclooctyl, cycloheptylmethyl, 3-methylbutyl, adamantyl, morpholinoethyl, piperidinylethyl, 4-tert-butylcyclohexyl, 3,3,5,5-tetramethylcyclohexyl, 3,5-difluorobenzyl, 3,5-difluorophenylethyl, 2-diphenylmethyl, methoxyethyl, dimethylaminoethyl, 3-pyridinylethyl, 3-pyridinylmethyl, and phenyloxyethyl.

12. The compound of claim 11 wherein R⁶ is selected from the group consisting of 2-indanyl, 5-fluoro-2-indanyl, 4,4-dimethylcyclohexyl, cyclohexylethyl, cyclohexylmethyl, 2-thiophenylethyl, 3-fluorophenylethyl, 3-methylbutyl, and 4,4-difluorocyclohexyl.

13. The compound of claim 1 wherein R⁵ is hydrogen and R⁶ is selected from the group consisting of arylmethyl, arylethyl, C₄₋₁₀ alkyl, cycloalkenyl, C₃₋₁₀cycloalkyl, heterocyclylmethyl, and heterocyclylethyl.

14. The compound of claim 3 wherein D is —CH₂— and is attached para to the bond joining Ring A to fused ring BC; J is a bond; T is N, V is NH, U is CH; R³ and R⁴ are each independently selected from the group consisting of —H, —F, —Cl, —CH₃, —CF₃, —OCH₃, and —OCF₃; R⁵ is hydrogen; and R⁶ is selected from the group consisting of arylmethyl, arylethyl, C₃₋₁₀ alkyl, C₃₋₁₀cycloalkyl, and heteroarylalkyl.

15. The compound of claim 1 wherein Ring A is phenyl or pyridyl; D is —O— attached meta or para to the bond joining Ring A and said fused ring BC; J is a bond or a C₁₋₂alkylene; T is N, V is NH, U is CH; R³ and R⁴ are each independently selected from the group consisting of —H, —F, —Cl, —CH₃, —CF₃, —OCH₃, and —OCF₃; R⁵ is hydrogen; and R⁶ is selected from the group consisting or arylmethyl, arylethyl, C₃₋₁₀alkyl, C₃₋₁₀cycloalkyl, and heteroarylalkyl.

16. The compound of claim 3 wherein D is —CH₂—, J is a bond, and R⁵ is a C₁₋₂alkyl joined to Ring A to form a tetrahydroisoquinoline.

17. The compound of claim 1 wherein fused ring BC is selected from the group consisting of

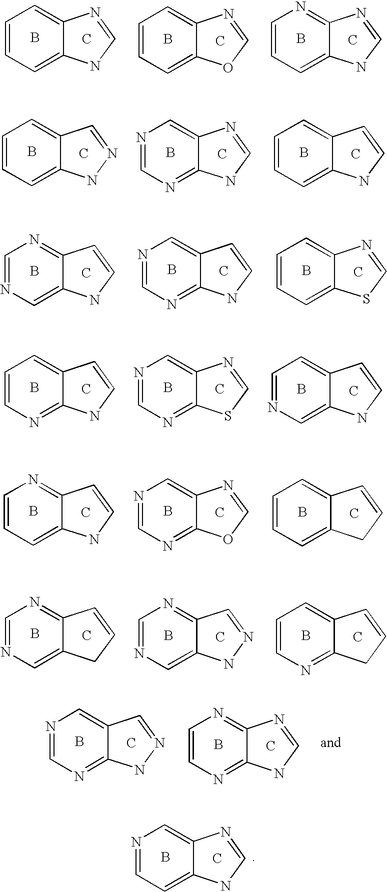

18. The compound of claim 17 wherein fused ring BC is selected from the group consisting of

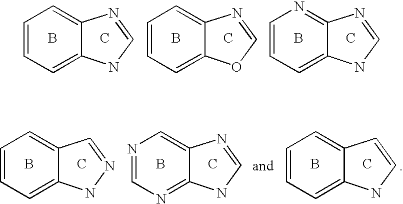

19. The compound of claim 18 wherein fused ring BC is

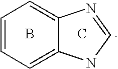

20. The compound of claim 1 selected from the group consisting of
{[4-(1H-benzimidazol-5-yl)phenyl]methyl}(4,4-dimethylcyclohexyl)amine,
N-{[4-(1H-benzimidazol-5-yl)phenyl]methyl}-2,3-dihydro-1H-inden-2-amine,
N-{[3-(1H-benzimidazol-5-yl)phenyl]methyl}-2,3-dihydro-1H-inden-2-amine,
N-{[4-(1H-benzimidazol-5-yl)-2-fluorophenyl]methyl}-2-cyclohexanamine,
N-{[4-(1H-benzimidazol-5-yl)-2-fluorophenyl]methyl}-2,3-dihydro-1H-inden-2-amine,
N-{[4-(1H-benzimidazol-5-yl)-2-fluorophenyl]methyl}-4,4-dimethylcyclohexanamine,
N-{[4-(1H-benzimidazol-5-yl)-3-fluorophenyl]methyl}-4,4-dimethylcyclohexanamine,
(2-cyclohexylethyl){[3-fluoro-4-(4-methyl-1H-benzimidazol-5-yl)phenyl]methyl}amine,
N-{[3-fluoro-4-(4-methyl-1H-benzimidazol-5-yl)phenyl]methyl}-4,4-dimethylcyclohexanamine,
N-{[2,6-difluoro-4-(4-methyl-1H-benzimidazol-5-yl)phenyl]methyl}-2,3-dihydro-1H-inden-2-amine,
(2-cyclohexylethyl){[2,6-difluoro-4-(4-methyl-1H-benzimidazol-5-yl)phenyl]methyl}amine,
N-{[4-(1H-benzimidazol-5-yl)-2,6-difluorophenyl]methyl}-2,3-dihydro-1H-inden-2-amine, and
N-{[4-(1H-benzimidazol-5-yl)-2,6-difluorophenyl]methyl}-4,4-dimethylcyclohexanamine.

21. The compound of claim 20 which is a hydrochloride salt.

22. The compound of claim 1, a salt, a solvate, or a physiologically functional derivative thereof in combination with at least one specie selected from the group consisting of a human ciliary neurotropic factor, a CB-1 antagonist, a neurotransmitter reuptake inhibitor, a lipase inhibitor, an MC4R agonist, a 5-HT2c agonist, a ghrelin receptor antagonist, a CCK-A receptor agonist, an NPY Y1 antagonist, a PYY3-36, and a PPAR activator.

23. (canceled)

24. A pharmaceutical composition comprising a compound of claim 1, a salt, solvate, or physiologically functional derivative thereof and one or more excipients.

25. A method of treatment comprising the administering to a human a pharmaceutical composition comprising (i) a compound of claim 1, a pharmaceutically acceptable salt, solvate, or physiologically functional derivative thereof and (ii) at least one carrier, wherein said treatment is selected from the group consisting of obesity, diabetes, hypertension, depression, anxiety, drug addiction, substance addiction, or a combination thereof.

26-32. (canceled)

33. A process for preparing a compound of claim 1, a salt, solvate, or physiologically functional derivative thereof comprising the reaction of a compound of Formula II 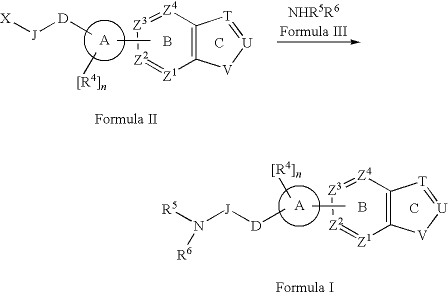
wherein J, D, ring A, R⁴, R⁵, R⁶, Z¹, Z², Z³, Z⁴, T, U, V, and n are as defined in claim 1 and X is a leaving group selected from the group consisting of halogen, triflate, and tosylate with a compound of Formula III in an organic solvent optionally in the presence of a promoter.

34. A process for preparing a compound of claim 1, a salt, solvate, or physiologically functional derivative thereof comprising the reaction of a compound of Formula VIII 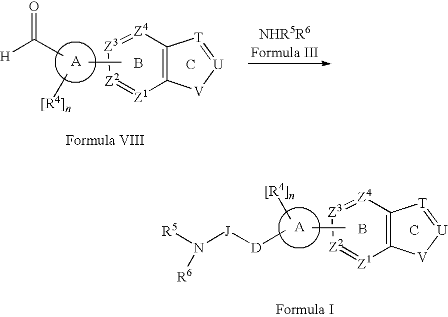
wherein J, D, ring A, R⁴, R⁵, R⁶, Z¹, Z², Z³, Z⁴, T, U, V, and n are as defined in claim 1 with a compound of Formula III in the presence of a reducing agent and an organic solvent optionally in the presence of acetic acid.
